(19) United States
(12) Patent Application Publication
(10) Pub. No.: US 2023/0025600 A1
(43) Pub. Date: Jan. 26, 2023

(54) TREATMENT OF CANCERS WITH ANTIBODY DRUG CONJUGATES (ADC) THAT BIND TO 191P4D12 PROTEINS

(71) Applicants: AGENSYS, INC., Northbrook (US); SEAGEN INC., Bothell, WA (US)

(72) Inventors: Elaina Marie GARTNER, Lake Forest Park, WA (US); Amal MELHEM-BERTRANDT, Wilmette, IL (US); Leonard Michael Jude REYNO, Mill Valley, CA (US); Jonathan Gregor DRACHMAN, Seattle, WA (US)

(73) Assignees: AGENSYS, INC., Northbrook, IL (US); SEAGEN INC., Bothell, WA (US)

(21) Appl. No.: 17/779,068

(22) PCT Filed: Nov. 24, 2020
(86) PCT No.: PCT/US2020/061923

§ 371 Date: May 23, 2022

Related U.S. Application Data
(60) Provisional application No. 62/944,890, filed on Dec. 6, 2019.
(60) Provisional application No. 62/940,209, filed on Nov. 25, 2019.

(51) Int. Cl. A61K 47/68; A61K 47/64; A61P 35/00

(52) U.S. Cl. CPC A61K 47/6849; A61K 47/6803; A61K 47/6851; A61K 47/64; A61P 35/00

(57) ABSTRACT

Provided herein are methods for the treatment of cancers with antibody drug conjugates (ADC) that bind to 191P4D12 proteins. Also provided herein are methods for the treatment of urothelial cancer using an antibody drug conjugate (ADC) that binds 191P4D12. Additionally provided herein are methods for the treatment of solid tumors using an antibody drug conjugate (ADC) that binds 191P4DI2.

### CROSS REFERENCE TO RELATED APPLICATIONS

This application claims the benefit of priority to U.S. Provisional Patent Application No. 62/940,209, filed Nov. 25, 2019, and U.S. Provisional Patent Application No. 62/944,890, filed Dec. 6, 2019, the disclosures of each of which are herein incorporated by reference in their entirety.

### SEQUENCE LISTING

The present specification is being filed with a computer readable form (CRF) copy of the Sequence Listing. The CRF entitled 14369-252-228_SEQ_LISTING.txt, which was created on Nov. 20, 2020, is 39,705 bytes in size, and is incorporated herein by reference in its entirety.

### 1. FIELD

Provided herein are methods for treating cancers with antibody drug conjugates (ADC) that bind to 191P4D12 proteins.

### 2. BACKGROUND

Cancer is the leading cause of death in the US for people 35 to 65 years of age and it is the second leading cause of death worldwide. It was estimated in 2019 that there would be approximately 1.7 million new cancer cases and approximately 610000 deaths from cancer in the US (National Cancer Institute. 2019. Cancer Stat Facts: Cancer of Any Site. https://seer.cancer.gov/statfacts/html/all.html. Accessed 5 Jun. 2019). Globally there were an estimated 18.1 million new cancer cases in 2018 and approximately 9.6 million deaths attributed to cancer in 2018 (World Health Organization. Press Release. September 2018. https://www.who.int/cancer/PRGlobocanFinal.pdf. Accessed 5 Jun. 2019). Most deaths now occur in patients with metastatic cancers. In fact, in the last 20 years, advances in treatment, including surgery, radiotherapy and adjuvant chemotherapy cured most patients with localized cancer. Patients whose cancer presented or recurred as metastatic disease obtained only modest benefit from conventional therapies in terms of overall survival (OS) and were rarely cured.

New therapeutic strategies for advanced and/or metastatic cancers include targeting molecular pathways important for cancer cell survival and novel cytotoxic compounds. The benefit of these novel drugs is reflected in prolonged survival; however, the outcome for most patients with distant metastases is still poor and novel therapies are needed.

191P4D12 (which is also known as Nectin-4) is a type I transmembrane protein and member of a family of related immunoglobulin-like adhesion molecules implicated in cell-to-cell adhesion. 191P4D12 belongs to the Nectin family of adhesion molecules. 191P4D12 is composed of an extracellular domain (ECD) containing 3 Ig-like subdomains, a transmembrane helix, and an intracellular region (Takai Y et al, Annu Rev Cell Dev Biol 2008; 24:309-42). Nectins are thought to mediate Ca2+-independent cell-cell adhesion via both homophilic and heterophilic trans interactions at adherens junctions where they can recruit cadherins and modulate cytoskeletal rearrangements (Rikitake & Takai, Cell Mol Life Sci. 2008; 65(2):253-63). Sequence identity of 191P4D12 to other Nectin family members is low and ranges between 25% to 30% in the ECD (Reymond N et al, J Biol Chem 2001; 43205-15). Nectin-facilitated adhesion supports several biological processes, such as immune modulation, host-pathogen interaction, and immune evasion (Sakisaka T et al, Current Opinion in Cell Biology 2007; 19:593-602).

There is a significant need for additional therapeutic methods for cancers. These include the use of antibodies and antibody drug conjugates as treatment modalities.

### 3. SUMMARY

Embodiment 1. A method of preventing or treating cancer in a human subject, comprising (a) administering to the subject a first regimen comprising an effective amount of an antibody drug conjugate (ADC),

wherein the ADC comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23;

wherein the subject has urothelial cancer; and

wherein the subject has received an immune checkpoint inhibitor therapy and received a chemotherapy.

Embodiment 2. The method of embodiment 1, wherein the ADC is administered three times within a 28 day cycle.

Embodiment 3. The method of embodiment 1 or 2, wherein the ADC is administered on Days 1, 8 and 15 of a 28 day cycle.

Embodiment 4. The method of any one of embodiments 1 to 3, wherein the urothelial cancer is locally advanced urothelial cancer.

Embodiment 5. The method of any one of embodiments 1 to 3, wherein the urothelial cancer is metastatic urothelial cancer.

Embodiment 6. The method of any one of embodiments 1 to 5, wherein the immune checkpoint inhibitor therapy is a programmed death receptor-1 (PD-1) inhibitor.

Embodiment 7. The method of any one of embodiments 1 to 5, wherein the immune checkpoint inhibitor therapy is programmed death-ligand 1 (PD-L1) inhibitor.

Embodiment 8. The method of any one of embodiments 1 to 7, wherein the chemotherapy is platinum-containing chemotherapy.

Embodiment 9. The method of embodiment 8, wherein the platinum-containing chemotherapy is platinum-containing chemotherapy in a neoadjuvant setting.

Embodiment 10. The method of embodiment 8, wherein the platinum-containing chemotherapy is platinum-containing chemotherapy in an adjuvant setting.

Embodiment 11. The method of any one of embodiments 8 to 10, wherein the platinum-containing chemotherapy is platinum-containing chemotherapy in a locally advanced setting.

Embodiment 12. The method of any one of embodiments 8 to 10, wherein the platinum-containing chemotherapy is platinum-containing chemotherapy in a metastatic setting.

Embodiment 13. The method of any one of embodiments 1 to 12, wherein the first regimen comprises an ADC dose of about 1.25 milligram/kilogram (mg/kg) of the subject's body weight.

Embodiment 14. The method of embodiment 13, wherein the subject has a body weight of less than 100 kg.

Embodiment 15. The method of any one of embodiments 1 to 12, wherein the first regimen comprises an ADC dose of about 125 mg to the subject, wherein the subject has a body weight of no less than 100 kg.

Embodiment 16. The method of any one of embodiments 1 to 15, further comprising

(b) determining blood glucose level in the subject, and

(c) if the blood glucose level from (b) is higher than 250 mg/dL, withholding the administration of the antibody drug conjugate.

Embodiment 17. The method of embodiment 16, further comprising

(d) waiting for a period sufficient for the blood glucose level to reduce to no more than 250 mg/dL.

Embodiment 18. The method of embodiment 16 or 17, further comprising

(e) determining blood glucose level in the subject, and

(f) if the blood glucose level from (e) is no more than 250 mg/dL, administering to the subject a second regimen comprising an effective amount of the antibody drug conjugate.

Embodiment 19. The method of any one of embodiments 16 to 18, wherein if the blood glucose level from (b) or (e) is more than 500 mg/dL, discontinuing the administration of the ADC permanently.

Embodiment 20. The method of any one of embodiments 16 to 19, further comprising repeating from (a) to (f).

Embodiment 21. The method of any one of embodiments 16 to 20, wherein the subject has hyperglycemia.

Embodiment 22. The method of embodiment 21, wherein the subject has diabetic ketoacidosis (DKA).

Embodiment 23. The method of any one of embodiments 16 to 22, wherein the subject additionally has higher body mass index and/or higher baseline A1C.

Embodiment 24. The method of any one of embodiments 18 to 23, wherein second regimen is identical to the first regimen.

Embodiment 25. The method of any one of embodiments 16 to 24, wherein the blood glucose level is determined daily.

Embodiment 26. The method of any one of embodiments 16 to 24, wherein the blood glucose level is determined once every two days, once every three days, once every four days, or once every five days, once every six days.

Embodiment 27. The method of any one of embodiments 16 to 24, wherein the blood glucose level is determined weekly, bi-weekly, once every three weeks, or once every four weeks.

Embodiment 28. The method of any one of embodiments 16 to 24, wherein the blood glucose level is determined monthly, once every two months, or once every three months.

Embodiment 29. The method of any one of embodiments 1 to 28, further comprising

(g) determining peripheral neuropathy in the subject, and

(h) if the peripheral neuropathy from (g) is no less than Grade 2, withholding the administration of the antibody drug conjugate.

Embodiment 30. The method of embodiment 29, further comprising

(i) waiting for a period sufficient for the peripheral neuropathy to reduce to no more than Grade 1.

Embodiment 31. The method of embodiment 29 or 30, further comprising

(j) determining peripheral neuropathy in the subject, and

(k) if the peripheral neuropathy (j) is no more than Grade 1, administering to the subject a second regimen comprising an effective amount of the ADC, wherein the second regimen comprises an ADC dose equal to or lower than the first regimen.

Embodiment 32. The method of any one of embodiments 29 to 31, wherein if the peripheral neuropathy from (g) or (j) is no less than Grade 3, discontinuing the administration of the ADC permanently.

Embodiment 33. The method of any one of embodiments 29 to 32, wherein the peripheral neuropathy is predominantly sensory neuropathy.

Embodiment 34. The method of any one of embodiments 29 to 31, and 33, further comprising repeating from (g) to (k).

Embodiment 35. The method of any one of embodiments 31, and 33 to 34, further comprising determining the number of times the condition for the administration of the second regimen has been satisfied.

Embodiment 36. The method of any one of embodiments 31, and 33 to 35, wherein in (k) if the second regimen is administered for the first time, the second regimen is identical to the first regimen.

Embodiment 37. The method of any one of embodiments 31, and 33 to 36, wherein in (k) if the second regimen has been administered once and the subject has a body weight of less than 100 kg, the ADC dose in the second regimen is lowered to about 1.0 mg/kg of the subject's body weight.

Embodiment 38. The method of any one of embodiments 31, and 33 to 36, wherein in (k) if the second regimen has been administered once and the subject has a body weight of no less than 100 kg, the ADC dose in the second regimen is lowered to about 100 mg to the subject.

Embodiment 39. The method of any one of embodiments 31, and 33 to 38, wherein in (k) if the second regimen has been administered twice and the subject has a body weight of less than 100 kg, the ADC dose in the second regimen is lowered to about 0.75 mg/kg of the subject's body weight.

Embodiment 40. The method of any one of embodiments 31, and 33 to 38, wherein in (k) if the second regimen has been administered once and the subject has a body weight of no less than 100 kg, the ADC dose in the second regimen is lowered to about 75 mg to the subject.

Embodiment 41. The method of any one of embodiments 31, and 33 to 40, wherein in (k) if the second regimen has been administered three times and the subject has a body weight of less than 100 kg, the ADC dose in the second regimen is lowered to about 0.5 mg/kg of the subject's body weight.

Embodiment 42. The method of any one of embodiments 31, and 33 to 40, wherein in (k) if the second regimen has been administered three times and the subject has a body weight of no less than 100 kg, the ADC dose in the second regimen is lowered to about 50 mg to the subject.

Embodiment 43. The method of any one of embodiments 31 and 33 to 42, wherein the ADC dose in the second regimen is increased by an amount of about 0.25 mg/kg for the subject having a body weight of less than 100 kg or increased by an amount of about 25 mg for the subject having a body weight of no less than 100 kg, if

(1) the administration of the ADC has not been discontinued permanently,

(2) the ADC dose in the second regimen is lower than the ADC dose in the first regimen, and

(3) the peripheral neuropathy has returned to no more than Grade 1.

Embodiment 44. The method of any one of embodiments 29 to 43, wherein the peripheral neuropathy is determined daily.

Embodiment 45. The method of any one of embodiments 29 to 43, wherein the peripheral neuropathy is determined once every two days, once every three days, once every four days, or once every five days, once every six days.

Embodiment 46. The method of any one of embodiments 29 to 43, wherein the peripheral neuropathy is determined weekly, bi-weekly, once every three weeks, or once every four weeks.

Embodiment 47. The method of any one of embodiments 29 to 43, wherein the peripheral neuropathy is determined monthly, once every two months, or once every three months.

Embodiment 48. The method of any one of embodiments 1 to 47, further comprising

(l) determining a skin reaction in the subject, and

(m) if the skin reaction from (l) is no less than Grade 3, withholding the administration of the ADC.

Embodiment 49. The method of embodiment 48, further comprising

(n) waiting for a period sufficient for the skin reaction to reduce to no more than

Grade 1.

Embodiment 50. The method of embodiment 48 or 49, further comprising

(o) determining the skin reaction in the subject, and

(p) if the skin reaction in (o) is no more than Grade 1, administering to the subject a second regimen comprising an effective amount of the ADC, wherein the second regimen comprises an ADC dose equal to or lower than the first regimen.

Embodiment 51. The method of any one of embodiments 48 to 50, wherein if the skin reaction from (l) or (o) is no less than Grade 4, discontinuing the administration of the ADC permanently.

Embodiment 52. The method of any one of embodiments 48 to 51, wherein the skin reaction is selected from the group consisting of maculopapular rash, pruritus, symmetrical drug-related intertriginous, flexural exanthema (SDRIFE), bullous dermatitis, exfoliative dermatitis, and palmar-plantar erythrodysesthesia.

Embodiment 53. The method of any one of embodiments 48 to 51, wherein the no less than Grade 3 skin reaction is selected from the group consisting of symmetrical drug-related intertriginous, flexural exanthema (SDRIFE), bullous dermatitis, exfoliative dermatitis, and palmar-plantar erythrodysesthesia.

Embodiment 54. The method of any one of embodiments 48 to 50, and 52 to 53, further comprising repeating from (l) to (p).

Embodiment 55. The method of 54, wherein if Grade 3 skin reaction reoccurs in (l) or (o), discontinuing the administration of the ADC permanently.

Embodiment 56. The method of any one of embodiments 48 to 50, and 52 to 55, further comprising determining the number of times the condition for the administration of the second regimen has been satisfied.

Embodiment 57. The method of any one of embodiments 48 to 50, and 52 to 56, wherein in (p) if the second regimen is administered for the first time, the second regimen is identical to the first regimen.

Embodiment 58. The method of any one of embodiments 48 to 50, and 52 to 57, wherein in (p) if the second regimen has been administered one or more times and the subject has a body weight of less than 100 kg, the ADC dose in the second regimen is lowered to about 1.0 mg/kg of the subject's body weight.

Embodiment 59. The method of any one of embodiments 48 to 50, and 52 to 57, wherein in (p) if the second regimen has been administered one or more times and the subject has a body weight of no less than 100 kg, the ADC dose in the second regimen is lowered to about 100 mg to the subject.

Embodiment 60. The method of any one of embodiments 48 to 50, and 52 to 59, wherein in (p) if the second regimen has been administered two or more times and the subject has a body weight of less than 100 kg, the ADC dose in the second regimen is lowered to about 0.75 mg/kg of the subject's body weight.

Embodiment 61. The method of any one of embodiments 48 to 50, and 52 to 59, wherein in (p) if the second regimen has been administered two or more times and the subject has a body weight of no less than 100 kg, the ADC dose in the second regimen is lowered to about 75 mg to the subject.

Embodiment 62. The method of any one of embodiments 48 to 50, and 52 to 61, wherein in (p) if the second regimen has been administered three or more times and the subject has a body weight of less than 100 kg, the ADC dose in the second regimen is lowered to about 0.5 mg/kg of the subject's body weight.

Embodiment 63. The method of any one of embodiments 48 to 50, and 52 to 61, wherein in (p) if the second regimen has been administered three or more times and the subject has a body weight of no less than 100 kg, the ADC dose in the second regimen is lowered to about 50 mg to the subject.

Embodiment 64. The method of any one of embodiments 48 to 50, and 52 to 56, wherein in (p) if the subject has a body weight of less than 100 kg, the second regimen comprises an ADC dose of about 1.0 mg/kg of the subject's body weight.

Embodiment 65. The method of any one of embodiments 48 to 50, and 52 to 56, wherein in (p) if the subject has a body weight of no less than 100 kg, the second regimen comprises an ADC dose of about 100 mg to the subject.

Embodiment 66. The method of any one of embodiments 48 to 50, 52 to 56, and 64 to 65, wherein in (p) if the second regimen has been administered one or more times and the subject has a body weight of less than 100 kg, the ADC dose in the second regimen is lowered to about 0.75 mg/kg of the subject's body weight.

Embodiment 67. The method of any one of embodiments 48 to 50, 52 to 56, and 64 to 65, wherein in (p) if the second regimen has been administered one or more times and the subject has a body weight of no less than 100 kg, the ADC dose in the second regimen is lowered to about 75 mg to the subject.

Embodiment 68. The method of any one of embodiments 48 to 50, 52 to 56, and 64 to 67, wherein in (p) if the second regimen has been administered two or more times and the subject has a body weight of less than 100 kg, the ADC dose in the second regimen is lowered to about 0.5 mg/kg of the subject's body weight.

Embodiment 69. The method of any one of embodiments 48 to 50, 52 to 56, and 64 to 67, wherein in (p) if the second regimen has been administered two or more times and the subject has a body weight of no less than 100 kg, the ADC dose in the second regimen is lowered to about 50 mg to the subject.

Embodiment 70. The method of any one of embodiments 50 and 52 to 69, wherein the ADC dose in the second regimen is increased by an amount of about 0.25 mg/kg for the subject having a body weight of less than 100 kg or increased by an amount of about 25 mg for the subject having a body weight of no less than 100 kg, if

(1) the administration of the ADC has not been discontinued permanently,

(2) the ADC dose in the second regimen is lower than the ADC dose in the first regimen, and

(3) the skin reaction has returned to no more than Grade 1.

Embodiment 71. The method of any one of embodiments 48 to 70, wherein the skin reaction is determined daily.

Embodiment 72. The method of any one of embodiments 48 to 70, wherein the skin reaction is determined once every two days, once every three days, once every four days, or once every five days, once every six days.

Embodiment 73. The method of any one of embodiments 48 to 70, wherein the skin reaction is determined weekly, bi-weekly, once every three weeks, or once every four weeks.

Embodiment 74. The method of any one of embodiments 48 to 70, wherein the skin reaction is determined monthly, once every two months, or once every three months.

Embodiment 75. The method of any one of embodiments 1 to 74, further comprising

(q) determining non-hematologic toxicity in the subject, and

(s) if the non-hematologic toxicity from (q) is no less than Grade 3, withholding the administration of the ADC.

Embodiment 76. The method of embodiment 75, further comprising

(t) waiting for a period sufficient for the non-hematologic toxicity to reduce to no more than Grade 1.

Embodiment 77. The method of embodiment 75 or 76, further comprising

(u) determining the non-hematologic toxicity in the subject, and

(v) if the non-hematologic toxicity in (u) is no more than Grade 1, administering to the subject a second regimen comprising an effective amount of the ADC, wherein the second regimen comprises an ADC dose equal to or lower than the first regimen.

Embodiment 78. The method of any one of embodiments 75 to 77, wherein if the non-hematologic toxicity in (q) or (u) is no less than Grade 4, discontinuing the administration of the ADC permanently.

Embodiment 79. The method of any one of embodiments 75 to 78, wherein the non-hematologic toxicity is dysgeusia.

Embodiment 80. The method of any one of embodiments 75 to 78, wherein the non-hematologic toxicity is anorexia.

Embodiment 81. The method of any one of embodiments 75 to 78, wherein the non-hematologic toxicity is loss of appetite.

Embodiment 82. The method of any one of embodiments 75 to 78, wherein the non-hematologic toxicity is an ocular disorder.

Embodiment 83. The method of embodiment 79, wherein the ocular disorder is one or more selected from the group consisting of punctate keratitis, keratitis, keratopathy, limbal stem cell deficiency, dry eye, and blurred vision.

Embodiment 84. The method of any one of embodiments 75 to 77, and 79 to 83, further comprising repeating from (q) to (v).

Embodiment 85. The method of any one of embodiments 75 to 77, and 79 to 84, further comprising determining the number of times the condition for the administration of the second regimen has been satisfied.

Embodiment 86. The method of any one of embodiments 75 to 77, and 79 to 85, wherein in (v) the second regimen is identical to the first regimen.

Embodiment 87. The method of any one of embodiments 75 to 77, and 79 to 86, wherein in (v) if the second regimen has been administered one or more times and the subject has a body weight of less than 100 kg, the ADC dose in the second regimen is lowered to about 1.0 mg/kg of the subject's body weight.

Embodiment 88. The method of any one of embodiments 75 to 77, and 79 to 86, wherein in (v) if the second regimen has been administered one or more times and the subject has a body weight of no less than 100 kg, the ADC dose in the second regimen is lowered to about 100 mg to the subject.

Embodiment 89. The method of any one of embodiments 75 to 77, and 79 to 88, wherein in (v) if the second regimen has been administered two or more times and the subject has a body weight of less than 100 kg, the ADC dose in the second regimen is lowered to about 0.75 mg/kg of the subject's body weight.

Embodiment 90. The method of any one of embodiments 75 to 77, and 79 to 88, wherein in (v) if the second regimen has been administered two or more times and the subject has a body weight of no less than 100 kg, the ADC dose in the second regimen is lowered to about 75 mg to the subject.

Embodiment 91. The method of any one of embodiments 75 to 77, and 79 to 90, wherein in (v) if the second regimen has been administered three or more times and the subject has a body weight of less than 100 kg, the ADC dose in the second regimen is lowered to about 0.5 mg/kg of the subject's body weight.

Embodiment 92. The method of any one of embodiments 75 to 77, and 79 to 90, wherein in (v) if the second regimen has been administered three or more times and the subject has a body weight of no less than 100 kg, the ADC dose in the second regimen is lowered to about 50 mg to the subject.

Embodiment 93. The method of any one of embodiments 75 to 77, and 79 to 85, wherein in (v) if the subject has a body weight of less than 100 kg, the second regimen comprises an ADC dose of about 1.0 mg/kg of the subject's body weight.

Embodiment 94. The method of any one of embodiments 75 to 77, and 79 to 85, wherein in (v) if the subject has a body weight of no less than 100 kg, the second regimen comprises an ADC dose of about 100 mg to the subject.

Embodiment 95. The method of any one of embodiments 75 to 77, and 79 to 85, 93 to 94, wherein in (v) if the second regimen has been administered one or more times and the subject has a body weight of less than 100 kg, the ADC dose in the second regimen is lowered to about 0.75 mg/kg of the subject's body weight.

Embodiment 96. The method of any one of embodiments 75 to 77, and 79 to 85, 93 to 94, wherein in (v) if the second regimen has been administered one or more times and the subject has a body weight of no less than 100 kg, the ADC dose in the second regimen is lowered to about 75 mg to the subject.

Embodiment 97. The method of any one of embodiments 75 to 77, and 79 to 85, 93 to 96, wherein in (v) if the second regimen has been administered two or more times and the subject has a body weight of less than 100 kg, the ADC dose in the second regimen is lowered to about 0.5 mg/kg of the subject's body weight.

Embodiment 98. The method of any one of embodiments 75 to 77, and 79 to 85, 93 to 96, wherein in (v) if the second regimen has been administered two or more times and the subject has a body weight of no less than 100 kg, the ADC dose in the second regimen is lowered to about 50 mg to the subject.

Embodiment 99. The method of any one of embodiments 77 and 79 to 98, wherein the ADC dose in the second regimen is increased by an amount of about 0.25 mg/kg for the subject having a body weight of less than 100 kg or increased by an amount of about 25 mg for the subject having a body weight of no less than 100 kg, if

(1) the administration of the ADC has not been discontinued permanently,

(2) the ADC dose in the second regimen is lower than the ADC dose in the first regimen, and

(3) the non-hematologic toxicity has returned to no more than Grade 1.

Embodiment 100. The method of any one of embodiments 75 to 99, wherein the non-hematologic toxicity is determined daily.

Embodiment 101. The method of any one of embodiments 75 to 99, wherein the non-hematologic toxicity is determined once every two days, once every three days, once every four days, or once every five days, once every six days.

Embodiment 102. The method of any one of embodiments 75 to 99, wherein the non-hematologic toxicity is determined weekly, bi-weekly, once every three weeks, or once every four weeks.

Embodiment 103. The method of any one of embodiments 75 to 99, wherein the non-hematologic toxicity is determined monthly, once every two months, or once every three months.

Embodiment 104. The method of any one of embodiments 1 to 103, further comprising

(w) determining hematologic toxicity in the subject, and

(x) if the hematologic toxicity from (w) is no less than Grade 2, withholding the administration of the ADC.

Embodiment 105. The method of embodiment 104, further comprising

(y) waiting for a period sufficient for the hematologic toxicity to reduce to no more than Grade 1.

Embodiment 106. The method of embodiment 104 or 105, further comprising

(z) determining the hematologic toxicity in the subject, and

(aa) if the hematologic toxicity in (z) is no more than Grade 1, administering to the subject a second regimen comprising an effective amount of the ADC, wherein the second regimen comprises an ADC dose equal to or lower than the first regimen.

Embodiment 107. The method of any one of embodiments 104 to 106, wherein if the hematologic toxicity in (w) or (z) is no less than Grade 4, discontinuing the administration of the ADC permanently.

Embodiment 108. The method of any one of embodiments 104 to 107, wherein the hematologic toxicity is thrombocytopenia.

Embodiment 109. The method of any one of embodiments 104 to 107, wherein the hematologic toxicity is selected from the group consisting of anemia, thrombocytopenia, neutropenia, and febrile neutropenia.

Embodiment 110. The method of any one of embodiments 104 to 106, and 108 to 109, further comprising repeating from (w) to (aa).

Embodiment 111. The method of any one of embodiments 106, and 108 to 110, wherein if the hematologic toxicity in (w) is no less than Grade 4 and the subject has a body weight of less than 100 kg, the ADC dose in the second regimen is lowered to about 1.0 mg/kg of the subject's body weight.

Embodiment 112. The method of any one of embodiments 106, and 108 to 110, wherein if the hematologic toxicity in (w) is no less than Grade 4 and the subject has a body weight of no less than 100 kg, the ADC dose in the second regimen is lowered to about 100 mg to the subject.

Embodiment 113. The method of any one of embodiments 106, and 108 to 110, wherein the hematologic toxicity in (w) is Grade 3 or Grade 2.

Embodiment 114. The method of any one of embodiments 106, and 108 to 110, wherein the hematologic toxicity in (w) is Grade 3 thrombocytopenia or Grade 2 thrombocytopenia.

Embodiment 115. The method of embodiment 113 or 114, further comprising determining the number of times the condition for the administration of the second regimen has been satisfied.

Embodiment 116. The method of any one of embodiments 113 to 115, wherein in (aa) the second regimen is identical to the first regimen.

Embodiment 117. The method of any one of embodiments 113 to 116, wherein in (aa) if the subject has a body weight of less than 100 kg, the ADC dose in the second regimen is lowered to about 1.0 mg/kg of the subject's body weight.

Embodiment 118. The method of any one of embodiments 113 to 116, wherein in (aa) if the subject has a body weight of no less than 100 kg, the ADC dose in the second regimen is lowered to about 100 mg to the subject.

Embodiment 119. The method of any one of embodiments 113 to 118, wherein in (aa) if the second regimen has been administered at the ADC dose of about 1.0 mg/kg or 100 mg and if the subject has a body weight of less than 100 kg, the ADC dose in the second regimen is lowered to about 0.75 mg/kg of the subject's body weight.

Embodiment 120. The method of any one of embodiments 113 to 118, wherein in (aa) if the second regimen has been administered at the ADC dose of about 1.0 mg/kg or 100 mg and if the subject has a body weight of no less than 100 kg, the ADC dose in the second regimen is lowered to about 75 mg to the subject.

Embodiment 121. The method of any one of embodiments 113 to 120, wherein in (aa) if the second regimen has been administered at the ADC dose of about 0.75 mg/kg or 75 mg and if the subject has a body weight of less than 100 kg, the ADC dose in the second regimen is lowered to about 0.5 mg/kg of the subject's body weight.

Embodiment 122. The method of any one of embodiments 113 to 120, wherein in (aa) if the second regimen has been administered at the ADC dose of about 0.75 mg/kg or 75 mg and if the subject has a body weight of no less than 100 kg, the ADC dose in the second regimen is lowered to about 50 mg to the subject.

Embodiment 123. The method of any one of embodiments 106 and 108 to 122, wherein the ADC dose in the second regimen is increased by an amount of about 0.25 mg/kg for the subject having a body weight of less than 100 kg or increased by an amount of about 25 mg for the subject having a body weight of no less than 100 kg, if

(1) the administration of the ADC has not been discontinued permanently,

(2) the ADC dose in the second regimen is lower than the ADC dose in the first regimen, and

(3) the hematologic toxicity has returned to no more than Grade 1.

Embodiment 124. The method of any one of embodiments 104 to 123, wherein the hematologic toxicity is determined daily.

Embodiment 125. The method of any one of embodiments 104 to 123, wherein the hematologic toxicity is determined once every two days, once every three days, once every four days, or once every five days, once every six days.

Embodiment 126. The method of any one of embodiments 104 to 123, wherein the hematologic toxicity is determined weekly, bi-weekly, once every three weeks, or once every four weeks.

Embodiment 127. The method of any one of embodiments 104 to 123, wherein the hematologic toxicity is determined monthly, once every two months, or once every three months.

Embodiment 128. The method of any one of embodiments 1 to 127, further comprising

(ab) determining fatigue in the subject, and

(ac) if the fatigue from (ab) is no less than Grade 3, withholding the administration of the ADC.

Embodiment 129. The method of embodiment 128, further comprising

(ad) waiting for a period sufficient for the fatigue to reduce to no more than Grade 1.

Embodiment 130. The method of embodiment 128 or 129, further comprising

(ae) determining the fatigue in the subject, and

(af) if the fatigue in (ae) is no more than Grade 1, administering to the subject a second regimen comprising an effective amount of the ADC, wherein the second regimen comprises an ADC dose equal to or lower than the first regimen.

Embodiment 131. The method of any one of embodiments 128 to 130, wherein if the fatigue in (ab) or (ae) is no less than Grade 4, discontinuing the administration of the ADC permanently.

Embodiment 132. The method of any one of embodiments 128 to 130, further comprising repeating from (ab) to (af).

Embodiment 133. The method of any one of embodiments 128 to 130 and 132, further comprising determining the number of times the condition for the administration of the second regimen has been satisfied.

Embodiment 134. The method of any one of embodiments 128 to 130 and 132 to 133, wherein in if the fatigue in (ab) is Grade 3, the second regimen is identical to the first regimen.

Embodiment 135. The method of any one of embodiments 128 to 130 and 132 to 134, wherein if the fatigue in (ab) is Grade 3 and the subject has a body weight of less than 100 kg, the ADC dose in the second regimen is lowered to about 1.0 mg/kg of the subject's body weight.

Embodiment 136. The method of any one of embodiments 128 to 130 and 132 to 134, wherein if the fatigue in (ab) is Grade 3 and the subject has a body weight of no less than 100 kg, the ADC dose in the second regimen is lowered to about 100 mg to the subject.

Embodiment 137. The method of any one of embodiments 128 to 130 and 132 to 136, wherein in (af) if the second regimen has been administered at the ADC dose of about 1.0 mg/kg or 100 mg and if the subject has a body weight of less than 100 kg, the ADC dose in the second regimen is lowered to about 0.75 mg/kg of the subject's body weight.

Embodiment 138. The method of any one of embodiments 128 to 130 and 132 to 136, wherein in (af) if the second regimen has been administered at the ADC dose of about 1.0 mg/kg or 100 mg and if the subject has a body weight of no less than 100 kg, the ADC dose in the second regimen is lowered to about 75 mg to the subject.

Embodiment 139. The method of any one of embodiments 128 to 130 and 132 to 138, wherein in (af) if the second regimen has been administered at the ADC dose of about 0.75 mg/kg or 75 mg and if the subject has a body weight of less than 100 kg, the ADC dose in the second regimen is lowered to about 0.5 mg/kg of the subject's body weight.

Embodiment 140. The method of any one of embodiments 128 to 130 and 132 to 138, wherein in (af) if the second regimen has been administered at the ADC dose of about 0.75 mg/kg or 75 mg and if the subject has a body weight of no less than 100 kg, the ADC dose in the second regimen is lowered to about 50 mg to the subject.

Embodiment 141. The method of any one of embodiments 130 and 132 to 140, wherein the ADC dose in the second regimen is increased by an amount of about 0.25 mg/kg for the subject having a body weight of less than 100 kg or increased by an amount of about 25 mg for the subject having a body weight of no less than 100 kg, if

(1) the administration of the ADC has not been discontinued permanently,

(2) the ADC dose in the second regimen is lower than the ADC dose in the first regimen, and

(3) the fatigue has returned to no more than Grade 1.

Embodiment 142. The method of any one of embodiments 128 to 141, wherein the fatigue is determined daily.

Embodiment 143. The method of any one of embodiments 128 to 141, wherein the fatigue is determined once every two days, once every three days, once every four days, or once every five days, once every six days.

Embodiment 144. The method of any one of embodiments 128 to 141, wherein the fatigue is determined weekly, bi-weekly, once every three weeks, or once every four weeks.

Embodiment The method of any one of embodiments 128 to 141, wherein the fatigue is determined monthly, once every two months, or once every three months.

Embodiment 146. The method of any one of embodiments 1 to 145, further comprising

(ag) determining diarrhea in the subject, and

(ah) if the diarrhea from (ag) is no less than Grade 3, withholding the administration of the ADC.

Embodiment 147. The method of embodiment 146, further comprising

(ai) waiting for a period sufficient for the diarrhea to reduce to no more than Grade 1.

Embodiment 148. The method of embodiment 146 or 147, further comprising

(aj) determining the diarrhea in the subject, and

(ak) if the diarrhea in (aj) is no more than Grade 1, administering to the subject a second regimen comprising an effective amount of the ADC, wherein the second regimen comprises an ADC dose equal to or lower than the first regimen.

Embodiment 149. The method of any one of embodiments 146 to 148, wherein if the diarrhea in (ag) or (ai) is no less than Grade 4 and the diarrhea does not improve to no more than Grade 2 within 72 hours with supportive management, discontinuing the administration of the ADC permanently.

Embodiment 150. The method of any one of embodiments 146 to 148, further comprising repeating from (ag) to (ak).

Embodiment 151. The method of any one of embodiments 146 to 148 and 150, further comprising determining the number of times the condition for the administration of the second regimen has been satisfied.

Embodiment 152. The method of any one of embodiments 146 to 148 and 150 to 151, wherein in (ak) the second regimen is identical to the first regimen.

Embodiment 153. The method of any one of embodiments 146 to 148 and 150 to 152, wherein in (ak) if the second regimen has been administered one or more times and the subject has a body weight of less than 100 kg, the ADC dose in the second regimen is lowered to about 1.0 mg/kg of the subject's body weight.

Embodiment 154. The method of any one of embodiments 146 to 148 and 150 to 152, wherein in (ak) if the second regimen has been administered one or more times and the subject has a body weight of no less than 100 kg, the ADC dose in the second regimen is lowered to about 100 mg to the subject.

Embodiment 155. The method of any one of embodiments 146 to 148 and 150 to 154, wherein in (ak) if the second regimen has been administered two or more times and the subject has a body weight of less than 100 kg, the ADC dose in the second regimen is lowered to about 0.75 mg/kg of the subject's body weight.

Embodiment 156. The method of any one of embodiments 146 to 148 and 150 to 154, wherein in (ak) if the second regimen has been administered two or more times and the subject has a body weight of no less than 100 kg, the ADC dose in the second regimen is lowered to about 75 mg to the subject.

Embodiment 157. The method of any one of embodiments 146 to 148 and 150 to 156, wherein in (ak) if the second regimen has been administered three or more times and the subject has a body weight of less than 100 kg, the ADC dose in the second regimen is lowered to about 0.5 mg/kg of the subject's body weight.

Embodiment 158. The method of any one of embodiments 146 to 148 and 150 to 156, wherein in (ak) if the second regimen has been administered three or more times and the subject has a body weight of no less than 100 kg, the ADC dose in the second regimen is lowered to about 50 mg to the subject.

Embodiment 159. The method of any one of embodiments 146 to 148 and 150 to 151, wherein in (ak) if the subject has a body weight of less than 100 kg, the second regimen comprises an ADC dose of about 1.0 mg/kg of the subject's body weight.

Embodiment 160. The method of any one of embodiments 146 to 148 and 150 to 151, wherein in (ak) if the subject has a body weight of no less than 100 kg, the second regimen comprises an ADC dose of about 100 mg to the subject.

Embodiment 161. The method of any one of embodiments 146 to 148, 150 to 151, and 159 to 160, wherein in (ak) if the second regimen has been administered one or more times and the subject has a body weight of less than 100 kg, the ADC dose in the second regimen is lowered to about 0.75 mg/kg of the subject's body weight.

Embodiment 162. The method of any one of embodiments 146 to 148, 150 to 151, and 159 to 160, wherein in (ak) if the second regimen has been administered one or more times and the subject has a body weight of no less than 100 kg, the ADC dose in the second regimen is lowered to about 75 mg to the subject.

Embodiment 163. The method of any one of embodiments 146 to 148, 150 to 151, and 159 to 162, wherein in (ak) if the second regimen has been administered two or more times and the subject has a body weight of less than 100 kg, the ADC dose in the second regimen is lowered to about 0.5 mg/kg of the subject's body weight.

Embodiment 164. The method of any one of embodiments 146 to 148, 150 to 151, and 159 to 162, wherein in (ak) if the second regimen has been administered two or more times and the subject has a body weight of no less than 100 kg, the ADC dose in the second regimen is lowered to about 50 mg to the subject.

Embodiment 165. The method of any one of embodiments 148 and 150 to 164, wherein the ADC dose in the second regimen is increased by an amount of about 0.25 mg/kg for the subject having a body weight of less than 100 kg or increased by an amount of about 25 mg for the subject having a body weight of no less than 100 kg, if

(1) the administration of the ADC has not been discontinued permanently,

(2) the ADC dose in the second regimen is lower than the ADC dose in the first regimen, and

(3) the diarrhea has returned to no more than Grade 1.

Embodiment 166. The method of any one of embodiments 146 to 165, wherein the diarrhea is determined daily.

Embodiment 167. The method of any one of embodiments 146 to 165, wherein the diarrhea is determined once every two days, once every three days, once every four days, or once every five days, once every six days.

Embodiment 168. The method of any one of embodiments 146 to 165, wherein the diarrhea is determined weekly, bi-weekly, once every three weeks, or once every four weeks.

Embodiment 169. The method of any one of embodiments 146 to 165, wherein the diarrhea is determined monthly, once every two months, or once every three months.

Embodiment 170. The method of any one of embodiments 1 to 169, wherein the antibody or antigen binding fragment thereof comprises CDR H1 comprising the amino acid sequence of SEQ ID NO:9, CDR H2 comprising the amino acid sequence of SEQ ID NO:10, CDR H3 comprising the amino acid sequence of SEQ ID NO:11; CDR L1 comprising the amino acid sequence of SEQ ID NO:12, CDR L2 comprising the amino acid sequence of SEQ ID NO:13, and CDR L3 comprising the amino acid sequence of SEQ ID NO:14.

Embodiment 171. The method of any one of embodiments 1 to 169, wherein the antibody or antigen binding fragment thereof comprises CDR H1 comprising the amino acid sequence of SEQ ID NO:16, CDR H2 comprising the amino acid sequence of SEQ ID NO:17, CDR H3 comprising the amino acid sequence of SEQ ID NO:18; CDR L1 comprising the amino acid sequence of SEQ ID NO:19, CDR L2 comprising the amino acid sequence of SEQ ID NO:20, and CDR L3 comprising the amino acid sequence of SEQ ID NO:21.

Embodiment 172. The method of any one of embodiments 1 to 169, wherein the antibody or antigen binding fragment thereof comprises CDR H1 consisting of the amino acid sequence of SEQ ID NO:9, CDR H2 consisting of the amino acid sequence of SEQ ID NO:10, CDR H3 consisting of the amino acid sequence of SEQ ID NO:11; CDR L1 consisting of the amino acid sequence of SEQ ID NO:12, CDR L2 consisting of the amino acid sequence of SEQ ID NO:13, and CDR L3 consisting of the amino acid sequence of SEQ ID NO:14.

Embodiment 173. The method of any one of embodiments 1 to 169, wherein the antibody or antigen binding fragment thereof comprises CDR H1 consisting of the amino acid sequence of SEQ ID NO:16, CDR H2 consisting of the amino acid sequence of SEQ ID NO:17, CDR H3 consisting of the amino acid sequence of SEQ ID NO:18; CDR L1 consisting of the amino acid sequence of SEQ ID NO:19, CDR L2 consisting of the amino acid sequence of SEQ ID NO:20, and CDR L3 consisting of the amino acid sequence of SEQ ID NO:21.

Embodiment 174. The method of any one of embodiments 1 to 173, wherein the antibody or antigen binding fragment thereof comprises a heavy chain variable region comprising the amino acid sequence of SEQ ID NO:22 and a light chain variable region comprising the amino acid sequence of SEQ ID NO:23.

Embodiment 175. The method of any one of embodiments 1 to 174, wherein the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

Embodiment 176. The method of any one of embodiments 1 to 175, wherein the antigen binding fragment is an Fab, F(ab′)2, Fv or scFv fragment.

Embodiment 177. The method of any one of embodiments 1 to 176, wherein the antibody is a fully human antibody.

Embodiment 178. The method of any one of embodiments 1 to 177, wherein the antibody or antigen binding fragment thereof is recombinantly produced.

Embodiment 179. The method of any one of embodiments 1 to 178, wherein the antibody or antigen binding fragment is linked to each unit of monomethyl auristatin E (MMAE) via a linker.

Embodiment 180. The method of embodiment 179, wherein the linker is an enzyme-cleavable linker, and wherein the linker forms a bond with a sulfur atom of the antibody or antigen binding fragment thereof.

Embodiment 181. The method of embodiment 179 or 180, wherein the linker has a formula of: -Aa-Ww-Yy-; wherein -A- is a stretcher unit, a is 0 or 1; —W— is an amino acid unit, w is an integer ranging from 0 to 12; and —Y— is a spacer unit, y is 0, 1, or 2.

Embodiment 182. The method of embodiment 181, wherein the stretcher unit has the structure of Formula (1) below; the amino acid unit is valine citrulline; and the spacer unit is a PAB group comprising the structure of Formula (2) below:

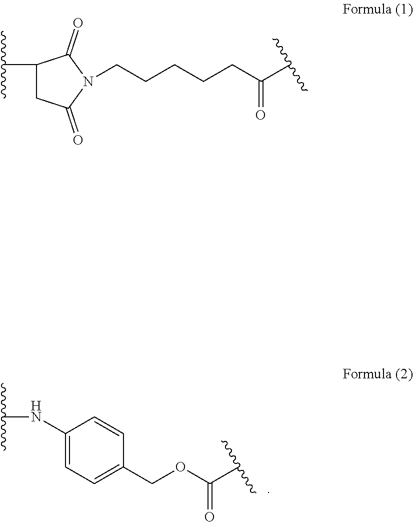 [SMILES: *C(=O)CCCCCN1C(=O)CC(*)C1=O ; *Nc1ccc(COC(*)=O)cc1]

Embodiment 183. The method of embodiment 181 or 182, wherein the stretcher unit forms a bond with a sulfur atom of the antibody or antigen binding fragment thereof; and wherein the spacer unit is linked to MMAE via a carbamate group.

Embodiment 184. The method of any one of embodiments 1 to 183, wherein the antibody is a fully human monoclonal antibody and wherein the antibody is an IgG1.

Embodiment 185. The method of any one of embodiments 1 to 184, wherein the ADC comprises from 1 to 10 units of MMAE per antibody or antigen binding fragment thereof.

Embodiment 186. The method of any one of embodiments 1 to 185, wherein the ADC comprises from 2 to 8 units of MMAE per antibody or antigen binding fragment thereof.

Embodiment 187. The method of any one of embodiments 1 to 186, wherein the ADC comprises from 3 to 5 units of MMAE per antibody or antigen binding fragment thereof.

Embodiment 188. The method of any one of embodiments 1 to 187, wherein the ADC comprises from 3 to 4 units of MMAE per antibody or antigen binding fragment thereof.

Embodiment 189. The method of any one of embodiments 1 to 188, wherein the ADC comprises about 4 units of MMAE per antibody or antigen binding fragment thereof.

Embodiment 190. The method of any one of embodiments 1 to 185, wherein the ADC has the following structure:

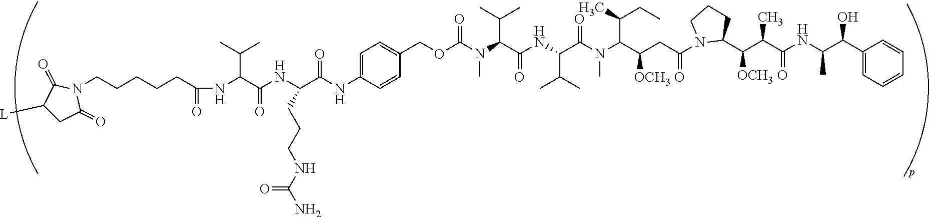

wherein L- represents the antibody or antigen binding fragment thereof and p is from 1 to 10.

Embodiment 191. The method of embodiment 190, wherein p is from 2 to 8.

Embodiment 192. The method of embodiment 190 or 191, wherein p is from 3 to 5.

Embodiment 193. The method of embodiment 190 to 192, wherein p is from 3 to 4.

Embodiment 194. The method of embodiment 190 to 193, wherein p is about 4.

Embodiment 195. The method of embodiment 190 to 193, wherein p is about 3.8.

Embodiment 196. The method of any one of embodiments 1 to 195, wherein the ADC is formulated in a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.

Embodiment 197. The method of any one of embodiments 1 to 195, wherein the ADC is formulated in a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.

Embodiment 198. The method of any one of embodiments 1 to 195, wherein the ADC is formulated at about 10 mg/ml in a pharmaceutical composition comprising about 1.4 mg/ml histidine, about 2.31 mg/ml histidine hydrochloride monohydrate, about 0.2 mg/ml polysorbate 20 (TWEEN-20), and about 55 mg/ml trehalose dihydrate, and wherein the pH of the pharmaceutical composition is about 6.0 at 25° C.

Embodiment 199. The method of any one of embodiments 1 to 195, wherein the ADC is formulated in a vial comprising a pharmaceutical composition comprising about 20 mg of the ADC, about 2.8 mg histidine, about 4.62 mg histidine hydrochloride monohydrate, about 0.4 mg polysorbate 20 (TWEEN-20), and about 110 mg trehalose dihydrate.

Embodiment 200. The method of any one of embodiments 1 to 195, wherein the ADC is formulated in a vial comprising a pharmaceutical composition comprising about 30 mg of the ADC, about 4.2 mg histidine, about 6.93 mg histidine hydrochloride monohydrate, about 0.6 mg polysorbate 20 (TWEEN-20), and about 165 mg trehalose dihydrate.

Embodiment 201. The method of any one of embodiments 1 to 200, wherein the ADC is administered by an intravenous (IV) injection or infusion.

Embodiment 202. The method of any one of embodiments 1 to 201, wherein the ADC or the ADC formulated in the pharmaceutical composition is administered by an intravenous (IV) injection or infusion over about 30 minutes.

Embodiment 203. A method for treating cancer in a subject, comprising administering a treatment regimen to the subject, wherein the treatment regimen comprises:
a. administering one or more doses of an antibody drug conjugate (ADC) to the subject, wherein the one or more doses are administered at a first dose level that contains an effective amount of the ADC;
b. determining whether the subject experiences an adverse reaction in response to administration of the ADC in (a), wherein the adverse reaction is selected from the group consisting of hyperglycemia, peripheral neuropathy, a skin reaction, a nonhematologic toxicity, and a hematologic toxicity;
c. administering one or more subsequent doses of the ADC, each containing an effective amount of the ADC, or discontinuing administration of the ADC based upon the determination in (b), wherein
   i. if the subject is determined not to have experienced an adverse reaction to the ADC or the adverse reaction is determined to be below a defined level, then the one or more subsequent doses of the ADC are administered to the subject at the first dose level;
   ii. if the subject is determined to have experienced an adverse reaction to the ADC at or above a defined level, the treatment regimen is permanently discontinued or administration of the one or more subsequent doses of the ADC are withheld for a period of time sufficient to reduce the adverse reaction to a desired level and then administration of the one or more subsequent doses of ADC are administered at the first dose level or a reduced dose level that is reduced relative to the first dose level; and
d. optionally repeating (a)-(c) one or more times, each repetition of (a)-(c) defining a treatment round, wherein the first dose level in (a) of each subsequent treatment round is either the first dose level from (a) from the immediately preceding round or the reduced dose level of c(ii) from the immediately preceding round, and wherein if the subject is found to have a recurrence of the adverse reaction in two successive treatment rounds, the one or more subsequent doses of the ADC administered in c(ii) is reduced relative to the dose administered in (a) during that treatment round, or administration of the ADC is permanently discontinued; and wherein:
   i. the subject has urothelial cancer, optionally selected from the group of locally advanced or metastatic urothelial cancer, and has previously been treated with an immune checkpoint inhibitor and a chemotherapy agent, wherein the immune checkpoint inhibitor is optionally a programmed death receptor-1 (PD-1) inhibitor, or a programmed death-ligand 1 (PD-L1) inhibitor, and wherein the immune checkpoint inhibitor was optionally administered in a neoadjuvant or adjuvant setting; and
   ii. the ADC comprises an antibody or antigen binding fragment thereof that binds to 191P4D12 and is conjugated to one or more units of monomethyl auristatin E (MMAE), wherein the antibody or antigen binding fragment thereof comprises a heavy chain variable region comprising complementarity determining regions (CDRs) comprising the CDRs of the heavy chain variable region set forth in SEQ ID NO:22 and a light chain variable region comprising the CDRs the light chain variable region set forth in SEQ ID NO:23.

Embodiment 204. The method of embodiment 203, wherein
A. the treatment regimen comprises (a)-(d);
B. the first dose level for the initial treatment round is the starting dose level as indicated in the dose reduction schedule below; and
C. the reduced dose level in c(ii) for each treatment round is reduced to the first dose reduction, the second dose reduction, or the third dose reduction level as set forth in the dose reduction schedule below depending upon whether the dose reduction in c(ii) is the first, second or third dose reduction in the collective treatment rounds, respectively.

| | Dose Level |
|---|---|
| Starting Dose | 1.25 mg/kg if the subject weighs less than 100 kg and |
| | up to 125 mg if the subject weighs 100 kg or more |
| First Dose | 1.0 mg/kg if the subject weighs less than 100 kg, and |
| Reduction | up to 100 mg if the subject weighs 100 kg or more |
| Second Dose | 0.75 mg/kg if the subject weighs less than 100 kg, and |
| Reduction | up to 75 mg if the subject weighs 100 kg or more |
| Third Dose | 0.5 mg/kg if the subject weighs less than 100 kg, and |
| Reduction | up to 50 mg if the subject weighs 100 kg or more |

Embodiment 205. The method of embodiment 203 or 204, wherein
I. the adverse reaction in (b) is hyperglycemia and determining comprises determining the blood glucose level of the subject;
II. the determination to continue or discontinue administration of the ADC in (c) is made as follows:
   i. if the blood glucose level of the subject is equal to or below 250 mg/dL, then the one or more subsequent doses are administered at the first dose level;
   ii. if the blood glucose level of the subject is greater than 250 mg/dL, then administration of the one or more subsequent doses of the ADC are withheld for a period of time sufficient to reduce the blood glucose level to less than or equal to 250 mg/dL, and then the one or more subsequent doses of the ADC are administered at the first dose level; and
   iii. if the blood glucose level of the subject is greater than 500 mg/dL, then the treatment regimen is permanently discontinued.

Embodiment 206. The method of embodiment 204, wherein
I. the determination of an adverse reaction in (b) comprises determining if the subject experiences new or worsening symptoms of peripheral neuropathy; and
II. the determination to continue or discontinue administration of the ADC in (c) is made as follows:
   i. if the subject experiences no symptoms of peripheral neuropathy or has symptoms of peripheral neuropathy below Grade 2, then the one or more subsequent doses of the ADC are administered at the first dose level;
   ii. if the subject experiences a first occurrence of symptoms of Grade 2 peripheral neuropathy at the first dose level administered in (a), then administration of the one or more subsequent doses of the ADC are withheld for a period sufficient to reduce the symptoms of peripheral neuropathy to Grade 1 or lower, and then administration of the one or more subsequent doses of the ADC at the dose level administered in (a) is resumed;
   iii. if the subject has recurrent symptoms of peripheral neuropathy after two successive treatment rounds at the same dose level in (a), then the dose is reduced by one dose level in accordance with the dose reduction schedule; and
   iv. If the subject experiences symptoms of peripheral neuropathy at Grade 3 or higher, then the treatment regimen is permanently discontinued.

Embodiment 207. The method of embodiment 204, wherein
I. the determination of an adverse reaction in (b) comprises determining if the subject experiences a skin reaction; and
II. the decision to continue or discontinue administration of the ADC in (c) is made as follows:
   i. if the subject experiences no skin reaction or has a skin reaction below Grade 3, then the one or more subsequent doses of the ADC are administered at the first dose level;
   ii. if the subject experiences a Grade 3 skin reaction, then the one or more subsequent doses of the ADC are withheld for a period sufficient to reduce the skin reaction to Grade 1 or less and then administration of the one or more subsequent doses of the ADC at the dose level administered in (a) is resumed or reduced by one dose level in accordance with the dose reduction schedule;
   iii. if the subject experiences a Grade 4 skin reaction or has recurrent Grade 3 skin reactions following multiple administrations of the ADC, then the treatment regimen is permanently discontinued.

Embodiment 208. The method of embodiment 204, wherein
I. the determination of an adverse reaction in (b) comprises determining if the subject has symptoms of a nonhematologic toxicity; and
II. the decision to continue or discontinue administration of the ADC in (c) is made as follows:
   i. if the subject experiences a nonhematological toxicity that is below Grade 3, then the one or more subsequent doses of the ADC are administered at the first dose level;
   ii. if the subject experiences a Grade 3 nonhematological toxicity, then the one or more subsequent doses of the ADC are withheld for a period sufficient to reduce the nonhematological to Grade 1 or less and then administration of the one or more subsequent doses of the ADC at the dose level administered in (a) is resumed or reduced by one dose level in accordance with the dose reduction schedule;
   iii. if the subject experiences a Grade 4 nonhematological toxicity, then the treatment regimen is permanently discontinued.

Embodiment 209. The method of embodiment 204, wherein
I. the determination of an adverse reaction in (b) comprises determining if the subject has symptoms of a hematologic toxicity, wherein the hematological toxicity is optionally thrombocytopenia; and
II. the decision to continue or discontinue administration of the ADC in (c) is made as follows:
   i. if the subject experiences a hematological toxicity that is below Grade 3 and the hematological toxicity is not thrombocytopenia, then the one or more subsequent doses of the ADC are administered at the first dose level;
   ii. if the subject experiences a Grade 2 or Grade 3 hematological toxicity, wherein the hematological toxicity is thrombocytopenia, then the one or more subsequent doses of the ADC are withheld for a period sufficient to reduce the thrombocytopenia to Grade 1 or less and then administration of the one or more subsequent doses of the ADC at the dose level administered in (a) is resumed or reduced by one dose level in accordance with the dose reduction schedule;
   iii. if the subject experiences a Grade 4 nonhematological toxicity that is not thrombocytopenia, then administration of the one or more subsequent doses of the ADC at the dose level administered in (a) is reduced by one dose level in accordance with the dose reduction schedule or the treatment regimen is permanently discontinued.

Embodiment 210. The method of any one of embodiments 203 to 209, wherein the antibody or antigen binding fragment thereof comprises a CDR H1 comprising the amino acid sequence of SEQ ID NO:9, a CDR H2 comprising the amino acid sequence of SEQ ID NO:10, a CDR H3 comprising the amino acid sequence of SEQ ID NO:11; a CDR L1 comprising the amino acid sequence of SEQ ID NO:12, a CDR L2 comprising the amino acid sequence of SEQ ID NO:13, and a CDR L3 comprising the amino acid sequence of SEQ ID NO:14.

Embodiment 211. The method of any one of embodiments 203 to 209, wherein the antibody or antigen binding fragment thereof comprises a CDR H1 comprising the amino acid sequence of SEQ ID NO:16, a CDR H2 comprising the amino acid sequence of SEQ ID NO:17, a CDR H3 comprising the amino acid sequence of SEQ ID NO:18; a CDR L1 comprising the amino acid sequence of SEQ ID NO:19, a CDR L2 comprising the amino acid sequence of SEQ ID NO:20, and a CDR L3 comprising the amino acid sequence of SEQ ID NO:21.

Embodiment 212. The method of any one of embodiments 203 to 209, wherein the antibody or antigen binding fragment thereof comprises a CDR H1 consisting of the amino acid sequence of SEQ ID NO:9, a CDR H2 consisting of the amino acid sequence of SEQ ID NO:10, a CDR H3 consisting of the amino acid sequence of SEQ ID NO:11; a CDR L1 consisting of the amino acid sequence of SEQ ID NO:12, a CDR L2 consisting of the amino acid sequence of SEQ ID NO:13, and a CDR L3 consisting of the amino acid sequence of SEQ ID NO:14.

Embodiment 213. The method of any one of embodiments 203 to 209, wherein the antibody or antigen binding fragment thereof comprises a CDR H1 consisting of the amino acid sequence of SEQ ID NO:16, a CDR H2 consisting of the amino acid sequence of SEQ ID NO:17, a CDR H3 consisting of the amino acid sequence of SEQ ID NO:18; a CDR L1 consisting of the amino acid sequence of SEQ ID NO:19, a CDR L2 consisting of the amino acid sequence of SEQ ID NO:20, and a CDR L3 consisting of the amino acid sequence of SEQ ID NO:21.

Embodiment 214. The method of any one of embodiments 203 to 213, wherein the antibody or antigen binding fragment thereof comprises a heavy chain variable region comprising the amino acid sequence of SEQ ID NO:22 and a light chain variable region comprising the amino acid sequence of SEQ ID NO:23.

Embodiment 215. The method of any one of embodiments 203 to 214, wherein the ADC has the following structure:

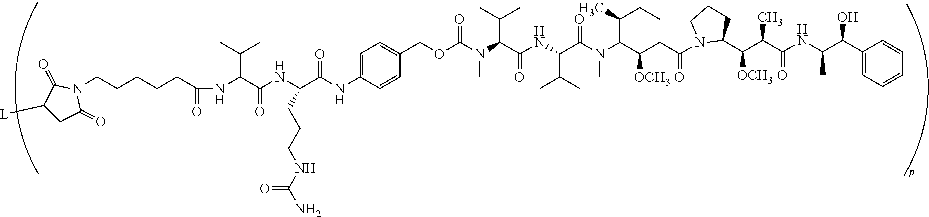

wherein L- represents the antibody or antigen binding fragment thereof and p is from 1 to 10.

Embodiment 216. The method of embodiment 215, wherein p is from 3 to 5.

Embodiment 217. The method of embodiment 215 or 216, wherein p is from 3 to 4.

Embodiment 218. The method of any one of embodiments 215 to 217, wherein p is about 4.

Embodiment 219. The method of any one of embodiments 215 to 217, wherein p is about 3.8.

### 4. BRIEF DESCRIPTION OF THE DRAWINGS

FIGS. 1A-E depict the nucleotide and amino acid sequences of 191P4D12 protein (FIG. 1A), the nucleotide and amino acid sequences of the heavy chain (FIG. 1B) and light chain (FIG. 1C) of Ha22-2(2.4)6.1, and the amino acid sequences of the heavy chain (FIG. 1D) and light chain of Ha22-2(2.4)6.1 (FIG. 1E).

FIG. 2 depicts the efficacy of Ha22-2(2,4)6.1-vcMMAE in subcutaneous established human lung cancer xenograft AG-L4 in SCID mice. The results show that treatment with Ha22-2(2,4)6.1-vcMMAE significantly inhibited the growth of AG-L4 lung cancer xenografts implanted subcutaneously in nude mice compared to both the treated and untreated control.

FIG. 3 depicts the efficacy of Ha22-2(2,4)6.1-vcMMAE in subcutaneous established human breast cancer xenograft BT-483 in SCID mice. The results show that treatment with Ha22-2(2,4)6.1-vcMMAE significantly inhibited the growth of BT-483 breast tumor xenografts implanted subcutaneously in SCID mice compared to the treated and untreated control ADCs.

FIGS. 4A-H. Detection of 191P4D12 protein in cancer patient specimens by IHC.

FIGS. 4A-B show breast cancer specimens. FIGS. 4C-D show lung cancer specimens. FIGS. 4E-F show esophageal cancer specimens. FIGS. 4G-H show head and neck cancer specimens.

FIG. 5. Duration of response (DOR) assessed by blinded independent central review (BICR).

### 5. DETAILED DESCRIPTION

Before the present disclosure is further described, it is to be understood that the disclosure is not limited to the particular embodiments set forth herein, and it is also to be understood that the terminology used herein is for the purpose of describing particular embodiments only, and is not intended to be limiting.

### 5.1 Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

The term “monoclonal antibody” refers to an antibody obtained from a population of substantially homogeneous antibodies, that is, the individual antibodies comprising the population are identical except for possible naturally occurring mutations that can be present in minor amounts. Monoclonal antibodies are highly specific, being directed against a single antigenic site. In contrast to polyclonal antibody preparations, which can include different antibodies directed against different determinants (epitopes), each monoclonal antibody is directed against a single determinant on the antigen.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell, for example, a cancer cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The terms “antigen binding fragment,” “antigen binding domain,” “antigen binding region,” and similar terms refer to that portion of an antibody, which comprises the amino acid residues that interact with an antigen and confer on the binding agent its specificity and affinity for the antigen (e.g., the CDRs). “Antigen-binding fragment” as used herein include “antibody fragment,” which comprise a portion of an intact antibody, such as the antigen-binding or variable region of the intact antibody. Examples of antibody fragments include, without limitation, Fab, Fab′, F(ab′)2, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single variable domain antibodies (sdAbs) (see, e.g., Woolven et al., 1999, Immunogenetics 50: 98-101; and Streltsov et al., 2004, Proc Natl Acad Sci USA. 101:12444-49); and multispecific antibodies formed from antibody fragments.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well-known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibody or antigen binding fragment thereof described herein terms such as “bind to,” “that specifically bind to,” and analogous terms are also used interchangeably herein and refer to binding molecules of antigen binding domains that specifically bind to an antigen, such as a polypeptide. An antibody or antigen binding fragment that binds to or specifically binds to an antigen may be cross-reactive with related antigens. In certain embodiments, an antibody or antigen binding fragment that binds to or specifically binds to an antigen does not cross-react with other antigens. An antibody or antigen binding fragment that binds to or specifically binds to an antigen can be identified, for example, by immunoassays, Octet®, Biacore®, or other techniques known to those of skill in the art. In some embodiments, an antibody or antigen binding fragment binds to or specifically binds to an antigen when it binds to an antigen with higher affinity than to any cross-reactive antigen as determined using experimental techniques, such as radioimmunoassays (MA) and enzyme linked immunosorbent assays (ELISAs). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of an antibody or antigen binding fragment to a “non-target” protein is less than about 10% of the binding of the binding molecule or antigen binding domain to its particular target antigen, for example, as determined by fluorescence activated cell sorting (FACS) analysis or MA. With regard terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. An antibody or antigen binding fragment that binds to an antigen includes one that is capable of binding the antigen with sufficient affinity such that the binding molecule is useful, for example, as a diagnostic agent in targeting the antigen. In certain embodiments, an antibody or antigen binding fragment that binds to an antigen has a dissociation constant (K_{D}) of less than or equal to 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody or antigen binding fragment binds to an epitope of an antigen that is conserved among the antigen from different species (e.g., between human and cyno species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using biolayer interferometry (BLI) or surface plasmon resonance (SPR) assays by Octet®, using, for example, a Octet®QK384 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®QK384, the Biacore®TM-2000, or the Biacore®TM-3000 system.

In certain embodiments, the antibodies or antigen binding fragments can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55).

In certain embodiments, the antibodies or antigen binding fragments can comprise portions of “humanized” forms of nonhuman (e.g., murine) antibodies that are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate comprising the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos. 6,800,738; 6,719,971; 6,639,055; 6,407,213; and 6,054,297.

In certain embodiments, the antibodies or antigen binding fragments can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies comprising variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1: 755-68). Also available for the preparation of human monoclonal antibodies are methods described in Cole et al., Monoclonal Antibodies and Cancer Therapy 77 (1985); Boerner et al., 1991, J. Immunol. 147(1):86-95; and van Dijk and van de Winkel, 2001, Curr. Opin. Pharmacol. 5: 368-74. Human antibodies can be prepared by administering the antigen to a transgenic animal that has been modified to produce such antibodies in response to antigenic challenge, but whose endogenous loci have been disabled, e.g., mice (see, e.g., Jakobovits, 1995, Curr. Opin. Biotechnol. 6(5):561-66; Bruggemann and Taussing, 1997, Curr. Opin. Biotechnol. 8(4):455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

In certain embodiments, the antibodies or antigen binding fragments can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the antibodies or antigen binding fragments can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well-known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and μ contain approximately 550 amino acids. When combined with a light chain, these distinct types of heavy chains give rise to five well-known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted below Table 1.

TABLE 1

| | Kabat | AbM | Chothia | Contact | IMGT |
|---|---|---|---|---|---|
| CDRL1 | L24-L34 | L24-L34 | L24-L34 | L30-L36 | L27-L38 |
| CDR L2 | L50-L56 | L50-L56 | L50-L56 | L46-L55 | L56-L65 |
| CDR L3 | L89-L97 | L89-L97 | L89-L97 | L89-L96 | L105- |
| | | | | | L117 |
| CDR HI | H31-H35B | H26- | H26- | H30- | H27-H38 |
| | (Kabat | H35B | H32..34 | H35B | |
| | Numbering) | | | | |
| CDR Hl | H31-H35 | H26-H35 | H26-H32 | H30-H35 | |
| | (Chothia | | | | |
| | Numbering) | | | | |
| CDRH2 | H50-H65 | H50-H58 | H52-H56 | H47-H58 | H56-H65 |
| CDR H3 | H95-H102 | H95- | H95- | H93- | H105- |
| | | H102 | H102 | H101 | H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule comprising a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations comprising a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

The abbreviation “MMAE” refers to monomethyl auristatin E.

Unless otherwise noted, the term “alkyl” refers to a saturated straight or branched hydrocarbon comprising from about 1 to about 20 carbon atoms (and all combinations and subcombinations of ranges and specific numbers of carbon atoms therein), with from about 1 to about 8 carbon atoms being preferred. Examples of alkyl groups are methyl, ethyl, n-propyl, iso-propyl, n-butyl, iso-butyl, sec-butyl, tert-butyl, n-pentyl, 2-pentyl, 3-pentyl, 2-methyl-2-butyl, n-hexyl, n-heptyl, n-octyl, n-nonyl, n-decyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. Alkyl groups, whether alone or as part of another group, can be optionally substituted with one or more groups, preferably 1 to 3 groups (and any additional substituents selected from halogen), including, but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl, and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be optionally further substituted with one or more groups including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the terms “alkenyl” and “alkynyl” refer to straight and branched carbon chains comprising from about 2 to about 20 carbon atoms (and all combinations and subcombinations of ranges and specific numbers of carbon atoms therein), with from about 2 to about 8 carbon atoms being preferred. An alkenyl chain has at least one double bond in the chain and an alkynyl chain has at least one triple bond in the chain. Examples of alkenyl groups include, but are not limited to, ethylene or vinyl, allyl, -1-butenyl, -2-butenyl, -isobutylenyl, -1-pentenyl, -2-pentenyl, -3-methyl-1-butenyl, -2-methyl-2-butenyl, and -2,3-dimethyl-2- butenyl. Examples of alkynyl groups include, but are not limited to, acetylenic, propargyl, acetylenyl, propynyl, -1-butynyl, -2-butynyl, -1-pentynyl, -2-pentynyl, and -3-methyl-1 butynyl. Alkenyl and alkynyl groups, whether alone or as part of another group, can be optionally substituted with one or more groups, preferably 1 to 3 groups (and any additional substituents selected from halogen), including but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkyenl, —C₂-C₈ alkynyl, or -aryl and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be optionally further substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the term “alkylene” refers to a saturated branched or straight chain hydrocarbon radical comprising from about 1 to about 20 carbon atoms (and all combinations and subcombinations of ranges and specific numbers of carbon atoms therein), with from about 1 to about 8 carbon atoms being preferred and having two monovalent radical centers derived by the removal of two hydrogen atoms from the same or two different carbon atoms of a parent alkane. Typical alkylenes include, but are not limited to, methylene, ethylene, propylene, butylene, pentylene, hexylene, heptylene, ocytylene, nonylene, decalene, 1,4-cyclohexylene, and the like. Alkylene groups, whether alone or as part of another group, can be optionally substituted with one or more groups, preferably 1 to 3 groups (and any additional substituents selected from halogen), including, but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the term “alkenylene” refers to an optionally substituted alkylene group containing at least one carbon-carbon double bond. Exemplary alkenylene groups include, for example, ethenylene (—CH═CH—) and propenylene (—CH═CHCH₂—).

Unless otherwise noted, the term “alkynylene” refers to an optionally substituted alkylene group containing at least one carbon-carbon triple bond. Exemplary alkynylene groups include, for example, acetylene (—C≡C—), propargyl (—CH₂C≡C—), and 4-pentynyl (—CH₂CH₂CH₂C≡CH—).

Unless otherwise noted, the term “aryl” refers to a monovalent aromatic hydrocarbon radical of 6-20 carbon atoms (and all combinations and subcombinations of ranges and specific numbers of carbon atoms therein) derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, phenyl, naphthalene, anthracene, biphenyl, and the like.

An aryl group, whether alone or as part of another group, can be optionally substituted with one or more, preferably 1 to 5, or even 1 to 2 groups including, but not limited to, -halogen, —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, —NO₂, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —C₁-C₈ alkyl, —C₂-C₈ alkenyl, —C₂-C₈ alkynyl, O—(C₁-C₈ alkyl), —O—(C₂-C₈ alkenyl), —O—(C₂-C₈ alkynyl), and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the term “arylene” refers to an optionally substituted aryl group which is divalent (i.e., derived by the removal of two hydrogen atoms from the same or two different carbon atoms of a parent aromatic ring system) and can be in the ortho, meta, or para configurations as shown in the following structures with phenyl as the exemplary aryl group.

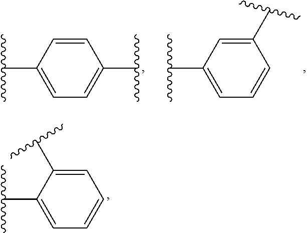

Typical “—(C₁-C₈ alkylene)aryl,” “—(C₂-C₈ alkenylene)aryl”, “and —(C₂-C₈ alkynylene)aryl” groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

Unless otherwise noted, the term “heterocycle,” refers to a monocyclic, bicyclic, or polycyclic ring system having from 3 to 14 ring atoms (also referred to as ring members) wherein at least one ring atom in at least one ring is a heteroatom selected from N, O, P, or S (and all combinations and subcombinations of ranges and specific numbers of carbon atoms and heteroatoms therein). The heterocycle can have from 1 to 4 ring heteroatoms independently selected from N, O, P, or S. One or more N, C, or S atoms in a heterocycle can be oxidized. A monocylic heterocycle preferably has 3 to 7 ring members (e.g., 2 to 6 carbon atoms and 1 to 3 heteroatoms independently selected from N, O, P, or S), and a bicyclic heterocycle preferably has 5 to 10 ring members (e.g., 4 to 9 carbon atoms and 1 to 3 heteroatoms independently selected from N, O, P, or S). The ring that includes the heteroatom can be aromatic or non-aromatic. Unless otherwise noted, the heterocycle is attached to its pendant group at any heteroatom or carbon atom that results in a stable structure. Heterocycles are described in Paquette, “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. 82:5566 (1960). Examples of “heterocycle” groups include by way of example and not limitation pyridyl, dihydropyridyl, tetrahydropyridyl (piperidyl), thiazolyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4H-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl. Preferred “heterocycle” groups include, but are not limited to, benzofuranyl, benzothiophenyl, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A heterocycle group, whether alone or as part of another group, can be optionally substituted with one or more groups, preferably 1 to 2 groups, including but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —O—(C₁-C₈ alkyl), —O—(C₂-C₈ alkenyl), —O—(C₂-C₈ alkynyl), —C₁-C₈ alkyl, —C₂-C₈ alkenyl, —C₂-C₈ alkynyl, and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or aryl.

By way of example and not limitation, carbon-bonded heterocycles can be bonded at the following positions: position 2, 3, 4, 5, or 6 of a pyridine; position 3, 4, 5, or 6 of a pyridazine; position 2, 4, 5, or 6 of a pyrimidine; position 2, 3, 5, or 6 of a pyrazine; position 2, 3, 4, or 5 of a furan, tetrahydrofuran, thiofuran, thiophene, pyrrole or tetrahydropyrrole; position 2, 4, or 5 of an oxazole, imidazole or thiazole; position 3, 4, or 5 of an isoxazole, pyrazole, or isothiazole; position 2 or 3 of an aziridine; position 2, 3, or 4 of an azetidine; position 2, 3, 4, 5, 6, 7, or 8 of a quinoline; or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles can be bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, or 1H-indazole; position 2 of a isoindole, or isoindoline; position 4 of a morpholine; and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

Unless otherwise noted, the term “carbocycle,” refers to a saturated or unsaturated non-aromatic monocyclic, bicyclic, or polycyclic ring system having from 3 to 14 ring atoms (and all combinations and subcombinations of ranges and specific numbers of carbon atoms therein) wherein all of the ring atoms are carbon atoms. Monocyclic carbocycles preferably have 3 to 6 ring atoms, still more preferably 5 or 6 ring atoms. Bicyclic carbocycles preferably have 7 to 12 ring atoms, e.g., arranged as a bicyclo [4,5], [5,5], [5,6] or [6,6] system, or 9 or 10 ring atoms arranged as a bicyclo [5,6] or [6,6] system. The term “carbocycle” includes, for example, a monocyclic carbocycle ring fused to an aryl ring (e.g., a monocyclic carbocycle ring fused to a benzene ring). Carbocyles preferably have 3 to 8 carbon ring atoms. Carbocycle groups, whether alone or as part of another group, can be optionally substituted with, for example, one or more groups, preferably 1 or 2 groups (and any additional substituents selected from halogen), including, but not limited to, -halogen, —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Examples of monocyclic carbocylic substituents include -cyclopropyl, -cyclobutyl, -cyclopentyl, -1-cyclopent-1-enyl, -1-cyclopent-2-enyl, -1-cyclopent-3-enyl, cyclohexyl, -1-cyclohex-1-enyl, -1-cyclohex-2-enyl, -1-cyclohex-3-enyl, -cycloheptyl, -cyclooctyl. -1,3-cyclohexadienyl, -1,4-cyclohexadienyl, -1,3-cycloheptadienyl, -1,3,5-cycloheptatrienyl, and -cyclooctadienyl.

A “carbocyclo,” whether used alone or as part of another group, refers to an optionally substituted carbocycle group as defined above that is divalent (i.e., derived by the removal of two hydrogen atoms from the same or two different carbon atoms of a parent carbocyclic ring system).

Unless otherwise indicated by context, a hyphen (-) designates the point of attachment to the pendant molecule. Accordingly, the term “—(C₁-C₈ alkylene)aryl” or “—C₁-C₈ alkylene(aryl)” refers to a C₁-C₈ alkylene radical as defined herein wherein the alkylene radical is attached to the pendant molecule at any of the carbon atoms of the alkylene radical and one of the hydrogen atoms bonded to a carbon atom of the alkylene radical is replaced with an aryl radical as defined herein.

When a particular group is “substituted”, that group may have one or more substituents, preferably from one to five substituents, more preferably from one to three substituents, most preferably from one to two substituents, independently selected from the list of substituents. The group can, however, generally have any number of substituents selected from halogen. Groups that are substituted are so indicated. It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

Protective groups as used herein refer to groups which selectively block, either temporarily or permanently, one reactive site in a multifunctional compound. Suitable hydroxy-protecting groups for use in the present invention are pharmaceutically acceptable and may or may not need to be cleaved from the parent compound after administration to a subject in order for the compound to be active. Cleavage is through normal metabolic processes within the body. Hydroxy protecting groups are well-known in the art, see, Protective Groups in Organic Synthesis by T. W. Greene and P. G. M. Wuts (John Wiley & sons, 3^{rd} Edition) incorporated herein by reference in its entirety and for all purposes and include, for example, ether (e.g., alkyl ethers and silyl ethers including, for example, dialkylsilylether, trialkylsilylether, dialkylalkoxysilylether), ester, carbonate, carbamates, sulfonate, and phosphate protecting groups. Examples of hydroxy protecting groups include, but are not limited to, methyl ether; methoxymethyl ether, methylthiomethyl ether, (phenyldimethylsilyl)methoxymethyl ether, benzyloxymethyl ether, p-methoxybenzyloxymethyl ether, p-nitrobenzyloxymethyl ether, o-nitrobenzyloxymethyl ether, (4-methoxyphenoxy)methyl ether, guaiacolmethyl ether, t-butoxymethyl ether, 4-pentenyloxymethyl ether, siloxymethyl ether, 2-methoxyethoxymethyl ether, 2,2,2-trichloroethoxymethyl ether, bis(2-chloroethoxy)methyl ether, 2-(trimethylsilyl)ethoxymethyl ether, menthoxymethyl ether, tetrahydropyranyl ether, 1-methoxycylcohexyl ether, 4-methoxytetrahydrothiopyranyl ether, 4-methoxytetrahydrothiopyranyl ether S,S-Dioxide, 1-[(2-choro-4-methyl)phenyl]-4-methoxypiperidin-4-yl ether, 1-(2-fluorophneyl)-4-methoxypiperidin-4-yl ether, 1,4-dioxan-2-yl ether, tetrahydrofuranyl ether, tetrahydrothiofuranyl ether; substituted ethyl ethers such as 1-ethoxyethyl ether, 1-(2-chloroethoxy)ethyl ether, 1-[2-(trimethylsilyl)ethoxy]ethyl ether, 1-methyl-1-methoxy ethyl ether, 1-methyl-1-benzyloxyethyl ether, 1-methyl-1-benzyloxy-2-fluoroethyl ether, 1-methyl-1phenoxyethyl ether, 2-trimethylsilyl ether, t-butyl ether, allyl ether, propargyl ethers, p-chlorophenyl ether, p-methoxyphenyl ether, benzyl ether, p-methoxybenzyl ether 3,4-dimethoxybenzyl ether, trimethylsilyl ether, triethylsilyl ether, tripropylsilylether, dimethylisopropylsilyl ether, diethylisopropylsilyl ether, dimethylhexylsilyl ether, t-butyldimethylsilyl ether, diphenylmethylsilyl ether, benzoylformate ester, acetate ester, chloroacetate ester, dichloroacetate ester, trichloroacetate ester, trifluoroacetate ester, methoxyacetate ester, triphneylmethoxyacetate ester, phenylacetate ester, benzoate ester, alkyl methyl carbonate, alkyl 9-fluorenylmethyl carbonate, alkyl ethyl carbonate, alkyl 2,2,2,-trichloroethyl carbonate, 1,1,-dimethyl-2,2,2-trichloroethyl carbonate, alkylsulfonate, methanesulfonate, benzylsulfonate, tosylate, methylene acetal, ethylidene acetal, and t-butylmethylidene ketal. Preferred protecting groups are represented by the formulas —R^{a}, —Si(R^{a})(R^{a})(R^{a}), —C(O)R^{a}, —C(O)R^{a}, —C(O)NH(R^{a}), —S(O)₂R^{a}, —S(O)₂OH, P(O)(OH)₂, and —P(O)(OH)OR^{a}, wherein R^{a} is C₁-C₂₀ alkyl, C₂-C₂₀ alkenyl, C₂-C₂₀ alkynyl, —C₁-C₂₀ alkylene(carbocycle), —C₂-C₂₀ alkenylene(carbocycle), —C₂-C₂₀ alkynylene(carbocycle), —C₆-C₁₀ aryl, —C₁-C₂₀ alkylene(aryl), —C₂-C₂₀ alkenylene(aryl), —C₂-C₂₀ alkynylene(aryl), —C₁-C₂₀ alkylene(heterocycle), —C₂-C₂₀ alkenylene(heterocycle), or —C₂-C₂₀ alkynylene(heterocycle) wherein said alkyl, alkenyl, alkynyl, alkylene, alkenylene, alkynylene, aryl, carbocycle, and heterocycle radicals whether alone or as part of another group are optionally substituted.

The term “Chemotherapeutic Agent” refers to all chemical compounds that are effective in inhibiting tumor growth. Non-limiting examples of chemotherapeutic agents include alkylating agents; for example, nitrogen mustards, ethyleneimine compounds and alkyl sulphonates; antimetabolites, for example, folic acid, purine or pyrimidine antagonists; mitotic inhibitors, for example, anti-tubulin agents such as vinca alkaloids, auristatins and derivatives of podophyllotoxin; cytotoxic antibiotics; compounds that damage or interfere with DNA expression or replication, for example, DNA minor groove binders; and growth factor receptor antagonists. In addition, chemotherapeutic agents include cytotoxic agents (as defined herein), antibodies, biological molecules and small molecules.

The term “compound” refers to and encompasses the chemical compound itself as well as, whether explicitly stated or not, and unless the context makes clear that the following are to be excluded: amorphous and crystalline forms of the compound, including polymorphic forms, where these forms may be part of a mixture or in isolation; free acid and free base forms of the compound, which are typically the forms shown in the structures provided herein; isomers of the compound, which refers to optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers; isotopes of the compound, including deuterium- and tritium-containing compounds, and including compounds containing radioisotopes, including therapeutically- and diagnostically-effective radioisotopes; multimeric forms of the compound, including dimeric, trimeric, etc. forms; salts of the compound, preferably pharmaceutically acceptable salts, including acid addition salts and base addition salts, including salts having organic counterions and inorganic counterions, and including zwitterionic forms, where if a compound is associated with two or more counterions, the two or more counterions may be the same or different; and solvates of the compound, including hemisolvates, monosolvates, disolvates, etc., including organic solvates and inorganic solvates, said inorganic solvates including hydrates; where if a compound is associated with two or more solvent molecules, the two or more solvent molecules may be the same or different. In some instances, reference made herein to a compound of the invention will include an explicit reference to one or of the above forms, e.g., salts and/or solvates; however, this reference is for emphasis only, and is not to be construed as excluding other of the above forms as identified above.

As used herein, the term “conservative substitution” refers to substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson, et al., MOLECULAR BIOLOGY OF THE GENE, The Benjamin/Cummings Pub. Co., p. 224 (4th Edition 1987)). Such exemplary substitutions are preferably made in accordance with those set forth in Table 2 and Table 3. For example, such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g. Table 3 herein; pages 13-15 “Biochemistry” 2nd ED. Lubert Stryer ed (Stanford University); Henikoff et al., PNAS 1992 Vol 89 10915-10919; Lei et al., J Biol Chem 1995 May 19; 270(20):11882-11886). Other substitutions are also permissible and may be determined empirically or in accord with known conservative substitutions.

TABLE 2
Amino Acid Abbreviations

| SINGLE LETTER | THREE LETTER | FULL NAME |
|---|---|---|
| F | Phe | phenylalanine |
| L | Leu | leucine |
| S | Ser | serine |
| Y | Tyr | tyrosine |
| C | Cys | cysteine |
| W | Trp | tryptophan |
| P | Pro | proline |
| H | His | histidine |
| Q | Gin | glutamine |
| R | Arg | arginine |
| I | Ile | isoleucine |
| M | Met | methionine |
| T | Thr | threonine |
| N | Asn | asparagine |
| K | Lys | lysine |
| V | Val | valine |
| A | Ala | alanine |
| D | Asp | aspartic acid |
| E | Glu | glutamic acid |
| G | Gly | glycine |

TABLE 3
Amino Acid Substitution or Similarity Matrix
Adapted from the GCG Software 9.0 BLOSUM62 amino acid substitution matrix (block substitution
matrix). The higher the value, the more likely a substitution is found in related, natural proteins.

| A | C | D | E | F | G | H | I | K | L | M | N | P | Q | R | S | T | V | W | Y | . |
|---|---|---|---|---|---|---|---|---|---|---|---|---|---|---|---|---|---|---|---|---|
| 4 | 0 | −2 | −1 | −2 | 0 | −2 | −1 | −1 | −1 | −1 | −2 | −1 | −1 | −1 | 1 | 0 | 0 | −3 | −2 | A |
| | 9 | −3 | −4 | −2 | −3 | −3 | −1 | −3 | −1 | −1 | −3 | −3 | −3 | −3 | −1 | −1 | −1 | −2 | −2 | C |
| | | 6 | 2 | −3 | −1 | −1 | −3 | −1 | −4 | −3 | 1 | −1 | 0 | −2 | 0 | −1 | −3 | −4 | −3 | D |
| | | | 5 | −3 | −2 | 0 | −3 | 1 | −3 | −2 | 0 | −1 | 2 | 0 | 0 | −1 | −2 | −3 | −2 | E |
| | | | | 6 | −3 | −1 | 0 | −3 | 0 | 0 | −3 | −4 | −3 | −3 | −2 | −2 | −1 | 1 | 3 | F |
| | | | | | 6 | −2 | −4 | −2 | −4 | −3 | 0 | −2 | −2 | −2 | 0 | −2 | −3 | −2 | −3 | G |
| | | | | | | 8 | −3 | −1 | −3 | −2 | 1 | −2 | 0 | 0 | −1 | −2 | −3 | −2 | 2 | H |
| | | | | | | | 4 | −3 | 2 | 1 | −3 | −3 | −3 | −3 | −2 | −1 | 3 | −3 | −1 | I |
| | | | | | | | | 5 | −2 | −1 | 0 | −1 | 1 | 2 | 0 | −1 | −2 | −3 | −2 | K |
| | | | | | | | | | 4 | 2 | −3 | −3 | −2 | −2 | −2 | −1 | 1 | −2 | −1 | L |
| | | | | | | | | | | 5 | −2 | −2 | 0 | −1 | −1 | −1 | 1 | −1 | −1 | M |
| | | | | | | | | | | | 6 | −2 | 0 | 0 | 1 | 0 | −3 | −4 | −2 | N |
| | | | | | | | | | | | | 7 | −1 | −2 | −1 | −1 | −2 | −4 | −3 | P |
| | | | | | | | | | | | | | 5 | 1 | 0 | −1 | −2 | −2 | −1 | Q |
| | | | | | | | | | | | | | | 5 | −1 | −1 | −3 | −3 | −2 | R |
| | | | | | | | | | | | | | | | 4 | 1 | −2 | −3 | −2 | S |
| | | | | | | | | | | | | | | | | 5 | 0 | −2 | −2 | T |
| | | | | | | | | | | | | | | | | | 4 | −3 | −1 | V |
| | | | | | | | | | | | | | | | | | | 11 | 2 | W |
| | | | | | | | | | | | | | | | | | | | 7 | Y |

The term “homology” or “homologous” is intended to mean a sequence similarity between two polynucleotides or between two polypeptides. Similarity can be determined by comparing a position in each sequence, which can be aligned for purposes of comparison. If a given position of two polypeptide sequences is not identical, the similarity or conservativeness of that position can be determined by assessing the similarity of the amino acid of the position, for example, according to Table 3. A degree of similarity between sequences is a function of the number of matching or homologous positions shared by the sequences. The alignment of two sequences to determine their percent sequence similarity can be done using software programs known in the art, such as, for example, those described in Ausubel et al., Current Protocols in Molecular Biology, John Wiley and Sons, Baltimore, Md. (1999). Preferably, default parameters are used for the alignment, examples of which are set forth below. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information.

The term “homologs” of to a given amino acid sequence or a nucleic acid sequence is intended to indicate that the corresponding sequences of the “homologs” having substantial identity or homology to the given amino acid sequence or nucleic acid sequence.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The term “cytotoxic agent” refers to a substance that inhibits or prevents the expression activity of cells, function of cells and/or causes destruction of cells. The term is intended to include radioactive isotopes, chemotherapeutic agents, and toxins such as small molecule toxins or enzymatically active toxins of bacterial, fungal, plant or animal origin, including fragments and/or variants thereof. Examples of cytotoxic agents include, but are not limited to auristatins (e.g., auristatin E, auristatin F, MMAE and MMAF), auromycins, maytansinoids, ricin, ricin A-chain, combrestatin, duocarmycins, dolastatins, doxorubicin, daunorubicin, taxols, cisplatin, cc1065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, and glucocorticoid and other chemotherapeutic agents, as well as radioisotopes such as At²¹¹, I¹³¹, I¹²⁵, Y⁹⁰, Re¹⁸⁶, Re¹⁸⁸, Sm¹⁵³, Bi²¹² or ²¹³, P³² and radioactive isotopes of Lu including Lu¹⁷⁷. Antibodies may also be conjugated to an anti-cancer pro-drug activating enzyme capable of converting the pro-drug to its active form.

The term “effective amount” or “therapeutically effective amount” as used herein refers to the amount of binding molecule (e.g., an antibody) or pharmaceutical composition provided herein which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., a cancer).

The term “cancer” or “cancer cell” is used herein to denote a tissue or cell found in a neoplasm which possesses characteristics which differentiate it from normal tissue or tissue cells. Among such characteristics include but are not limited to: degree of anaplasia, irregularity in shape, indistinctness of cell outline, nuclear size, changes in structure of nucleus or cytoplasm, other phenotypic changes, presence of cellular proteins indicative of a cancerous or pre-cancerous state, increased number of mitoses, and ability to metastasize. Words pertaining to “cancer” include carcinoma, sarcoma, tumor, epithelioma, leukemia, lymphoma, polyp, and scirrus, transformation, neoplasm, and the like.

As used herein, a “locally advanced” cancer refers to a cancer that has spread from where it started to nearby tissue or lymph nodes.

As used herein, a “metastatic” cancer refers to a cancer that has spread from where it started to different part of the body.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

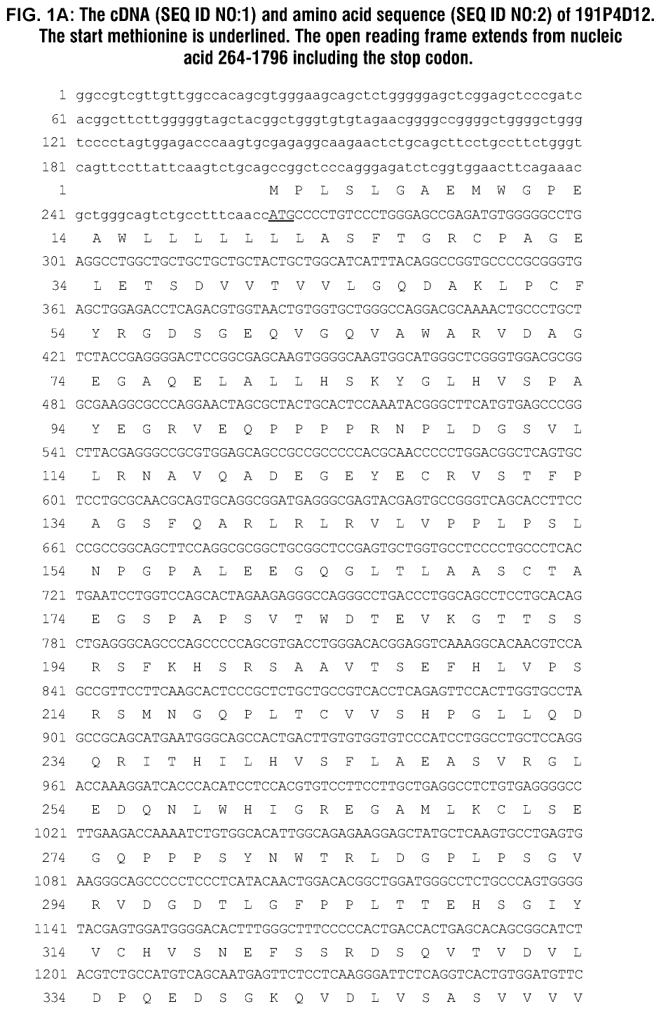

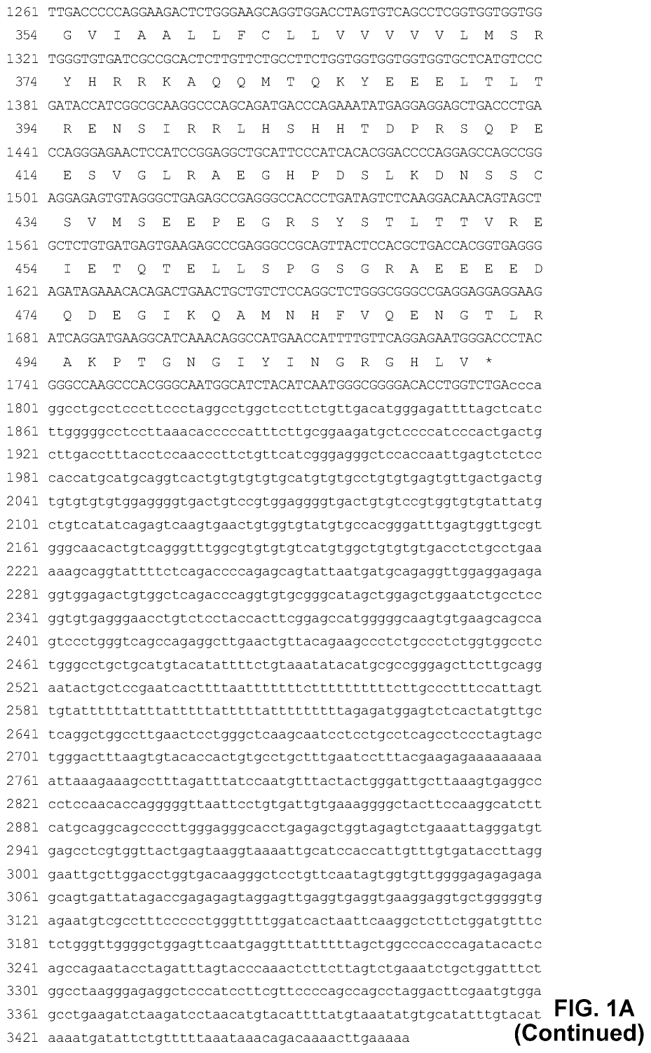

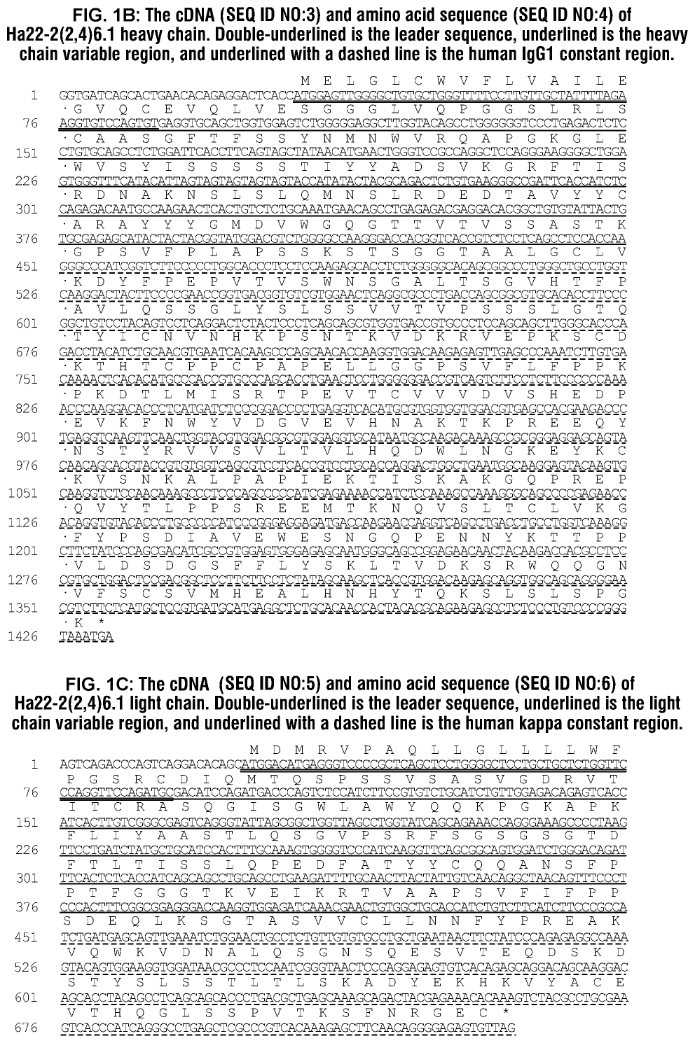

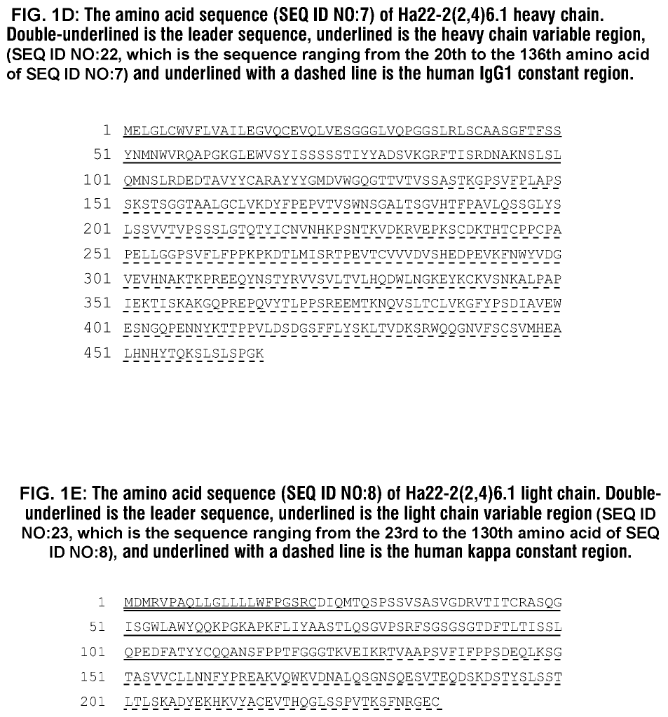

The term “variant” refers to a molecule that exhibits a variation from a described type or norm, such as a protein that has one or more different amino acid residues in the corresponding position(s) of a specifically described protein (e.g. the 191P4D12 protein shown in FIG. 1.) An analog is an example of a variant protein. Splice isoforms and single nucleotides polymorphisms (SNPs) are further examples of variants.

The “191P4D12 proteins” and/or “191P4D12 related proteins” of the invention include those specifically identified herein (see, FIG. 1), as well as allelic variants, conservative substitution variants, analogs and homologs that can be isolated/generated and characterized without undue experimentation following the methods outlined herein or readily available in the art. Fusion proteins that combine parts of different 191P4D12 proteins or fragments thereof, as well as fusion proteins of a 191P4D12 protein and a heterologous polypeptide are also included. Such 191P4D12 proteins are collectively referred to as the 191P4D12-related proteins, the proteins of the invention, or 191P4D12. The term “191P4D12-related protein” refers to a polypeptide fragment or a 191P4D12 protein sequence of 4, 5, 6, 7, 8, 9, 10, 11, 12, 13, 14, 15, 16, 17, 18, 19, 20, 21, 22, 23, 24, 25, or more than 25 amino acids; or, at least 30, 35, 40, 45, 50, 55, 60, 65, 70, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 330, 335, 339 or more amino acids.

As used herein, “peripheral neuropathy” refers to a disorder characterized by inflammation or degeneration of the peripheral sensory or motor nerves. The disorder characterized by inflammation or degeneration of the peripheral sensory nerves is referred to as peripheral sensory neuropathy. The disorder characterized by inflammation or degeneration of the peripheral motor nerves is referred to as peripheral motor neuropathy. Peripheral neuropathy, which is a disorder in the nerves, can have various manifestation or symptoms in the subject having peripheral neuropathy. Peripheral neuropathy, for example when used in the context of a subject, is a grouped term and include: hypoesthesia, gait disturbance, muscular weakness, neuralgia, paresthesia, peripheral motor neuropathy, peripheral sensory neuropathy and peripheral sensorimotor neuropathy. As described further below, peripheral neuropathy can be assessed, evaluated, described, and categorized in accordance with Common Terminology Criteria for Adverse Events (CTCAE) Grading v4.0. In some embodiments, peripheral neuropathy can be assessed, evaluated, described, and categorized in accordance with Table 6 below.

As used herein, “hyperglycemia” refers to a disorder characterized by laboratory test results that indicate an elevation in the concentration of blood sugar. Hyperglycemia is usually an indication of diabetes mellitus or glucose intolerance. As described further below, hyperglycemia can be assessed, evaluated, described, and categorized in accordance with CTCAE Grading v4.0. In some embodiments, hyperglycemia can be assessed, evaluated, described, and categorized in accordance with Table 5 below.

As used herein, a “skin reaction” or “skin reactions” refers to a response to the ADC treatment manifested in the subject's skin. Such a response can be a direct result of the ADC treatment, for example, damages and other pathologies caused by the ADC to the subject's skin. Such a response can also be a indirect result of the ADC treatment, for example, a result of the inflammation, necrosis, apoptosis, and/or immune response to the primary or direct damages or pathologies caused by the ADC. The skin reactions include, for example and not by way of limitation, maculopapular rash, pruritus, symmetrical drug-related intertriginous, flexural exanthema (SDRIFE), bullous dermatitis, exfoliative dermatitis, palmar-plantar erythrodysesthesia, rash pustula, rash acneiform, papulopustular rash, and/or dry skin. As described further below, skin reactions can be assessed, evaluated, described, and categorized in accordance with CTCAE Grading v4.0. In some embodiments, skin reactions can be assessed, evaluated, described, and categorized in accordance with Table 9 below.

As used herein, “dysgeusia” refers to a disorder characterized by abnormal sensual experience with the taste of foodstuffs; it can be related to a decrease in the sense of smell. “Anorexia” refers to a disorder characterized by a loss of appetite. As described further below, dysgeusia and anorexia can be assessed, evaluated, described, and categorized in accordance with Table 11 below.

As used herein, “keratitis” refers to a disorder characterized by inflammation to the cornea of the eye. “Dry eye” refers to a disorder characterized by dryness of the cornea and conjunctiva. “Blurred vision” refers to a disorder characterized by visual perception of unclear or fuzzy images. As described further below, keratitis, dry eye, and blurred vision can be assessed, evaluated, described, and categorized in accordance with CTCAE Grading v4.0 and/or Table 11 below.

As used herein, “febrile neutropenia” refers to a disorder characterized by an ANC<1000/mm³ and a single temperature of >38.3 degrees C. (101 degrees F.) or a sustained temperature of ≥38 degrees C. (100.4 degrees F.) for more than one hour. As described further below, febrile neutropenia can be assessed, evaluated, described, and categorized in accordance with CTCAE Grading v4.0 and/or Table 13 below.

As used herein, “thrombocytopenia” refers to a condition characterized by abnormally low levels of platelets in the blood. Thrombocytopenia is often determined based on the counts of platelets. Accordingly, thrombocytopenia is a condition in a subject characterized by a decrease in number of platelets in a blood specimen to a range that is below normal. As used herein, “anemia” refers to a disorder characterized by an reduction in the amount of hemoglobin in 100 ml of blood. Signs and symptoms of anemia may include pallor of the skin and mucous membranes, shortness of breath, palpitations of the heart, soft systolic murmurs, lethargy, and fatigability. As described further below, thrombocytopenia and anemia can be assessed, evaluated, described, and categorized in accordance with CTCAE Grading v4.0 and/or Table 13 below.

As used herein, “fatigue” refers to a disorder characterized by a state of generalized weakness with a pronounced inability to summon sufficient energy to accomplish daily activities. As described further below, fatigue can be assessed, evaluated, described, and categorized in accordance with CTCAE Grading v4.0 and/or Table 15 below.

As used herein, “diarrhea” refers to a disorder characterized by frequent and watery bowel movements. As described further below, diarrhea can be assessed, evaluated, described, and categorized in accordance with CTCAE Grading v4.0 and/or Table 17 below. Additionally, diarrhea can be assessed, evaluated, described, and categorized in accordance with National Cancer Institute, Gastrointestinal Complications (PDQ®)—Health Professional Version. https://www.cancer.gov/about-cancer/treatment/side-effects/constipation/gi-complications-hp-pdq as updated on Nov. 28, 2018, which is incorporated herein in its entirety by reference.

### 5.2 Methods of Treating Cancer

### 5.2.1 Methods of Treating Cancer in Various Settings

Provided herein are methods for the treatment of various cancers, including bladder cancer, using an antibody drug conjugate (ADC) that binds 191P4D12. Also provided herein are methods for the treatment of urothelial cancer using an antibody drug conjugate (ADC) that binds 191P4D12. Additionally provided herein are methods for the treatment of solid tumors using an antibody drug conjugate (ADC) that binds 191P4D12. In certain embodiments, the urothelial cancer is locally advanced or metastatic urothelial cancer. In certain embodiments, the bladder cancer is locally advanced or metastatic bladder cancer. In certain embodiments, the solid tumors are locally advanced or metastatic solid tumors. In other embodiments, the urothelial cancer is locally advanced or metastatic urothelial cancer in a patient who have received a programmed death receptor-1 (PD-1) or programmed death-ligand 1 (PD-L1) inhibitor and who have received a platinum-containing chemotherapy in the neoadjuvant/adjuvant, locally advanced or metastatic setting. In some embodiments, the bladder cancer is locally advanced or metastatic bladder cancer in a patient who have received a programmed death receptor-1 (PD-1) or programmed death-ligand 1 (PD-L1) inhibitor and who have received a platinum-containing chemotherapy in the neoadjuvant/adjuvant, locally advanced or metastatic setting. In some embodiments, the solid tumors are locally advanced or metastatic solid tumors in a patient who have received a programmed death receptor-1 (PD-1) or programmed death-ligand 1 (PD-L1) inhibitor and who have received a platinum-containing chemotherapy in the neoadjuvant/adjuvant, locally advanced or metastatic setting. In some embodiments the ADC is enfortumab vedotin (also known as anti-191P4D12-ADC, Ha22-2(2,4)6.1vcMMAE, ASG-22CE, ASG-22ME, or AGS-22M6E). In some embodiments, the ADC is administered three times every 28 day cycle. In some specific embodiments, the ADC is administered on Days 1, 8 and 15 of every 28 day cycle.

In one aspect, provided herein is a method of preventing or treating cancer in a subject, comprising (a) administering to the subject a first regimen comprising an effective amount of an antibody drug conjugate (ADC), wherein the ADC comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; wherein the subject has urothelial cancer or bladder cancer; and wherein the subject has received an immune checkpoint inhibitor therapy and received a chemotherapy.

In some embodiments of the methods provided herein, the ADC is administered three times within a 28 day cycle. In some embodiments of the methods provided herein, the ADC is administered on Days 1, 8 and 15 of a 28 day cycle. In some embodiments of the methods provided herein, the urothelial cancer is locally advanced urothelial cancer. In some embodiments of the methods provided herein, the bladder cancer is locally advanced bladder cancer. In some embodiments of the methods provided herein, the urothelial cancer is metastatic urothelial cancer. In some embodiments of the methods provided herein, the bladder cancer is metastatic bladder cancer. In some embodiments of the methods provided herein, the immune checkpoint inhibitor therapy is a PD-1 inhibitor. In some embodiments of the methods provided herein, the immune checkpoint inhibitor therapy is PD-L1 inhibitor. In some embodiments of the methods provided herein, the chemotherapy is platinum-containing chemotherapy. In some embodiments of the methods provided herein, the platinum-containing chemotherapy is platinum-containing chemotherapy in a neoadjuvant setting. In some embodiments of the methods provided herein, the platinum-containing chemotherapy is platinum-containing chemotherapy in a neoadjuvant and locally advanced setting. In some embodiments of the methods provided herein, the platinum-containing chemotherapy is platinum-containing chemotherapy in a neoadjuvant and metastatic setting. In some embodiments of the methods provided herein, the platinum-containing chemotherapy is platinum-containing chemotherapy in an adjuvant setting. In some embodiments of the methods provided herein, the platinum-containing chemotherapy is platinum-containing chemotherapy in an adjuvant and locally advanced setting. In some embodiments of the methods provided herein, the platinum-containing chemotherapy is platinum-containing chemotherapy in an adjuvant and metastatic setting. In some embodiments of the methods provided herein, the platinum-containing chemotherapy is platinum-containing chemotherapy in a locally advanced setting. In some embodiments of the methods provided herein, the platinum-containing chemotherapy is platinum-containing chemotherapy in a metastatic setting. In some embodiments of the methods provided herein, the first regimen comprises an ADC dose of about 1.25 milligram/kilogram (mg/kg) of the subject's body weight. In some embodiments of the methods provided herein, the subject has a body weight of less than 100 kg. In some embodiments of the methods provided herein, the first regimen comprises an ADC dose of about 1.25 milligram/kilogram (mg/kg) of the subject's body weight, wherein the subject has a body weight of less than 100 kg. In some embodiments of the methods provided herein, the first regimen comprises an ADC dose of about 125 mg to the subject, wherein the subject has a body weight of no less than 100 kg. The disclosure provides that the embodiments in this paragraph can be but are not limited to specific embodiments of the aspect in the preceding paragraph.

In some aspects, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; wherein the subject has locally advanced or metastatic urothelial cancer; wherein the subject has received a PD-1 or PD-L1 inhibitor; and wherein the subject has received a platinum-containing chemotherapy in the neoadjuvant/adjuvant, locally advanced or metastatic setting. In some aspects, also provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; wherein the subject has locally advanced or metastatic bladder cancer; wherein the subject has received a PD-1 or PD-L1 inhibitor; and wherein the subject has received a platinum-containing chemotherapy in the neoadjuvant/adjuvant, locally advanced or metastatic setting.

In all the methods provided herein and specifically those described in the previous two paragraphs: the therapeutic agents that can be used are described in this Section (Section 5.2) and Section 5.3, selection of patients for treatment is described herein and exemplified in this Section (Section 5.2) and Section 6, dosing regimens and pharmaceutical composition for administering the therapeutic agent are described in this Section (Section 5.2), Section 5.4 and Section 6 below, the biomarkers that can be used for identifying the therapeutic agents, selecting the patients, determining the outcome of these methods, and/or serving as criteria in any way for these methods are described herein and exemplified in this Section (Section 5.2) and Section 6, therapeutic outcomes for the methods provided herein can be improvement of the biomarkers described herein, for example, those described and exemplified in this Section (Section 5.2) and Section 6. Therefore, a person skilled in the art would understand that the methods provided herein include all permutations and combinations of the patients, therapeutic agents, dosing regiments, biomarkers, and therapeutic outcomes as described above and below.

In certain embodiments, the methods provided herein are used for treating subjects having cancers that express 191P4D12 RNA, express 191P4D12 protein, or express both 191P4D12 RNA and 191P4D12 protein. In certain embodiments, the methods provided herein are used for treating subjects having cancers that express both 191P4D12 RNA and 191P4D12 protein, including for example, locally advanced or metastatic urothelial cancer or locally advanced or metastatic bladder cancer in a subject who has received a PD-1 or PD-L1 inhibitor and who have received a platinum-containing chemotherapy in the neoadjuvant/adjuvant, locally advanced or metastatic setting. In some embodiments, the 191P4D12 RNA expression in the cancers is determined by polynucleotide hybridization, sequencing (assessing the relative abundance of the sequences), and/or PCR (including RT-PCR). In some embodiments, the 191P4D12 protein expression in the cancers is determined by IHC, analysis in fluorescence-activated cell sorting (FACS), and/or western blotting. In some embodiments, the 191P4D12 protein expression in the cancers is determined by more than one method. In some embodiments, the 191P4D12 protein expression in the cancers is determined by two methods of IHC.

In certain embodiments, the methods provided herein are used for treating subjects having cancers, wherein the cancers express 191P4D12 RNA, express 191P4D12 protein, or express both 191P4D12 RNA and 191P4D12 protein, and wherein the cancers are sensitive to cytotoxic agents (such as Vinca and MMAE) blocking microtubule polymerization. In certain embodiments, the methods provided herein are used for treating subjects having cancers that express both 191P4D12 RNA and 191P4D12 protein and that are sensitive to cytotoxic agents (such as Vinca and MMAE) blocking microtubule polymerization, which cancers include for example, locally advanced or metastatic urothelial cancer or locally advanced or metastatic bladder cancer in an adult who has received a PD-1 or PD-L1 inhibitor and who have received a platinum-containing chemotherapy in the neoadjuvant/adjuvant, locally advanced or metastatic setting.

In some embodiments, the subjects that can be treated in the methods provided herein are subjects having locally advanced or metastatic urothelial cancer, wherein the subjects have received a PD-1 or PD-L1 inhibitor and have received a platinum-containing chemotherapy in the neoadjuvant/adjuvant, locally advanced or metastatic setting. In some embodiments, the subjects that can be treated in the methods provided herein are subjects having locally advanced or metastatic bladder cancer, wherein the subjects have received a PD-1 or PD-L1 inhibitor and have received a platinum-containing chemotherapy in the neoadjuvant/adjuvant, locally advanced or metastatic setting.

In certain embodiments, the cancers that can be treated in the methods provided herein include locally advanced or metastatic urothelial cancer in subjects, wherein the subjects have received a PD-1 or PD-L1 inhibitor and have received a platinum-containing chemotherapy in the neoadjuvant/adjuvant, locally advanced or metastatic setting. In certain embodiments, the cancers that can be treated in the methods provided herein include locally advanced or metastatic bladder cancer in subjects, wherein the subjects have received a PD-1 or PD-L1 inhibitor and have received a platinum-containing chemotherapy in the neoadjuvant/adjuvant, locally advanced or metastatic setting.

In some embodiments, the locally advanced or metastatic urothelial cancers are confirmed histologically, cytologically, or both histologically and cytologically. In some embodiments, the locally advanced or metastatic bladder cancers are confirmed histologically, cytologically, or both histologically and cytologically

In some embodiments, the subjects that can be treated in the methods provided herein include subjects who received one or more other treatments for cancer. In some embodiments, the subjects that can be treated in the methods provided herein include subjects who received one or more other treatments for cancer and whose cancer progressed or relapsed following the one or more treatments. Such one or more treatments include, for example, one or more lines of immune checkpoint inhibitor therapies, chemotherapies, and both immune checkpoint inhibitor therapies and chemotherapies. In some embodiments, the subjects that can be treated in the methods provided herein include subjects whose cancers progressed or relapsed following a therapy with an inhibitor of programmed cell death protein-1 (PD-1), an inhibitor of programmed cell death-ligand 1 (PD-L1), a platinum-containing chemotherapy or any permutation or combination of two or more of the therapies provided in this paragraph and those described herein. In some embodiments, the subjects that can be treated in the methods provided herein include subjects whose cancers progressed or relapsed following a therapy with an inhibitor of PD-1. In some embodiments, the subjects that can be treated in the methods provided herein include subjects whose cancers progressed or relapsed following a therapy with an inhibitor of PD-L1. In some embodiments, the subjects that can be treated in the methods provided herein include subjects whose cancers progressed or relapsed following a platinum-containing chemotherapy. In some embodiments, the subjects that can be treated in the methods provided herein include subjects whose cancers progressed or relapsed following a platinum-containing chemotherapy in the neoadjuvant setting. In some embodiments, the subjects that can be treated in the methods provided herein include subjects whose cancers progressed or relapsed following a platinum-containing chemotherapy in the adjuvant setting. In some embodiments, the subjects that can be treated in the methods provided herein include subjects whose cancers progressed or relapsed following a platinum-containing chemotherapy in the neoadjuvant, locally advanced setting. In some embodiments, the subjects that can be treated in the methods provided herein include subjects whose cancers progressed or relapsed following a platinum-containing chemotherapy in the neoadjuvant, metastatic setting. In some embodiments, the subjects that can be treated in the methods provided herein include subjects whose cancers progressed or relapsed following a platinum-containing chemotherapy in the adjuvant, locally advanced setting. In some embodiments, the subjects that can be treated in the methods provided herein include subjects whose cancers progressed or relapsed following a platinum-containing chemotherapy in the adjuvant, metastatic setting. In some embodiments, the subjects that can be treated in the methods provided herein include subjects whose cancers progressed or relapsed following a platinum-containing chemotherapy in metastatic setting. In some embodiments, the subjects that can be treated in the methods provided herein include subjects whose cancers progressed or relapsed following a platinum-containing chemotherapy in the locally advanced setting.

In some specific embodiments, the subjects that can be treated in the methods provided herein include subjects whose cancers progressed or relapsed following a therapy with an inhibitor of PD-1 and a platinum-containing chemotherapy. In other specific embodiments, the subjects that can be treated in the methods provided herein include subjects whose cancers progressed or relapsed following a therapy with an inhibitor of PD-L1 and a platinum-containing chemotherapy. In some specific embodiments, the subjects that can be treated in the methods provided herein include subjects whose cancer progressed or relapsed following a therapy with an inhibitor of PD-1 and a platinum-containing chemotherapy in the neoadjuvant setting. In some specific embodiments, the subjects that can be treated in the methods provided herein include subjects whose cancer progressed or relapsed following a therapy with an inhibitor of PD-1 and a platinum-containing chemotherapy in the adjuvant setting. In some specific embodiments, the subjects that can be treated in the methods provided herein include subjects whose cancer progressed or relapsed following a therapy with an inhibitor of PD-1 and a platinum-containing chemotherapy in the locally advanced setting. In some specific embodiments, the subjects that can be treated in the methods provided herein include subjects whose cancer progressed or relapsed following a therapy with an inhibitor of PD-1 and a platinum-containing chemotherapy in the metastatic setting. In some specific embodiments, the subjects that can be treated in the methods provided herein include subjects whose cancer progressed or relapsed following a therapy with an inhibitor of PD-1 and a platinum-containing chemotherapy in the neoadjuvant, locally advanced setting. In some specific embodiments, the subjects that can be treated in the methods provided herein include subjects whose cancer progressed or relapsed following a therapy with an inhibitor of PD-1 and a platinum-containing chemotherapy in the neoadjuvant, metastatic setting. In some specific embodiments, the subjects that can be treated in the methods provided herein include subjects whose cancer progressed or relapsed following a therapy with an inhibitor of PD-1 and a platinum-containing chemotherapy in the adjuvant, locally advanced setting. In some specific embodiments, the subjects that can be treated in the methods provided herein include subjects whose cancer progressed or relapsed following a therapy with an inhibitor of PD-1 and a platinum-containing chemotherapy in the adjuvant, metastatic setting.

In some specific embodiments, the subjects that can be treated in the methods provided herein include subjects whose cancer progressed or relapsed following a therapy with an inhibitor of PD-L1 and a platinum-containing chemotherapy in the neoadjuvant setting. In some specific embodiments, the subjects that can be treated in the methods provided herein include subjects whose cancer progressed or relapsed following a therapy with an inhibitor of PD-L1 and a platinum-containing chemotherapy in the adjuvant setting. In some specific embodiments, the subjects that can be treated in the methods provided herein include subjects whose cancer progressed or relapsed following a therapy with an inhibitor of PD-L1 and a platinum-containing chemotherapy in the locally advanced setting. In some specific embodiments, the subjects that can be treated in the methods provided herein include subjects whose cancer progressed or relapsed following a therapy with an inhibitor of PD-L1 and a platinum-containing chemotherapy in the metastatic setting. In some specific embodiments, the subjects that can be treated in the methods provided herein include subjects whose cancer progressed or relapsed following a therapy with an inhibitor of PD-L1 and a platinum-containing chemotherapy in the neoadjuvant, locally advanced setting. In some specific embodiments, the subjects that can be treated in the methods provided herein include subjects whose cancer progressed or relapsed following a therapy with an inhibitor of PD-L1 and a platinum-containing chemotherapy in the neoadjuvant, metastatic setting. In some specific embodiments, the subjects that can be treated in the methods provided herein include subjects whose cancer progressed or relapsed following a therapy with an inhibitor of PD-L1 and a platinum-containing chemotherapy in the adjuvant, locally advanced setting. In some specific embodiments, the subjects that can be treated in the methods provided herein include subjects whose cancer progressed or relapsed following a therapy with an inhibitor of PD-L1 and a platinum-containing chemotherapy in the adjuvant, metastatic setting.

In certain embodiments, the subjects that can be treated in the methods provided herein include those whose cancers have progressed or relapsed other treatments for cancers within 1, 2, 3, 4, 5, 6, 7, 8, 9, 10, 11, 12, 13, 14, 15, 16, 17, 18, 19, 20, 21, 22, 23, or 24 months after the other treatments, including for example and not by way of limitation, any or any combination of the treatments described in the preceding 13 paragraphs. In some particular embodiment, the cancers in the subjects have progressed or relapsed within 6 months after the platinum-based therapy. In further embodiments, the cancers in the subjects have progressed or relapsed within 12 months after a platinum-based therapy.

In some embodiments, the subjects that can be treated in the methods provided herein have certain phenotypic or genotypic characteristics. In some embodiments, the subjects have any permutation and combination of the phenotypic or genotypic characteristics described herein.

In some embodiments, the phenotypic or genotypic characteristics are determined histologically, cytologically, or both histologically and cytologically. In some embodiments of methods provided herein, the histological and/or the cytological determination of the phenotypic and/or genotypic characteristics are performed as described in American Society of Clinical Oncology/College of American Pathologists (ASCO/CAP) guidelines based on the most recently analyzed tissue, which is incorporated herein in their entirety by reference. In some embodiments, the phenotypic or genotypic characteristics are determined by sequencing including the next generation sequencing (e.g. NGS from Illumina, Inc), DNA hybridization, and/or RNA hybridization.

In some embodiments, the one or more other treatments for cancer, which the subjects have received or from which the cancers of the subjects have progressed or relapsed, are a PD-1 inhibitor or a PD-L1 inhibitor. In certain embodiments, the PD-1 inhibitor is pembrolizumab. In certain embodiments, the PD-1 inhibitor is nivolumab. In other embodiments, the PD-L1 inhibitor is atezolizumab. In some embodiments, the PD-L1 inhibitor is avelumab. In certain embodiments, the PD-L1 inhibitor is durvalumab. Other examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In certain embodiment, the PD-1 inhibitor is an anti-PD-1 antibody. In one embodiment, the anti-PD-1 antibody is BGB-A317, nivolumab (also known as ONO-4538, BMS-936558, or MDX1106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™ In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity.

In further embodiment, the PD-L1 inhibitor is an anti-PD-L1 antibody. In one embodiment, the anti-PD-L1 antibody is MEDI4736 (durvalumab). In one embodiment, the anti-PD-L1 antibody is avelumab (BAVENCIO®). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®).

In some embodiments, the subjects that can be treated in the methods provided herein is a mammal. In some embodiments, the subjects that can be treated in the methods provided herein is a human.

### 5.2.2 Methods of Treatment Including Dose Modification Based on Hyperglycemia and/or Blood Glucose Levels

The disclosure further provides that the ADC dose administered for treating the cancer in the subject can be modified based on certain criteria, for example the hyperglycemia in the subject. In some embodiments, the subject treated with the methods provided herein has hyperglycemia. In some specific embodiments, the subject treated with the methods provided herein has diabetic ketoacidosis (DKA). In other specific embodiments, the subject treated with the methods provided herein has conditions that increase the risks for hyperglycemia, such as higher body mass index and/or higher baseline A1C.

Hyperglycemia can be assessed based on the blood glucose levels. In some embodiments, the methods provided herein further comprises (b) determining blood glucose level in the subject, and (c) if the blood glucose level from (b) is higher than 250 mg/dL, withholding the administration of the antibody drug conjugate. In certain embodiments, the methods provided herein further comprises (d) waiting for a period sufficient for the blood glucose level to reduce to no more than 250 mg/dL. In certain further embodiments, the methods provided herein further comprises (e) determining blood glucose level in the subject, and (f) if the blood glucose level from (e) is no more than 250 mg/dL, administering to the subject a second regimen comprising an effective amount of the antibody drug conjugate.

The disclosure provides that under certain criteria of severe adverse events in the subject, the administration of the ADC for the cancer treatment should be discontinued permanently. In some embodiments of the methods provided herein, if the blood glucose level determined in any of the method steps is more than 500 mg/dL, including, for example, in step (b) or (e) determining blood glucose level in the subject described in the preceding paragraph, the administration of the ADC is permanently discontinued. In certain embodiments, if the blood glucose level determined is more than 500 mg/dL, the administration of the ADC is permanently discontinued regardless any other criteria.

The disclosure provides that the method steps for the dose modification based on the criteria of blood glucose can be iterated. The disclosure further provides that the method steps for the dose modification based on the criteria of blood glucose can be iterated according to the rules set forth and provided herein. In some embodiments of the methods provided herein, the method steps (a) to (f) can be repeated, which are (a) administering to the subject a first regimen comprising an effective amount of an ADC, (b) determining blood glucose level in the subject, (c) if the blood glucose level from (b) is higher than 250 mg/dL, withholding the administration of the antibody drug conjugate, (d) waiting for a period sufficient for the blood glucose level to reduce to no more than 250 mg/dL, (e) determining blood glucose level in the subject, and (f) if the blood glucose level from (e) is no more than 250 mg/dL, administering to the subject a second regimen comprising an effective amount of the antibody drug conjugate. In some embodiments of the methods provided herein, the method steps (a), (b), (c), (e) and (f) can be repeated, which are (a) administering to the subject a first regimen comprising an effective amount of an ADC, (b) determining blood glucose level in the subject, (c) if the blood glucose level from (b) is higher than 250 mg/dL, withholding the administration of the antibody drug conjugate, (e) determining blood glucose level in the subject, and (f) if the blood glucose level from (e) is no more than 250 mg/dL, administering to the subject a second regimen comprising an effective amount of the antibody drug conjugate. In some embodiments of the methods provided herein, the method steps (b), (c), (d), (e) and (f) can be repeated, which are (b) determining blood glucose level in the subject, (c) if the blood glucose level from (b) is higher than 250 mg/dL, withholding the administration of the antibody drug conjugate, (d) waiting for a period sufficient for the blood glucose level to reduce to no more than 250 mg/dL, (e) determining blood glucose level in the subject, and (f) if the blood glucose level from (e) is no more than 250 mg/dL, administering to the subject a second regimen comprising an effective amount of the antibody drug conjugate. In some embodiments of the methods provided herein, the method steps (b), (c), (e) and (f) can be repeated, which are (b) determining blood glucose level in the subject, (c) if the blood glucose level from (b) is higher than 250 mg/dL, withholding the administration of the antibody drug conjugate, (e) determining blood glucose level in the subject, and (f) if the blood glucose level from (e) is no more than 250 mg/dL, administering to the subject a second regimen comprising an effective amount of the antibody drug conjugate.

The disclosure provides that the ADC dose in the second regimen based on the blood glucose level is kept identical as the ADC dose in the first regimen. In some embodiments, when the second regimen is administered following the returning of the blood glucose level to no more than 250 mg/dL, the ADC dose in the second regimen is identical to the ADC dose in the first regimen. In some specific embodiments, when the second regimen is administered following the returning of the blood glucose to no more than 250 mg/dL, the ADC dose in the second regimen is about 1.25 milligram/kilogram (mg/kg) of the subject's body weight for a subject having a body weight of less than 100 kg or about 125 mg to a subject having a body weight of no less than 100 kg.

Alternatively, the hyperglycemia as the dose modification criteria as described in the preceding paragraphs can be determined based on the CTCAE Grading v4.0 as set forth in National Cancer Institute: Common Terminology Criteria for Adverse Events (CTCAE) version 4.03. https://evs.nci.nih.gov/ftp1/CTCAE/CTCAE_4.03/CTCAE_4.03_2010-06-14_QuickReference_5×7.pdf, which is incorporated hereby in its entirety by reference. Accordingly, hyperglycemia can be categorized as 5 grades indicated in the following Table 4

TABLE 4
Common Terminology Criteria for Adverse Events (CTCAE)
Grading v4.0 for Hyperglycemia

| | Grade 1 | Grade 2 | Grade 3 | Grade 4 | Grade 5 |
|---|---|---|---|---|---|
| Fasting | ULN—160 | 160-250 | 250-500 | Over 500 | Death |
| glucose | mg/dL | mg/dL | mg/dL | mg/dL | |
| value | ULN—8.9 | 8.9-13.9 | 13.9-27.8 | Over 27.8 | |
| | mmol/L | mmol/L | mmol/L | mmol/L | |
| | | | Hospitalization | Life- | |
| | | | indicated | threatening | |
| | | | | consequences | |

ULN: Upper limit of normal

Alternatively, in some embodiments, the grade of hyperglycemia is determined according to a scale in which Grade 1 is mild, Grade 2 is moderate, Grade 3 is severe, and Grade 4 is life-threatening.

Based on the hyperglycemia grade in Table 4, in some embodiments, the methods provided herein further comprises (b′) determining hyperglycemia, e.g. hyperglycemia grade, in the subject, and (c′) if the hyperglycemia grade from (b′) is no less than Grade 3, withholding the administration of the antibody drug conjugate. In certain embodiments, the methods provided herein further comprises (d′) waiting for a period sufficient for the hyperglycemia to reduce to no more than Grade 2. In some further embodiments, the methods provided herein further comprises (e′) determining hyperglycemia, e.g. hyperglycemia grade, in the subject, and (f) if the hyperglycemia from (e′) is no more than Grade 2, administering to the subject a second regimen comprising an effective amount of the antibody drug conjugate.

Similarly, under certain criteria of severe hyperglycemia in the subject, the administration of the ADC for the cancer treatment should be discontinued permanently. In some embodiments of the methods provided herein, if the hyperglycemia determined in any of the method steps is no less than Grad 4, including, for example, in step (b′) or (e′) determining hyperglycemia, e.g. hyperglycemia grade, in the subject described in the preceding paragraph, the administration of the ADC is permanently discontinued. In certain embodiments, if the hyperglycemia is no less than Grad 4, the administration of the ADC is permanently discontinued regardless any other criteria.

The method steps for the dose modification based on the criteria of hyperglycemia can also be iterated. The disclosure further provides that the method steps for the dose modification based on the criteria of hyperglycemia can be iterated according to the rules set forth and provided herein. In some embodiments of the methods provided herein, the method steps (a), (b′), (c′), (d′), (e′) and (f) as described above can be repeated. In some embodiments of the methods provided herein, the method steps (a), (b′), (c′), (e′) and (f) as described above can be repeated. In some embodiments of the methods provided herein, the method steps (b′), (c′), (d′), (e′) and (f) as described above can be repeated. In some embodiments of the methods provided herein, the method steps (b′), (c′), (e′) and (f) as described above can be repeated.

The disclosure provides that the ADC dose in the second regimen based on hyperglycemia is kept identical as the ADC dose in the first regimen. In some embodiments, when the second regimen is administered following the returning of the hyperglycemia to no more than Grade 2, the ADC dose in the second regimen is identical to the ADC dose in the first regimen. In some specific embodiments, when the second regimen is administered following the returning of the hyperglycemia to no more Grade 2, the ADC dose in the second regimen is about 1.25 milligram/kilogram (mg/kg) of the subject's body weight for a subject having a body weight of less than 100 kg or about 125 mg to a subject having a body weight of no less than 100 kg.

Based on some embodiments provided herein and described above, the dose modification schemes for the methods provided herein based on blood glucose level and/or hyperglycemia are summarized in the Table 5 below:

TABLE 5
dose modification schemes for the methods provided herein
based on blood glucose level and/or hyperglycemia

| Grade 1 | Grade 2 | Grade 3 | Grade 4 |
|---|---|---|---|
| Continue at | Continue at | Withhold treatment. | Discontinue |
| same dose | same dose | Resume treatment at the same | treatment* |
| level | level | dose level once hyperglycemia/ | |
| | | elevated blood glucose has | |
| | | improved to ≤ Grade 2 and | |
| | | patient is clinically and | |
| | | metabolically stable. | |

*Patients with blood glucose >500 mg/dL (Grade 4) considered unrelated to ADC treatment may continue dosing once the patient's blood glucose has improved to ≤250 mg/dL (≤ Grade 2) and the patient is clinically and metabolically stable.

As described in the Table 5 above, in some embodiments, the administration of the ADC does not need to be discontinued permanently when the hyperglycemia determined in any of the method steps is no less than Grad 4, if the subject with Grade 4 hyperglycemia is considered unrelated to the ADC treatment and the subject's blood glucose has improved to ≤250 mg/dL (or hyperglycemia improved to no more than Grade 2) and the patient is clinically and metabolically stable. As described in the Table 5 above, in some embodiments, the administration of the ADC does not need to be discontinued permanently when the blood glucose determined in any of the method steps is more than 500 mg/dL, if the subject with Grade 4 hyperglycemia is considered unrelated to the ADC treatment and the subject's blood glucose has improved to ≤250 mg/dL or hyperglycemia improved to no more than Grade 2) and the patient is clinically and metabolically stable. In some embodiments, the administration of the ADC can resume at the same dose level as that in the first regimen even after the hyperglycemia determined in any of the method steps is no less than Grad 4, if the subject with Grade 4 hyperglycemia is considered unrelated to the ADC treatment and if the subject's blood glucose has improved to ≤250 mg/dL (or hyperglycemia has improved to no more than Grade 2) and the patient is clinically and metabolically stable. In some embodiments, the administration of the ADC can resume at the same dose level as that in the first regimen even after the blood glucose level determined in any of the method steps is more than 500 mg/dL, if the blood glucose level is considered unrelated to the ADC and if the subject's blood glucose has improved to ≤250 mg/dL (or hyperglycemia has improved to no more than Grade 2) and the patient is clinically and metabolically stable.

In some embodiments of the methods provided herein, the time period sufficient for the hyperglycemia to improve to no more than Grade 2 or blood glucose to reduce to no more than 250 mg/dL is 1 to 10 days. In some embodiments of the methods provided herein, the time period sufficient for the hyperglycemia to improve to no more than Grade 2 or blood glucose to reduce to no more than 250 mg/dL is 1 to 10 weeks. In some embodiments of the methods provided herein, the time period sufficient for the hyperglycemia to improve to no more than Grade 2 or blood glucose to reduce to no more than 250 mg/dL is 1 to 4 months. In some embodiments of the methods provided herein, the time period sufficient for the hyperglycemia to improve to no more than Grade 2 or blood glucose to reduce to no more than 250 mg/dL is 1 day. In some embodiments of the methods provided herein, the time period sufficient for the hyperglycemia to improve to no more than Grade 2 or blood glucose to reduce to no more than 250 mg/dL is 2 days. In some embodiments of the methods provided herein, the time period sufficient for the hyperglycemia to improve to no more than Grade 2 or blood glucose to reduce to no more than 250 mg/dL is 3 days. In some embodiments of the methods provided herein, the time period sufficient for the hyperglycemia to improve to no more than Grade 2 or blood glucose to reduce to no more than 250 mg/dL is 4 days. In some embodiments of the methods provided herein, the time period sufficient for the hyperglycemia to improve to no more than Grade 2 or blood glucose to reduce to no more than 250 mg/dL is 5 days. In some embodiments of the methods provided herein, the time period sufficient for the hyperglycemia to improve to no more than Grade 2 or blood glucose to reduce to no more than 250 mg/dL is 6 days. In some embodiments of the methods provided herein, the time period sufficient for the hyperglycemia to improve to no more than Grade 2 or blood glucose to reduce to no more than 250 mg/dL is 7 days. In some embodiments of the methods provided herein, the time period sufficient for the hyperglycemia to improve to no more than Grade 2 or blood glucose to reduce to no more than 250 mg/dL is 8 days. In some embodiments of the methods provided herein, the time period sufficient for the hyperglycemia to improve to no more than Grade 2 or blood glucose to reduce to no more than 250 mg/dL is 9 days. In some embodiments of the methods provided herein, the time period sufficient for the hyperglycemia to improve to no more than Grade 2 or blood glucose to reduce to no more than 250 mg/dL is 10 days. In some embodiments of the methods provided herein, the time period sufficient for the hyperglycemia to improve to no more than Grade 2 or blood glucose to reduce to no more than 250 mg/dL is 1 week. In some embodiments of the methods provided herein, the time period sufficient for the hyperglycemia to improve to no more than Grade 2 or blood glucose to reduce to no more than 250 mg/dL is 2 weeks. In some embodiments of the methods provided herein, the time period sufficient for the hyperglycemia to improve to no more than Grade 2 or blood glucose to reduce to no more than 250 mg/dL is 3 weeks. In some embodiments of the methods provided herein, the time period sufficient for the hyperglycemia to improve to no more than Grade 2 or blood glucose to reduce to no more than 250 mg/dL is 4 weeks. In some embodiments of the methods provided herein, the time period sufficient for the hyperglycemia to improve to no more than Grade 2 or blood glucose to reduce to no more than 250 mg/dL is 5 weeks. In some embodiments of the methods provided herein, the time period sufficient for the hyperglycemia to improve to no more than Grade 2 or blood glucose to reduce to no more than 250 mg/dL is 6 weeks. In some embodiments of the methods provided herein, the time period sufficient for the hyperglycemia to improve to no more than Grade 2 or blood glucose to reduce to no more than 250 mg/dL is 7 weeks. In some embodiments of the methods provided herein, the time period sufficient for the hyperglycemia to improve to no more than Grade 2 or blood glucose to reduce to no more than 250 mg/dL is 8 weeks. In some embodiments of the methods provided herein, the time period sufficient for the hyperglycemia to improve to no more than Grade 2 or blood glucose to reduce to no more than 250 mg/dL is 1 month. In some embodiments of the methods provided herein, the time period sufficient for the hyperglycemia to improve to no more than Grade 2 or blood glucose to reduce to no more than 250 mg/dL is 2 months. In some embodiments of the methods provided herein, the time period sufficient for the hyperglycemia to improve to no more than Grade 2 or blood glucose to reduce to no more than 250 mg/dL is 3 months. In some embodiments of the methods provided herein, the time period sufficient for the hyperglycemia to improve to no more than Grade 2 or blood glucose to reduce to no more than 250 mg/dL is 4 months.

The disclosure provides that the blood glucose levels and the hyperglycemia in the methods provided herein can be determined at various frequencies and intervals according to the need of the methods and the practice of the art. In some embodiments of the methods provided herein, the blood glucose level is determined daily. In some embodiments of the methods provided herein, the blood glucose level is determined once every two days, once every three days, once every four days, or once every five days, once every six days. In some embodiments of the methods provided herein, the blood glucose level is determined weekly, bi-weekly, once every three weeks, or once every four weeks. In some embodiments of the methods provided herein, the blood glucose level is determined monthly, once every two months, or once every three months. In some embodiments of the methods provided herein, the hyperglycemia is determined daily. In some embodiments of the methods provided herein, the hyperglycemia is determined once every two days, once every three days, once every four days, or once every five days, once every six days. In some embodiments of the methods provided herein, the hyperglycemia is determined weekly, bi-weekly, once every three weeks, or once every four weeks. In some embodiments of the methods provided herein, the hyperglycemia is determined monthly, once every two months, or once every three months.

### 5.2.3 Methods of Treatment Including Dose Modification Based on Peripheral Neuropathy

The disclosure also provides that the ADC dose administered for treating the cancer in the subject can be modified based on other criteria, for example peripheral neuropathy in the subject. In some embodiments, the subject treated with the methods provided herein has peripheral neuropathy. In some embodiments, the subject treated with the methods provided herein has peripheral sensory neuropathy. In some embodiments, the subject treated with the methods provided herein has peripheral motor neuropathy. In some embodiments, the subject treated with the methods provided herein has peripheral sensorimotor neuropathy. In some embodiments, peripheral neuropathy is used as the criteria for modifying the ADC dose. In some embodiments, peripheral sensory neuropathy is used as the criteria for modifying the ADC dose. In some embodiments, peripheral motor neuropathy is used as the criteria for modifying the ADC dose. In some embodiments, peripheral sensorimotor neuropathy is used as the criteria for modifying the ADC dose. In some embodiments, peripheral neuropathy, predominantly peripheral sensory neuropathy, is used as the criteria for modifying the ADC dose. In some embodiments, peripheral neuropathy, about 50% of which is peripheral sensory neuropathy, is used as the criteria for modifying the ADC dose. In some embodiments, peripheral neuropathy, 49% of which is peripheral sensory neuropathy, is used as the criteria for modifying the ADC dose.

Peripheral neuropathy, including peripheral motor neuropathy, peripheral sensory neuropathy, and/or peripheral sensorimotor neuropathy, as the dose modification criteria can be determined based on the CTCAE Grading v4.0 as set forth in National Cancer Institute: Common Terminology Criteria for Adverse Events (CTCAE) version 4.03. https://evs.nci.nih.gov/ftp1/CTCAE/CTCAE_4.03/CTCAE_4.03_2010-06-14_QuickReference_5×7.pdf, which is incorporated hereby in its entirety by reference. Accordingly, peripheral neuropathy can be categorized as 5 grades indicated in the following Table 6

TABLE 6
Common Terminology Criteria for Adverse Events (CTCAE) Grading v4.0 for
Peripheral Neuropathy*

| Adverse | | | | | |
|---|---|---|---|---|---|
| Event | Grade 1 | Grade 2 | Grade 3 | Grade 4 | Grade 5 |
| Peripheral | Asymptomatic; | Moderate | Severe | Life- | Death |
| Motor | clinical or | symptoms; | symptoms; | threatening | |
| Neuropathy | diagnostic | limiting | limiting self | consequences; | |
| | observations | instrumental | care ADL; | urgent | |
| | only; | ADL | assistive device | intervention | |
| | intervention | | indicated | indicated | |
| | not indicated | | | | |
| Peripheral | Asymptomatic; | Moderate | Severe | Life- | Death |
| Sensory | loss of deep | symptoms; | symptoms; | threatening | |
| Neuropathy | tendon reflexes | limiting | limiting self | consequences; | |
| | or paresthesia | instrumental | care ADL | urgent | |
| | | ADL | | intervention | |
| | | | | indicated | |

*peripheral neuropathy is a grouped term and includes: hypoesthesia, gait disturbance, muscular weakness, neuralgia, paresthesia, peripheral motor neuropathy, peripheral sensory neuropathy and/or peripheral sensorimotor neuropathy.
ADL: Activities of daily living

Alternatively, in some embodiments, the grade of peripheral neuropathy, including for example the grade for peripheral motor neuropathy, peripheral sensory neuropathy, and/or peripheral sensorimotor neuropathy, is determined according to a scale in which Grade 1 is mild, Grade 2 is moderate, Grade 3 is severe, and Grade 4 is life-threatening.

Based on the peripheral neuropathy grade, for example the peripheral neuropathy grade as set forth in Table 6, in some embodiments, the methods provided herein further comprises (g) determining peripheral neuropathy in the subject, and (h) if the peripheral neuropathy from (g) is no less than Grade 2, withholding the administration of the antibody drug conjugate. In certain embodiments, the methods provided herein further comprises (i) waiting for a period sufficient for the peripheral neuropathy to reduce to no more than Grade 1. In some further embodiments, the methods provided herein further comprises (j) determining peripheral neuropathy in the subject, and (k) if the peripheral neuropathy (j) is no more than Grade 1, administering to the subject a second regimen comprising an effective amount of the ADC, wherein the second regimen comprises an ADC dose equal to or lower than the first regimen.

The disclosure provides that under certain criteria of severe adverse events in the subject, such as Grade 3 or higher Grade peripheral neuropathy, the administration of the ADC for the cancer treatment is discontinued permanently. In some embodiments of the methods provided herein, if the peripheral neuropathy from (g) or (j) is no less than Grade 3, the administration of the ADC is discontinued permanently. In certain embodiments, if the peripheral neuropathy is no less than Grade 3, the administration of the ADC is permanently discontinued regardless any other criteria.

The disclosure provides that the method steps for the dose modification based on the criteria of peripheral neuropathy can be iterated. The disclosure further provides that the method steps for the dose modification based on the criteria of peripheral neuropathy can be iterated according to the rules set forth and provided herein. In some embodiments of the methods provided herein, the method steps (a), (g), (h), (i), (j), and (k) can be repeated, which are (a) administering to the subject a first regimen comprising an effective amount of an ADC, (g) determining peripheral neuropathy in the subject, (h) if the peripheral neuropathy from (g) is no less than Grade 2, withholding the administration of the antibody drug conjugate, (i) waiting for a period sufficient for the peripheral neuropathy to reduce to no more than Grade 1, (j) determining peripheral neuropathy in the subject, and (k) if the peripheral neuropathy (j) is no more than Grade 1, administering to the subject a second regimen comprising an effective amount of the ADC, wherein the second regimen comprises an ADC dose equal to or lower than the first regimen. In some embodiments of the methods provided herein, the method steps (a), (g), (h), (j), and (k) can be repeated, which are (a) administering to the subject a first regimen comprising an effective amount of an ADC, (g) determining peripheral neuropathy in the subject, (h) if the peripheral neuropathy from (g) is no less than Grade 2, withholding the administration of the antibody drug conjugate, (j) determining peripheral neuropathy in the subject, and (k) if the peripheral neuropathy (j) is no more than Grade 1, administering to the subject a second regimen comprising an effective amount of the ADC, wherein the second regimen comprises an ADC dose equal to or lower than the first regimen. In some embodiments of the methods provided herein, the method steps (g), (h), (i), (j), and (k) can be repeated, which are (g) determining peripheral neuropathy in the subject, (h) if the peripheral neuropathy from (g) is no less than Grade 2, withholding the administration of the antibody drug conjugate, (i) waiting for a period sufficient for the peripheral neuropathy to reduce to no more than Grade 1, (j) determining peripheral neuropathy in the subject, and (k) if the peripheral neuropathy (j) is no more than Grade 1, administering to the subject a second regimen comprising an effective amount of the ADC, wherein the second regimen comprises an ADC dose equal to or lower than the first regimen. In some embodiments of the methods provided herein, the method steps (g), (h), (j), and (k) can be repeated, which are (g) determining peripheral neuropathy in the subject, (h) if the peripheral neuropathy from (g) is no less than Grade 2, withholding the administration of the antibody drug conjugate, (j) determining peripheral neuropathy in the subject, and (k) if the peripheral neuropathy (j) is no more than Grade 1, administering to the subject a second regimen comprising an effective amount of the ADC, wherein the second regimen comprises an ADC dose equal to or lower than the first regimen.

In some embodiments of the methods provided herein, the time period sufficient for the peripheral neuropathy to reduce to no more than Grade 1 is 1 to 10 days. In some embodiments of the methods provided herein, the time period sufficient for the peripheral neuropathy to reduce to no more than Grade 1 is 1 to 10 weeks. In some embodiments of the methods provided herein, the time period sufficient for the peripheral neuropathy to reduce to no more than Grade 1 is 1 to 4 months. In some embodiments of the methods provided herein, the time period sufficient for the peripheral neuropathy to reduce to no more than Grade 1 is 1 day. In some embodiments of the methods provided herein, the time period sufficient for the peripheral neuropathy to reduce to no more than Grade 1 is 2 days. In some embodiments of the methods provided herein, the time period sufficient for the peripheral neuropathy to reduce to no more than Grade 1 is 3 days. In some embodiments of the methods provided herein, the time period sufficient for the peripheral neuropathy to reduce to no more than Grade 1 is 4 days. In some embodiments of the methods provided herein, the time period sufficient for the peripheral neuropathy to reduce to no more than Grade 1 is 5 days. In some embodiments of the methods provided herein, the time period sufficient for the peripheral neuropathy to reduce to no more than Grade 1 is 6 days. In some embodiments of the methods provided herein, the time period sufficient for the peripheral neuropathy to reduce to no more than Grade 1 is 7 days. In some embodiments of the methods provided herein, the time period sufficient for the peripheral neuropathy to reduce to no more than Grade 1 is 8 days. In some embodiments of the methods provided herein, the time period sufficient for the peripheral neuropathy to reduce to no more than Grade 1 is 9 days. In some embodiments of the methods provided herein, the time period sufficient for the peripheral neuropathy to reduce to no more than Grade 1 is 10 days. In some embodiments of the methods provided herein, the time period sufficient for the peripheral neuropathy to reduce to no more than Grade 1 is 1 week. In some embodiments of the methods provided herein, the time period sufficient for the peripheral neuropathy to reduce to no more than Grade 1 is 2 weeks. In some embodiments of the methods provided herein, the time period sufficient for the peripheral neuropathy to reduce to no more than Grade 1 is 3 weeks. In some embodiments of the methods provided herein, the time period sufficient for the peripheral neuropathy to reduce to no more than Grade 1 is 4 weeks. In some embodiments of the methods provided herein, the time period sufficient for the peripheral neuropathy to reduce to no more than Grade 1 is 5 weeks. In some embodiments of the methods provided herein, the time period sufficient for the peripheral neuropathy to reduce to no more than Grade 1 is 6 weeks. In some embodiments of the methods provided herein, the time period sufficient for the peripheral neuropathy to reduce to no more than Grade 1 is 7 weeks. In some embodiments of the methods provided herein, the time period sufficient for the peripheral neuropathy to reduce to no more than Grade 1 is 8 weeks. In some embodiments of the methods provided herein, the time period sufficient for the peripheral neuropathy to reduce to no more than Grade 1 is 1 month. In some embodiments of the methods provided herein, the time period sufficient for the peripheral neuropathy to reduce to no more than Grade 1 is 2 months. In some embodiments of the methods provided herein, the time period sufficient for the peripheral neuropathy to reduce to no more than Grade 1 is 3 months. In some embodiments of the methods provided herein, the time period sufficient for the peripheral neuropathy to reduce to no more than Grade 1 is 4 months.

The disclosure provides that the modified dose can depend on the number of times the condition for the administration of the second regimen based on the criteria of peripheral neuropathy have been satisfied. As such, in some embodiments, the methods further comprises determining the number of times the condition for the administration of the second regimen based on the criteria of peripheral neuropathy have been satisfied. The disclosure provides that the ADC dose can be modified according to the scheme described in Table 7 below:

TABLE 7
dose modification schemes for the methods provided herein
based on peripheral neuropathy*

| Grade 1 | Grade 2 | Grade 3 | Grade 4 |
|---|---|---|---|
| Continue at | Withhold dose until toxicity is | Discontinue | Discontinue |
| same dose | ≤ Grade 1 or has returned to | treatment | treatment |
| level | baseline, then resume | | |
| | treatment at the same | | |
| | dose level. For the second | | |
| | occurrence of Grade 2 | | |
| | neuropathy, withhold | | |
| | dose until toxicity is ≤ | | |
| | Grade 1, then reduce the | | |
| | dose by 1 dose | | |
| | level and resume treatment. | | |

*See e.g. Rosenberg JE, et al. J Clin Oncol. 2019; 37:2592-2600; Rosenberg JE, et al. J Clin Oncol. 2019; 37:2592-2600 (Protocol), both of which are hereby incorporated in their entireties by reference.

The dose reduction or modification referenced to in the Table 8 above and the paragraphs related to peripheral neuropathy above and below, are set forth as follows:

TABLE 8
Starting Dose and Dose Reduction Schedule *

| | ADC Dose Level |
|---|---|
| Starting dose | 1.25 mg/kg for subject having a body weight of less than 100 kg or 125 |
| | mg for subject having a body weight of no less than 100 kg |
| First dose reduction | 1.0 mg/kg for subject having a body weight of less than 100 kg or 100 |
| | mg for subject having a body weight of no less than 100 kg |
| Second dose reduction | 0.75 mg/kg for subject having a body weight of less than 100 kg or 75 |
| | mg for subject having a body weight of no less than 100 kg |
| Third dose reduction | 0.5 mg/kg for subject having a body weight of less than 100 kg or 50 |
| | mg for subject having a body weight of no less than 100 kg |

* Patients requiring a dose reduction may be re-escalated by 1 dose level (e.g., patients reduced to 0.75 mg/kg may only be re-escalated to 1 mg/kg) provided the toxicity does not require study drug discontinuation and has returned to baseline or ≤ Grade 1. If the toxicity recurs, re-escalation will not be permitted.

In some embodiments of the methods provided herein, the ADC dose in the first regimen is the starting dose before any dose reduction or dose modification based on peripheral neuropathy. In some embodiments, such ADC dose in the first regimen and the starting dose is 1.25 mg/kg for subject having a body weight of less than 100 kg or 125 mg for subject having a body weight of no less than 100 kg.

As is clear from the description and Table 7 and Table 8 above, in some embodiments of the methods provided herein, if the second regimen is administered for the first time in (k), the second regimen can be identical to the first regimen. In some embodiments of the methods provided herein, the second regimen in (k) can be identical to the first regimen when the second regimen is administered for the first time or has been administered one or more times in (k). In some embodiments of the methods provided herein, if the second regimen has been administered once in (k) and the subject has a body weight of less than 100 kg, the ADC dose in the second regimen is lowered to about 1.0 mg/kg of the subject's body weight. In some embodiments of the methods provided herein, if the second regimen has been administered once in (k) and the subject has a body weight of no less than 100 kg, the ADC dose in the second regimen is lowered to about 100 mg to the subject. In some embodiments of the methods provided herein, if the second regimen has been administered twice in (k) and the subject has a body weight of less than 100 kg, the ADC dose in the second regimen is lowered to about 0.75 mg/kg of the subject's body weight. In some embodiments of the methods provided herein, if the second regimen has been administered twice in (k) and the subject has a body weight of no less than 100 kg, the ADC dose in the second regimen is lowered to about 75 mg to the subject. In some embodiments of the methods provided herein, if the second regimen has been administered three times in (k) and the subject has a body weight of less than 100 kg, the ADC dose in the second regimen is lowered to about 0.5 mg/kg of the subject's body weight. In some embodiments of the methods provided herein, if the second regimen has been administered three times in (k) and the subject has a body weight of no less than 100 kg, the ADC dose in the second regimen is lowered to about 50 mg to the subject.

The disclosure provides that requiring a dose reduction may be re-escalated by 1 dose level according to Table 8 (e.g., patients reduced to 0.75 mg/kg may only be re-escalated to 1 mg/kg) if the peripheral neuropathy does not require study drug discontinuation and the peripheral neuropathy has returned to baseline or ≤Grade 1. Accordingly, in some embodiments of the methods provided herein, the ADC dose in the second regimen is increased by an amount of about 0.25 mg/kg for the subject having a body weight of less than 100 kg or increased by an amount of about 25 mg for the subject having a body weight of no less than 100 kg, if (1) the administration of the ADC has not been discontinued permanently, (2) the ADC dose in the second regimen is lower than the ADC dose in the first regimen, and (3) the peripheral neuropathy has returned to no more than Grade 1. Specifically, in some embodiments of the methods provided herein, the ADC dose in the second regimen is increased from 0.5 mg/ml to 0.75 mg/ml for the subject having a body weight of less than 100 kg or increased 50 mg to 75 mg for the subject having a body weight of no less than 100 kg, if (1) the administration of the ADC has not been discontinued permanently, (2) the ADC dose in the second regimen is lower than the ADC dose in the first regimen, and (3) the peripheral neuropathy has returned to no more than Grade 1. In some embodiments of the methods provided herein, the ADC dose in the second regimen is increased from 0.75 mg/ml to 1 mg/ml for the subject having a body weight of less than 100 kg or increased 75 mg to 100 mg for the subject having a body weight of no less than 100 kg, if (1) the administration of the ADC has not been discontinued permanently, (2) the ADC dose in the second regimen is lower than the ADC dose in the first regimen, and (3) the peripheral neuropathy has returned to no more than Grade 1. In some embodiments of the methods provided herein, the ADC dose in the second regimen is increased from 1 mg/ml to 1.25 mg/ml for the subject having a body weight of less than 100 kg or increased 100 mg to 125 mg for the subject having a body weight of no less than 100 kg, if (1) the administration of the ADC has not been discontinued permanently, (2) the ADC dose in the second regimen is lower than the ADC dose in the first regimen, and (3) the peripheral neuropathy has returned to no more than Grade 1.

The disclosure provides that the peripheral neuropathy, including peripheral motor neuropathy, peripheral sensory neuropathy, and/or peripheral sensorimotor neuropathy, in the methods provided herein, can be determined at various frequencies and intervals according to the need of the methods and/or the practice of the art. In some embodiments of the methods provided herein, the peripheral neuropathy is determined daily. In some embodiments of the methods provided herein, the peripheral neuropathy is determined once every two days, once every three days, once every four days, or once every five days, once every six days. In some embodiments of the methods provided herein, the peripheral neuropathy is determined weekly, bi-weekly, once every three weeks, or once every four weeks. In some embodiments of the methods provided herein, the peripheral neuropathy is determined monthly, once every two months, or once every three months. In some embodiments of the methods provided herein, the peripheral sensory neuropathy is determined daily. In some embodiments of the methods provided herein, the peripheral sensory neuropathy is determined once every two days, once every three days, once every four days, or once every five days, once every six days. In some embodiments of the methods provided herein, the peripheral sensory neuropathy is determined weekly, bi-weekly, once every three weeks, or once every four weeks. In some embodiments of the methods provided herein, the peripheral sensory neuropathy is determined monthly, once every two months, or once every three months. In some embodiments of the methods provided herein, the peripheral motor neuropathy is determined daily. In some embodiments of the methods provided herein, the peripheral motor neuropathy is determined once every two days, once every three days, once every four days, or once every five days, once every six days. In some embodiments of the methods provided herein, the peripheral motor neuropathy is determined weekly, bi-weekly, once every three weeks, or once every four weeks. In some embodiments of the methods provided herein, the peripheral motor neuropathy is determined monthly, once every two months, or once every three months. In some embodiments of the methods provided herein, the peripheral sensorimotor neuropathy is determined daily. In some embodiments of the methods provided herein, the peripheral sensorimotor neuropathy is determined once every two days, once every three days, once every four days, or once every five days, once every six days. In some embodiments of the methods provided herein, the peripheral sensorimotor neuropathy is determined weekly, bi-weekly, once every three weeks, or once every four weeks. In some embodiments of the methods provided herein, the peripheral sensorimotor neuropathy is determined monthly, once every two months, or once every three months.

### 5.2.4 Methods of Treatment Including Dose Modification Based on Skin Reaction

The disclosure also provides that the ADC dose administered for treating the cancer in the subject can be modified based on other criteria, for example a skin reaction in the subject. In some embodiments, the subject treated with the methods provided herein has a skin reaction. In some embodiments, the subject treated with the methods provided herein has one or more skin reactions. In some embodiments, the subject treated with the methods provided herein has maculopapular rash. In some embodiments, the subject treated with the methods provided herein has pruritus. In some embodiments, the subject treated with the methods provided herein has symmetrical drug-related intertriginous. In some embodiments, the subject treated with the methods provided herein has flexural exanthema (SDRIFE). In some embodiments, the subject treated with the methods provided herein has bullous dermatitis. In some embodiments, the subject treated with the methods provided herein has exfoliative dermatitis. In some embodiments, the subject treated with the methods provided herein has dermatitis. In some embodiments, the subject treated with the methods provided herein has palmar-plantar erythrodysesthesia. In some embodiments, the subject treated with the methods provided herein has rash pustula. In some embodiments, the subject treated with the methods provided herein has rash acneiform. In some embodiments, the subject treated with the methods provided herein has papulopustular rash. In some embodiments, the subject treated with the methods provided herein has dry skin. In some embodiments, the subject treated with the methods provided herein has any permutation or combination of one or more of the skin reactions selected from the group consisting of maculopapular rash, pruritus, symmetrical drug-related intertriginous, flexural exanthema (SDRIFE), bullous dermatitis, exfoliative dermatitis, palmar-plantar erythrodysesthesia, rash pustula, rash acneiform, papulopustular rash, and dry skin.

In some embodiments, a skin reaction is used as the criteria for modifying the ADC dose in the methods provided herein. In some embodiments, one or more skin reactions are used as the criteria for modifying the ADC dose in the methods provided herein. In some embodiments, maculopapular rash is used as the criteria for modifying the ADC dose in the methods provided herein. In some embodiments, pruritus is used as the criteria for modifying the ADC dose in the methods provided herein. In some embodiments, symmetrical drug-related intertriginous is used as the criteria for modifying the ADC dose in the methods provided herein. In some embodiments, flexural exanthema (SDRIFE) is used as the criteria for modifying the ADC dose in the methods provided herein. In some embodiments, bullous dermatitis is used as the criteria for modifying the ADC dose in the methods provided herein. In some embodiments, exfoliative dermatitis is used as the criteria for modifying the ADC dose in the methods provided herein. In some embodiments, dermatitis is used as the criteria for modifying the ADC dose in the methods provided herein. In some embodiments, palmar-plantar erythrodysesthesia is used as the criteria for modifying the ADC dose in the methods provided herein. In some embodiments, rash pustula is used as the criteria for modifying the ADC dose in the methods provided herein. In some embodiments, rash acneiform is used as the criteria for modifying the ADC dose in the methods provided herein. In some embodiments, papulopustular rash is used as the criteria for modifying the ADC dose in the methods provided herein. In some embodiments, dry skin is used as the criteria for modifying the ADC dose in the methods provided herein. In some embodiments, any permutation or combination of one or more of the skin reactions selected from the group consisting of maculopapular rash, pruritus, symmetrical drug-related intertriginous, SDRIFE, bullous dermatitis, exfoliative dermatitis, palmar-plantar erythrodysesthesia, rash pustula, rash acneiform, papulopustular rash, and dry skin, are used as the criteria for modifying the ADC dose in the methods provided herein.

Skin reactions, including maculopapular rash, pruritus, symmetrical drug-related intertriginous, SDRIFE, bullous dermatitis, exfoliative dermatitis, palmar-plantar erythrodysesthesia, rash pustula, rash acneiform, papulopustular rash, and/or dry skin, as the dose modification criteria can be determined based on the Grade scale as set forth in National Cancer Institute: Common Terminology Criteria for Adverse Events (CTCAE) version 4.03. https://evs.nci.nih.gov/ftp_1/CTCAE/CTCAE_4.03/CTCAE_4.03_2010-06-14_QuickReference_5×7.pdf, which is incorporated hereby in its entirety by reference. Accordingly, skin reactions can be categorized as 5 grades indicated in the following Table 9

TABLE 9
CTCAE Definition and Grading v4.0 for skin reactions

| Adverse Event | Grade 1 | Grade 2 | Grade 3 | Grade 4 | Grade 5 |
|---|---|---|---|---|---|
| Rash | Macule s/papules | Macule s/papules | Macule s/papules | — | — |
| maculopapular | covering <10% | covering 10- | covering >30% BSA | | |
| | BSA with or | 30% BSA with or | with or without | | |
| | without | without | associated | | |
| | symptoms (e.g., | symptoms (e.g., | symptoms; limiting | | |
| | pruritus, burning, | pruritus, burning, | self care ADL | | |
| | tightness) | tightness); | | | |
| | | limiting | | | |
| | | instrumental ADL | | | |
| Rash acneiform | Papules and/or | Papules and/or | Papules and/or | Papules and/or | Death |
| or | pustules covering | pustules covering | pustules covering | pustules | |
| Papulopustular | <10% BSA, | 10-30% BSA, | >30% BSA, which | covering any | |
| rash | which may or | which may or | may or may not be | % BSA, which | |
| | may not be | may not be | associated with | may or may | |
| | associated with | associated with | symptoms of pruritus | not be | |
| | symptoms of | symptoms of | or tenderness; | associated with | |
| | pruritus or | pruritus or | limiting self care | symptoms of | |
| | tenderness | tenderness; | ADL; associated with | pruritus or | |
| | | associated with | local superinfection | tenderness and | |
| | | psychosocial | with oral antibiotics | are associated | |
| | | impact; limiting | indicated | with extensive | |
| | | instrumental ADL | | superinfection | |
| | | | | with IV | |
| | | | | antibiotics | |
| | | | | indicated; life | |
| | | | | threatening | |
| | | | | consequences | |
| Rash pustular | — | Localized; local | IV antibiotic, | — | — |
| | | intervention | antifungal, or | | |
| | | indicated (e.g., | antiviral intervention | | |
| | | topical antibiotic, | indicated; radiologic | | |
| | | antifungal, or | or operative | | |
| | | antiviral) | intervention | | |
| | | | indicated | | |
| Dry skin | Covering <10% | Covering 10- | Covering >30% BSA | | |
| | BSA and not | 30% BSA and | and associated with | | |
| | associated with | associated with | pruritus; limiting self | | |
| | erythema or | erythema or | care ADL | | |
| | pruritis | pruritus; limiting | | | |
| | | instrumental ADL | | | |
| Pruritis | Mild or localized; | Intense or | Intense or | | |
| | topical | widespread; | widespread; constant; | | |
| | intervention | intermittent; skin | limiting self care | | |
| | indicated | changes from | ADL or sleep; oral | | |
| | | scratching (e.g., | corticosteroid or | | |
| | | edema, | immunosuppressive | | |
| | | papulation, | therapy indicated | | |
| | | excoriations, | | | |
| | | lichenification, | | | |
| | | oozing/crusts); | | | |
| | | oral intervention | | | |
| | | indicated; | | | |
| | | limiting | | | |
| | | instrumental ADL | | | |

ADL: Activities of daily living

Alternatively, in some embodiments, the grade of skin reactions, including for example the grade for maculopapular rash, pruritus, symmetrical drug-related intertriginous, SDRIFE, bullous dermatitis, exfoliative dermatitis, palmar-plantar erythrodysesthesia, rash pustula, rash acneiform, papulopustular rash, and/or dry skin, is determined according to a scale in which Grade 1 is mild, Grade 2 is moderate, Grade 3 is severe, and Grade 4 is life-threatening.

Based on the skin reaction grade, for example the skin reaction grade described in Table 9, in some embodiments, the methods provided herein further comprises (l) determining a skin reaction in the subject, and (m) if the skin reaction from (l) is no less than Grade 3, withholding the administration of the ADC. In certain embodiments, the methods provided herein further comprises (n) waiting for a period sufficient for the skin reaction to reduce to no more than Grade 1. In some further embodiments, the methods provided herein comprises (o) determining the skin reaction in the subject, and (p) if the skin reaction in (o) is no more than Grade 1, administering to the subject a second regimen comprising an effective amount of the ADC, wherein the second regimen comprises an ADC dose equal to or lower than the first regimen.

The disclosure provides that under certain criteria of severe adverse events in the subject, such as skin reactions no less than Grade 4, the administration of the ADC for the cancer treatment is discontinued permanently. In some embodiments of the methods provided herein, if the skin reactions from the method step (l) or (o) as described above is no less than Grade 4, the administration of the ADC is discontinued permanently. In certain embodiments, if the skin reactions is no less than Grade 4, the administration of the ADC is permanently discontinued regardless any other criteria.

In other embodiments of the methods provided herein, if a Grade 3 skin reaction occurs more than once in the methods, the administration of the ADC for the cancer treatment is discontinued permanently. In other embodiments of the methods provided herein, if a Grade 3 skin reaction occurs more than once in the method step (l) or (o), the administration of the ADC for the cancer treatment is discontinued permanently. In some embodiments of the methods provided herein, if the Grade 3 skin reaction reoccurs in the method step (l) or (o), the administration of the ADC is discontinued permanently. In some embodiments of the methods provided herein, if the Grade 3 skin reaction reoccurs in the methods, the administration of the ADC is discontinued permanently. In certain embodiments, if a Grade 3 skin reaction occurs more than once, the administration of the ADC is permanently discontinued regardless any other criteria.

The disclosure provides that the method steps for the dose modification based on the criteria of skin reactions can be iterated. The disclosure further provides that the method steps for the dose modification based on the criteria of skin reactions can be iterated according to the rules set forth and provided herein. In some embodiments of the methods provided herein, the method steps (a), (l), (m), (n), (o), and (p) can be repeated, which are (a) administering to the subject a first regimen comprising an effective amount of an ADC, (l) determining a skin reaction in the subject, (m) if the skin reaction from (l) is no less than Grade 3, withholding the administration of the ADC, (n) waiting for a period sufficient for the skin reaction to reduce to no more than Grade 1, (o) determining the skin reaction in the subject, and (p) if the skin reaction in (o) is no more than Grade 1, administering to the subject a second regimen comprising an effective amount of the ADC, wherein the second regimen comprises an ADC dose equal to or lower than the first regimen. In some embodiments of the methods provided herein, the method steps (a), (l), (m), (o), and (p) can be repeated, which are (a) administering to the subject a first regimen comprising an effective amount of an ADC, (l) determining a skin reaction in the subject, (m) if the skin reaction from (l) is no less than Grade 3, withholding the administration of the ADC, (o) determining the skin reaction in the subject, and (p) if the skin reaction in (o) is no more than Grade 1, administering to the subject a second regimen comprising an effective amount of the ADC, wherein the second regimen comprises an ADC dose equal to or lower than the first regimen. In some embodiments of the methods provided herein, the method steps (l), (m), (n), (o), and (p) can be repeated, which are (l) determining a skin reaction in the subject, (m) if the skin reaction from (l) is no less than Grade 3, withholding the administration of the ADC, (n) waiting for a period sufficient for the skin reaction to reduce to no more than Grade 1, (o) determining the skin reaction in the subject, and (p) if the skin reaction in (o) is no more than Grade 1, administering to the subject a second regimen comprising an effective amount of the ADC, wherein the second regimen comprises an ADC dose equal to or lower than the first regimen. In some embodiments of the methods provided herein, the method steps (l), (m), (o), and (p) can be repeated, which are (l) determining a skin reaction in the subject, (m) if the skin reaction from (l) is no less than Grade 3, withholding the administration of the ADC, (o) determining the skin reaction in the subject, and (p) if the skin reaction in (o) is no more than Grade 1, administering to the subject a second regimen comprising an effective amount of the ADC, wherein the second regimen comprises an ADC dose equal to or lower than the first regimen.

In some embodiments of the methods provided herein, the time period sufficient for the skin reaction to reduce to no more than Grade 1 is 1 to 10 days. In some embodiments of the methods provided herein, the time period sufficient for the skin reaction to reduce to no more than Grade 1 is 1 to 10 weeks. In some embodiments of the methods provided herein, the time period sufficient for the skin reaction to reduce to no more than Grade 1 is 1 to 4 months. In some embodiments of the methods provided herein, the time period sufficient for the skin reaction to reduce to no more than Grade 1 is 1 day. In some embodiments of the methods provided herein, the time period sufficient for the skin reaction to reduce to no more than Grade 1 is 2 days. In some embodiments of the methods provided herein, the time period sufficient for the skin reaction to reduce to no more than Grade 1 is 3 days. In some embodiments of the methods provided herein, the time period sufficient for the skin reaction to reduce to no more than Grade 1 is 4 days. In some embodiments of the methods provided herein, the time period sufficient for the skin reaction to reduce to no more than Grade 1 is 5 days. In some embodiments of the methods provided herein, the time period sufficient for the skin reaction to reduce to no more than Grade 1 is 6 days. In some embodiments of the methods provided herein, the time period sufficient for the skin reaction to reduce to no more than Grade 1 is 7 days. In some embodiments of the methods provided herein, the time period sufficient for the skin reaction to reduce to no more than Grade 1 is 8 days. In some embodiments of the methods provided herein, the time period sufficient for the skin reaction to reduce to no more than Grade 1 is 9 days. In some embodiments of the methods provided herein, the time period sufficient for the skin reaction to reduce to no more than Grade 1 is 10 days. In some embodiments of the methods provided herein, the time period sufficient for the skin reaction to reduce to no more than Grade 1 is 1 week. In some embodiments of the methods provided herein, the time period sufficient for the skin reaction to reduce to no more than Grade 1 is 2 weeks. In some embodiments of the methods provided herein, the time period sufficient for the skin reaction to reduce to no more than Grade 1 is 3 weeks. In some embodiments of the methods provided herein, the time period sufficient for the skin reaction to reduce to no more than Grade 1 is 4 weeks. In some embodiments of the methods provided herein, the time period sufficient for the skin reaction to reduce to no more than Grade 1 is 5 weeks. In some embodiments of the methods provided herein, the time period sufficient for the skin reaction to reduce to no more than Grade 1 is 6 weeks. In some embodiments of the methods provided herein, the time period sufficient for the skin reaction to reduce to no more than Grade 1 is 7 weeks. In some embodiments of the methods provided herein, the time period sufficient for the skin reaction to reduce to no more than Grade 1 is 8 weeks. In some embodiments of the methods provided herein, the time period sufficient for the skin reaction to reduce to no more than Grade 1 is 1 month. In some embodiments of the methods provided herein, the time period sufficient for the skin reaction to reduce to no more than Grade 1 is 2 months. In some embodiments of the methods provided herein, the time period sufficient for the skin reaction to reduce to no more than Grade 1 is 3 months. In some embodiments of the methods provided herein, the time period sufficient for the skin reaction to reduce to no more than Grade 1 is 4 months.

The disclosure provides that the modified dose can depend on the number of times the condition for the administration of the second regimen based on the criteria of skin reactions have been satisfied. As such, in some embodiments, the methods further comprises determining the number of times the condition for the administration of the second regimen based on the criteria of skin reactions have been satisfied. The disclosure provides that the ADC dose can be modified according to the scheme described in Table 10 below:

TABLE 10
dose modification schemes for the methods
provided herein based on skin reactions¹

| Grade 1² | Grade 2² | Grade 3 | Grade 4 |
|---|---|---|---|
| Continue at | Continue at | Withhold until Grade ≤1 | Discontinue |
| same dose | same dose | or has returned to | treatment |
| level | level. | baseline, then resume | |
| | | treatment at the same | |
| | | dose level or consider | |
| | | dose reduction by 1 | |
| | | dose level | |
| | | Discontinue treatment for | |
| | | recurrent Grade 3 | |
| | | skin reactions ³ | |

¹See e.g. Rosenberg JE, et al. J C in Oncol. 2019; 37:2592-2600; Rosenberg JE, et a . J Clin Oncol. 2019; 37:2592-2600 (Protocol), both of which are hereby incorporated in their entireties by reference.
²Mild rash related to ADC treatment can be treated using local supportive care as ne eded. Topical corticosteroids have been used along with antihistamines for pruritus as needed.
³Grade 3 rash that is not limiting self-care activities of daily living or associated with infection requiring systemic antibiotics does not require treatment interruption, provided symptoms are not severe and can be managed with supportive treatment.

In some embodiments, the dose reduction or modification referenced to in the Table 10 above and the paragraphs related to skin reaction above and below, are set forth in Table 8 above.

In some embodiments of the methods provided herein, the ADC dose in the first regimen is the starting dose before any dose reduction or dose modification based on skin reactions. Based on Table 8, in some embodiments, such ADC dose in the first regimen and the starting dose is 1.25 mg/kg for subject having a body weight of less than 100 kg or 125 mg for subject having a body weight of no less than 100 kg.

As is clear from the description and Table 10 and Table 8 above, in some embodiments of the methods provided herein, if the second regimen is administered for the first time in (p), the second regimen can be identical to the first regimen. In some embodiments of the methods provided herein, the second regimen in (p) can be identical to the first regimen when the second regimen is administered for the first time or has been administered one or more times in (p). In some embodiments of the methods provided herein, if the second regimen has been administered one or more times in (p) and the subject has a body weight of less than 100 kg, the ADC dose in the second regimen is lowered to about 1.0 mg/kg of the subject's body weight. In some embodiments of the methods provided herein, if the second regimen has been administered one or more times in (p) and the subject has a body weight of no less than 100 kg, the ADC dose in the second regimen is lowered to about 100 mg to the subject. In some embodiments of the methods provided herein, if the second regimen has been administered two or more times in (p) and the subject has a body weight of less than 100 kg, the ADC dose in the second regimen is lowered to about 0.75 mg/kg of the subject's body weight. In some embodiments of the methods provided herein, if the second regimen has been administered two or more times in (p) and the subject has a body weight of no less than 100 kg, the ADC dose in the second regimen is lowered to about 75 mg to the subject. In some embodiments of the methods provided herein, if the second regimen has been administered three or more times in (p) and the subject has a body weight of less than 100 kg, the ADC dose in the second regimen is lowered to about 0.5 mg/kg of the subject's body weight. In some embodiments of the methods provided herein, if the second regimen has been administered three or more times in (p) and the subject has a body weight of no less than 100 kg, the ADC dose in the second regimen is lowered to about 50 mg to the subject.

Alternatively, according to Table 10 and Table 8 above, in some embodiments of the methods provided herein and based on the criteria of skin reactions, if the subject has a body weight of less than 100 kg, the ADC dose in the second regimen is about 1.0 mg/kg of the subject's body weight. In some embodiments of the methods provided herein and based on the criteria of skin reactions, if the subject has a body weight of no less than 100 kg, the ADC dose in the second regimen is about 100 mg to the subject. In some embodiments of the methods provided herein and based on the criteria of skin reactions, if the subject has a body weight of less than 100 kg, the ADC dose in the second regimen is about 1.0 mg/kg of the subject's body weight when the second regimen is administered for the first time or has been administered one or more times. In some embodiments of the methods provided herein and based on the criteria of skin reactions, if the subject has a body weight of no less than 100 kg, the ADC dose in the second regimen is about 100 mg to the subject when the second regimen is administered for the first time or has been administered one or more times. In some embodiments of the methods provided herein, if the second regimen has been administered one or more times in (p) and the subject has a body weight of less than 100 kg, the ADC dose in the second regimen is lowered to about 0.75 mg/kg of the subject's body weight. In some embodiments of the methods provided herein, if the second regimen has been administered one or more times in (p) and the subject has a body weight of no less than 100 kg, the ADC dose in the second regimen is lowered to about 75 mg to the subject. In some embodiments of the methods provided herein, if the second regimen has been administered two or more times in (p) and the subject has a body weight of less than 100 kg, the ADC dose in the second regimen is lowered to about 0.5 mg/kg of the subject's body weight. In some embodiments of the methods provided herein, if the second regimen has been administered two or more times in (p) and the subject has a body weight of no less than 100 kg, the ADC dose in the second regimen is lowered to about 50 mg to the subject.

The disclosure provides that requiring a dose reduction may be re-escalated by 1 dose level according to Table 8 (e.g., patients reduced to 0.75 mg/kg may only be re-escalated to 1 mg/kg) if the skin reaction does not require study drug discontinuation and the skin reaction has returned to baseline or ≤Grade 1. Accordingly, in some embodiments of the methods provided herein, the ADC dose in the second regimen is increased by an amount of about 0.25 mg/kg for the subject having a body weight of less than 100 kg or increased by an amount of about 25 mg for the subject having a body weight of no less than 100 kg, if (1) the administration of the ADC has not been discontinued permanently, (2) the ADC dose in the second regimen is lower than the ADC dose in the first regimen, and (3) the skin reaction has returned to no more than Grade 1. Specifically, in some embodiments of the methods provided herein, the ADC dose in the second regimen is increased from 0.5 mg/ml to 0.75 mg/ml for the subject having a body weight of less than 100 kg or increased 50 mg to 75 mg for the subject having a body weight of no less than 100 kg, if (1) the administration of the ADC has not been discontinued permanently, (2) the ADC dose in the second regimen is lower than the ADC dose in the first regimen, and (3) the skin reaction has returned to no more than Grade 1. In some embodiments of the methods provided herein, the ADC dose in the second regimen is increased from 0.75 mg/ml to 1 mg/ml for the subject having a body weight of less than 100 kg or increased 75 mg to 100 mg for the subject having a body weight of no less than 100 kg, if (1) the administration of the ADC has not been discontinued permanently, (2) the ADC dose in the second regimen is lower than the ADC dose in the first regimen, and (3) the skin reaction has returned to no more than Grade 1. In some embodiments of the methods provided herein, the ADC dose in the second regimen is increased from 1 mg/ml to 1.25 mg/ml for the subject having a body weight of less than 100 kg or increased 100 mg to 125 mg for the subject having a body weight of no less than 100 kg, if (1) the administration of the ADC has not been discontinued permanently, (2) the ADC dose in the second regimen is lower than the ADC dose in the first regimen, and (3) the skin reaction has returned to no more than Grade 1.

The disclosure provides that the skin reaction, including maculopapular rash, pruritus, symmetrical drug-related intertriginous, flexural exanthema (SDRIFE), bullous dermatitis, exfoliative dermatitis, palmar-plantar erythrodysesthesia, rash pustula, rash acneiform, papulopustular rash, and/or dry skin, in the methods provided herein, can be determined at various frequencies and intervals according to the need of the methods and/or the practice of the art. In some embodiments of the methods provided herein, the skin reaction is determined daily. In some embodiments of the methods provided herein, the skin reaction is determined once every two days, once every three days, once every four days, or once every five days, once every six days. In some embodiments of the methods provided herein, the skin reaction is determined weekly, bi-weekly, once every three weeks, or once every four weeks. In some embodiments of the methods provided herein, the skin reaction is determined monthly, once every two months, or once every three months.

In some embodiments of the methods provided herein, any one or more of the specific skin reactions including for example, maculopapular rash, pruritus, symmetrical drug-related intertriginous, flexural exanthema (SDRIFE), bullous dermatitis, exfoliative dermatitis, palmar-plantar erythrodysesthesia, rash pustula, rash acneiform, papulopustular rash, and/or dry skin, are determined daily. In some embodiments of the methods provided herein, any one or more of the specific skin reactions including for example, maculopapular rash, pruritus, symmetrical drug-related intertriginous, flexural exanthema (SDRIFE), bullous dermatitis, exfoliative dermatitis, palmar-plantar erythrodysesthesia, rash pustula, rash acneiform, papulopustular rash, and/or dry skin, are determined once every two days, once every three days, once every four days, or once every five days, once every six days. In some embodiments of the methods provided herein, any one or more of the specific skin reactions including for example, maculopapular rash, pruritus, symmetrical drug-related intertriginous, flexural exanthema (SDRIFE), bullous dermatitis, exfoliative dermatitis, palmar-plantar erythrodysesthesia, rash pustula, rash acneiform, papulopustular rash, and/or dry skin, are determined weekly, bi-weekly, once every three weeks, or once every four weeks. In some embodiments of the methods provided herein, any one or more of the specific skin reactions including for example, maculopapular rash, pruritus, symmetrical drug-related intertriginous, flexural exanthema (SDRIFE), bullous dermatitis, exfoliative dermatitis, palmar-plantar erythrodysesthesia, rash pustula, rash acneiform, papulopustular rash, and/or dry skin, are determined monthly, once every two months, or once every three months.

### 5.2.5 Methods of Treatment Including Dose Modification Based on Non-Hematologic Toxicity

The disclosure also provides that the ADC dose administered for treating the cancer in the subject can be modified based on other criteria, for example non-hematologic toxicity in the subject. In some embodiments, the subject treated with the methods provided herein has non-hematologic toxicity. In some embodiments, the subject treated with the methods provided herein has one or more non-hematologic toxicities. In some embodiments, the subject treated with the methods provided herein has dysgeusia. In some embodiments, the subject treated with the methods provided herein has anorexia. In some embodiments, the subject treated with the methods provided herein has loss of appetite. In some embodiments, the subject treated with the methods provided herein has an ocular disorder. In some embodiments, the subject treated with the methods provided herein has punctate keratitis. In some embodiments, the subject treated with the methods provided herein has keratitis. In some embodiments, the subject treated with the methods provided herein has keratopathy. In some embodiments, the subject treated with the methods provided herein has limbal stem cell deficiency. In some embodiments, the subject treated with the methods provided herein has dry eye. In some embodiments, the subject treated with the methods provided herein has blurred vision. In some embodiments, the subject treated with the methods provided herein has any permutation or combination of one or more of the non-hematologic toxicities selected from the group consisting of dysgeusia, anorexia, loss of appetite, and ocular disorder. In some embodiments, the subject treated with the methods provided herein has any permutation or combination of one or more of the non-hematologic toxicities selected from the group consisting of dysgeusia, anorexia, loss of appetite, punctate keratitis, keratitis, keratopathy, limbal stem cell deficiency, dry eye, and blurred vision.

In some embodiments, non-hematologic toxicity is used as the criteria for modifying the ADC dose in the methods provided herein. In some embodiments, one or more non-hematologic toxicities are used as the criteria for modifying the ADC dose in the methods provided herein. In some embodiments, dysgeusia is used as the criteria for modifying the ADC dose in the methods provided herein. In some embodiments, anorexia is used as the criteria for modifying the ADC dose in the methods provided herein. In some embodiments, loss of appetite is used as the criteria for modifying the ADC dose in the methods provided herein. In some embodiments, an ocular disorder is used as the criteria for modifying the ADC dose in the methods provided herein. In some embodiments, punctate keratitis is used as the criteria for modifying the ADC dose in the methods provided herein. In some embodiments, keratitis is used as the criteria for modifying the ADC dose in the methods provided herein. In some embodiments, keratopathy is used as the criteria for modifying the ADC dose in the methods provided herein. In some embodiments, limbal stem cell deficiency is used as the criteria for modifying the ADC dose in the methods provided herein. In some embodiments, dry eye is used as the criteria for modifying the ADC dose in the methods provided herein. In some embodiments, blurred vision is used as the criteria for modifying the ADC dose in the methods provided herein. In some embodiments, any permutation or combination of one or more of the non-hematologic toxicities selected from the group consisting of dysgeusia, anorexia, loss of appetite, and ocular disorder are used as the criteria for modifying the ADC dose in the methods provided herein. In some embodiments, any permutation or combination of one or more of the non-hematologic toxicities selected from the group consisting of dysgeusia, anorexia, loss of appetite, punctate keratitis, keratitis, keratopathy, limbal stem cell deficiency, dry eye, and blurred vision are used as the criteria for modifying the ADC dose in the methods provided herein.

As is clear from the above description, in some embodiments, the non-hematologic toxicity includes, for example but not limited to, dysgeusia, anorexia, loss of appetite, and ocular disorder. In some embodiments, the ocular disorder (i.e. eye disorder) includes, for example but not limited to, punctate keratitis, keratitis, keratopathy, limbal stem cell deficiency, dry eye, and blurred vision. Accordingly, in certain embodiments, the non-hematologic toxicity includes, for example but not limited to, dysgeusia, anorexia, loss of appetite, punctate keratitis, keratitis, keratopathy, limbal stem cell deficiency, dry eye, and blurred vision. The non-hematologic toxicity including dysgeusia, anorexia, loss of appetite, and ocular disorder (i.e. eye disorder such as punctate keratitis, keratitis, keratopathy, limbal stem cell deficiency, dry eye, and blurred vision), as the dose modification criteria can be determined based on the Grade scale as set forth in National Cancer Institute: Common Terminology Criteria for Adverse Events (CTCAE) version 4.03. https://evs.nci.nih.gov/ftp1/CTCAE/CTCAE_4.03/CTCAE_4.03_2010-06-14_QuickReference_5×7.pdf, which is incorporated hereby in its entirety by reference. In some specific embodiments, the Grade for non-hematologic toxicity is determined as set forth in the following Table 11

TABLE 11
CTCAE Definition and Grading v4.0 for different kinds of Non-Hematologic Toxicity

| Non- | | | | | |
|---|---|---|---|---|---|
| Hematologic | | | | | |
| Toxicity | | | | | |
| (Adverse | | | | | |
| Event) | Grade 1 | Grade 2 | Grade 3 | Grade 4 | Grade 5 |
| Dysgeusia | Altered taste but | Altered taste with | N/A | N/A | N/A |
| | no change in diet | change in diet; | | | |
| | | noxious or | | | |
| | | unpleasant taste; | | | |
| | | loss of taste | | | |
| Anorexia | Loss of appetite | Oral intake | Associated with | Life- | Death |
| | without alteration | altered without | significant weight | threatening | |
| | in eating habits | significant weight | loss or malnutrition; | consequences; | |
| | | loss or | tube feeding or TPN | urgent | |
| | | malnutrition; oral | indicated | intervention | |
| | | nutritional | | indicated | |
| | | supplements | | | |
| | | indicated | | | |
| dry eye | Asymptomatic; | Symptomatic; | Decrease in visual | — | — |
| | clinical or | multiple agents | acuity (<20/40); | | |
| | diagnostic | indicated; | limiting self care | | |
| | observations | limiting | ADL | | |
| | only; mild | instrumental ADL | | | |
| | symptoms | | | | |
| | relieved by | | | | |
| | lubricants | | | | |
| blurred vision | Intervention not | Symptomatic; | Limiting self care | — | — |
| | indicated | limiting | ADL | | |
| | | instrumental | | | |
| | | ADL | | | |
| keratitis | — | Symptomatic; | Decline in vision | Perforation or | |
| | | medical | (worse than 20/40 | blindness | |
| | | intervention | but better than | (20/200 or | |
| | | indicated (e.g., | 20/200); limiting | worse) in the | |
| | | topical agents); | self care ADL | affected eye | |
| | | limiting | | | |
| | | instrumental | | | |
| | | ADL | | | |

ADL: Activities of daily living;
TPN: total parenteral nutrition

Alternatively, in some embodiments, the grade of non-hematologic toxicity, including for example the grade for dysgeusia, anorexia, loss of appetite, ocular disorder, punctate keratitis, keratitis, keratopathy, limbal stem cell deficiency, dry eye, and/or blurred vision, is determined according to a scale in which Grade 1 is mild, Grade 2 is moderate, Grade 3 is severe, and Grade 4 is life-threatening.

In some embodiments of the methods provided herein, the non-hematologic toxicity is dysgeusia. In some embodiments of the methods provided herein, the non-hematologic toxicity is anorexia. In some embodiments of the methods provided herein, the non-hematologic toxicity is loss of appetite. In some embodiments of the methods provided herein, the non-hematologic toxicity is ocular disorder. In some embodiments of the methods provided herein, the non-hematologic toxicity is punctate keratitis. In some embodiments of the methods provided herein, the non-hematologic toxicity is keratitis. In some embodiments of the methods provided herein, the non-hematologic toxicity is keratopathy. In some embodiments of the methods provided herein, the non-hematologic toxicity is limbal stem cell deficiency. In some embodiments of the methods provided herein, the non-hematologic toxicity is dry eye. In some embodiments of the methods provided herein, the non-hematologic toxicity is blurred vision.

Based on the non-hematologic toxicity grade, for example the non-hematologic toxicity grade described in Table 11, in some embodiments, the methods provided herein further comprises (q) determining non-hematologic toxicity in the subject, and (s) if the non-hematologic toxicity from (q) is no less than Grade 3, withholding the administration of the ADC. In certain embodiments, the methods provided herein further comprises (t) waiting for a period sufficient for the non-hematologic toxicity to reduce to no more than Grade 1. In some further embodiments, the methods provided herein comprises (u) determining the non-hematologic toxicity in the subject, and (v) if the non-hematologic toxicity in (u) is no more than Grade 1, administering to the subject a second regimen comprising an effective amount of the ADC, wherein the second regimen comprises an ADC dose equal to or lower than the first regimen.

The disclosure provides that under certain criteria of severe adverse events in the subject, such as non-hematologic toxicity no less than Grade 4, the administration of the ADC for the cancer treatment is discontinued permanently. In some embodiments of the methods provided herein, if the non-hematologic toxicity from the method step (q) or (u) as described above is no less than Grade 4, the administration of the ADC is discontinued permanently. In certain embodiments, if the non-hematologic toxicity is no less than Grade 4, the administration of the ADC is permanently discontinued regardless any other criteria.

The disclosure provides that the method steps for the dose modification based on the criteria of non-hematologic toxicity can be iterated. The disclosure further provides that the method steps for the dose modification based on the criteria of non-hematologic toxicity can be iterated according to the rules set forth and provided herein. In some embodiments of the methods provided herein, the method steps (a), (q), (s), (t), (u) and (v) can be repeated, which are (a) administering to the subject a first regimen comprising an effective amount of an ADC, (q) determining non-hematologic toxicity in the subject, (s) if the non-hematologic toxicity from (q) is no less than Grade 3, withholding the administration of the ADC, (t) waiting for a period sufficient for the non-hematologic toxicity to reduce to no more than Grade 1, (u) determining the non-hematologic toxicity in the subject, and (v) if the non-hematologic toxicity in (u) is no more than Grade 1, administering to the subject a second regimen comprising an effective amount of the ADC, wherein the second regimen comprises an ADC dose equal to or lower than the first regimen. In some embodiments of the methods provided herein, the method steps (a), (q), (s), (u) and (v) can be repeated, which are (a) administering to the subject a first regimen comprising an effective amount of an ADC, (q) determining non-hematologic toxicity in the subject, (s) if the non-hematologic toxicity from (q) is no less than Grade 3, withholding the administration of the ADC, (u) determining the non-hematologic toxicity in the subject, and (v) if the non-hematologic toxicity in (u) is no more than Grade 1, administering to the subject a second regimen comprising an effective amount of the ADC, wherein the second regimen comprises an ADC dose equal to or lower than the first regimen. In some embodiments of the methods provided herein, the method steps (q), (s), (t), (u) and (v) can be repeated, which are (q) determining non-hematologic toxicity in the subject, (s) if the non-hematologic toxicity from (q) is no less than Grade 3, withholding the administration of the ADC, (t) waiting for a period sufficient for the non-hematologic toxicity to reduce to no more than Grade 1, (u) determining the non-hematologic toxicity in the subject, and (v) if the non-hematologic toxicity in (u) is no more than Grade 1, administering to the subject a second regimen comprising an effective amount of the ADC, wherein the second regimen comprises an ADC dose equal to or lower than the first regimen. In some embodiments of the methods provided herein, the method steps (q), (s), (u) and (v) can be repeated, which are (q) determining non-hematologic toxicity in the subject, (s) if the non-hematologic toxicity from (q) is no less than Grade 3, withholding the administration of the ADC, (u) determining the non-hematologic toxicity in the subject, and (v) if the non-hematologic toxicity in (u) is no more than Grade 1, administering to the subject a second regimen comprising an effective amount of the ADC, wherein the second regimen comprises an ADC dose equal to or lower than the first regimen.

In some embodiments of the methods provided herein, the time period sufficient for the non-hematologic toxicity to reduce to no more than Grade 1 is 1 to 10 days. In some embodiments of the methods provided herein, the time period sufficient for the non-hematologic toxicity to reduce to no more than Grade 1 is 1 to 10 weeks. In some embodiments of the methods provided herein, the time period sufficient for the non-hematologic toxicity to reduce to no more than Grade 1 is 1 to 4 months. In some embodiments of the methods provided herein, the time period sufficient for the non-hematologic toxicity to reduce to no more than Grade 1 is 1 day. In some embodiments of the methods provided herein, the time period sufficient for the non-hematologic toxicity to reduce to no more than Grade 1 is 2 days. In some embodiments of the methods provided herein, the time period sufficient for the non-hematologic toxicity to reduce to no more than Grade 1 is 3 days. In some embodiments of the methods provided herein, the time period sufficient for the non-hematologic toxicity to reduce to no more than Grade 1 is 4 days. In some embodiments of the methods provided herein, the time period sufficient for the non-hematologic toxicity to reduce to no more than Grade 1 is 5 days. In some embodiments of the methods provided herein, the time period sufficient for the non-hematologic toxicity to reduce to no more than Grade 1 is 6 days. In some embodiments of the methods provided herein, the time period sufficient for the non-hematologic toxicity to reduce to no more than Grade 1 is 7 days. In some embodiments of the methods provided herein, the time period sufficient for the non-hematologic toxicity to reduce to no more than Grade 1 is 8 days. In some embodiments of the methods provided herein, the time period sufficient for the non-hematologic toxicity to reduce to no more than Grade 1 is 9 days. In some embodiments of the methods provided herein, the time period sufficient for the non-hematologic toxicity to reduce to no more than Grade 1 is 10 days. In some embodiments of the methods provided herein, the time period sufficient for the non-hematologic toxicity to reduce to no more than Grade 1 is 1 week. In some embodiments of the methods provided herein, the time period sufficient for the non-hematologic toxicity to reduce to no more than Grade 1 is 2 weeks. In some embodiments of the methods provided herein, the time period sufficient for the non-hematologic toxicity to reduce to no more than Grade 1 is 3 weeks. In some embodiments of the methods provided herein, the time period sufficient for the non-hematologic toxicity to reduce to no more than Grade 1 is 4 weeks. In some embodiments of the methods provided herein, the time period sufficient for the non-hematologic toxicity to reduce to no more than Grade 1 is 5 weeks. In some embodiments of the methods provided herein, the time period sufficient for the non-hematologic toxicity to reduce to no more than Grade 1 is 6 weeks. In some embodiments of the methods provided herein, the time period sufficient for the non-hematologic toxicity to reduce to no more than Grade 1 is 7 weeks. In some embodiments of the methods provided herein, the time period sufficient for the non-hematologic toxicity to reduce to no more than Grade 1 is 8 weeks. In some embodiments of the methods provided herein, the time period sufficient for the non-hematologic toxicity to reduce to no more than Grade 1 is 1 month. In some embodiments of the methods provided herein, the time period sufficient for the non-hematologic toxicity to reduce to no more than Grade 1 is 2 months. In some embodiments of the methods provided herein, the time period sufficient for the non-hematologic toxicity to reduce to no more than Grade 1 is 3 months. In some embodiments of the methods provided herein, the time period sufficient for the non-hematologic toxicity to reduce to no more than Grade 1 is 4 months.

The disclosure provides that the modified dose can depend on the number of times the condition for the administration of the second regimen based on the criteria of non-hematologic toxicity have been satisfied. As such, in some embodiments, the methods further comprises determining the number of times the condition for the administration of the second regimen based on the criteria of non-hematologic toxicity have been satisfied. The disclosure provides that the ADC dose can be modified according to the scheme described in Table 12 below:

TABLE 12
dose modification schemes for the methods provided
herein based on non-hematologic toxicity¹

| Grade 1 | Grade 2 | Grade 3 | Grade 4 |
|---|---|---|---|
| Continue at | Continue at | Withhold dose until toxicity | Discontinue |
| same dose | same dose | is ≤ Grade 1 or has | treatment |
| level | level. | returned to baseline, then | |
| | | resume treatment at the same | |
| | | dose level or consider | |
| | | dose reduction by 1 dose level. | |

¹See e.g. Rosenberg JE, et al. J C in Oncol. 2019; 37:2592-2600; Rosenberg JE, et a . J Clin Oncol. 2019; 37:2592-2600 (Protocol), both of which are hereby incorporated in their entireties by reference.

In some embodiments, the dose reduction or modification referenced to in the Table 12 above and the paragraphs related to non-hematologic toxicity above and below, are set forth in Table 8 above.

In some embodiments of the methods provided herein, the ADC dose in the first regimen is the starting dose before any dose reduction or dose modification based on non-hematologic toxicity. Based on Table 8, in some embodiments, such ADC dose in the first regimen and the starting dose is 1.25 mg/kg for subject having a body weight of less than 100 kg or 125 mg for subject having a body weight of no less than 100 kg.

As is clear from the description and Table 12 and Table 8 above, in some embodiments of the methods provided herein, if the second regimen is administered for the first time in (v), the second regimen can be identical to the first regimen. In some embodiments of the methods provided herein, the second regimen in (v) can be identical to the first regimen when the second regimen is administered for the first time or has been administered one or more times in (v). In some embodiments of the methods provided herein, if the second regimen has been administered one or more times in (v) and the subject has a body weight of less than 100 kg, the ADC dose in the second regimen is lowered to about 1.0 mg/kg of the subject's body weight. In some embodiments of the methods provided herein, if the second regimen has been administered one or more times in (v) and the subject has a body weight of no less than 100 kg, the ADC dose in the second regimen is lowered to about 100 mg to the subject. In some embodiments of the methods provided herein, if the second regimen has been administered two or more times in (v) and the subject has a body weight of less than 100 kg, the ADC dose in the second regimen is lowered to about 0.75 mg/kg of the subject's body weight. In some embodiments of the methods provided herein, if the second regimen has been administered two or more times in (v) and the subject has a body weight of no less than 100 kg, the ADC dose in the second regimen is lowered to about 75 mg to the subject. In some embodiments of the methods provided herein, if the second regimen has been administered three or more times in (v) and the subject has a body weight of less than 100 kg, the ADC dose in the second regimen is lowered to about 0.5 mg/kg of the subject's body weight. In some embodiments of the methods provided herein, if the second regimen has been administered three or more times in (v) and the subject has a body weight of no less than 100 kg, the ADC dose in the second regimen is lowered to about 50 mg to the subject.

Alternatively, according to Table 12 and Table 8 above, in some embodiments of the methods provided herein and based on the criteria of non-hematologic toxicity, if the subject has a body weight of less than 100 kg, the ADC dose in the second regimen is about 1.0 mg/kg of the subject's body weight in (v). In some embodiments of the methods provided herein and based on the criteria of non-hematologic toxicity, if the subject has a body weight of no less than 100 kg, the ADC dose in the second regimen is about 100 mg to the subject in (v). In some embodiments of the methods provided herein and based on the criteria of non-hematologic toxicity, if the subject has a body weight of less than 100 kg, the ADC dose in the second regimen is about 1.0 mg/kg of the subject's body weight when the second regimen is administered for the first time or has been administered one or more times in (v). In some embodiments of the methods provided herein and based on the criteria of non-hematologic toxicity, if the subject has a body weight of no less than 100 kg, the ADC dose in the second regimen is about 100 mg to the subject when the second regimen is administered for the first time or has been administered one or more times in (v). In some embodiments of the methods provided herein, if the second regimen has been administered one or more times in (v) and the subject has a body weight of less than 100 kg, the ADC dose in the second regimen is lowered to about 0.75 mg/kg of the subject's body weight. In some embodiments of the methods provided herein, if the second regimen has been administered one or more times in (v) and the subject has a body weight of no less than 100 kg, the ADC dose in the second regimen is lowered to about 75 mg to the subject. In some embodiments of the methods provided herein, if the second regimen has been administered two or more times in (v) and the subject has a body weight of less than 100 kg, the ADC dose in the second regimen is lowered to about 0.5 mg/kg of the subject's body weight. In some embodiments of the methods provided herein, if the second regimen has been administered two or more times in (v) and the subject has a body weight of no less than 100 kg, the ADC dose in the second regimen is lowered to about 50 mg to the subject.

The disclosure provides that requiring a dose reduction may be re-escalated by 1 dose level according to Table 8 (e.g., patients reduced to 0.75 mg/kg may only be re-escalated to 1 mg/kg) if the non-hematologic toxicity does not require study drug discontinuation and the non-hematologic toxicity has returned to baseline or ≤Grade 1. Accordingly, in some embodiments of the methods provided herein, the ADC dose in the second regimen is increased by an amount of about 0.25 mg/kg for the subject having a body weight of less than 100 kg or increased by an amount of about 25 mg for the subject having a body weight of no less than 100 kg, if (1) the administration of the ADC has not been discontinued permanently, (2) the ADC dose in the second regimen is lower than the ADC dose in the first regimen, and (3) the non-hematologic toxicity has returned to no more than Grade 1. Specifically, in some embodiments of the methods provided herein, the ADC dose in the second regimen is increased from 0.5 mg/ml to 0.75 mg/ml for the subject having a body weight of less than 100 kg or increased 50 mg to 75 mg for the subject having a body weight of no less than 100 kg, if (1) the administration of the ADC has not been discontinued permanently, (2) the ADC dose in the second regimen is lower than the ADC dose in the first regimen, and (3) the non-hematologic toxicity has returned to no more than Grade 1. In some embodiments of the methods provided herein, the ADC dose in the second regimen is increased from 0.75 mg/ml to 1 mg/ml for the subject having a body weight of less than 100 kg or increased 75 mg to 100 mg for the subject having a body weight of no less than 100 kg, if (1) the administration of the ADC has not been discontinued permanently, (2) the ADC dose in the second regimen is lower than the ADC dose in the first regimen, and (3) the non-hematologic toxicity has returned to no more than Grade 1. In some embodiments of the methods provided herein, the ADC dose in the second regimen is increased from 1 mg/ml to 1.25 mg/ml for the subject having a body weight of less than 100 kg or increased 100 mg to 125 mg for the subject having a body weight of no less than 100 kg, if (1) the administration of the ADC has not been discontinued permanently, (2) the ADC dose in the second regimen is lower than the ADC dose in the first regimen, and (3) the non-hematologic toxicity has returned to no more than Grade 1.

The disclosure provides that the non-hematologic toxicity, including dysgeusia, anorexia, loss of appetite, ocular disorder, punctate keratitis, keratitis, keratopathy, limbal stem cell deficiency, dry eye, and/or blurred vision, in the methods provided herein, can be determined at various frequencies and intervals according to the need of the methods and/or the practice of the art. In some embodiments of the methods provided herein, the non-hematologic toxicity is determined daily. In some embodiments of the methods provided herein, the non-hematologic toxicity is determined once every two days, once every three days, once every four days, or once every five days, once every six days. In some embodiments of the methods provided herein, the non-hematologic toxicity is determined weekly, bi-weekly, once every three weeks, or once every four weeks. In some embodiments of the methods provided herein, the non-hematologic toxicity is determined monthly, once every two months, or once every three months.

### 5.2.6 Methods of Treatment Including Dose Modification Based on Non-Hematologic Toxicity

The disclosure also provides that the ADC dose administered for treating the cancer in the subject can be modified based on other criteria, for example hematologic toxicity in the subject. In some embodiments, the subject treated with the methods provided herein has hematologic toxicity. In some embodiments, the subject treated with the methods provided herein has one or more hematologic toxicities. In some embodiments, the subject treated with the methods provided herein has anemia. In some embodiments, the subject treated with the methods provided herein has thrombocytopenia. In some embodiments, the subject treated with the methods provided herein has neutropenia. In some embodiments, the subject treated with the methods provided herein has febrile neutropenia. In some embodiments, the subject treated with the methods provided herein has any permutation or combination of one or more of the hematologic toxicities selected from the group consisting of anemia, thrombocytopenia, neutropenia and febrile neutropenia.

In some embodiments, hematologic toxicity is used as the criteria for modifying the ADC dose in the methods provided herein. In some embodiments, one or more hematologic toxicities are used as the criteria for modifying the ADC dose in the methods provided herein. In some embodiments, anemia is used as the criteria for modifying the ADC dose in the methods provided herein. In some embodiments, thrombocytopenia is used as the criteria for modifying the ADC dose in the methods provided herein. In some embodiments, neutropenia is used as the criteria for modifying the ADC dose in the methods provided herein. In some embodiments, febrile neutropenia is used as the criteria for modifying the ADC dose in the methods provided herein. In some embodiments, any permutation or combination of one or more of the hematologic toxicities selected from the group consisting of anemia, thrombocytopenia, neutropenia and febrile neutropenia are used as the criteria for modifying the ADC dose in the methods provided herein.

As is clear from the above description, in some embodiments, the hematologic toxicity includes, for example but not limited to, anemia, thrombocytopenia, neutropenia and febrile neutropenia. The hematologic toxicity, including anemia, thrombocytopenia, neutropenia and febrile neutropenia, as the dose modification criteria can be determined based on the Grade scale as set forth in National Cancer Institute: Common Terminology Criteria for Adverse Events (CTCAE) version 4.03. https://evs.nci.nih.gov/ftp1/CTCAE/CTCAE_4.03/CTCAE_4.03_2010-06-14_QuickReference_5×7.pdf, which is incorporated hereby in its entirety by reference. In some specific embodiments, the Grade for hematologic toxicity is determined as set forth in the following Table 13.

TABLE 13
CTCAE Definition and Grading v4.0 for different kinds of Hematologic Toxicity

| Hematologic | | | | | |
|---|---|---|---|---|---|
| Toxicity (Adverse | | | | | |
| Event) | Grade 1 | Grade 2 | Grade 3 | Grade 4 | Grade 5 |
| Febrile | | | ANC <1000/mm³ | Life- | Death |
| neutropenia | | | with a single | threatening | |
| | | | temperature of | consequences; | |
| | | | >38.3 degrees C. | urgent | |
| | | | (101 degrees F.) | intervention | |
| | | | or a sustained | indicated | |
| | | | temperature of | | |
| | | | >=38 degrees C. | | |
| | | | (100.4 degrees F.) | | |
| | | | for more than one | | |
| | | | hour. | | |
| Thrombocytopenia | <LLN − | <75,000 − | 50,000 − | <25,000/mm3; | — |
| | 75,000/mm³; or | 50,000/mm³; or | 25,000/mm³; or | or <25.0 × | |
| | <LLN − 75.0 × | <75.0 − 50.0 × | <50.0 − 25.0 × | 10⁹/L | |
| | 10⁹/L | 10⁹/L | 10⁹/L | | |
| Anemia | Hemoglobin | Hgb <10.0 − 8.0 | Hgb <8.0 g/dL; | Life- | Death |
| | (Hgb) <LLN − | g/dL; <6.2 − 4.9 | <4.9 mmol/L; | threatening | |
| | 10.0 g/dL; | mmol/L; <100 − | <80 g/L; | consequences; | |
| | <LLN − 6.2 | 80g/L | transfusion | urgent | |
| | mmol/L; <LLN − | | indicated | intervention | |
| | 100 g/L | | | indicated | |

LLN: lower limit of normal;
ANC: absoute neutrophil count;
Hgb: hemoglobin

Alternatively, in some embodiments, the grade of hematologic toxicity, including for example the grade for anemia, thrombocytopenia, neutropenia and febrile neutropenia, is determined according to a scale in which Grade 1 is mild, Grade 2 is moderate, Grade 3 is severe, and Grade 4 is life-threatening.

In some embodiments of the methods provided herein, the hematologic toxicity is anemia. In some embodiments of the methods provided herein, the hematologic toxicity is thrombocytopenia. In some embodiments of the methods provided herein, the hematologic toxicity is neutropenia. In some embodiments of the methods provided herein, the hematologic toxicity is febrile neutropenia.

Based on the hematologic toxicity grade, for example the hematologic toxicity grade described in Table 13, in some embodiments, the methods provided herein further comprises (w) determining hematologic toxicity in the subject, and (x) if the hematologic toxicity from (w) is no less than Grade 2, withholding the administration of the ADC. In certain embodiments, the methods provided herein further comprises (y) waiting for a period sufficient for the hematologic toxicity to reduce to no more than Grade 1. In some further embodiments, the methods provided herein comprises (z) determining the hematologic toxicity in the subject, and (aa) if the hematologic toxicity in (z) is no more than Grade 1, administering to the subject a second regimen comprising an effective amount of the ADC, wherein the second regimen comprises an ADC dose equal to or lower than the first regimen.

The disclosure provides that under certain criteria of severe adverse events in the subject, such as hematologic toxicity no less than Grade 4, the administration of the ADC for the cancer treatment is discontinued permanently. In some embodiments of the methods provided herein, if the hematologic toxicity from the method step (w) or (z) as described above is no less than Grade 4, the administration of the ADC is discontinued permanently. In certain embodiments, if the hematologic toxicity is no less than Grade 4, the administration of the ADC is permanently discontinued regardless any other criteria.

The disclosure provides that the method steps for the dose modification based on the criteria of hematologic toxicity can be iterated. The disclosure further provides that the method steps for the dose modification based on the criteria of hematologic toxicity can be iterated according to the rules set forth and provided herein. In some embodiments of the methods provided herein, the method steps (a), (w), (x), (y), (z) and (aa) can be repeated, which are (a) administering to the subject a first regimen comprising an effective amount of an ADC, (w) determining hematologic toxicity in the subject, (x) if the hematologic toxicity from (w) is no less than Grade 2, withholding the administration of the ADC, (y) waiting for a period sufficient for the hematologic toxicity to reduce to no more than Grade 1, (z) determining the hematologic toxicity in the subject, and (aa) if the hematologic toxicity in (z) is no more than Grade 1, administering to the subject a second regimen comprising an effective amount of the ADC, wherein the second regimen comprises an ADC dose equal to or lower than the first regimen. In some embodiments of the methods provided herein, the method steps (a), (w), (x), (z) and (aa) can be repeated, which are (a) administering to the subject a first regimen comprising an effective amount of an ADC, (w) determining hematologic toxicity in the subject, (x) if the hematologic toxicity from (w) is no less than Grade 2, withholding the administration of the ADC, (z) determining the hematologic toxicity in the subject, and (aa) if the hematologic toxicity in (z) is no more than Grade 1, administering to the subject a second regimen comprising an effective amount of the ADC, wherein the second regimen comprises an ADC dose equal to or lower than the first regimen. In some embodiments of the methods provided herein, the method steps (w), (x), (y), (z) and (aa) can be repeated, which are (w) determining hematologic toxicity in the subject, (x) if the hematologic toxicity from (w) is no less than Grade 2, withholding the administration of the ADC, (y) waiting for a period sufficient for the hematologic toxicity to reduce to no more than Grade 1, (z) determining the hematologic toxicity in the subject, and (aa) if the hematologic toxicity in (z) is no more than Grade 1, administering to the subject a second regimen comprising an effective amount of the ADC, wherein the second regimen comprises an ADC dose equal to or lower than the first regimen. In some embodiments of the methods provided herein, the method steps (w), (x), (z) and (aa) can be repeated, which are (w) determining hematologic toxicity in the subject, (x) if the hematologic toxicity from (w) is no less than Grade 2, withholding the administration of the ADC, (z) determining the hematologic toxicity in the subject, and (aa) if the hematologic toxicity in (z) is no more than Grade 1, administering to the subject a second regimen comprising an effective amount of the ADC, wherein the second regimen comprises an ADC dose equal to or lower than the first regimen.

In some embodiments of the methods provided herein, the time period sufficient for the hematologic toxicity to reduce to no more than Grade 1 is 1 to 10 days. In some embodiments of the methods provided herein, the time period sufficient for the hematologic toxicity to reduce to no more than Grade 1 is 1 to 10 weeks. In some embodiments of the methods provided herein, the time period sufficient for the hematologic toxicity to reduce to no more than Grade 1 is 1 to 4 months. In some embodiments of the methods provided herein, the time period sufficient for the hematologic toxicity to reduce to no more than Grade 1 is 1 day. In some embodiments of the methods provided herein, the time period sufficient for the hematologic toxicity to reduce to no more than Grade 1 is 2 days. In some embodiments of the methods provided herein, the time period sufficient for the hematologic toxicity to reduce to no more than Grade 1 is 3 days. In some embodiments of the methods provided herein, the time period sufficient for the hematologic toxicity to reduce to no more than Grade 1 is 4 days. In some embodiments of the methods provided herein, the time period sufficient for the hematologic toxicity to reduce to no more than Grade 1 is 5 days. In some embodiments of the methods provided herein, the time period sufficient for the hematologic toxicity to reduce to no more than Grade 1 is 6 days. In some embodiments of the methods provided herein, the time period sufficient for the hematologic toxicity to reduce to no more than Grade 1 is 7 days. In some embodiments of the methods provided herein, the time period sufficient for the hematologic toxicity to reduce to no more than Grade 1 is 8 days. In some embodiments of the methods provided herein, the time period sufficient for the hematologic toxicity to reduce to no more than Grade 1 is 9 days. In some embodiments of the methods provided herein, the time period sufficient for the hematologic toxicity to reduce to no more than Grade 1 is 10 days. In some embodiments of the methods provided herein, the time period sufficient for the hematologic toxicity to reduce to no more than Grade 1 is 1 week. In some embodiments of the methods provided herein, the time period sufficient for the hematologic toxicity to reduce to no more than Grade 1 is 2 weeks. In some embodiments of the methods provided herein, the time period sufficient for the hematologic toxicity to reduce to no more than Grade 1 is 3 weeks. In some embodiments of the methods provided herein, the time period sufficient for the hematologic toxicity to reduce to no more than Grade 1 is 4 weeks. In some embodiments of the methods provided herein, the time period sufficient for the hematologic toxicity to reduce to no more than Grade 1 is 5 weeks. In some embodiments of the methods provided herein, the time period sufficient for the hematologic toxicity to reduce to no more than Grade 1 is 6 weeks. In some embodiments of the methods provided herein, the time period sufficient for the hematologic toxicity to reduce to no more than Grade 1 is 7 weeks. In some embodiments of the methods provided herein, the time period sufficient for the hematologic toxicity to reduce to no more than Grade 1 is 8 weeks. In some embodiments of the methods provided herein, the time period sufficient for the hematologic toxicity to reduce to no more than Grade 1 is 1 month. In some embodiments of the methods provided herein, the time period sufficient for the hematologic toxicity to reduce to no more than Grade 1 is 2 months. In some embodiments of the methods provided herein, the time period sufficient for the hematologic toxicity to reduce to no more than Grade 1 is 3 months. In some embodiments of the methods provided herein, the time period sufficient for the hematologic toxicity to reduce to no more than Grade 1 is 4 months.

The disclosure provides that the modified dose can depend on the number of times the condition for the administration of the second regimen based on the criteria of hematologic toxicity have been satisfied. As such, in some embodiments, the methods further comprises determining the number of times the condition for the administration of the second regimen based on the criteria of hematologic toxicity have been satisfied. The disclosure provides that the ADC dose can be modified according to the scheme described in Table 14 below:

TABLE 14
dose modification schemes for the methods provided herein
based on hematologic toxicity

| Grade 1 | Grade 2 | Grade 3 | Grade 4 |
|---|---|---|---|
| Continue at | Withhold until | Withhold until Grade ≤ | Withhold |
| same dose level | Grade ≤ 1, then | 1, then resume | until Grade ≤ |
| | resume | treatment at the same | 1, then reduce |
| | treatment at the | dose level or consider | dose by one |
| | same dose | dose reduction by one | dose level or |
| | level or | dose level. | discontinue |
| | consider dose | | treatment. |
| | reduction by | | |
| | one dose level. | | |

In some embodiments, the dose reduction or modification referenced to in the Table 14 above and the paragraphs related to hematologic toxicity above and below, are set forth in Table 8 above.

In some embodiments of the methods provided herein, the ADC dose in the first regimen is the starting dose before any dose reduction or dose modification based on hematologic toxicity. Based on Table 8, in some embodiments, such ADC dose in the first regimen and the starting dose is 1.25 mg/kg for subject having a body weight of less than 100 kg or 125 mg for subject having a body weight of no less than 100 kg.

As is clear from the description and Table 14 and Table 8 above, in some embodiments of the methods provided herein, if the hematologic toxicity in (w) is no less than Grade 4 and the subject has a body weight of less than 100 kg, the ADC dose in the second regimen is lowered to about 1.0 mg/kg of the subject's body weight. In some embodiments of the methods provided herein, if the hematologic toxicity in (w) is no less than Grade 4 and the subject has a body weight of no less than 100 kg, the ADC dose in the second regimen is lowered to about 100 mg to the subject.

In some embodiments of the methods provided herein, if the hematologic toxicity in (w) is Grade 3 or Grade 2, the second regimen in (aa) is identical to the first regimen. In some embodiments of the methods provided herein, if the hematologic toxicity in (w) is Grade 3 or Grade 2 and the second regimen is administered for the first time, the second regimen in (aa) is identical to the first regimen. In some embodiments of the methods provided herein, if the hematologic toxicity in (w) is Grade 3 or Grade 2, the second regimen in (aa) is identical to the first regimen when the second regimen is administered for the first time or has been administered one or more times in (aa).

In some embodiments of the methods provided herein, if the hematologic toxicity in (w) is Grade 3 or Grade 2 and if the subject has a body weight of less than 100 kg, the ADC dose in the second regimen in (aa) is lowered to about 1.0 mg/kg of the subject's body weight. In some embodiments of the methods provided herein, if the hematologic toxicity in (w) is Grade 3 or Grade 2 and if the subject has a body weight of no less than 100 kg, the ADC dose in the second regimen in (aa) is lowered to about 100 mg to the subject. In some embodiments of the methods provided herein, if the hematologic toxicity in (w) is Grade 3 or Grade 2 and if the subject has a body weight of less than 100 kg, the ADC dose in the second regimen in (aa) is lowered to about 1.0 mg/kg of the subject's body weight when the second regimen is administered for the first time or has been administered one or more times in (aa). In some embodiments of the methods provided herein, if the hematologic toxicity in (w) is Grade 3 or Grade 2 and if the subject has a body weight of no less than 100 kg, the ADC dose in the second regimen in (aa) is lowered to about 100 mg to the subject when the second regimen is administered for the first time or has been administered one or more times in (aa).

In some embodiments of the methods provided herein, if the second regimen has been modified to the ADC dose of about 1.0 mg/kg or 100 mg based on hematologic toxicity and if the subject has a body weight of less than 100 kg, the ADC dose in the second regimen in (aa) is lowered to about 0.75 mg/kg of the subject's body weight. In some embodiments of the methods provided herein, if the second regimen has been modified to the ADC dose of about 1.0 mg/kg or 100 mg based on hematologic toxicity and if the subject has a body weight of no less than 100 kg, the ADC dose in the second regimen in (aa) is lowered to about 75 mg to the subject. In some embodiments of the methods provided herein, if the second regimen has been modified to the ADC dose of about 1.0 mg/kg or 100 mg based on hematologic toxicity and if the subject has a body weight of less than 100 kg, the ADC dose in the second regimen in (aa) is lowered to about 0.75 mg/kg of the subject's body weight regardless how many times the second regimen has been administered. In some embodiments of the methods provided herein, if the second regimen has been modified to the ADC dose of about 1.0 mg/kg or 100 mg based on hematologic toxicity and if the subject has a body weight of no less than 100 kg, the ADC dose in the second regimen in (aa) is lowered to about 75 mg to the subject regardless how many times the second regimen has been administered.

In some embodiments of the methods provided herein, if the second regimen has been modified to the ADC dose of about 0.75 mg/kg or 75 mg based on hematologic toxicity and if the subject has a body weight of less than 100 kg, the ADC dose in the second regimen in (aa) is lowered to about 0.5 mg/kg of the subject's body weight. In some embodiments of the methods provided herein, if the second regimen has been modified to the ADC dose of about 0.75 mg/kg or 75 mg based on hematologic toxicity and if the subject has a body weight of no less than 100 kg, the ADC dose in the second regimen in (aa) is lowered to about 50 mg to the subject. In some embodiments of the methods provided herein, if the second regimen has been modified to the ADC dose of about 0.75 mg/kg or 75 mg based on hematologic toxicity and if the subject has a body weight of less than 100 kg, the ADC dose in the second regimen in (aa) is lowered to about 0.5 mg/kg of the subject's body weight regardless how many times the second regimen has been administered. In some embodiments of the methods provided herein, if the second regimen has been modified to the ADC dose of about 0.75 mg/kg or 75 mg based on hematologic toxicity and if the subject has a body weight of no less than 100 kg, the ADC dose in the second regimen in (aa) is lowered to about 50 mg to the subject regardless how many times the second regimen has been administered.

The disclosure provides that requiring a dose reduction may be re-escalated by 1 dose level according to Table 8 (e.g., patients reduced to 0.75 mg/kg may only be re-escalated to 1 mg/kg) if the hematologic toxicity does not require study drug discontinuation and the hematologic toxicity has returned to baseline or ≤Grade 1. Accordingly, in some embodiments of the methods provided herein, the ADC dose in the second regimen is increased by an amount of about 0.25 mg/kg for the subject having a body weight of less than 100 kg or increased by an amount of about 25 mg for the subject having a body weight of no less than 100 kg, if (1) the administration of the ADC has not been discontinued permanently, (2) the ADC dose in the second regimen is lower than the ADC dose in the first regimen, and (3) the hematologic toxicity has returned to no more than Grade 1. Specifically, in some embodiments of the methods provided herein, the ADC dose in the second regimen is increased from 0.5 mg/ml to 0.75 mg/ml for the subject having a body weight of less than 100 kg or increased 50 mg to 75 mg for the subject having a body weight of no less than 100 kg, if (1) the administration of the ADC has not been discontinued permanently, (2) the ADC dose in the second regimen is lower than the ADC dose in the first regimen, and (3) the hematologic toxicity has returned to no more than Grade 1. In some embodiments of the methods provided herein, the ADC dose in the second regimen is increased from 0.75 mg/ml to 1 mg/ml for the subject having a body weight of less than 100 kg or increased 75 mg to 100 mg for the subject having a body weight of no less than 100 kg, if (1) the administration of the ADC has not been discontinued permanently, (2) the ADC dose in the second regimen is lower than the ADC dose in the first regimen, and (3) the hematologic toxicity has returned to no more than Grade 1. In some embodiments of the methods provided herein, the ADC dose in the second regimen is increased from 1 mg/ml to 1.25 mg/ml for the subject having a body weight of less than 100 kg or increased 100 mg to 125 mg for the subject having a body weight of no less than 100 kg, if (1) the administration of the ADC has not been discontinued permanently, (2) the ADC dose in the second regimen is lower than the ADC dose in the first regimen, and (3) the hematologic toxicity has returned to no more than Grade 1.

The disclosure provides that the hematologic toxicity, including for example the grade for anemia, thrombocytopenia, neutropenia and/or febrile neutropenia, in the methods provided herein, can be determined at various frequencies and intervals according to the need of the methods and/or the practice of the art. In some embodiments of the methods provided herein, the hematologic toxicity is determined daily. In some embodiments of the methods provided herein, the hematologic toxicity is determined once every two days, once every three days, once every four days, or once every five days, once every six days. In some embodiments of the methods provided herein, the hematologic toxicity is determined weekly, bi-weekly, once every three weeks, or once every four weeks. In some embodiments of the methods provided herein, the hematologic toxicity is determined monthly, once every two months, or once every three months.

### 5.2.7 Methods of Treatment Including Dose Modification Based on Fatigue

The disclosure also provides that the ADC dose administered for treating the cancer in the subject can be modified based on other criteria, for example fatigue in the subject. In some embodiments, the subject treated with the methods provided herein has fatigue. In some embodiments, fatigue is used as the criteria for modifying the ADC dose in the methods provided herein.

The fatigue as the dose modification criteria can be determined based on the Grade scale as set forth in National Cancer Institute: Common Terminology Criteria for Adverse Events (CTCAE) version 4.03. https://evs.nci.nih.gov/ftp1/CTCAE/CTCAE_4.03/CTCAE_4.03_2010-06-14_QuickReference_5×7.pdf, which is incorporated hereby in its entirety by reference. In some specific embodiments, the Grade for fatigue is determined as set forth in the following Table 15.

TABLE 15
CTCAE Definition and Grading v4.0 for Fatigue

| Grade 1 | Grade 2 | Grade 3 | Grade 4 | Grade 5 |
|---|---|---|---|---|
| Fatigue | Fatigue not | Fatigue not | N/A | N/A |
| relieved | relieved by | relieved | | |
| by rest | rest; limiting | by rest; | | |
| | instrumental | limiting self | | |
| | ADL | care ADL | | |

ADL: Activities of daily living;
N/A: not available

Based on the fatigue grade, in some embodiments, the methods provided herein further comprises (ab) determining fatigue in the subject, and (ac) if the fatigue from (ab) is no less than Grade 3, withholding the administration of the ADC. In certain embodiments, the methods provided herein further comprises (ad) waiting for a period sufficient for the fatigue to reduce to no more than Grade 1. In some further embodiments, the methods provided herein comprises (ae) determining the fatigue in the subject, and (af) if the fatigue in (ae) is no more than Grade 1, administering to the subject a second regimen comprising an effective amount of the ADC, wherein the second regimen comprises an ADC dose equal to or lower than the first regimen.

The disclosure provides that under certain criteria of severe adverse events in the subject, such as fatigue no less than Grade 4, the administration of the ADC for the cancer treatment is discontinued permanently. In some embodiments of the methods provided herein, if the fatigue from the method step (ab) or (ae) as described above is no less than Grade 4, the administration of the ADC is discontinued permanently. In certain embodiments, if the fatigue is no less than Grade 4, the administration of the ADC is permanently discontinued regardless any other criteria.

The disclosure provides that the method steps for the dose modification based on the criteria of fatigue can be iterated. The disclosure further provides that the method steps for the dose modification based on the criteria of fatigue can be iterated according to the rules set forth and provided herein. In some embodiments of the methods provided herein, the method steps (a), (ab), (ac), (ad), (ae) and (af) can be repeated, which are (a) administering to the subject a first regimen comprising an effective amount of an ADC, (ab) determining fatigue in the subject, and (ac) if the fatigue from (ab) is no less than Grade 3, withholding the administration of the ADC, (ad) waiting for a period sufficient for the fatigue to reduce to no more than Grade 1, (ae) determining the fatigue in the subject, and (af) if the fatigue in (ae) is no more than Grade 1, administering to the subject a second regimen comprising an effective amount of the ADC, wherein the second regimen comprises an ADC dose equal to or lower than the first regimen. In some embodiments of the methods provided herein, the method steps (a), (ab), (ac), (ae) and (af) can be repeated, which are (a) administering to the subject a first regimen comprising an effective amount of an ADC, (ab) determining fatigue in the subject, and (ac) if the fatigue from (ab) is no less than Grade 3, withholding the administration of the ADC, (ae) determining the fatigue in the subject, and (af) if the fatigue in (ae) is no more than Grade 1, administering to the subject a second regimen comprising an effective amount of the ADC, wherein the second regimen comprises an ADC dose equal to or lower than the first regimen. In some embodiments of the methods provided herein, the method steps (ab), (ac), (ad), (ae) and (af) can be repeated, which are (ab) determining fatigue in the subject, and (ac) if the fatigue from (ab) is no less than Grade 3, withholding the administration of the ADC, (ad) waiting for a period sufficient for the fatigue to reduce to no more than Grade 1, (ae) determining the fatigue in the subject, and (af) if the fatigue in (ae) is no more than Grade 1, administering to the subject a second regimen comprising an effective amount of the ADC, wherein the second regimen comprises an ADC dose equal to or lower than the first regimen. In some embodiments of the methods provided herein, the method steps (ab), (ac), (ae) and (af) can be repeated, which are (ab) determining fatigue in the subject, and (ac) if the fatigue from (ab) is no less than Grade 3, withholding the administration of the ADC, (ae) determining the fatigue in the subject, and (af) if the fatigue in (ae) is no more than Grade 1, administering to the subject a second regimen comprising an effective amount of the ADC, wherein the second regimen comprises an ADC dose equal to or lower than the first regimen.

In some embodiments of the methods provided herein, the time period sufficient for the fatigue to reduce to no more than Grade 1 is 1 to 10 days. In some embodiments of the methods provided herein, the time period sufficient for the fatigue to reduce to no more than Grade 1 is 1 to 10 weeks. In some embodiments of the methods provided herein, the time period sufficient for the fatigue to reduce to no more than Grade 1 is 1 to 4 months. In some embodiments of the methods provided herein, the time period sufficient for the fatigue to reduce to no more than Grade 1 is 1 day. In some embodiments of the methods provided herein, the time period sufficient for the fatigue to reduce to no more than Grade 1 is 2 days. In some embodiments of the methods provided herein, the time period sufficient for the fatigue to reduce to no more than Grade 1 is 3 days. In some embodiments of the methods provided herein, the time period sufficient for the fatigue to reduce to no more than Grade 1 is 4 days. In some embodiments of the methods provided herein, the time period sufficient for the fatigue to reduce to no more than Grade 1 is 5 days. In some embodiments of the methods provided herein, the time period sufficient for the fatigue to reduce to no more than Grade 1 is 6 days. In some embodiments of the methods provided herein, the time period sufficient for the fatigue to reduce to no more than Grade 1 is 7 days. In some embodiments of the methods provided herein, the time period sufficient for the fatigue to reduce to no more than Grade 1 is 8 days. In some embodiments of the methods provided herein, the time period sufficient for the fatigue to reduce to no more than Grade 1 is 9 days. In some embodiments of the methods provided herein, the time period sufficient for the fatigue to reduce to no more than Grade 1 is 10 days. In some embodiments of the methods provided herein, the time period sufficient for the fatigue to reduce to no more than Grade 1 is 1 week. In some embodiments of the methods provided herein, the time period sufficient for the fatigue to reduce to no more than Grade 1 is 2 weeks. In some embodiments of the methods provided herein, the time period sufficient for the fatigue to reduce to no more than Grade 1 is 3 weeks. In some embodiments of the methods provided herein, the time period sufficient for the fatigue to reduce to no more than Grade 1 is 4 weeks. In some embodiments of the methods provided herein, the time period sufficient for the fatigue to reduce to no more than Grade 1 is 5 weeks. In some embodiments of the methods provided herein, the time period sufficient for the fatigue to reduce to no more than Grade 1 is 6 weeks. In some embodiments of the methods provided herein, the time period sufficient for the fatigue to reduce to no more than Grade 1 is 7 weeks. In some embodiments of the methods provided herein, the time period sufficient for the fatigue to reduce to no more than Grade 1 is 8 weeks. In some embodiments of the methods provided herein, the time period sufficient for the fatigue to reduce to no more than Grade 1 is 1 month. In some embodiments of the methods provided herein, the time period sufficient for the fatigue to reduce to no more than Grade 1 is 2 months. In some embodiments of the methods provided herein, the time period sufficient for the fatigue to reduce to no more than Grade 1 is 3 months. In some embodiments of the methods provided herein, the time period sufficient for the fatigue to reduce to no more than Grade 1 is 4 months.

The disclosure provides that the modified dose can depend on the number of times the condition for the administration of the second regimen based on the criteria of fatigue have been satisfied. As such, in some embodiments, the methods further comprises determining the number of times the condition for the administration of the second regimen based on the criteria of fatigue have been satisfied. The disclosure provides that the ADC dose can be modified according to the scheme described in Table 16 below:

TABLE 16
dose modification schemes for the methods provided
herein based on fatigue¹

| Grade 1 | Grade 2 | Grade 3 | Grade 4 |
|---|---|---|---|
| Continue at | Continue at | Withhold dose until | Discontinue |
| same dose level | same dose | toxicity (fatigue) is ≤ | treatment |
| | level | Grade 1 or has returned | |
| | | to baseline, then | |
| | | resume treatment at the | |
| | | same dose level or | |
| | | consider dose reduction | |
| | | by 1 dose level. | |

¹See e.g. Rosenberg JE, et a . J Clin Oncol. 2019; 37:2592-2600; Rosenberg JE, et al. J Clin Oncol. 2019; 37: 2592-2600 (Protocol), both of which are hereby incorporated in their entireties by reference.

In some embodiments, the dose reduction or modification referenced to in the Table 16 above and the paragraphs related to fatigue above and below, are set forth in Table 8 above.

In some embodiments of the methods provided herein, the ADC dose in the first regimen is the starting dose before any dose reduction or dose modification based on fatigue. Based on Table 8, in some embodiments, such ADC dose in the first regimen and the starting dose is 1.25 mg/kg for subject having a body weight of less than 100 kg or 125 mg for subject having a body weight of no less than 100 kg.

As is clear from the description and Table 16 and Table 8 above, in some embodiments of the methods provided herein, if the fatigue in (ab) is Grade 3, the second regimen in (af) is identical to the first regimen. In some embodiments of the methods provided herein, if the fatigue in (ab) is Grade 3 and the second regimen is administered for the first time, the second regimen in (af) is identical to the first regimen. In some embodiments of the methods provided herein, if the fatigue in (ab) is Grade 3, the second regimen in (af) is identical to the first regimen when the second regimen is administered for the first time or has been administered one or more times in (af).

In some embodiments of the methods provided herein, if the fatigue in (ab) is Grade 3 and if the subject has a body weight of less than 100 kg, the ADC dose in the second regimen in (af) is lowered to about 1.0 mg/kg of the subject's body weight. In some embodiments of the methods provided herein, if the fatigue in (ab) is Grade 3 and if the subject has a body weight of no less than 100 kg, the ADC dose in the second regimen in (af) is lowered to about 100 mg to the subject. In some embodiments of the methods provided herein, if the fatigue in (ab) is Grade 3 and if the subject has a body weight of less than 100 kg, the ADC dose in the second regimen in (af) is lowered to about 1.0 mg/kg of the subject's body weight when the second regimen is administered for the first time or has been administered one or more times in (af). In some embodiments of the methods provided herein, if the fatigue in (ab) is Grade 3 and if the subject has a body weight of no less than 100 kg, the ADC dose in the second regimen in (af) is lowered to about 100 mg to the subject when the second regimen is administered for the first time or has been administered one or more times in (af).

In some embodiments of the methods provided herein, if the second regimen has been modified to the ADC dose of about 1.0 mg/kg or 100 mg based on fatigue and if the subject has a body weight of less than 100 kg, the ADC dose in the second regimen in (af) is lowered to about 0.75 mg/kg of the subject's body weight. In some embodiments of the methods provided herein, if the second regimen has been modified to the ADC dose of about 1.0 mg/kg or 100 mg based on fatigue and if the subject has a body weight of no less than 100 kg, the ADC dose in the second regimen in (af) is lowered to about 75 mg to the subject. In some embodiments of the methods provided herein, if the second regimen has been modified to the ADC dose of about 1.0 mg/kg or 100 mg based on fatigue and if the subject has a body weight of less than 100 kg, the ADC dose in the second regimen in (af) is lowered to about 0.75 mg/kg of the subject's body weight regardless how many times the second regimen has been administered. In some embodiments of the methods provided herein, if the second regimen has been modified to the ADC dose of about 1.0 mg/kg or 100 mg based on fatigue and if the subject has a body weight of no less than 100 kg, the ADC dose in the second regimen in (af) is lowered to about 75 mg to the subject regardless how many times the second regimen has been administered.

In some embodiments of the methods provided herein, if the second regimen has been modified to the ADC dose of about 0.75 mg/kg or 75 mg based on fatigue and if the subject has a body weight of less than 100 kg, the ADC dose in the second regimen in (af) is lowered to about 0.5 mg/kg of the subject's body weight. In some embodiments of the methods provided herein, if the second regimen has been modified to the ADC dose of about 0.75 mg/kg or 75 mg based on fatigue and if the subject has a body weight of no less than 100 kg, the ADC dose in the second regimen in (af) is lowered to about 50 mg to the subject. In some embodiments of the methods provided herein, if the second regimen has been modified to the ADC dose of about 0.75 mg/kg or 75 mg based on fatigue and if the subject has a body weight of less than 100 kg, the ADC dose in the second regimen in (af) is lowered to about 0.5 mg/kg of the subject's body weight regardless how many times the second regimen has been administered. In some embodiments of the methods provided herein, if the second regimen has been modified to the ADC dose of about 0.75 mg/kg or 75 mg based on fatigue and if the subject has a body weight of no less than 100 kg, the ADC dose in the second regimen in (af) is lowered to about 50 mg to the subject regardless how many times the second regimen has been administered.

The disclosure provides that requiring a dose reduction may be re-escalated by 1 dose level according to Table 8 (e.g., patients reduced to 0.75 mg/kg may only be re-escalated to 1 mg/kg) if the fatigue does not require study drug discontinuation and the fatigue has returned to baseline or ≤Grade 1. Accordingly, in some embodiments of the methods provided herein, the ADC dose in the second regimen is increased by an amount of about 0.25 mg/kg for the subject having a body weight of less than 100 kg or increased by an amount of about 25 mg for the subject having a body weight of no less than 100 kg, if (1) the administration of the ADC has not been discontinued permanently, (2) the ADC dose in the second regimen is lower than the ADC dose in the first regimen, and (3) the fatigue has returned to no more than Grade 1. Specifically, in some embodiments of the methods provided herein, the ADC dose in the second regimen is increased from 0.5 mg/ml to 0.75 mg/ml for the subject having a body weight of less than 100 kg or increased 50 mg to 75 mg for the subject having a body weight of no less than 100 kg, if (1) the administration of the ADC has not been discontinued permanently, (2) the ADC dose in the second regimen is lower than the ADC dose in the first regimen, and (3) the fatigue has returned to no more than Grade 1. In some embodiments of the methods provided herein, the ADC dose in the second regimen is increased from 0.75 mg/ml to 1 mg/ml for the subject having a body weight of less than 100 kg or increased 75 mg to 100 mg for the subject having a body weight of no less than 100 kg, if (1) the administration of the ADC has not been discontinued permanently, (2) the ADC dose in the second regimen is lower than the ADC dose in the first regimen, and (3) the fatigue has returned to no more than Grade 1. In some embodiments of the methods provided herein, the ADC dose in the second regimen is increased from 1 mg/ml to 1.25 mg/ml for the subject having a body weight of less than 100 kg or increased 100 mg to 125 mg for the subject having a body weight of no less than 100 kg, if (1) the administration of the ADC has not been discontinued permanently, (2) the ADC dose in the second regimen is lower than the ADC dose in the first regimen, and (3) the fatigue has returned to no more than Grade 1.

The disclosure provides that the fatigue in the methods provided herein can be determined at various frequencies and intervals according to the need of the methods and/or the practice of the art. In some embodiments of the methods provided herein, the fatigue is determined daily. In some embodiments of the methods provided herein, the fatigue is determined once every two days, once every three days, once every four days, or once every five days, once every six days. In some embodiments of the methods provided herein, the fatigue is determined weekly, bi-weekly, once every three weeks, or once every four weeks. In some embodiments of the methods provided herein, the fatigue is determined monthly, once every two months, or once every three months.

### 5.2.8 Methods of Treatment Including Dose Modification Based on Diarrhea

The disclosure also provides that the ADC dose administered for treating the cancer in the subject can be modified based on other criteria, for example diarrhea in the subject. In some embodiments, the subject treated with the methods provided herein has diarrhea. In some embodiments, diarrhea is used as the criteria for modifying the ADC dose in the methods provided herein.

The diarrhea as the dose modification criteria can be determined based on the Grade scale as set forth in National Cancer Institute: Common Terminology Criteria for Adverse Events (CTCAE) version 4.03. https://evs.nci.nih.gov/ftp1/CTCAE/CTCAE_4.03/CTCAE_4.03_2010-06-14_QuickReference_5×7.pdf, which is incorporated hereby in its entirety by reference. In some more specific embodiments, the Grade for diarrhea is determined as set forth in the following Table 17.

TABLE 17
CTCAE Definition and Grading v4.0 for Diarrhea

| Grade 1 | Grade 2 | Grade 3 | Grade 4 | Grade 5 |
|---|---|---|---|---|
| Increase of <4 | Increase of 4-6 | Increase of >=7 | Life-threatening | Death |
| stools per day | stools per day | stools per day | consequences; urgent | |
| over baseline; | over baseline; | over baseline; | intervention indicated | |
| mild increase in | moderate | incontinence; | | |
| ostomy output | increase in | hospitalization | | |
| compared to | ostomy output | indicated; severe | | |
| baseline | compared to | increase in | | |
| | baseline | ostomy output | | |
| | | compared to | | |
| | | baseline; limiting | | |
| | | self care ADL¹ | | |

ADL: Activities of daily living
¹Self-care ADL refers to bathing, dressing and undressing, feeding self, using the toilet, taking medications, and not bedridden.

Additionally, in some embodiments, diarrhea can be assessed, evaluated, described, and categorized in accordance with National Cancer Institute, Gastrointestinal Complications (PDQ®)—Health Professional Version. https://www.cancer.gov/about-cancer/treatment/side-effects/constipation/gi-complications-hp-pdq as updated on Nov. 28, 2018.

Alternatively, in some embodiments, the grade of diarrhea is determined according to a scale in which Grade 1 is mild, Grade 2 is moderate, Grade 3 is severe, and Grade 4 is life-threatening.

Based on the diarrhea grade, for example the diarrhea grade described in Table 17 and/or the preceding several paragraphs, in some embodiments, the methods provided herein further comprises (ag) determining diarrhea in the subject, and (ah) if the diarrhea from (ag) is no less than Grade 3, withholding the administration of the ADC. In certain embodiments, the methods provided herein further comprises (ai) waiting for a period sufficient for the diarrhea to reduce to no more than Grade 1. In some further embodiments, the methods provided herein comprises (aj) determining the diarrhea in the subject, and (ak) if the diarrhea in (aj) is no more than Grade 1, administering to the subject a second regimen comprising an effective amount of the ADC, wherein the second regimen comprises an ADC dose equal to or lower than the first regimen.

The disclosure provides that under certain criteria of severe adverse events in the subject, such as diarrhea no less than Grade 4, the administration of the ADC for the cancer treatment is discontinued permanently. In some embodiments of the methods provided herein, if the diarrhea in (ag) or (ai) is no less than Grade 4, the administration of the ADC is discontinued permanently. In certain embodiments, if the diarrhea in (ag) or (ai) is no less than Grade 4, the administration of the ADC is permanently discontinued regardless any other criteria. In some embodiments of the methods provided herein, if the diarrhea is no less than Grade 4, the administration of the ADC is discontinued permanently. In certain embodiments, if the diarrhea is no less than Grade 4, the administration of the ADC is permanently discontinued regardless any other criteria. In some embodiments of the methods provided herein, if the diarrhea in (ag) or (ai) is no less than Grade 4 and the diarrhea does not improve to no more than Grade 2 within 72 hours with supportive management, the administration of the ADC is discontinued permanently. In certain embodiments, if the diarrhea in (ag) or (ai) is no less than Grade 4 and the diarrhea does not improve to no more than Grade 2 within 72 hours with supportive management, the administration of the ADC is permanently discontinued regardless any other criteria.

The disclosure provides that the method steps for the dose modification based on the criteria of diarrhea can be iterated. The disclosure further provides that the method steps for the dose modification based on the criteria of diarrhea can be iterated according to the rules set forth and provided herein. In some embodiments of the methods provided herein, the method steps (a), (ag), (ah), (ai), (aj) and (ak) can be repeated, which are (a) administering to the subject a first regimen comprising an effective amount of an ADC, (ag) determining diarrhea in the subject, (ah) if the diarrhea from (ag) is no less than Grade 3, withholding the administration of the ADC, (ai) waiting for a period sufficient for the diarrhea to reduce to no more than Grade 1, (aj) determining the diarrhea in the subject, and (ak) if the diarrhea in (aj) is no more than Grade 1, administering to the subject a second regimen comprising an effective amount of the ADC, wherein the second regimen comprises an ADC dose equal to or lower than the first regimen. In some embodiments of the methods provided herein, the method steps (a), (ag), (ah), (aj) and (ak) can be repeated, which are (a) administering to the subject a first regimen comprising an effective amount of an ADC, (ag) determining diarrhea in the subject, (ah) if the diarrhea from (ag) is no less than Grade 3, withholding the administration of the ADC, (aj) determining the diarrhea in the subject, and (ak) if the diarrhea in (aj) is no more than Grade 1, administering to the subject a second regimen comprising an effective amount of the ADC, wherein the second regimen comprises an ADC dose equal to or lower than the first regimen. In some embodiments of the methods provided herein, the method steps (ag), (ah), (ai), (aj) and (ak) can be repeated, which are (ag) determining diarrhea in the subject, (ah) if the diarrhea from (ag) is no less than Grade 3, withholding the administration of the ADC, (ai) waiting for a period sufficient for the diarrhea to reduce to no more than Grade 1, (aj) determining the diarrhea in the subject, and (ak) if the diarrhea in (aj) is no more than Grade 1, administering to the subject a second regimen comprising an effective amount of the ADC, wherein the second regimen comprises an ADC dose equal to or lower than the first regimen. In some embodiments of the methods provided herein, the method steps (ag), (ah), (aj) and (ak) can be repeated, which are (ag) determining diarrhea in the subject, (ah) if the diarrhea from (ag) is no less than Grade 3, withholding the administration of the ADC, (aj) determining the diarrhea in the subject, and (ak) if the diarrhea in (aj) is no more than Grade 1, administering to the subject a second regimen comprising an effective amount of the ADC, wherein the second regimen comprises an ADC dose equal to or lower than the first regimen.

In some embodiments of the methods provided herein, the time period sufficient for the diarrhea to reduce to no more than Grade 1 is 1 to 10 days. In some embodiments of the methods provided herein, the time period sufficient for the diarrhea to reduce to no more than Grade 1 is 1 to 10 weeks. In some embodiments of the methods provided herein, the time period sufficient for the diarrhea to reduce to no more than Grade 1 is 1 to 4 months. In some embodiments of the methods provided herein, the time period sufficient for the diarrhea to reduce to no more than Grade 1 is 1 day. In some embodiments of the methods provided herein, the time period sufficient for the diarrhea to reduce to no more than Grade 1 is 2 days. In some embodiments of the methods provided herein, the time period sufficient for the diarrhea to reduce to no more than Grade 1 is 3 days. In some embodiments of the methods provided herein, the time period sufficient for the diarrhea to reduce to no more than Grade 1 is 4 days. In some embodiments of the methods provided herein, the time period sufficient for the diarrhea to reduce to no more than Grade 1 is 5 days. In some embodiments of the methods provided herein, the time period sufficient for the diarrhea to reduce to no more than Grade 1 is 6 days. In some embodiments of the methods provided herein, the time period sufficient for the diarrhea to reduce to no more than Grade 1 is 7 days. In some embodiments of the methods provided herein, the time period sufficient for the diarrhea to reduce to no more than Grade 1 is 8 days. In some embodiments of the methods provided herein, the time period sufficient for the diarrhea to reduce to no more than Grade 1 is 9 days. In some embodiments of the methods provided herein, the time period sufficient for the diarrhea to reduce to no more than Grade 1 is 10 days. In some embodiments of the methods provided herein, the time period sufficient for the diarrhea to reduce to no more than Grade 1 is 1 week. In some embodiments of the methods provided herein, the time period sufficient for the diarrhea to reduce to no more than Grade 1 is 2 weeks. In some embodiments of the methods provided herein, the time period sufficient for the diarrhea to reduce to no more than Grade 1 is 3 weeks. In some embodiments of the methods provided herein, the time period sufficient for the diarrhea to reduce to no more than Grade 1 is 4 weeks. In some embodiments of the methods provided herein, the time period sufficient for the diarrhea to reduce to no more than Grade 1 is 5 weeks. In some embodiments of the methods provided herein, the time period sufficient for the diarrhea to reduce to no more than Grade 1 is 6 weeks. In some embodiments of the methods provided herein, the time period sufficient for the diarrhea to reduce to no more than Grade 1 is 7 weeks. In some embodiments of the methods provided herein, the time period sufficient for the diarrhea to reduce to no more than Grade 1 is 8 weeks. In some embodiments of the methods provided herein, the time period sufficient for the diarrhea to reduce to no more than Grade 1 is 1 month. In some embodiments of the methods provided herein, the time period sufficient for the diarrhea to reduce to no more than Grade 1 is 2 months. In some embodiments of the methods provided herein, the time period sufficient for the diarrhea to reduce to no more than Grade 1 is 3 months. In some embodiments of the methods provided herein, the time period sufficient for the diarrhea to reduce to no more than Grade 1 is 4 months.

The disclosure provides that the modified dose can depend on the number of times the condition for the administration of the second regimen based on the criteria of diarrhea have been satisfied. As such, in some embodiments, the methods further comprises determining the number of times the condition for the administration of the second regimen based on the criteria of diarrhea have been satisfied. The disclosure provides that the ADC dose can be modified according to the scheme described in Table 18 below:

TABLE 18
dose modification schemes for the methods provided herein based on diarrhea¹

| Grade 1 | Grade 2 | Grade 3 | Grade 4 |
|---|---|---|---|
| Continue at | Continue at | Withhold dose until diarrhea | Discontinue treatment. |
| same dose | same dose | is ≤Grade 1 or has returned | Grade 4 diarrhea that improves |
| level | level. | to baseline, then resume | to ≤Grade 2 within 72 hours |
| | | treatment at the same dose | with supportive management |
| | | level or consider dose | does not require |
| | | reduction by 1 dose level. | discontinuation. |

¹See e.g. Rosenberg JE, et al. J Clin Oncol. 2019; 37:2592-2600; Rosenberg JE, et al. J Clin Oncol.2019; 37:2592-2600 (Protocol), both of which are hereby incorporated in their entireties by reference.

In some embodiments, the dose reduction or modification referenced to in the Table 18 above and the paragraphs related to diarrhea above and below, are set forth in Table 8 above.

In some embodiments of the methods provided herein, the ADC dose in the first regimen is the starting dose before any dose reduction or dose modification based on diarrhea. Based on Table 8, in some embodiments, such ADC dose in the first regimen and the starting dose is 1.25 mg/kg for subject having a body weight of less than 100 kg or 125 mg for subject having a body weight of no less than 100 kg.

As is clear from the description and Table 18 and Table 8 above, in some embodiments of the methods provided herein, if the second regimen is administered for the first time in (ak), the second regimen can be identical to the first regimen. In some embodiments of the methods provided herein, the second regimen in (ak) can be identical to the first regimen when the second regimen is administered for the first time or has been administered one or more times in (ak). In some embodiments of the methods provided herein, if the second regimen has been administered one or more times in (ak) and the subject has a body weight of less than 100 kg, the ADC dose in the second regimen is lowered to about 1.0 mg/kg of the subject's body weight. In some embodiments of the methods provided herein, if the second regimen has been administered one or more times in (ak) and the subject has a body weight of no less than 100 kg, the ADC dose in the second regimen is lowered to about 100 mg to the subject. In some embodiments of the methods provided herein, if the second regimen has been administered two or more times in (ak) and the subject has a body weight of less than 100 kg, the ADC dose in the second regimen is lowered to about 0.75 mg/kg of the subject's body weight. In some embodiments of the methods provided herein, if the second regimen has been administered two or more times in (ak) and the subject has a body weight of no less than 100 kg, the ADC dose in the second regimen is lowered to about 75 mg to the subject. In some embodiments of the methods provided herein, if the second regimen has been administered three or more times in (ak) and the subject has a body weight of less than 100 kg, the ADC dose in the second regimen is lowered to about 0.5 mg/kg of the subject's body weight. In some embodiments of the methods provided herein, if the second regimen has been administered three or more times in (ak) and the subject has a body weight of no less than 100 kg, the ADC dose in the second regimen is lowered to about 50 mg to the subject.

Alternatively, according to Table 18 and Table 8 above, in some embodiments of the methods provided herein and based on the criteria of diarrhea, if the subject has a body weight of less than 100 kg, the ADC dose in the second regimen is about 1.0 mg/kg of the subject's body weight. In some embodiments of the methods provided herein and based on the criteria of diarrhea, if the subject has a body weight of no less than 100 kg, the ADC dose in the second regimen is about 100 mg to the subject. In some embodiments of the methods provided herein and based on the criteria of diarrhea if the subject has a body weight of less than 100 kg, the ADC dose in the second regimen is about 1.0 mg/kg of the subject's body weight when the second regimen is administered for the first time or has been administered one or more times in (ak). In some embodiments of the methods provided herein and based on the criteria of diarrhea, if the subject has a body weight of no less than 100 kg, the ADC dose in the second regimen is about 100 mg to the subject when the second regimen is administered for the first time or has been administered one or more times in (ak). In some embodiments of the methods provided herein, if the second regimen has been administered one or more times in (ak) and the subject has a body weight of less than 100 kg, the ADC dose in the second regimen is lowered to about 0.75 mg/kg of the subject's body weight. In some embodiments of the methods provided herein, if the second regimen has been administered one or more times in (ak) and the subject has a body weight of no less than 100 kg, the ADC dose in the second regimen is lowered to about 75 mg to the subject. In some embodiments of the methods provided herein, if the second regimen has been administered two or more times in (ak) and the subject has a body weight of less than 100 kg, the ADC dose in the second regimen is lowered to about 0.5 mg/kg of the subject's body weight. In some embodiments of the methods provided herein, if the second regimen has been administered two or more times in (ak) and the subject has a body weight of no less than 100 kg, the ADC dose in the second regimen is lowered to about 50 mg to the subject.

The disclosure provides that requiring a dose reduction may be re-escalated by 1 dose level according to Table 8 (e.g., patients reduced to 0.75 mg/kg may only be re-escalated to 1 mg/kg) if the diarrhea does not require study drug discontinuation and the diarrhea has returned to baseline or ≤Grade 1. Accordingly, in some embodiments of the methods provided herein, the ADC dose in the second regimen is increased by an amount of about 0.25 mg/kg for the subject having a body weight of less than 100 kg or increased by an amount of about 25 mg for the subject having a body weight of no less than 100 kg, if (1) the administration of the ADC has not been discontinued permanently, (2) the ADC dose in the second regimen is lower than the ADC dose in the first regimen, and (3) the diarrhea has returned to no more than Grade 1. Specifically, in some embodiments of the methods provided herein, the ADC dose in the second regimen is increased from 0.5 mg/ml to 0.75 mg/ml for the subject having a body weight of less than 100 kg or increased 50 mg to 75 mg for the subject having a body weight of no less than 100 kg, if (1) the administration of the ADC has not been discontinued permanently, (2) the ADC dose in the second regimen is lower than the ADC dose in the first regimen, and (3) the diarrhea has returned to no more than Grade 1. In some embodiments of the methods provided herein, the ADC dose in the second regimen is increased from 0.75 mg/ml to 1 mg/ml for the subject having a body weight of less than 100 kg or increased 75 mg to 100 mg for the subject having a body weight of no less than 100 kg, if (1) the administration of the ADC has not been discontinued permanently, (2) the ADC dose in the second regimen is lower than the ADC dose in the first regimen, and (3) the diarrhea has returned to no more than Grade 1. In some embodiments of the methods provided herein, the ADC dose in the second regimen is increased from 1 mg/ml to 1.25 mg/ml for the subject having a body weight of less than 100 kg or increased 100 mg to 125 mg for the subject having a body weight of no less than 100 kg, if (1) the administration of the ADC has not been discontinued permanently, (2) the ADC dose in the second regimen is lower than the ADC dose in the first regimen, and (3) the diarrhea has returned to no more than Grade 1.

The disclosure provides that the diarrhea can be determined at various frequencies and intervals according to the need of the methods and/or the practice of the art. In some embodiments of the methods provided herein, the diarrhea is determined daily. In some embodiments of the methods provided herein, the diarrhea is determined once every two days, once every three days, once every four days, or once every five days, once every six days. In some embodiments of the methods provided herein, the diarrhea is determined weekly, bi-weekly, once every three weeks, or once every four weeks. In some embodiments of the methods provided herein, the diarrhea is determined monthly, once every two months, or once every three months.

### 5.2.9 Additional Embodiments of Methods of Treatment Including Dose Modifications

In view of the above description, in some embodiments, the methods provided herein further comprises (I) determining one or more dose modification criteria and one or more dose discontinuation criteria in the subject, and (II) if the one or more dose modification criteria from (I) are satisfied, withholding the administration of the ADC. In certain embodiments, the methods provided herein further comprises (III) waiting for a period sufficient for the subject to reach one or more dose continuation criteria. In some further embodiments, the methods provided herein comprises (IV) determining the one or more dose continuation criteria in the subject, and (V) if the one or more dose continuation criteria from (IV) are satisfied, administering the subject a second regimen comprising an effective amount of the ADC, wherein the second regimen comprises a ADC dose equal to or lower than the first regimen.

The disclosure provides that under one or more dose discontinuation criteria in the subject the administration of the ADC for the cancer treatment is discontinued permanently. In some embodiments of the methods provided herein, if the one or more dose discontinuation criteria are satisfied, the administration of the ADC is discontinued permanently. In certain embodiments, if the one or more dose discontinuation criteria, the administration of the ADC is permanently discontinued regardless any other criteria.

The disclosure provides that the method steps for the dose modification can be iterated. The disclosure further provides that the method steps for the dose modification can be iterated according to the rules set forth and provided herein. In some embodiments of the methods provided herein, the method steps (a), (I), (II), (III), (IV) and (V) can be repeated, which are (a) administering to the subject a first regimen comprising an effective amount of an ADC, (I) determining one or more dose modification criteria and one or more dose discontinuation criteria in the subject, (II) if the one or more dose modification criteria from (I) are satisfied, withholding the administration of the ADC, (III) waiting for a period sufficient for the subject to reach one or more dose continuation criteria, (IV) determining the one or more dose continuation criteria in the subject, and (V) if the one or more dose continuation criteria from (IV) are satisfied, administering the subject a second regimen comprising an effective amount of the ADC, wherein the second regimen comprises a ADC dose equal to or lower than the first regimen. In some embodiments of the methods provided herein, the method steps (a), (I), (II), (IV) and (V) can be repeated, which are (a) administering to the subject a first regimen comprising an effective amount of an ADC, (I) determining one or more dose modification criteria and one or more dose discontinuation criteria in the subject, (II) if the one or more dose modification criteria from (I) are satisfied, withholding the administration of the ADC, (IV) determining the one or more dose continuation criteria in the subject, and (V) if the one or more dose continuation criteria from (IV) are satisfied, administering the subject a second regimen comprising an effective amount of the ADC, wherein the second regimen comprises a ADC dose equal to or lower than the first regimen. In some embodiments of the methods provided herein, the method steps (I), (II), (III), (IV) and (V) can be repeated, which are (I) determining one or more dose modification criteria and one or more dose discontinuation criteria in the subject, (II) if the one or more dose modification criteria from (I) are satisfied, withholding the administration of the ADC, (III) waiting for a period sufficient for the subject to reach one or more dose continuation criteria, (IV) determining the one or more dose continuation criteria in the subject, and (V) if the one or more dose continuation criteria from (IV) are satisfied, administering the subject a second regimen comprising an effective amount of the ADC, wherein the second regimen comprises a ADC dose equal to or lower than the first regimen. In some embodiments of the methods provided herein, the method steps (I), (II), (IV) and (V) can be repeated, which are (I) determining one or more dose modification criteria and one or more dose discontinuation criteria in the subject, (II) if the one or more dose modification criteria from (I) are satisfied, withholding the administration of the ADC, (IV) determining the one or more dose continuation criteria in the subject, and (V) if the one or more dose continuation criteria from (IV) are satisfied, administering the subject a second regimen comprising an effective amount of the ADC, wherein the second regimen comprises a ADC dose equal to or lower than the first regimen.

In some embodiments of the methods provided herein, the time period sufficient for the subject to reach one or more dose continuation criteria is 1 to 10 days. In some embodiments of the methods provided herein, the time period sufficient for the subject to reach one or more dose continuation criteria is 1 to 10 weeks. In some embodiments of the methods provided herein, the time period sufficient for the subject to reach one or more dose continuation criteria is 1 to 4 months. In some embodiments of the methods provided herein, the time period sufficient for the subject to reach one or more dose continuation criteria is 1 day. In some embodiments of the methods provided herein, the time period sufficient for the subject to reach one or more dose continuation criteria is 2 days. In some embodiments of the methods provided herein, the time period sufficient for the subject to reach one or more dose continuation criteria is 3 days. In some embodiments of the methods provided herein, the time period sufficient for the subject to reach one or more dose continuation criteria is 4 days. In some embodiments of the methods provided herein, the time period sufficient for the subject to reach one or more dose continuation criteria is 5 days. In some embodiments of the methods provided herein, the time period sufficient for the subject to reach one or more dose continuation criteria is 6 days. In some embodiments of the methods provided herein, the time period sufficient for the subject to reach one or more dose continuation criteria is 7 days. In some embodiments of the methods provided herein, the time period sufficient for the subject to reach one or more dose continuation criteria is 8 days. In some embodiments of the methods provided herein, the time period sufficient for the subject to reach one or more dose continuation criteria is 9 days. In some embodiments of the methods provided herein, the time period sufficient for the subject to reach one or more dose continuation criteria is 10 days. In some embodiments of the methods provided herein, the time period sufficient for the subject to reach one or more dose continuation criteria is 1 week. In some embodiments of the methods provided herein, the time period sufficient for the subject to reach one or more dose continuation criteria is 2 weeks. In some embodiments of the methods provided herein, the time period sufficient for the subject to reach one or more dose continuation criteria is 3 weeks. In some embodiments of the methods provided herein, the time period sufficient for the subject to reach one or more dose continuation criteria is 4 weeks. In some embodiments of the methods provided herein, the time period sufficient for the subject to reach one or more dose continuation criteria is 5 weeks. In some embodiments of the methods provided herein, the time period sufficient for the subject to reach one or more dose continuation criteria is 6 weeks. In some embodiments of the methods provided herein, the time period sufficient for the subject to reach one or more dose continuation criteria is 7 weeks. In some embodiments of the methods provided herein, the time period sufficient for the subject to reach one or more dose continuation criteria is 8 weeks. In some embodiments of the methods provided herein, the time period sufficient for the subject to reach one or more dose continuation criteria is 1 month. In some embodiments of the methods provided herein, the time period sufficient for the subject to reach one or more dose continuation criteria is 2 months. In some embodiments of the methods provided herein, the time period sufficient for the subject to reach one or more dose continuation criteria is 3 months. In some embodiments of the methods provided herein, the time period sufficient for the subject to reach one or more dose continuation criteria is 4 months.

The disclosure provides that the modified dose can depend on the number of times the condition for the administration of the second regimen based on the one or more dose continuation criteria have been satisfied. As such, in some embodiments, the methods further comprises determining the number of times the condition for the administration of the second regimen based on the one or more dose continuation criteria have been satisfied.

In some embodiments, the dose reduction or modification referenced to in the paragraphs related to the one or more dose continuation criteria above and below, are set forth in Table 8 above.

In some embodiments of the methods provided herein, the ADC dose in the first regimen is the starting dose before any dose reduction or dose modification based on the one or more dose continuation criteria. Based on Table 8, in some embodiments, such ADC dose in the first regimen and the starting dose is 1.25 mg/kg for subject having a body weight of less than 100 kg or 125 mg for subject having a body weight of no less than 100 kg.

In some embodiments of the methods provided herein, if the second regimen is administered for the first time in (V), the second regimen can be identical to the first regimen. In some embodiments of the methods provided herein, the second regimen in (V) is identical to the first regimen when the second regimen is administered for the first time or has been administered one or more times in (V). In some embodiments of the methods provided herein, if the second regimen has been administered one or more times in (V) and the subject has a body weight of less than 100 kg, the ADC dose in the second regimen is lowered to about 1.0 mg/kg of the subject's body weight. In some embodiments of the methods provided herein, if the second regimen has been administered one or more times in (V) and the subject has a body weight of no less than 100 kg, the ADC dose in the second regimen is lowered to about 100 mg to the subject. In some embodiments of the methods provided herein, if the second regimen has been administered two or more times in (V) and the subject has a body weight of less than 100 kg, the ADC dose in the second regimen is lowered to about 0.75 mg/kg of the subject's body weight. In some embodiments of the methods provided herein, if the second regimen has been administered two or more times in (V) and the subject has a body weight of no less than 100 kg, the ADC dose in the second regimen is lowered to about 75 mg to the subject. In some embodiments of the methods provided herein, if the second regimen has been administered three or more times in (V) and the subject has a body weight of less than 100 kg, the ADC dose in the second regimen is lowered to about 0.5 mg/kg of the subject's body weight. In some embodiments of the methods provided herein, if the second regimen has been administered three or more times in (V) and the subject has a body weight of no less than 100 kg, the ADC dose in the second regimen is lowered to about 50 mg to the subject.

Alternatively, in some embodiments of the methods provided herein, if the subject has a body weight of less than 100 kg, the ADC dose in the second regimen is about 1.0 mg/kg of the subject's body weight. In some embodiments of the methods provided herein, if the subject has a body weight of no less than 100 kg, the ADC dose in the second regimen is about 100 mg to the subject. In some embodiments of the methods provided herein if the subject has a body weight of less than 100 kg, the ADC dose in the second regimen is about 1.0 mg/kg of the subject's body weight when the second regimen is administered for the first time or has been administered one or more times in (V). In some embodiments of the methods provided herein, if the subject has a body weight of no less than 100 kg, the ADC dose in the second regimen is about 100 mg to the subject when the second regimen is administered for the first time or has been administered one or more times in (V). In some embodiments of the methods provided herein, if the second regimen has been administered one or more times in (V) and the subject has a body weight of less than 100 kg, the ADC dose in the second regimen is lowered to about 0.75 mg/kg of the subject's body weight. In some embodiments of the methods provided herein, if the second regimen has been administered one or more times in (V) and the subject has a body weight of no less than 100 kg, the ADC dose in the second regimen is lowered to about 75 mg to the subject. In some embodiments of the methods provided herein, if the second regimen has been administered two or more times in (V) and the subject has a body weight of less than 100 kg, the ADC dose in the second regimen is lowered to about 0.5 mg/kg of the subject's body weight. In some embodiments of the methods provided herein, if the second regimen has been administered two or more times in (V) and the subject has a body weight of no less than 100 kg, the ADC dose in the second regimen is lowered to about 50 mg to the subject.

The disclosure provides that the one or more dose modification criteria, the one or more dose discontinuation criteria, and/or the one or more dose continuation criteria can be determined at various frequencies and intervals according to the need of the methods and/or the practice of the art. In some embodiments of the methods provided herein, the one or more dose modification criteria, the one or more dose discontinuation criteria, and/or the one or more dose continuation criteria are determined daily. In some embodiments of the methods provided herein, the one or more dose modification criteria, the one or more dose discontinuation criteria, and/or the one or more dose continuation criteria are determined once every two days, once every three days, once every four days, or once every five days, once every six days. In some embodiments of the methods provided herein, the one or more dose modification criteria, the one or more dose discontinuation criteria, and/or the one or more dose continuation criteria are determined weekly, bi-weekly, once every three weeks, or once every four weeks. In some embodiments of the methods provided herein, the one or more dose modification criteria, the one or more dose discontinuation criteria, and/or the one or more dose continuation criteria are determined monthly, once every two months, or once every three months.

As is clear from the above descriptions, in some embodiments of the methods provided herein, the one or more dose modification criteria, the one or more dose discontinuation criteria, and/or the one or more dose continuation criteria are used in corresponding combinations. Accordingly, in some embodiments of the methods provided herein, certain one or more dose modification criteria correspond to and/or are used in combination with certain specific one or more dose discontinuation criteria and certain specific one or more dose continuation criteria. Similarly, in some embodiments of the methods provided herein, certain one or more dose discontinuation criteria correspond to and/or are used in combination with certain specific one or more dose modification criteria and certain specific one or more dose continuation criteria. Additionally, certain one or more dose continuation criteria correspond to and/or are used in combination with certain specific one or more dose discontinuation criteria and certain specific one or more dose modification criteria.

Specifically, in some embodiments of the methods provided herein, the dose modification criteria, dose discontinuation criteria, and the dose continuation criteria are selected from any one or more of the following combinations:
i. the one or more dose modification criteria are blood glucose level higher than 250 mg/dL; the one or more dose discontinuation criteria are none; and the one or more dose continuation criteria are blood glucose level no more than 250 mg/dL;
ii. the one or more dose modification criteria are blood glucose level higher than 250 mg/dL; the one or more dose discontinuation criteria are blood glucose level higher than 500; and the one or more dose continuation criteria are blood glucose level no more than 250 mg/dL;
iii. the one or more dose modification criteria are hyperglycemia no less than Grade 3; the one or more dose discontinuation criteria are hyperglycemia no less than Grade 4; and the one or more dose continuation criteria are hyperglycemia no more than Grade 2;
iv. the one or more dose modification criteria are peripheral neuropathy no less than Grade 2; the one or more dose discontinuation criteria are peripheral neuropathy no less than Grade 3; and the one or more dose continuation criteria are peripheral neuropathy no more than Grade 1;
v. the one or more dose modification criteria are skin reaction from no less than Grade 3; the one or more dose discontinuation criteria are skin reaction no less than Grade 4 or recurrent Grade 3 skin reaction; and the one or more dose continuation criteria are skin reaction no more than Grade 1;
vi. the one or more dose modification criteria are non-hematologic toxicity no less than Grade 3; the one or more dose discontinuation criteria are non-hematologic toxicity no less than Grade 4; and the one or more dose continuation criteria are non-hematologic toxicity no more than Grade 1;
vii. the one or more dose modification criteria are ocular disorder no less than Grade 3; the one or more dose discontinuation criteria are ocular disorder no less than Grade 4; and the one or more dose continuation criteria are ocular disorder no more than Grade 1;
viii. the one or more dose modification criteria are dysgeusia no less than Grade 3; the one or more dose discontinuation criteria are dysgeusia no less than Grade 4; and the one or more dose continuation criteria are dysgeusia no more than Grade 1;
ix. the one or more dose modification criteria are loss of appetite no less than Grade 3; the one or more dose discontinuation criteria are loss of appetite no less than Grade 4; and the one or more dose continuation criteria are loss of appetite no more than Grade 1;
x. the one or more dose modification criteria are anorexia no less than Grade 3; the one or more dose discontinuation criteria are anorexia no less than Grade 4; and the one or more dose continuation criteria are anorexia no more than Grade 1;
xi. the one or more dose modification criteria are fatigue no less than Grade 3; the one or more dose discontinuation criteria are fatigue no less than Grade 4; and the one or more dose continuation criteria are fatigue no more than Grade 1;
xii. the one or more dose modification criteria are diarrhea no less than Grade 3; the one or more dose discontinuation criteria are diarrhea no less than Grade 4; and the one or more dose continuation criteria are diarrhea no more than Grade 1;
xiii. the one or more dose modification criteria are hematologic toxicity no less than Grade 2; the one or more dose discontinuation criteria are hematologic toxicity no less than Grade 4; and the one or more dose continuation criteria are the hematologic toxicity no more than Grade 1;
xiv. the one or more dose modification criteria are hematologic toxicity no less than Grade 2; the one or more dose discontinuation criteria are none; and the one or more dose continuation criteria are the hematologic toxicity no more than Grade 1;
xv. the one or more dose modification criteria are thrombocytopenia no less than Grade 2; the one or more dose discontinuation criteria are thrombocytopenia no less than Grade 4; and the one or more dose continuation criteria are the thrombocytopenia no more than Grade 1; and
xvi. the one or more dose modification criteria are thrombocytopenia no less than Grade 2; the one or more dose discontinuation criteria are none; and the one or more dose continuation criteria are the thrombocytopenia no more than Grade 1;

In some embodiments of the methods provided herein, the combinations of the one or more dose modification criteria, the one or more dose discontinuation criteria, and/or the one or more dose continuation criteria are used in corresponding combinations are independently. In some embodiments of the methods provided herein, the combinations of the one or more dose modification criteria, the one or more dose discontinuation criteria, and/or the one or more dose continuation criteria are used in corresponding combinations are determined in parallel. In some embodiments of the methods provided herein, the combinations of the one or more dose modification criteria, the one or more dose discontinuation criteria, and/or the one or more dose continuation criteria are used in corresponding combinations are determined independently and in parallel.

### 5.3 Anti-191P4D12 Antibody Drug Conjugate

In general the methods provided herein utilize an anti-191P4D12 ADC described herein and/or in U.S. Pat. No. 8,637,642, which is herein incorporated in its entirety by reference. The anti-191P4D12 antibody drug conjugate provided herein comprises an antibody or antigen binding fragment thereof that binds to 191P4D12 conjugated to one or more units of cytotoxic agents (or drug units). The cytotoxic agents (or drug units) can be covalently linked directly or via a linker unit (LU).

In some embodiments, the antibody drug conjugate compound has the following formula:

L-(LU-D)ₚ (I)
or a pharmaceutically acceptable salt or solvate thereof; wherein:
L is the antibody unit, e.g., the anti-191P4D12 antibody or an antigen binding fragment thereof as provided in Section 5.3.1 below, and
(LU-D) is a linker unit-drug unit moiety, wherein:
LU- is a linker unit, and
D is a drug unit having cytostatic or cytotoxic activity against a target cell; and
p is an integer from 1 to 20.

In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20.

In some embodiments, the antibody drug conjugate compound has the following formula:

L-(Aₐ-W_{w}—Y_{y}-D)ₚ (II)
or a pharmaceutically acceptable salt or solvate thereof, wherein:
L is the Antibody unit, e.g., the anti-191P4D12 antibody or an antigen binding fragment thereof as provided in Section 5.3.1 below; and
-Aₐ-W_{w}—Y_{y}— is a linker unit (LU), wherein:
-A- is a stretcher unit,
a is 0 or 1,
each —W— is independently an amino acid unit,
w is an integer ranging from 0 to 12,
—Y— is a self-immolative spacer unit,
y is 0, 1 or 2;
D is a drug units having cytostatic or cytotoxic activity against the target cell; and
p is an integer from 1 to 20.

In some embodiments, a is 0 or 1, w is 0 or 1, and y is 0, 1 or 2. In some embodiments, a is 0 or 1, w is 0 or 1, and y is 0 or 1. In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20. In some embodiments, when w is not zero, y is 1 or 2. In some embodiments, when w is 1 to 12, y is 1 or 2. In some embodiments, w is 2 to 12 and y is 1 or 2. In some embodiments, a is 1 and w and y are 0.

For compositions comprising a plurality antibodies or antigen binding fragments thereof, the drug loading is represented by p, the average number of drug molecules per antibody unit. Drug loading may range from 1 to 20 drugs (D) per antibody. The average number of drugs per antibody in preparation of conjugation reactions may be characterized by conventional means such as mass spectroscopy, ELISA assay, and HPLC. The quantitative distribution of antibody drug conjugates in terms of p may also be determined. In some instances, separation, purification, and characterization of homogeneous antibody drug conjugates where p is a certain value from antibody drug conjugates with other drug loadings may be achieved by means such as reverse phase HPLC or electrophoresis. In exemplary embodiments, p is from 2 to 8.

### 5.3.1 Anti-191P4D12 Antibodies or Antigen Binding Fragments

In one embodiment, the antibody or antigen binding fragment thereof that binds to 191P4D12-related proteins is an antibody or antigen binding fragment that specifically binds to 191P4D12 protein comprising amino acid sequence of SEQ ID NO:2 (see FIG. 1A). The corresponding cDNA encoding the 191P4D12 protein has a sequence of SEQ ID NO:1 (see FIG. 1A).

The antibody that specifically binds to 191P4D12 protein comprising amino acid sequence of SEQ ID NO:2 includes antibodies that can bind to other 191P4D12-related proteins. For example, antibodies that bind 191P4D12 protein comprising amino acid sequence of SEQ ID NO:2 can bind 191P4D12-related proteins such as 191P4D12 variants and the homologs or analogs thereof.

In some embodiments, the anti-191P4D12 antibody provided herein is a monoclonal antibody.

In some embodiments, the antibody comprises a heavy chain comprising an amino acid sequence of SEQ ID NO:4 (cDNA sequence of SEQ ID NO:3), and/or a light chain comprising an amino acid sequence of SEQ ID NO: 6 (cDNA sequence of SEQ ID NO:5), as shown in FIGS. 1B and 1C.

In some embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising CDRs comprising the amino acid sequences of the CDRs of the light chain variable region set forth in SEQ ID NO:23 (which is the amino acid sequence ranging from the 23rd amino acid (aspartic acid) to the 130th amino acid (arginine) of SEQ ID NO:8). SEQ ID NO: 22, SEQ ID NO:23, SEQ ID NO:7 and SEQ ID NO:8 are as shown in FIGS. 1D and 1E and listed below:

SEQ ID NO: 22
EVQLVESGGGLVQPGGSLRLSCAASGFTFSSYNMN
WVRQAPGKGLEWVSYISSSSSTIYYADSVKGRFTI
SRDNAKNSLSLQMNSLRDEDTAVYYCARAYYYGMD
VWGQGTTVTVSS
SEQ ID NO: 23
DIQMTQSPSSVSASVGDRVTITCRASQGISGWLAW
YQQKPGKAPKFLIYAASTLQSGVPSRFSGSGSGTD
FTLTISSLQPEDFATYYCQQANSFPPTFGGGTKVE
IKR
SEQ ID NO: 7
MELGLCWVFLVAILEGVQCEVQLVESGGGLVQPGG
SLRLSCAASGFTFSSYNMNWVRQAPGKGLEWVSYI
SSSSSTIYYADSVKGRFTISRDNAKNSLSLQMNSL
RDEDTAVYYCARAYYYGMDVWGQGTTVTVSSASTK
GPSVFPLAPSSKSTSGGTAALGCLVKDYFPEPVTV
SWNSGALTSGVHTFPAVLQSSGLYSLSSWTVPSSS
LGTQTYICNVNHKPSNTKVDKRVEPKSCDKTHTCP
PCPAPELLGGPSVFLFPPKPKDTLMISRTPEVTCV
VVDVSHEDPEVKFNWYVDGVEVHNAKTKPREEQYN
STYRWSVLTVLHQDWLNGKEYKCKVSNKALPAPIE
KTISKAKGQPREPQVYTLPPSREEMTKNQVSLTCL
VKGFYPSDIAVEWESNGQPENNYKTTPPVLDSDGS
FFLYSKLTVDKSRWQQGNVFSCSVMHEALHNHYTQ
KSLSLSPGK
SEQ ID NO: 8
MDMRVPAQLLGLLLLWFPGSRCDIQMTQSPSSVSA
SVGDRVTITCRASQGISGWLAWYQQKPGKAPKFLI
YAASTLQSGVPSRFSGSGSGTDFTLTISSLQPEDF
ATYYCQQANSFPPTFGGGTKVEIKRTVAAPSVFIF
PPSDEQLKSGTASVVCLLNNFYPREAKVQWKVDNA
LQSGNSQESVTEQDSKDSTYSLSSTLTLSKADYEK
HKVYACEVTHQGLSSPVTKSFNRGEC

CDR sequences can be determined according to well-known numbering systems. As described above, CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted in Table 1 above.

In some embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:22 according to Kabat numbering and a light chain variable region comprising CDRs comprising the amino acid sequences of the CDRs of the light chain variable region set forth in SEQ ID NO:23 according to Kabat numbering.

In some embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:22 according to AbM numbering and a light chain variable region comprising CDRs comprising the amino acid sequences of the CDRs of the light chain variable region set forth in SEQ ID NO:23 according to AbM numbering.

In other embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:22 according to Chothia numbering and a light chain variable region comprising CDRs comprising the amino acid sequences of the CDRs of the light chain variable region set forth in SEQ ID NO:23 according to Chothia numbering.

In other embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:22 according to Contact numbering and a light chain variable region comprising CDRs comprising the amino acid sequences of the CDRs of the light chain variable region set forth in SEQ ID NO:23 according to Contact numbering.

In yet other embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:22 according to IMGT numbering and a light chain variable region comprising CDRs comprising the amino acid sequences of the CDRs of the light chain variable region set forth in SEQ ID NO:23 according to IMGT numbering.

As described above, the CDR sequences according to different numbering systems can be readily determined, e.g., using online tools such as the one provided by Antigen receptor Numbering And Receptor Classification (ANARCI). For example, the heavy chain CDR sequences within SEQ ID NO:22, and the light chain CDR sequences within SEQ ID NO:23 according to Kabat numbering as determined by ANARCI are listed in Table 19 below.

TABLE 19

| | VH of SEQ ID NO: 22 | VL of SEQ ID NO: 23 |
|---|---|---|
| CDR1 | SYNMN | RASQGISGWLA |
| | (SEQ ID NO: 9) | (SEQ ID NO: 12) |
| CDR2 | YISSSSSTIYYADSVKG | AASTLQS |
| | (SEQ ID NO: 10) | (SEQ ID NO: 13) |
| CDR3 | AYYYGMDV | QQANSFPPT |
| | (SEQ ID NO: 11) | (SEQ ID NO: 14) |

For another example, the heavy chain CDR sequences within SEQ ID NO:22, and the light chain CDR sequences within SEQ ID NO:23 according to IMGT numbering as determined by ANARCI are listed in Table 20 below.

TABLE 20

| | VH of SEQ ID NO: 22 | VL of SEQ ID NO: 23 |
|---|---|---|
| CDR1 | GFTFSSYN | QGISGW |
| | (SEQ ID NO: 16) | (SEQ ID NO: 19) |
| CDR2 | ISSSSSTI | AAS |
| | (SEQ ID NO: 17) | (SEQ ID NO: 20) |
| CDR3 | ARAYYYGMDV | QQANSFPPT |
| | (SEQ ID NO: 18) | (SEQ ID NO: 21) |

In some embodiments, the antibody or antigen binding fragment thereof comprises CDR H1 comprising an amino acid sequence of SEQ ID NO:9, CDR H2 comprising an amino acid sequence of SEQ ID NO:10, CDR H3 comprising an amino acid sequence of SEQ ID NO:11, CDR L1 comprising an amino acid sequence of SEQ ID NO:NO:12, CDR L2 comprising an amino acid sequence of SEQ ID NO:NO:13, and CDR L3 comprising an amino acid sequence of SEQ ID NO:NO:14.

In some embodiments, the antibody or antigen binding fragment thereof comprises CDR H1 comprising an amino acid sequence of SEQ ID NO:16, CDR H2 comprising an amino acid sequence of SEQ ID NO:17, CDR H3 comprising an amino acid sequence of SEQ ID NO:18, CDR L1 comprising an amino acid sequence of SEQ ID NO:NO:19, CDR L2 comprising an amino acid sequence of SEQ ID NO:NO:20, and CDR L3 comprising an amino acid sequence of SEQ ID NO:NO:21.

In some embodiments, the antibody or antigen binding fragment thereof comprises CDR H1 consisting of an amino acid sequence of SEQ ID NO:9, CDR H2 consisting of an amino acid sequence of SEQ ID NO:10, CDR H3 consisting of an amino acid sequence of SEQ ID NO:11, CDR L1 consisting of an amino acid sequence of SEQ ID NO:NO:12, CDR L2 consisting of an amino acid sequence of SEQ ID NO:NO:13, and CDR L3 consisting of an amino acid sequence of SEQ ID NO:NO:14.

In some embodiments, the antibody or antigen binding fragment thereof comprises CDR H1 consisting of an amino acid sequence of SEQ ID NO:16, CDR H2 consisting of an amino acid sequence of SEQ ID NO:17, CDR H3 consisting of an amino acid sequence of SEQ ID NO:18, CDR L1 consisting of an amino acid sequence of SEQ ID NO:NO:19, CDR L2 consisting of an amino acid sequence of SEQ ID NO:NO:20, and CDR L3 consisting of an amino acid sequence of SEQ ID NO:NO:21.

In some embodiments, the antibody or antigen binding fragment thereof comprises a heavy chain variable region comprising the amino acid sequence of SEQ ID NO:22 and a light chain variable region comprising the amino acid sequence of SEQ ID NO:23.

In some embodiments, the antibody or antigen binding fragment thereof comprises a heavy chain variable region consisting of the amino acid sequence of SEQ ID NO:22 and a light chain variable region consisting of the amino acid sequence of SEQ ID NO:23.

In some embodiments, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

In some embodiments, the antibody comprises a heavy chain consisting of the amino acid sequence ranging from the 20th amino acid (glutamic acid) to the 466th amino acid (lysine) of SEQ ID NO:7 and a light chain consisting of the amino acid sequence ranging from the 23rd amino acid (aspartic acid) to the 236th amino acid (cysteine) of SEQ ID NO:8.

In some embodiments, amino acid sequence modification(s) of antibodies described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art who appreciate that amino acid changes may alter post-translational processes of the antibody, such as changing the number or position of glycosylation sites or altering the membrane anchoring characteristics.

In some embodiments, the antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

Variations may be a substitution, deletion, or insertion of one or more codons encoding the single domain antibody or polypeptide that results in a change in the amino acid sequence as compared with the original antibody or polypeptide. Amino acid substitutions can be the result of replacing one amino acid with another amino acid comprising similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Antibodies generated by conservative amino acid substitutions are included in the present disclosure. In a conservative amino acid substitution, an amino acid residue is replaced with an amino acid residue comprising a side chain with a similar charge. As described above, families of amino acid residues comprising side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined conservative (e.g., within an amino acid group with similar properties and/or side chains) substitutions may be made, so as to maintain or not significantly change the properties.

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

For example, any cysteine residue not involved in maintaining the proper conformation of the antibody also may be substituted, for example, with another amino acid, such as alanine or serine, to improve the oxidative stability of the molecule and to prevent aberrant crosslinking.

The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the anti-anti-MSLN antibody variant DNA.

Covalent modifications of antibodies are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

In some embodiments, the antibody or antigen binding fragment provided herein comprises a heavy chain having more than 70% homology or identity to the heavy chain as set forth in SEQ ID NO:7. In some embodiments, the antibody or antigen binding fragment provided herein comprises a heavy chain having more than 75% homology or identity to the heavy chain as set forth in SEQ ID NO:7. In some embodiments, the antibody or antigen binding fragment provided herein comprises a heavy chain having more than 80% homology or identity to the heavy chain as set forth in SEQ ID NO:7. In some embodiments, the antibody or antigen binding fragment provided herein comprises a heavy chain having more than 85% homology or identity to the heavy chain as set forth in SEQ ID NO:7. In some embodiments, the antibody or antigen binding fragment provided herein comprises a heavy chain having more than 90% homology or identity to the heavy chain as set forth in SEQ ID NO:7. In some embodiments, the antibody or antigen binding fragment provided herein comprises a heavy chain having more than 95% homology or identity to the heavy chain as set forth in SEQ ID NO:7.

In some embodiments, the antibody or antigen binding fragment provided herein comprises a light chain having more than 70% homology or identity to the light chain as set forth in SEQ ID NO:8. In some embodiments, the antibody or antigen binding fragment provided herein comprises a light chain having more than 75% homology or identity to the light chain as set forth in SEQ ID NO:8. In some embodiments, the antibody or antigen binding fragment provided herein comprises a light chain having more than 80% homology or identity to the light chain as set forth in SEQ ID NO:8. In some embodiments, the antibody or antigen binding fragment provided herein comprises a light chain having more than 85% homology or identity to the light chain as set forth in SEQ ID NO:8. In some embodiments, the antibody or antigen binding fragment provided herein comprises a light chain having more than 90% homology or identity to the light chain as set forth in SEQ ID NO:8. In some embodiments, the antibody or antigen binding fragment provided herein comprises a light chain having more than 95% homology or identity to the light chain as set forth in SEQ ID NO:8.

In some embodiments, the anti-191P4D12 antibody provided herein comprises heavy and light chain CDR regions of an antibody designated Ha22-2(2,4)6.1 produced by a hybridoma deposited under the American Type Culture Collection (ATCC) Accession NO: PTA-11267, or heavy and light chain CDR regions comprising amino acid sequences that are homologous to the amino acid sequences of the heavy and light chain CDR regions of Ha22-2(2,4)6.1, and wherein the antibodies retain the desired functional properties of the anti-191P4D12 antibody designated Ha22-2(2,4)6.1 produced by a hybridoma deposited under the American Type Culture Collection (ATCC) Accession NO: PTA-11267.

In some embodiments, the anti-191P4D12 antibody provided herein comprises heavy and light chain CDR regions of an antibody designated Ha22-2(2,4)6.1 produced by a hybridoma deposited under the American Type Culture Collection (ATCC) Accession NO: PTA-11267, or heavy and light chain CDR regions consisting of amino acid sequences that are homologous to the amino acid sequences of the heavy and light chain CDR regions of Ha22-2(2,4)6.1, and wherein the antibodies retain the desired functional properties of the anti-191P4D12 antibody designated Ha22-2(2,4)6.1 produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:

(a) the heavy chain variable region comprises CDRs comprising the amino acid sequences of the heavy chain variable region CDRs set forth in the antibody produced by a hybridoma deposited under the American Type Culture Collection (ATCC) Accession NO: PTA-11267;

(b) the light chain variable region comprises CDRs comprising the amino acid sequences of the light chain variable region CDRs set forth in the antibody produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:

(a) the heavy chain variable region comprises CDRs consisting of the amino acid sequences of the heavy chain variable region CDRs set forth in the antibody produced by a hybridoma deposited under the American Type Culture Collection (ATCC) Accession NO: PTA-11267;

(b) the light chain variable region comprises CDRs consisting of the amino acid sequences of the light chain variable region CDRs set forth in the antibody produced by a hybridoma deposited under the American Type Culture Collection (ATCC) Accession NO: PTA-11267.

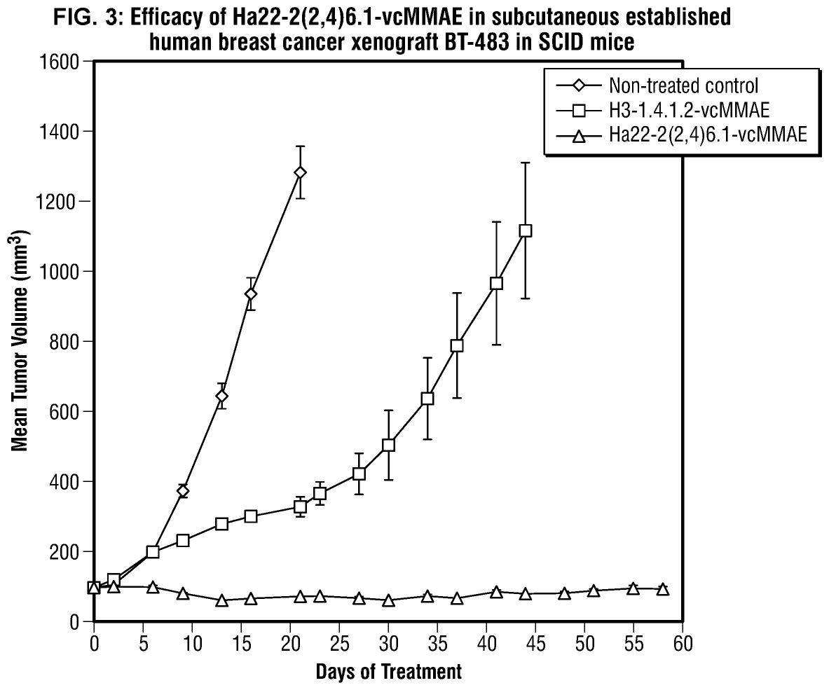

In some embodiments, the anti-191P4D12 antibody provided herein comprises heavy and light chain variable regions of an antibody designated Ha22-2(2,4)6.1 produced by a hybridoma deposited under the American Type Culture Collection (ATCC) Accession NO: PTA-11267 (See, FIG. 3), or heavy and light variable regions comprising amino acid sequences that are homologous to the amino acid sequences of the heavy and light chain variable regions of Ha22-2(2,4)6.1, and wherein the antibodies retain the desired functional properties of the anti-191P4D12 antibody provided herein. In some embodiments, the anti-191P4D12 antibody provided herein comprises heavy and light chain variable regions of an antibody designated Ha22-2(2,4)6.1 produced by a hybridoma deposited under the American Type Culture Collection (ATCC) Accession NO: PTA-11267 (See, FIG. 3), or heavy and light variable regions consisting of amino acid sequences that are homologous to the amino acid sequences of the heavy and light chain variable regions of Ha22-2(2,4)6.1, and wherein the antibodies retain the desired functional properties of the anti-191P4D12 antibody provided herein. As the constant region of the antibody of the invention, any subclass of constant region can be chosen. In one embodiment, human IgG1 constant region as the heavy chain constant region and human Ig kappa constant region as the light chain constant region can be used.

In some embodiments, the anti-191P4D12 antibody provided herein comprises heavy and light chains of an antibody designated Ha22-2(2,4)6.1 produced by a hybridoma deposited under the American Type Culture Collection (ATCC) Accession NO: PTA-11267 (See, FIG. 3), or heavy and light chains comprising amino acid sequences that are homologous to the amino acid sequences of the heavy and light chains of Ha22-2(2,4)6.1, and wherein the antibodies retain the desired functional properties of the anti-191P4D12 antibody provided herein. In some embodiments, the anti-191P4D12 antibody provided herein comprises heavy and light chains of an antibody designated Ha22-2(2,4)6.1 produced by a hybridoma deposited under the American Type Culture Collection (ATCC) Accession NO: PTA-11267 (See, FIG. 3), or heavy and light chains consisting of amino acid sequences that are homologous to the amino acid sequences of the heavy and light chains of Ha22-2(2,4)6.1, and wherein the antibodies retain the desired functional properties of the anti-191P4D12 antibody provided herein.

In some embodiments, the antibody or antigen binding fragment thereof provided herein comprises a heavy chain variable region and a light chain variable region, wherein:

(a) the heavy chain variable region comprises an amino acid sequence that is at least 80% homologous or identical to the heavy chain variable region amino acid sequence of the antibody produced by a hybridoma deposited under the American Type Culture Collection (ATCC) Accession NO: PTA-11267; and

(b) the light chain variable region comprises an amino acid sequence that is at least 80% homologous or identical to the light chain variable region amino acid sequence of the antibody produced by a hybridoma deposited under the American Type Culture Collection (ATCC) Accession NO: PTA-11267.

In some embodiments, the heavy chain variable region comprises an amino acid sequence that is at least 85% homologous or identical to the heavy chain variable region amino acid sequence of the antibody produced by a hybridoma deposited under the American Type Culture Collection (ATCC) Accession NO: PTA-11267. In other embodiments, the heavy chain variable region comprises an amino acid sequence that is at least 90% homologous or identical to the heavy chain variable region amino acid sequence of the antibody produced by a hybridoma deposited under the American Type Culture Collection (ATCC) Accession NO: PTA-11267. In yet other embodiments, the heavy chain variable region comprises an amino acid sequence that is at least 95% homologous or identical to the heavy chain variable region amino acid sequence of the antibody produced by a hybridoma deposited under the American Type Culture Collection (ATCC) Accession NO: PTA-11267. In other embodiments, the heavy chain variable region may be 85%, 86%, 87%, 88%, 89%, 90%, 91%, 92%, 93%, 94%, 95%, 96%, 97%, 98% or 99% homologous or identical to the heavy chain variable region amino acid sequence of the antibody produced by a hybridoma deposited under the American Type Culture Collection (ATCC) Accession NO: PTA-11267.

In some embodiments, the light chain variable region comprises an amino acid sequence that is at least 85% homologous or identical to the light chain variable region amino acid sequence of the antibody produced by a hybridoma deposited under the American Type Culture Collection (ATCC) Accession NO: PTA-11267. In other embodiments, the light chain variable region comprises an amino acid sequence that is at least 90% homologous or identical to the light chain variable region amino acid sequence of the antibody produced by a hybridoma deposited under the American Type Culture Collection (ATCC) Accession NO: PTA-11267. In yet other embodiments, the light chain variable region comprises an amino acid sequence that is at least 95% homologous or identical to the light chain variable region amino acid sequence of the antibody produced by a hybridoma deposited under the American Type Culture Collection (ATCC) Accession NO: PTA-11267. In other embodiments, the light chain variable region may be 85%, 86%, 87%, 88%, 89%, 90%, 91%, 92%, 93%, 94%, 95%, 96%, 97%, 98% or 99% homologous or identical to the light chain variable region amino acid sequence of the antibody produced by a hybridoma deposited under the American Type Culture Collection (ATCC) Accession NO: PTA-11267.

In other embodiments, the antibody or antigen binding fragment thereof provided herein comprises a heavy chain and a light chain, wherein:

(a) the heavy chain comprises an amino acid sequence that is at least 80% homologous or identical to the heavy chain amino acid sequence of the antibody produced by a hybridoma deposited under the American Type Culture Collection (ATCC) Accession NO: PTA-11267; and

(b) the light chain comprises an amino acid sequence that is at least 80% homologous or identical to the light chain amino acid sequence of the antibody produced by a hybridoma deposited under the American Type Culture Collection (ATCC) Accession NO: PTA-11267.

In some embodiments, the heavy chain comprises an amino acid sequence that is at least 85% homologous or identical to the heavy chain amino acid sequence of the antibody produced by a hybridoma deposited under the American Type Culture Collection (ATCC) Accession NO: PTA-11267. In other embodiments, the heavy chain comprises an amino acid sequence that is at least 90% homologous or identical to the heavy chain amino acid sequence of the antibody produced by a hybridoma deposited under the American Type Culture Collection (ATCC) Accession NO: PTA-11267. In yet other embodiments, the heavy chain comprises an amino acid sequence that is at least 95% homologous or identical to the heavy chain amino acid sequence of the antibody produced by a hybridoma deposited under the American Type Culture Collection (ATCC) Accession NO: PTA-11267. In other embodiments, the heavy chain may be 85%, 86%, 87%, 88%, 89%, 90%, 91%, 92%, 93%, 94%, 95%, 96%, 97%, 98% or 99% homologous or identical to the heavy chain amino acid sequence of the antibody produced by a hybridoma deposited under the American Type Culture Collection (ATCC) Accession NO: PTA-11267.

In some embodiments, the light chain comprises an amino acid sequence that is at least 85% homologous or identical to the light chain amino acid sequence of the antibody produced by a hybridoma deposited under the American Type Culture Collection (ATCC) Accession NO: PTA-11267. In other embodiments, the light chain comprises an amino acid sequence that is at least 90% homologous or identical to the light chain amino acid sequence of the antibody produced by a hybridoma deposited under the American Type Culture Collection (ATCC) Accession NO: PTA-11267. In yet other embodiments, the light chain comprises an amino acid sequence that is at least 95% homologous or identical to the light chain amino acid sequence of the antibody produced by a hybridoma deposited under the American Type Culture Collection (ATCC) Accession NO: PTA-11267. In other embodiments, the light chain may be 85%, 86%, 87%, 88%, 89%, 90%, 91%, 92%, 93%, 94%, 95%, 96%, 97%, 98% or 99% homologous or identical to the light chain amino acid sequence of the antibody produced by a hybridoma deposited under the American Type Culture Collection (ATCC) Accession NO: PTA-11267.

In some embodiments, the antibody or antigen binding fragment thereof provided herein binds to a specific epitope in 191P4D12. In some embodiments, the antibody or antigen binding fragment thereof provided herein binds to VC1 domain of 191P4D12. In some embodiments, the antibody or antigen binding fragment thereof provided herein binds to VC1 domain but not to C1C2 domain of 191P4D12. In some embodiments, the antibody or antigen binding fragment thereof provided herein binds to the 1st to 147th amino acid residues of 191P4D12. In some embodiments, the antibody or antigen binding fragment thereof provided herein binds to an epitope located in the 1st to 147th amino acid residues of 191P4D12. In some embodiments, the antibody or antigen binding fragment thereof provided herein binds to the 1st to 10th amino acid residues of 191P4D12. In some embodiments, the antibody or antigen binding fragment thereof provided herein binds to the 11th to 20th amino acid residues of 191P4D12. In some embodiments, the antibody or antigen binding fragment thereof provided herein binds to the 21st to 30th amino acid residues of 191P4D12. In some embodiments, the antibody or antigen binding fragment thereof provided herein binds to the 31st to 40th amino acid residues of 191P4D12. In some embodiments, the antibody or antigen binding fragment thereof provided herein binds to the 41st to 50th amino acid residues of 191P4D12. In some embodiments, the antibody or antigen binding fragment thereof provided herein binds to the 51st to 60th amino acid residues of 191P4D12. In some embodiments, the antibody or antigen binding fragment thereof provided herein binds to the 61st to 70th amino acid residues of 191P4D12. In some embodiments, the antibody or antigen binding fragment thereof provided herein binds to the 71st to 80th amino acid residues of 191P4D12. In some embodiments, the antibody or antigen binding fragment thereof provided herein binds to the 81st to 90th amino acid residues of 191P4D12. In some embodiments, the antibody or antigen binding fragment thereof provided herein binds to the 91st to 100th amino acid residues of 191P4D12. In some embodiments, the antibody or antigen binding fragment thereof provided herein binds to the 101st to 110th amino acid residues of 191P4D12. In some embodiments, the antibody or antigen binding fragment thereof provided herein binds to the 111th to 120th amino acid residues of 191P4D12. In some embodiments, the antibody or antigen binding fragment thereof provided herein binds to the 121st to 130th amino acid residues of 191P4D12. In some embodiments, the antibody or antigen binding fragment thereof provided herein binds to the 131st to 140th amino acid residues of 191P4D12. In some embodiments, the antibody or antigen binding fragment thereof provided herein binds to the 141st to 147th amino acid residues of 191P4D12. The binding epitopes of certain embodiments the antibodies or antigen binding fragments thereof provided herein have been determined and described in WO 2012/047724, which is incorporated herein in its entirety by reference.

In some embodiments, the antibody or antigen binding fragment thereof provided herein binds to epitopes in 191P4D12 that are common between the 191P4D12 variants observed in human. In some embodiments, the antibody or antigen binding fragment thereof provided herein binds to epitopes in 191P4D12 that are common between the 191P4D12 polymorphysm observed in human. In some embodiments, the antibody or antigen binding fragment thereof provided herein binds to epitopes in 191P4D12 that are common between the 191P4D12 polymorphysm observed in human cancers. In some embodiments, the antibody or antigen binding fragment thereof provided herein binds to epitopes in 191P4D12 that would bind, internalize, disrupt or modulate the biological function of 191P4D12 or 191P4D12 variants. In some embodiments, the antibody or antigen binding fragment thereof provided herein binds to epitopes in 191P4D12 that would disrupt the interaction between 191P4D12 with ligands, substrates, and binding partners.

Engineered antibodies provided herein include those in which modifications have been made to framework residues within VH and/or VL (e.g. to improve the properties of the antibody). Typically, such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis (e.g., “backmutated” from leucine to methionine). Such “backmutated” antibodies are also intended to be encompassed by the invention.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 2003/0153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an anti-191P4D12 antibody provided herein may be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the anti-191P4D12 antibody.

In another embodiment, the Fc hinge region of an antibody is mutated to decrease the biological half-life of the anti-191P4D12 antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the anti-191P4D12 antibody is modified to increase its biological half-life. Various approaches are possible. For example, mutations can be introduced as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid specific residues can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

Reactivity of the anti-191P4D12 antibodies with a 191P4D12-related protein can be established by a number of well-known means, including Western blot, immunoprecipitation, ELISA, and FACS analyses using, as appropriate, 191P4D12-related proteins, 191P4D12-expressing cells or extracts thereof. A 191P4D12 antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more 191P4D12 epitopes are generated using methods generally known in the art. Homodimeric antibodies can also be generated by cross-linking techniques known in the art (e.g., Wolff et al., Cancer Res. 53: 2560-2565).

In yet another specific embodiment, the anti-191P4D12 antibody provided herein is an antibody comprising heavy and light chain of an antibody designated Ha22-2(2,4)6.1. The heavy chain of Ha22-2(2,4)6.1 consists of the amino acid sequence ranging from 20^{th} E residue to the 466^{th} K residue of SEQ ID NO:7 and the light chain of Ha22-2(2,4)6.1 consists of amino acid sequence ranging from 23^{rd} D residue to the 236^{th} C residue of SEQ ID NO:8 sequence.

The hybridoma producing the antibody designated Ha22-2(2,4)6.1 was sent (via Federal Express) to the American Type Culture Collection (ATCC), P.O. Box 1549, Manassas, Va. 20108 on 18 Aug. 2010 and assigned Accession number PTA-11267.

### 5.3.2 Cytotoxic Agents (Drug Units)

In some embodiments, the ADC comprises an antibody or antigen binding fragment thereof conjugated to dolastatins or dolostatin peptidic analogs and derivatives, the auristatins (U.S. Pat. Nos. 5,635,483; 5,780,588). Dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin or auristatin drug unit may be attached to the antibody through the N (amino) terminus or the C (carboxyl) terminus of the peptidic drug unit (WO 02/088172).

Exemplary auristatin embodiments include the N-terminus linked monomethylauristatin drug units DE and DF, disclosed in “Senter et al, Proceedings of the American Association for Cancer Research, Volume 45, Abstract Number 623, presented Mar. 28, 2004 and described in United States Patent Publication No. 2005/0238649, the disclosure of which is expressly incorporated by reference in its entirety.

In some embodiments, the auristatin is MMAE (wherein the wavy line indicates the covalent attachment to a linker of an antibody drug conjugate).

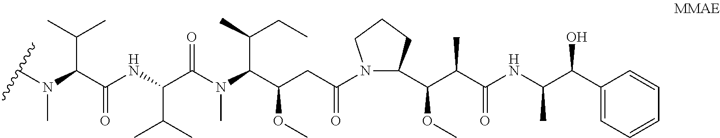 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, an exemplary embodiment comprising MMAE and a linker component (described further herein) has the following structure (wherein L presents the antibody and p ranges from 1 to 12):

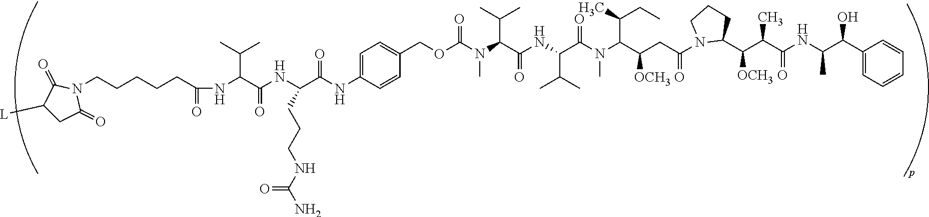

In some embodiments of the formula described in the preceding paragraph, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments of the formula described in the preceding paragraph, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments of the formula described in the preceding paragraph, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments of the formula described in the preceding paragraph, p is about 1. In some embodiments of the formula described in the preceding paragraph, p is about 2. In some embodiments of the formula described in the preceding paragraph, p is about 3. In some embodiments of the formula described in the preceding paragraph, p is about 4. In some embodiments of the formula described in the preceding paragraph, p is about 3.8. In some embodiments of the formula described in the preceding paragraph, p is about 5. In some embodiments of the formula described in the preceding paragraph, p is about 6. In some embodiments of the formula described in the preceding paragraph, p is about 7. In some embodiments of the formula described in the preceding paragraph, p is about 8. In some embodiments of the formula described in the preceding paragraph, p is about 9. In some embodiments of the formula described in the preceding paragraph, p is about 10. In some embodiments of the formula described in the preceding paragraph, p is about 11. In some embodiments of the formula described in the preceding paragraph, p is about 12. In some embodiments of the formula described in the preceding paragraph, p is about 13. In some embodiments of the formula described in the preceding paragraph, p is about 14. In some embodiments of the formula described in the preceding paragraph, p is about 15. In some embodiments of the formula described in the preceding paragraph, p is about 16. In some embodiments of the formula described in the preceding paragraph, p is about 17. In some embodiments of the formula described in the preceding paragraph, p is about 18. In some embodiments of the formula described in the preceding paragraph, p is about 19. In some embodiments of the formula described in the preceding paragraph, p is about 20.

Typically, peptide-based drug units can be prepared by forming a peptide bond between two or more amino acids and/or peptide fragments. Such peptide bonds can be prepared, for example, according to the liquid phase synthesis method (see E. Schroder and K. Lake, “The Peptides”, volume 1, pp 76-136, 1965, Academic Press) that is well-known in the field of peptide chemistry. The auristatin/dolastatin drug units may be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat Biotechnol 21(7):778-784.

### 5.3.3 Linkers

Typically, the antibody drug conjugates comprise a linker unit between the drug unit (e.g., MMAE) and the antibody unit (e.g., the anti-191P4D12 antibody or antigen binding fragment thereof). In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation.

In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in 191P4D12-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker (SEQ ID NO:15)). Other examples of such linkers are described, e.g., in U.S. Pat. No. 6,214,345, incorporated herein by reference in its entirety and for all purposes. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the Val-Cit linker). One advantage of using intracellular proteolytic release of the therapeutic agent is that the agent is typically attenuated when conjugated and the serum stabilities of the conjugates are typically high.

In other embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In yet other specific embodiments, the linker is a malonate linker (Johnson et al., 1995, Anticancer Res. 15:1387-93), a maleimidobenzoyl linker (Lau et al., 1995, Bioorg-Med- Chem. 3(10):1299-1304), or a 3′-N-amide analog (Lau et al., 1995, Bioorg-Med-Chem. 3(10):1305-12).

In yet other embodiments, the linker unit is not cleavable and the drug is released by antibody degradation. (See U.S. Publication No. 2005/0238649 incorporated by reference herein in its entirety and for all purposes).

Typically, the linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of antibody drug conjugate, are cleaved when the antibody drug conjugate presents in an extracellular environment (e.g., in plasma). Whether a linker is not substantially sensitive to the extracellular environment can be determined, for example, by incubating with plasma the antibody-drug conjugate compound for a predetermined time period (e.g., 2, 4, 8, 16, or 24 hours) and then quantitating the amount of free drug present in the plasma.

In other non-mutually exclusive embodiments, the linker promotes cellular internalization. In certain embodiments, the linker promotes cellular internalization when conjugated to the therapeutic agent (i.e., in the milieu of the linker-therapeutic agent moiety of the antibody-drug conjugate compound as described herein). In yet other embodiments, the linker promotes cellular internalization when conjugated to both the auristatin compound and the anti-191P4D12 antibody or antigen binding fragment thereof.

A variety of exemplary linkers that can be used with the present compositions and methods are described in WO 2004-010957, U.S. Publication No. 2006/0074008, U.S. Publication No. 20050238649, and U.S. Publication No. 2006/0024317 (each of which is incorporated by reference herein in its entirety and for all purposes).

A “linker unit” (LU) is a bifunctional compound that can be used to link a drug unit and an antibody unit to form an antibody drug conjugate. In some embodiments, the linker unit has the formula:

-Aₐ-W_{w}—Y_{y}—

wherein: -A- is a stretcher unit,

a is 0 or 1,

each —W— is independently an amino acid unit,

w is an integer ranging from 0 to 12,

—Y— is a self-immolative spacer unit, and

y is 0, 1 or 2.

In some embodiments, a is 0 or 1, w is 0 or 1, and y is 0, 1 or 2. In some embodiments, a is 0 or 1, w is 0 or 1, and y is 0 or 1. In some embodiments, when w is 1 to 12, y is 1 or 2. In some embodiments, w is 2 to 12 and y is 1 or 2. In some embodiments, a is 1 and w and y are 0.

5.3.3.1 Stretcher Unit

The stretcher unit (A), when present, is capable of linking an antibody unit to an amino acid unit (—W—), if present, to a spacer unit (—Y—), if present; or to a drug unit (-D). Useful functional groups that can be present on an anti-191P4D12 antibody or an antigen binding fragment thereof (e.g. Ha22-2(2,4)6.1), either naturally or via chemical manipulation include, but are not limited to, sulfhydryl, amino, hydroxyl, the anomeric hydroxyl group of a carbohydrate, and carboxyl. Suitable functional groups are sulfhydryl and amino. In one example, sulfhydryl groups can be generated by reduction of the intramolecular disulfide bonds of an anti-191P4D12 antibody or an antigen binding fragment thereof. In another embodiment, sulfhydryl groups can be generated by reaction of an amino group of a lysine moiety of an anti-191P4D12 antibody or an antigen binding fragment with 2-iminothiolane (Traut's reagent) or other sulfhydryl generating reagents. In certain embodiments, the anti-191P4D12 antibody or antigen binding fragment thereof is a recombinant antibody and is engineered to carry one or more lysines. In certain other embodiments, the recombinant anti-191P4D12 antibody is engineered to carry additional sulfhydryl groups, e.g., additional cysteines.

In one embodiment, the stretcher unit forms a bond with a sulfur atom of the antibody unit. The sulfur atom can be derived from a sulfhydryl group of an antibody. Representative stretcher units of this embodiment are depicted within the square brackets of Formulas IIIa and IIIb below, wherein L-, —W—, —Y—, -D, w and y are as defined above, and R¹⁷ is selected from —C₁-C₁₀ alkylene-, —C₁-C₁₀ alkenylene-, alkynylene-, carbocyclo-, —O—(C₁-C₈ alkylene)-, O—(C₁-C₈ alkenylene)-, —O—(C₁-C₈ alkynylene)-, -arylene-, alkylene-arylene-, —C₂-C₁₀ alkenylene-arylene, —C₂-C₁₀ alkynylene-arylene, -arylene-C₁-C₁₀ alkylene-, -arylene-C₂-C₁₀ alkenylene-, -arylene-C₂-C₁₀ alkynylene-, —C₁-C₁₀ alkylene-(carbocyclo)-, —C₂-C₁₀ alkenylene-(carbocyclo)-, —C₂-C₁₀ alkynylene-(carbocyclo)-, -(carbocyclo)-C₁-C₁₀ alkylene-, -(carbocyclo)-C₂-C₁₀ alkenylene-, -(carbocyclo)-C₂-C₁₀ alkynylene, -heterocyclo-, alkylene-(heterocyclo)-, —C₂-C₁₀ alkenylene-(heterocyclo)-, —C₂-C₁₀ alkynylene-(heterocyclo)-, -(heterocyclo)-C₁-C₁₀ alkylene-, -(heterocyclo)-C₂-C₁₀ alkenylene-, -(heterocyclo)-C₁-C₁₀ alkynylene-, —(CH₂CH₂O)ᵣ—, or —(CH₂CH₂O)ᵣ—CH₂—, and r is an integer ranging from 1-10, wherein said alkyl, alkenyl, alkynyl, alkylene, alkenylene, alkynyklene, aryl, carbocycle, carbocyclo, heterocyclo, and arylene radicals, whether alone or as part of another group, are optionally substituted. In some embodiments, said alkyl, alkenyl, alkynyl, alkylene, alkenylene, alkynyklene, aryl, carbocyle, carbocyclo, heterocyclo, and arylene radicals, whether alone or as part of another group, are unsubstituted.

In some embodiments, R¹⁷ is selected from —C₁-C₁₀ alkylene-, -carbocyclo-, —O—(C₁-C₈ alkylene)-, -arylene-, —C₁-C₁₀ alkylene-arylene-, -arylene-C₁-C₁₀ alkylene-, alkylene-(carbocyclo)-, -(carbocyclo)-C₁-C₁₀ alkylene-, —C₃-C₈ heterocyclo-, —C₁-C₁₀ alkylene-(heterocyclo)-, -(heterocyclo)-C₁-C₁₀ alkylene-, —(CH₂CH₂O)ᵣ—, and —(CH₂CH₂O)ᵣ—CH₂—; and r is an integer ranging from 1-10, wherein said alkylene groups are unsubstituted and the remainder of the groups are optionally substituted.

It is to be understood from all the exemplary embodiments that even where not denoted expressly, 1 to 20 drug units can be linked to an antibody unit (p=1-20).

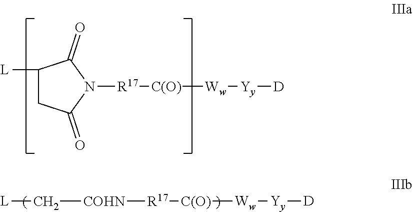

An illustrative stretcher unit is that of Formula IIIa wherein R¹⁷ is —(CH₂)₅—:

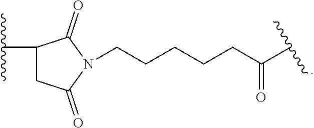 [SMILES: *C(=O)CCCCCN1C(=O)CC(*)C1=O]

Another illustrative stretcher unit is that of Formula IIIa wherein R¹⁷ is —(CH₂CH₂O)ᵣ—CH₂—; and r is 2:

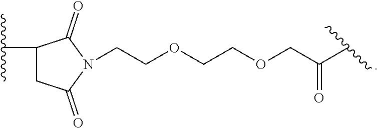 [SMILES: *C(=O)COCCOCCN1C(=O)CC(*)C1=O]

An illustrative Stretcher unit is that of Formula IIIa wherein R¹⁷ is arylene- or arylene-C₁-C₁₀ alkylene-. In some embodiments, the aryl group is an unsubstituted phenyl group.

Still another illustrative stretcher unit is that of Formula IIIb wherein R¹⁷ is —(CH₂)₅—:

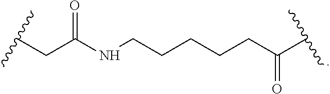 [SMILES: *CC(=O)NCCCCCC(*)=O]

In certain embodiments, the stretcher unit is linked to the antibody unit via a disulfide bond between a sulfur atom of the antibody unit and a sulfur atom of the stretcher unit. A representative stretcher unit of this embodiment is depicted within the square brackets of Formula IV, wherein R¹⁷, L-, —W—, —Y—, -D, w and y are as defined above.

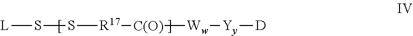

It should be noted that throughout this application, the S moiety in the formula below refers to a sulfur atom of the antibody unit, unless otherwise indicated by context.

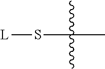

In certain of the structural descriptions of sulfur linked ADC herein the antibody is represented as “L”. It could also be indicated as “Ab-S”. The inclusion of “S” merely indicated the sulfur-linkage feature, and does not indicate that a particular sulfur atom bears multiple linker-drug moieties. The left parentheses of the structures using the “Ab-S” description may also be placed to the left of the sulfur atom, between Ab and S, which would be an equivalent description of the ADC of the invention described throughout herein.

In yet other embodiments, the stretcher contains a reactive site that can form a bond with a primary or secondary amino group of an antibody unit. Examples of these reactive sites include, but are not limited to, activated esters such as succinimide esters, 4 nitrophenyl esters, pentafluorophenyl esters, tetrafluorophenyl esters, anhydrides, acid chlorides, sulfonyl chlorides, isocyanates and isothiocyanates. Representative stretcher units of this embodiment are depicted within the square brackets of Formulas Va and Vb, wherein —R¹⁷—, L-, —W—, —Y—, -D, w and y are as defined above;

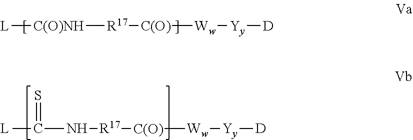

In some embodiments, the stretcher contains a reactive site that is reactive to a modified carbohydrate's (—CHO) group that can be present on an antibody unit. For example, a carbohydrate can be mildly oxidized using a reagent such as sodium periodate and the resulting (—CHO) unit of the oxidized carbohydrate can be condensed with a Stretcher that contains a functionality such as a hydrazide, an oxime, a primary or secondary amine, a hydrazine, a thiosemicarbazone, a hydrazine carboxylate, and an arylhydrazide such as those described by Kaneko et al., 1991, Bioconjugate Chem. 2:133-41. Representative stretcher units of this embodiment are depicted within the square brackets of Formulas VIa, VIb, and VIc, wherein —R¹⁷—, L-, —W—, —Y—, -D, w and y are as defined as above.

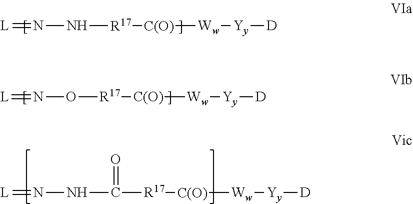

5.3.3.2 Amino Acid Unit

The amino acid unit (—W—), when present, links the stretcher unit to the spacer unit if the spacer unit is present, links the stretcher unit to the drug unit if the spacer unit is absent, and links the antibody unit to the drug unit if the stretcher unit and spacer unit are absent.

W_{w}— can be, for example, a monopeptide, dipeptide, tripeptide, tetrapeptide, pentapeptide, hexapeptide, heptapeptide, octapeptide, nonapeptide, decapeptide, undecapeptide or dodecapeptide unit. Each —W— unit independently has the formula denoted below in the square brackets, and w is an integer ranging from 0 to 12:

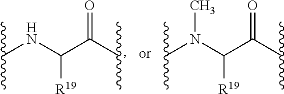

wherein R¹⁹ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

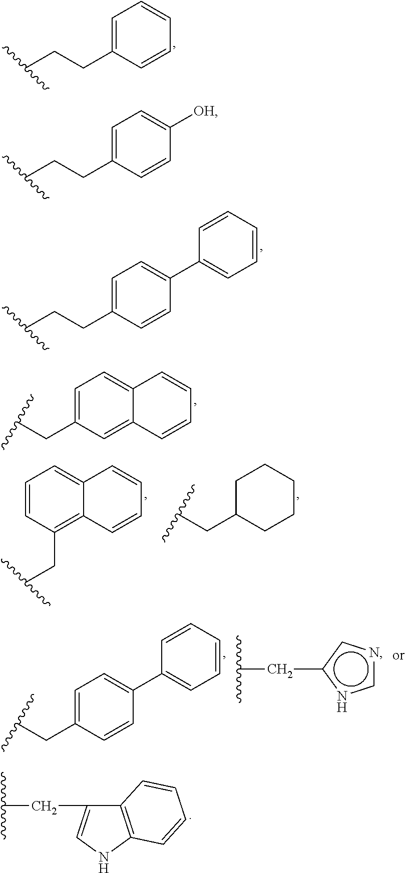

In some embodiments, the amino acid unit can be enzymatically cleaved by one or more enzymes, including a cancer or tumor-associated protease, to liberate the drug unit (-D), which in one embodiment is protonated in vivo upon release to provide a drug (D).

In certain embodiments, the amino acid unit comprises natural amino acids. In other embodiments, the amino acid unit comprises non-natural amino acids. Illustrative Ww units are represented by Formulas VII-IX below:

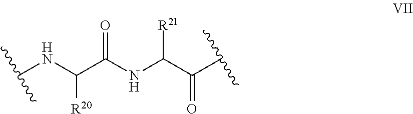

wherein R²⁰ and R²¹ are as follows:

| R²⁰ | R²¹ |
|---|---|
| Benzyl | (CH₂)₄NH₂; |
| Methyl | (CH₂)₄NH₂; |
| Isopropyl | (CH₂)₄NH₂; |
| Isopropyl | (CH₂)₃NHCONH₂; |
| Benzyl | (CH₂)₃NHCONH₂; |
| Isobutyl | (CH₂)₃NHCONH₂; |
| sec-butyl | (CH₂)₃NHCONH₂; |
| 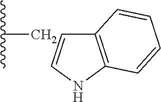 | (CH₂)₃NHCONH₂; |
| Benzyl | methyl; |
| Benzyl | (CH₂)₃NHC(═NH)NH₂; |

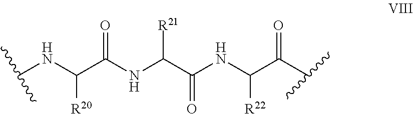

wherein R²⁰, R²¹ and R²² are as follows:

| R²⁰ | R²¹ | R²² |
|---|---|---|
| Benzyl | benzyl | (CH₂)₄NH₂; |
| Isopropyl | benzyl | (CH₂)₄NH₂; and |
| H | benzyl | (CH₂)₄NH₂; |

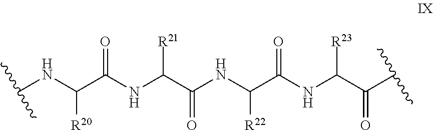

wherein R²⁰, R²¹, R²² and R²³ are as follows:

| R²⁰ | R²¹ | R²² | R²³ |
|---|---|---|---|
| H | benzyl | isobutyl | H; and |
| Methyl | isobutyl | methyl | isobutyl. |

Exemplary amino acid units include, but are not limited to, units of Formula VII above where: R²⁰ is benzyl and R²¹ is —(CH)₄NH₂; R²⁰ is isopropyl and R²¹ is —(CH₂)₄NH₂; or R²⁰ is isopropyl and R²¹ is —(CH₂)₃NHCONH₂.

Another exemplary amino acid unit is a unit of Formula VIII wherein R²⁰ is benzyl, R²¹ is benzyl, and R²² is —(CH₂)₄NH₂.

Useful —W_{w}— units can be designed and optimized in their selectivity for enzymatic cleavage by a particular enzyme, for example, a tumor-associated protease. In one embodiment, a —W_{w}— unit is that whose cleavage is catalyzed by cathepsin B, C and D, or a plasmin protease.

In one embodiment, —W_{w}— is a dipeptide, tripeptide, tetrapeptide or pentapeptide. When R¹⁹, R²⁰, R²¹, R²² or R²³ is other than hydrogen, the carbon atom to which R¹⁹, R²⁰, R²¹, R²² or R²³ is attached is chiral.

Each carbon atom to which R¹⁹, R²⁰, R²¹, R²² or R²³ is attached is independently in the (S) or (R) configuration.

In one specific embodiment, the amino acid unit is valine-citrulline (vc or Val-Cit). In another specific embodiment, the amino acid unit is phenylalanine-lysine (i.e., fk). In yet another specific embodiment, the amino acid unit is N-methylvaline-citrulline. In yet another specific embodiment, the amino acid unit is 5-aminovaleric acid, homo phenylalanine lysine, tetraisoquinolinecarboxylate lysine, cyclohexylalanine lysine, isonepecotic acid lysine, beta-alanine lysine, glycine serine valine glutamine and isonepecotic acid.

5.3.3.3 Spacer Unit

The spacer unit (—Y—), when present, links an amino acid unit to the drug unit when an amino acid unit is present. Alternately, the spacer unit links the stretcher unit to the drug unit when the amino acid unit is absent. The spacer unit also links the drug unit to the antibody unit when both the amino acid unit and stretcher unit are absent.

Spacer units are of two general types: non self-immolative or self-immolative. A non self-immolative spacer unit is one in which part or all of the spacer unit remains bound to the drug unit after cleavage, particularly enzymatic, of an amino acid unit from the antibody drug conjugate. Examples of a non self-immolative spacer unit include, but are not limited to a (glycine-glycine) spacer unit and a glycine spacer unit (both depicted in Scheme 1) (infra). When a conjugate containing a glycine-glycine spacer unit or a glycine Spacer unit undergoes enzymatic cleavage via an enzyme (e.g., a tumor-cell associated-protease, a cancer-cell-associated protease or a lymphocyte-associated protease), a glycine-glycine-drug unit or a glycine-drug unit is cleaved from L-Aa-Ww-. In one embodiment, an independent hydrolysis reaction takes place within the target cell, cleaving the glycine-drug unit bond and liberating the drug.

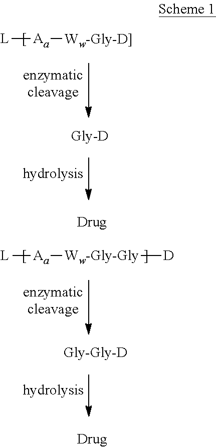

In some embodiments, a non self-immolative spacer unit (—Y—) is -Gly-. In some embodiments, a non self-immolative spacer unit (—Y—) is -Gly-Gly-.

In one embodiment, the spacer unit is absent (—Y_{y}— where y=0).

Alternatively, an antibody drug conjugate containing a self-immolative spacer unit can release -D. As used herein, the term “self-immolative spacer” refers to a bifunctional chemical moiety that is capable of covalently linking together two spaced chemical moieties into a stable tripartite molecule. It will spontaneously separate from the second chemical moiety if its bond to the first moiety is cleaved.

In some embodiments, —Y_{y}— is a p-aminobenzyl alcohol (PAB) unit (see Schemes 2 and 3) whose phenylene portion is substituted with Qₘ wherein Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted.

In some embodiments, —Y— is a PAB group that is linked to —W_{w}- via the amino nitrogen atom of the PAB group, and connected directly to -D via a carbonate, carbamate or ether group. Without being bound by any particular theory or mechanism, Scheme 2 depicts a possible mechanism of Drug release of a PAB group which is attached directly to -D via a carbamate or carbonate group as described by Toki et al., 2002, J Org. Chem. 67:1866-1872.

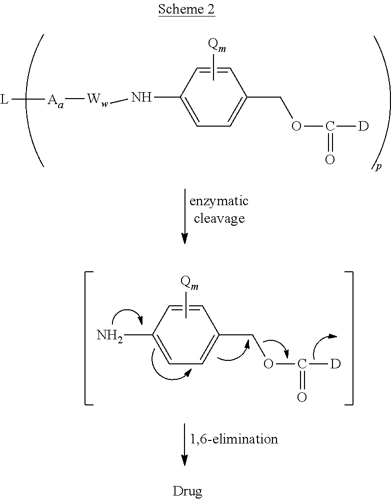

In Scheme 2, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4; and p ranges from 1 to about 20. The alkyl, alkenyl and alkynyl groups, whether alone or as part of another group, can be optionally substituted.

Without being bound by any particular theory or mechanism, Scheme 3 depicts a possible mechanism of drug release of a PAB group which is attached directly to -D via an ether or amine linkage, wherein D includes the oxygen or nitrogen group that is part of the drug unit.

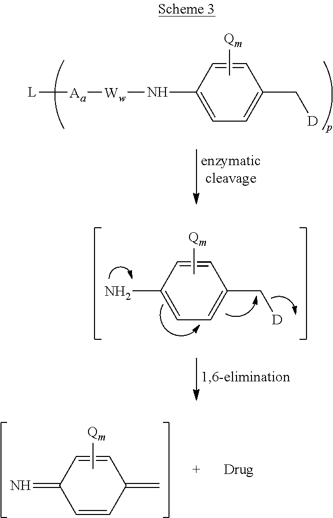

In Scheme 3, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4; and p ranges from 1 to about 20. The alkyl, alkenyl and alkynyl groups, whether alone or as part of another group, can be optionally substituted.

Other examples of self-immolative spacers include, but are not limited to, aromatic compounds that are electronically similar to the PAB group such as 2-aminoimidazol-5-methanol derivatives (Hay et al., 1999, Bioorg. Med. Chem. Lett. 9:2237) and ortho or para-aminobenzylacetals. Spacers can be used that undergo cyclization upon amide bond hydrolysis, such as substituted and unsubstituted 4-aminobutyric acid amides (Rodrigues et al., 1995, Chemistry Biology 2:223), appropriately substituted bicyclo[2.2.1] and bicyclo[2.2.2] ring systems (Storm et al., 1972, J. Amer. Chem. Soc. 94:5815) and 2-aminophenylpropionic acid amides (Amsberry et al., 1990, J. Org. Chem. 55:5867). Elimination of amine-containing drugs that are substituted at the α-position of glycine (Kingsbury et al., 1984, J. Med Chem. 27:1447) are also examples of self-immolative spacers.

In one embodiment, the spacer unit is a branched bis(hydroxymethyl)-styrene (BHMS) unit as depicted in Scheme 4, which can be used to incorporate and release multiple drugs.

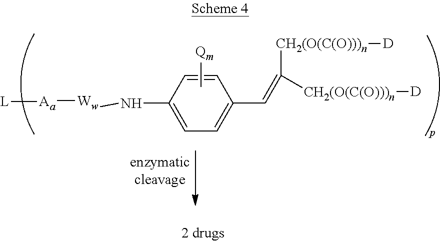

In Scheme 4, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4; n is 0 or 1; and p ranges ranging from 1 to about 20. The alkyl, alkenyl and alkynyl groups, whether alone or as part of another group, can be optionally substituted.

In some embodiments, the -D units are the same. In yet another embodiment, the -D moieties are different.

In one aspect, spacer units (—Y_{y}—) are represented by Formulas X-XII:

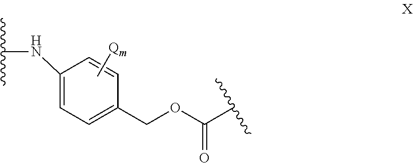

wherein Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted.

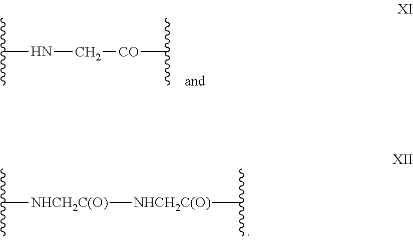

Embodiments of the Formula I and II comprising antibody-drug conjugate compounds can include:

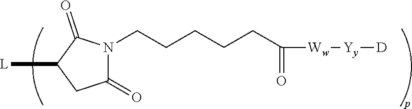

wherein w and y are each 0, 1 or 2, and,

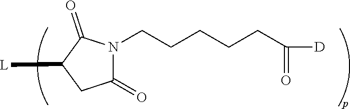

wherein w and y are each 0,

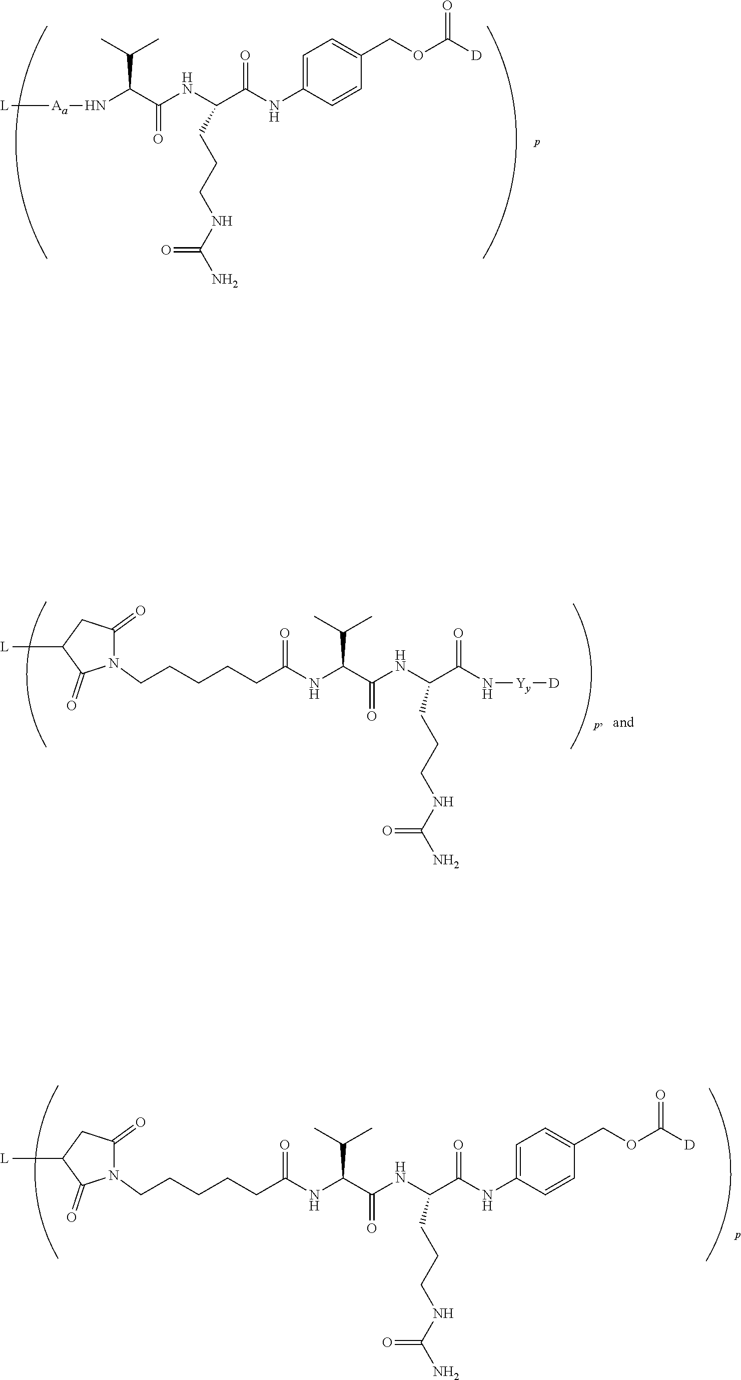

5.3.3.4 Drug Loading

Drug loading is represented by p and is the average number of drug units per antibody in a molecule. Drug loading may range from 1 to 20 drug units (D) per antibody. The ADCs provided herein include collections of antibodies or antigen binding fragments conjugated with a range of drug units, e.g., from 1 to 20. The average number of drug units per antibody in preparations of ADC from conjugation reactions may be characterized by conventional means such as mass spectroscopy and, ELISA assay. The quantitative distribution of ADC in terms of p may also be determined. In some instances, separation, purification, and characterization of homogeneous ADC where p is a certain value from ADC with other drug loadings may be achieved by means such as electrophoresis.

In certain embodiments, the drug loading for an ADC provided herein ranges from 1 to 20. In certain embodiments, the drug loading for an ADC provided herein ranges from 1 to 18. In certain embodiments, the drug loading for an ADC provided herein ranges from 1 to 15. In certain embodiments, the drug loading for an ADC provided herein ranges from 1 to 12. In certain embodiments, the drug loading for an ADC provided herein ranges from 1 to 10. In certain embodiments, the drug loading for an ADC provided herein ranges from 1 to 9. In certain embodiments, the drug loading for an ADC provided herein ranges from 1 to 8. In certain embodiments, the drug loading for an ADC provided herein ranges from 1 to 7. In certain embodiments, the drug loading for an ADC provided herein ranges from 1 to 6. In certain embodiments, the drug loading for an ADC provided herein ranges from 1 to 5. In certain embodiments, the drug loading for an ADC provided herein ranges from 1 to 4. In certain embodiments, the drug loading for an ADC provided herein ranges from 1 to 3. In certain embodiments, the drug loading for an ADC provided herein ranges from 2 to 12. In certain embodiments, the drug loading for an ADC provided herein ranges from 2 to 10. In certain embodiments, the drug loading for an ADC provided herein ranges from 2 to 9. In certain embodiments, the drug loading for an ADC provided herein ranges from 2 to 8. In certain embodiments, the drug loading for an ADC provided herein ranges from 2 to 7. In certain embodiments, the drug loading for an ADC provided herein ranges from 2 to 6. In certain embodiments, the drug loading for an ADC provided herein ranges from 2 to 5. In certain embodiments, the drug loading for an ADC provided herein ranges from 2 to 4. In certain embodiments, the drug loading for an ADC provided herein ranges from 3 to 12. In certain embodiments, the drug loading for an ADC provided herein ranges from 3 to 10. In certain embodiments, the drug loading for an ADC provided herein ranges from 3 to 9. In certain embodiments, the drug loading for an ADC provided herein ranges from 3 to 8. In certain embodiments, the drug loading for an ADC provided herein ranges from 3 to 7. In certain embodiments, the drug loading for an ADC provided herein ranges from 3 to 6. In certain embodiments, the drug loading for an ADC provided herein ranges from 3 to 5. In certain embodiments, the drug loading for an ADC provided herein ranges from 3 to 4.

In certain embodiments, the drug loading for an ADC provided herein ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7.

In certain embodiments, the drug loading for an ADC provided herein is about 1, about 2, about 3, about 4, about 5, about 6, about 7, about 8, about 9, about 10, about 11, about 12, or more. In some embodiments, the drug loading for an ADC provided herein is about 3.1, about 3.2, about 3.3, about 3.4, about 3.5, about 3.6, about 3.7, about 3.8, or about 3.9.

In some embodiments, the drug loading for an ADC provided herein ranges from 2 to 20, 2 to 19, 2 to 18, 2 to 17, 2 to 16, 2 to 15, 2 to 14, or 2 to 13. In some embodiments, the drug loading for an ADC provided herein ranges from 3 to 20, 3 to 19, 3 to 18, 3 to 17, 3 to 16, 3 to 15, 3 to 14, or 3 to 13. In some embodiments, the drug loading for an ADC provided herein is about 1. In some embodiments, the drug loading for an ADC provided herein is about 2. In some embodiments, the drug loading for an ADC provided herein is about 3. In some embodiments, the drug loading for an ADC provided herein is about 4. In some embodiments, the drug loading for an ADC provided herein is about 3.8. In some embodiments, the drug loading for an ADC provided herein is about 5. In some embodiments, the drug loading for an ADC provided herein is about 6. In some embodiments, the drug loading for an ADC provided herein is about 7. In some embodiments, the drug loading for an ADC provided herein is about 8. In some embodiments, the drug loading for an ADC provided herein is about 9. In some embodiments, the drug loading for an ADC provided herein is about 10. In some embodiments, the drug loading for an ADC provided herein is about 11. In some embodiments, the drug loading for an ADC provided herein is about 12. In some embodiments, the drug loading for an ADC provided herein is about 13. In some embodiments, the drug loading for an ADC provided herein is about 14. In some embodiments, the drug loading for an ADC provided herein is about 15. In some embodiments, the drug loading for an ADC provided herein is about 16. In some embodiments, the drug loading for an ADC provided herein is about 17. In some embodiments, the drug loading for an ADC provided herein is about 18. In some embodiments, the drug loading for an ADC provided herein is about 19. In some embodiments, the drug loading for an ADC provided herein is about 20.

In certain embodiments, fewer than the theoretical maximum of drug units are conjugated to an antibody during a conjugation reaction. An antibody may contain, for example, lysine residues that do not react with the drug-linker intermediate or linker reagent. Generally, antibodies do not contain many free and reactive cysteine thiol groups which may be linked to a drug unit; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the linker unit or a drug unit is conjugated via a lysine residue on the antibody unit. In some embodiments, the linker unit or a drug unit is conjugated via a cysteine residue on the antibody unit.

In some embodiments, the amino acid that attaches to a linker unit or a drug unit is in the heavy chain of an antibody or antigen binding fragment thereof. In some embodiments, the amino acid that attaches to a linker unit or a drug unit is in the light chain of an antibody or antigen binding fragment thereof. In some embodiments, the amino acid that attaches to a linker unit or a drug unit is in the hinge region of an antibody or antigen binding fragment thereof. In some embodiments, the amino acid that attaches to a linker unit or a drug unit is in the Fc region of an antibody or antigen binding fragment thereof. In other embodiments, the amino acid that attaches to a linker unit or a drug unit is in the constant region (e.g., CH1, CH2, or CH3 of a heavy chain, or CH1 of a light chain) of an antibody or antigen binding fragment thereof. In yet other embodiments, the amino acid that attaches to a linker unit or a drug unit is in the VH framework regions of an antibody or antigen binding fragment thereof. In yet other embodiments, the amino acid that attaches to a linker unit or a drug unit is in the VL framework regions of an antibody or antigen binding fragment thereof.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments (such as thioMab or thioFab prepared as disclosed herein and in WO2006/034488 (herein incorporated by reference in its entirety)).

It is to be understood that where more than one nucleophilic group reacts with a drug-linker intermediate or linker reagent followed by drug unit reagent, then the resulting product is a mixture of ADC compounds with a distribution of one or more drug unit attached to an antibody unit. The average number of drugs per antibody may be calculated from the mixture by a dual ELISA antibody assay, which is specific for antibody and specific for the drug. Individual ADC molecules may be identified in the mixture by mass spectroscopy and separated by HPLC, e.g. hydrophobic interaction chromatography (see, e.g., Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value may be isolated from the conjugation mixture by electrophoresis or chromatography.

Methods for preparing, screening, and characterizing the antibody drug conjugates are known to a person of ordinary skill in the art, for example, as described in U.S. Pat. No. 8,637,642, which is herein incorporated in its entirety by reference.

In some embodiments, the antibody drug conjugate for the methods provided herein is AGS-22M6E, which is prepared according to the methods described in U.S. Pat. No. 8,637,642 and has the following formula:

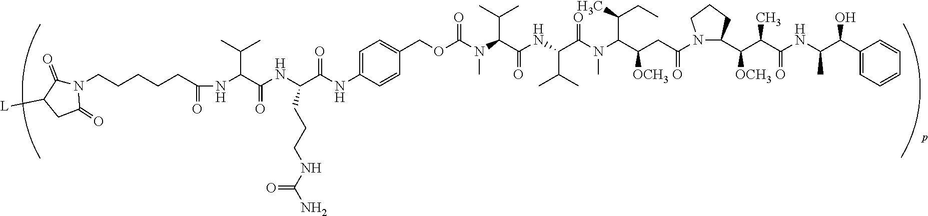

wherein L is Ha22-2(2,4)6.1 and p is from 1 to 20.

In some embodiments, p ranges from 1 to 20, 1 to 10, 1 to 9, 1 to 8, 1 to 7, 1 to 6, 1 to 5, 1 to 4, 1 to 3, or 1 to 2. In some embodiments, p ranges from 2 to 10, 2 to 9, 2 to 8, 2 to 7, 2 to 6, 2 to 5, 2 to 4 or 2 to 3. In other embodiments, p is about 1. In other embodiments, p is about 2. In other embodiments, p is about 3. In other embodiments, p is about 4. In other embodiments, p is about 5. In other embodiments, p is about 6. In other embodiments, p is about 7. In other embodiments, p is about 8. In other embodiments, p is about 9. In other embodiments, p is about 10. In some embodiments, p is about 3.1. In some embodiments, p is about 3.2. In some embodiments, p is about 3.3. In some embodiments, p is about 3.4. In some embodiments, p is about 3.5. In other embodiments, p is about 3.6. In some embodiments, p is about 3.7. In some embodiments, p is about 3.8. In some embodiments, p is about 3.9. In some embodiments, p is about 4.0. In some embodiments, p is about 4.1. In some embodiments, p is about 4.2. In some embodiments, p is about 4.3. In some embodiments, p is about 4.4. In some embodiments, p is about 4.5. In other embodiments, p is about 4.6. In some embodiments, p is about 4.7. In some embodiments, p is about 4.8. In some embodiments, p is about 4.9. In some embodiments, p is about 5.0.

In some embodiments, the ADC used in the methods provided herein is enfortumab vedotin. Enfortumab vedotin is an ADC comprised of a fully human immunoglobulin G1 kappa (IgG1κ) antibody conjugated to the microtubule-disrupting agent (MMAE) via a protease-cleavable linker (Challita-Eid P M et al, Cancer Res. 2016; 76(10):3003-13]. Enfortumab vedotin induces antitumor activity by binding to 191P4D12 protein on the cell surface leading to internalization of the ADC-191P4D12 complex, which then traffics to the lysosomal compartment where MMAE is released via proteolytic cleavage of the linker. Intracellular release of MMAE subsequently disrupts tubulin polymerization resulting in G2/M phase cell cycle arrest and apoptotic cell death (Francisco J A et al, Blood. 2003 Aug. 15; 102(4):1458-65).

As described above and in in U.S. Pat. No. 8,637,642, AGS-22M6E is an ADC derived from a murine hybridoma cell line. Enfortumab vedotin is the a Chinese hamster ovary (CHO) cell line-derived equivalent of AGS-22M6E ADC and is an exemplary product used for human treatment. Enfortumab vedotin has the same amino acid sequence, linker and cytotoxic drug as AGS-22M6E. The comparability between enfortumab vedotin and AGS-22M6E was confirmed through extensive analytical and biological characterization studies, such as binding affinity to 191P4D12, in vitro cytotoxicity, and in vivo antitumor activity.

### 5.4 Pharmaceutical Compositions

In certain embodiments of the methods provided herein, the ADC used in the methods is provided in “pharmaceutical compositions.” Such pharmaceutical compositions include an antibody drug conjugate provided herein, and one or more pharmaceutically acceptable or physiologically acceptable excipients. In certain embodiments, the antibody drug conjugate are provided in combination with, or separate from, one or more additional agents. Also provided is a composition comprising such one or more additional agents and one or more pharmaceutically acceptable or physiologically acceptable excipients. In particular embodiments, the antibody drug conjugate and an additional agent(s) are present in a therapeutically acceptable amount. The pharmaceutical compositions may be used in accordance with the methods and uses provided herein. Thus, for example, the pharmaceutical compositions can be administered ex vivo or in vivo to a subject in order to practice treatment methods and uses provided herein. Pharmaceutical compositions provided herein can be formulated to be compatible with the intended method or route of administration; exemplary routes of administration are set forth herein.

In some embodiments, provided are pharmaceutical compositions of antibody drug conjugates that modulate a cancer or tumor.

In certain embodiments of the methods provided herein, the pharmaceutical compositions comprising the ADCs may further comprise other therapeutically active agents or compounds disclosed herein or known to the skilled artisan which can be used in the treatment or prevention of various diseases and disorders as set forth herein (e.g., a cancer). As set forth above, the additional therapeutically active agents or compounds may be present in a separate pharmaceutical composition(s).

Pharmaceutical compositions typically comprise a therapeutically effective amount of at least one of the antibody drug conjugates provided herein and one or more pharmaceutically acceptable formulation agents. In certain embodiments, the pharmaceutical composition further comprises one or more additional agents described herein.

In one embodiment, a pharmaceutical composition comprises an antibody drug conjugate provided herein. In some embodiments, a pharmaceutical composition comprises a therapeutically effective amount of an antibody drug conjugate provided herein. In certain embodiments, the pharmaceutical composition comprises a pharmaceutically acceptable excipient.

In some embodiments, the antibody drug conjugate in the pharmaceutical composition provided herein is selected from the antibody drug conjugates described in Section 5.3 below.

In certain embodiments, the pharmaceutical composition comprises the antibody drug conjugate at a concentration of from 0.1-100 mg/mL. In some embodiments, the pharmaceutical composition comprises the antibody drug conjugate at a concentration of from 1 to 20 mg/mL. In other embodiments, the pharmaceutical composition comprises the antibody drug conjugate at a concentration of from 5 to 15 mg/mL. In other embodiments, the pharmaceutical composition comprises the antibody drug conjugate at a concentration of from 8 to 12 mg/mL. In other embodiments, the pharmaceutical composition comprises the antibody drug conjugate at a concentration of from 9 to 11 mg/mL. In some embodiments, the pharmaceutical composition comprises the antibody drug conjugate at a concentration of about 9.5 mg/mL. In some embodiments, the pharmaceutical composition comprises the antibody drug conjugate at a concentration of about 9.6 mg/mL. In some embodiments, the pharmaceutical composition comprises the antibody drug conjugate at a concentration of about 9.7 mg/mL. In some embodiments, the pharmaceutical composition comprises the antibody drug conjugate at a concentration of about 9.8 mg/mL. In some embodiments, the pharmaceutical composition comprises the antibody drug conjugate at a concentration of about 9.9 mg/mL. In yet other embodiments, the pharmaceutical composition comprises the antibody drug conjugate at a concentration of about 10 mg/mL. In yet other embodiments, the pharmaceutical composition comprises the antibody drug conjugate at a concentration of about 10.1 mg/mL. In some embodiments, the pharmaceutical composition comprises the antibody drug conjugate at a concentration of about 10.2 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.4 mg/mL. In some embodiments, the pharmaceutical composition comprises the antibody drug conjugate at a concentration of about 10.5 mg/mL.

In some embodiments, the pharmaceutical composition provided herein comprises L-histidine, TWEEN-20, and at least one of trehalose dihydrate or sucrose. In some embodiments, the pharmaceutical composition provided herein further comprises hydrochloric acid (HCl) or succinic acid.

In some embodiments, the concentration of L-histidine useful in the pharmaceutical compositions provided herein is in the range of between 5 and 50 mM. In some embodiments, the concentration of L-histidine in the pharmaceutical compositions provided herein is in the range of between 10 and 40 mM. In some embodiments, the concentration of L-histidine in the pharmaceutical compositions provided herein is in the range of between 15 and 35 mM. In some embodiments, the concentration of L-histidine in the pharmaceutical compositions provided herein is in the range of between 15 and 30 mM. In some embodiments, the concentration of L-histidine in the pharmaceutical compositions provided herein is in the range of between 15 and 25 mM. In some embodiments, the concentration of L-histidine in the pharmaceutical compositions provided herein is in the range of between 15 and 35 mM. In some embodiments, the concentration of L-histidine in the pharmaceutical compositions provided herein is about 16 mM. In some embodiments, the concentration of L-histidine in the pharmaceutical compositions provided herein is about 17 mM. In some embodiments, the concentration of L-histidine in the pharmaceutical compositions provided herein is about 18 mM. In some embodiments, the concentration of L-histidine in the pharmaceutical compositions provided herein is about 19 mM. In some embodiments, the concentration of L-histidine in the pharmaceutical compositions provided herein is about 20 mM. In some embodiments, the concentration of L-histidine in the pharmaceutical compositions provided herein is about 21 mM. In some embodiments, the concentration of L-histidine in the pharmaceutical compositions provided herein is about 22 mM. In some embodiments, the concentration of L-histidine in the pharmaceutical compositions provided herein is about 23 mM. In some embodiments, the concentration of L-histidine in the pharmaceutical compositions provided herein is about 24 mM. In some embodiments, the concentration of L-histidine in the pharmaceutical compositions provided herein is about 25 mM.

In some embodiments, the concentration of TWEEN-20 useful in the pharmaceutical compositions provided herein is in the range of from 0.001 to 0.1% (v/v). In another embodiment, the concentration of TWEEN-20 is in the range of from 0.0025 to 0.075% (v/v). In one embodiment, the concentration of TWEEN-20 is in the range of from 0.005 to 0.05% (v/v). In another embodiment, the concentration of TWEEN-20 is in the range of from 0.0075 to 0.025% (v/v). In another embodiment, the concentration of TWEEN-20 is in the range of from 0.0075 to 0.05% (v/v). In another embodiment, the concentration of TWEEN-20 is in the range of from 0.01 to 0.03% (v/v). In one particular embodiment, the concentration of TWEEN-20 is about 0.01% (v/v). In one particular embodiment, the concentration of TWEEN-20 is about 0.015% (v/v). In one particular embodiment, the concentration of TWEEN-20 is about 0.016% (v/v). In one particular embodiment, the concentration of TWEEN-20 is about 0.017% (v/v). In one particular embodiment, the concentration of TWEEN-20 is about 0.018% (v/v). In one particular embodiment, the concentration of TWEEN-20 is about 0.019% (v/v). In one particular embodiment, the concentration of TWEEN-20 is about 0.02% (v/v). In one particular embodiment, the concentration of TWEEN-20 is about 0.021% (v/v). In one particular embodiment, the concentration of TWEEN-20 is about 0.022% (v/v). In one particular embodiment, the concentration of TWEEN-20 is about 0.023% (v/v). In one particular embodiment, the concentration of TWEEN-20 is about 0.024% (v/v). In one particular embodiment, the concentration of TWEEN-20 is about 0.025% (v/v).

In one embodiment, the concentration of trehalose dihydrate useful in the pharmaceutical compositions provided herein is in the range of between 1% and 20% (w/v). In another embodiment, the concentration of trehalose dihydrate is in the range of 2% and 15% (w/v). In one embodiment, the concentration of trehalose dihydrate is in the range of 3% and 10% (w/v). In another embodiment, the concentration of trehalose dihydrate is in the range of 4% and 9% (w/v). In another embodiment, the concentration of trehalose dihydrate is in the range of 4% and 8% (w/v). In another embodiment, the concentration of trehalose dihydrate is in the range of 4% and 7% (w/v). In another embodiment, the concentration of trehalose dihydrate is in the range of 4% and 6% (w/v). In another embodiment, the concentration of trehalose dihydrate is in the range of 4.5% and 6% (w/v). In another embodiment, the concentration of trehalose dihydrate is about 4.6% (w/v). In another embodiment, the concentration of trehalose dihydrate is about 4.7% (w/v). In another embodiment, the concentration of trehalose dihydrate is about 4.8% (w/v). In another embodiment, the concentration of trehalose dihydrate is about 4.9% (w/v). In another embodiment, the concentration of trehalose dihydrate is about 5.0% (w/v). In another embodiment, the concentration of trehalose dihydrate is about 5.1% (w/v). In another embodiment, the concentration of trehalose dihydrate is about 5.2% (w/v). In another embodiment, the concentration of trehalose dihydrate is about 5.3% (w/v). In another embodiment, the concentration of trehalose dihydrate is about 5.4% (w/v). In another embodiment, the concentration of trehalose dihydrate is about 5.5% (w/v). In another embodiment, the concentration of trehalose dihydrate is about 5.6% (w/v). In another embodiment, the concentration of trehalose dihydrate is about 5.7% (w/v). In another embodiment, the concentration of trehalose dihydrate is about 5.8% (w/v). In another embodiment, the concentration of trehalose dihydrate is about 5.9% (w/v). In another embodiment, the concentration of trehalose dihydrate is about 6.0% (w/v). In another embodiment, the concentration of trehalose dihydrate is about 6.1% (w/v). In another embodiment, the concentration of trehalose dihydrate is about 6.2% (w/v). In another embodiment, the concentration of trehalose dihydrate is about 6.3% (w/v). In another embodiment, the concentration of trehalose dihydrate is about 6.4% (w/v). In another embodiment, the concentration of trehalose dihydrate is about 6.5% (w/v).

In certain embodiments, the molarity of the trehalose dihydrate is from 50 to 300 mM. In other embodiments, the molarity of the trehalose dihydrate is from 75 to 250 mM. In some embodiments, the molarity of the trehalose dihydrate is from 100 to 200 mM. In other embodiments, the molarity of the trehalose dihydrate is from 130 to 150 mM. In some embodiments, the molarity of the trehalose dihydrate is from 135 to 150 mM. In certain embodiments, the molarity of the trehalose dihydrate is about 135 mM. In certain embodiments, the molarity of the trehalose dihydrate is about 136 mM. In certain embodiments, the molarity of the trehalose dihydrate is about 137 mM. In certain embodiments, the molarity of the trehalose dihydrate is about 138 mM. In certain embodiments, the molarity of the trehalose dihydrate is about 139 mM. In certain embodiments, the molarity of the trehalose dihydrate is about 140 mM. In certain embodiments, the molarity of the trehalose dihydrate is about 141 mM. In certain embodiments, the molarity of the trehalose dihydrate is about 142 mM. In certain embodiments, the molarity of the trehalose dihydrate is about 143 mM. In certain embodiments, the molarity of the trehalose dihydrate is about 144 mM. In certain embodiments, the molarity of the trehalose dihydrate is about 145 mM. In certain embodiments, the molarity of the trehalose dihydrate is about 146 mM. In certain embodiments, the molarity of the trehalose dihydrate is about 150 mM. In certain embodiments, the molarity of the trehalose dihydrate is about 151 mM. In certain embodiments, the molarity of the trehalose dihydrate is about 151 mM. In certain embodiments, the molarity of the trehalose dihydrate is about 152 mM. In certain embodiments, the molarity of the trehalose dihydrate is about 153 mM. In certain embodiments, the molarity of the trehalose dihydrate is about 154 mM. In certain embodiments, the molarity of the trehalose dihydrate is about 155 mM.

In one embodiment, the concentration of sucrose useful in the pharmaceutical compositions provided herein is in the range of between 1% and 20% (w/v). In another embodiment, the concentration of sucrose is in the range of 2% and 15% (w/v). In one embodiment, the concentration of sucrose is in the range of 3% and 10% (w/v). In another embodiment, the concentration of sucrose is in the range of 4% and 9% (w/v). In another embodiment, the concentration of sucrose is in the range of 4% and 8% (w/v). In another embodiment, the concentration of sucrose is in the range of 4% and 7% (w/v). In another embodiment, the concentration of sucrose is in the range of 4% and 6% (w/v). In another embodiment, the concentration of sucrose is in the range of 4.5% and 6% (w/v). In another embodiment, the concentration of sucrose is about 4.6% (w/v). In another embodiment, the concentration of sucrose is about 4.7% (w/v). In another embodiment, the concentration of sucrose is about 4.8% (w/v). In another embodiment, the concentration of sucrose is about 4.9% (w/v). In another embodiment, the concentration of sucrose is about 5.0% (w/v). In another embodiment, the concentration of sucrose is about 5.1% (w/v). In another embodiment, the concentration of sucrose is about 5.2% (w/v). In another embodiment, the concentration of sucrose is about 5.3% (w/v). In another embodiment, the concentration of sucrose is about 5.4% (w/v). In another embodiment, the concentration of sucrose is about 5.5% (w/v). In another embodiment, the concentration of sucrose is about 5.6% (w/v). In another embodiment, the concentration of sucrose is about 5.7% (w/v). In another embodiment, the concentration of sucrose is about 5.8% (w/v). In another embodiment, the concentration of sucrose is about 5.9% (w/v). In another embodiment, the concentration of sucrose is about 6.0% (w/v). In another embodiment, the concentration of sucrose is about 6.1% (w/v). In another embodiment, the concentration of sucrose is about 6.2% (w/v). In another embodiment, the concentration of sucrose is about 6.3% (w/v). In another embodiment, the concentration of sucrose is about 6.4% (w/v). In another embodiment, the concentration of sucrose is about 6.5% (w/v).

In certain embodiments, the molarity of the sucrose is from 50 to 300 mM. In other embodiments, the molarity of the sucrose is from 75 to 250 mM. In some embodiments, the molarity of the sucrose is from 100 to 200 mM. In other embodiments, the molarity of the sucrose is from 130 to 150 mM. In some embodiments, the molarity of the sucrose is from 135 to 150 mM. In certain embodiments, the molarity of the sucrose is about 135 mM. In certain embodiments, the molarity of the sucrose is about 136 mM. In certain embodiments, the molarity of the sucrose is about 137 mM. In certain embodiments, the molarity of the sucrose is about 138 mM. In certain embodiments, the molarity of the sucrose is about 139 mM. In certain embodiments, the molarity of the sucrose is about 140 mM. In certain embodiments, the molarity of the sucrose is about 141 mM. In certain embodiments, the molarity of the sucrose is about 142 mM. In certain embodiments, the molarity of the sucrose is about 143 mM. In certain embodiments, the molarity of the sucrose is about 144 mM. In certain embodiments, the molarity of the sucrose is about 145 mM. In certain embodiments, the molarity of the sucrose is about 146 mM. In certain embodiments, the molarity of the sucrose is about 150 mM. In certain embodiments, the molarity of the sucrose is about 151 mM. In certain embodiments, the molarity of the sucrose is about 151 mM. In certain embodiments, the molarity of the sucrose is about 152 mM. In certain embodiments, the molarity of the sucrose is about 153 mM. In certain embodiments, the molarity of the sucrose is about 154 mM. In certain embodiments, the molarity of the sucrose is about 155 mM.

In some embodiments, the pharmaceutical composition provided herein comprises HCl. In other embodiments, the pharmaceutical composition provided herein comprises succinic acid.

In some embodiments, the pharmaceutical composition provided herein has a pH in a range of 5.5 to 6.5. In other embodiments, the pharmaceutical composition provided herein has a pH in a range of 5.7 to 6.3. In some embodiments, the pharmaceutical composition provided herein has a pH of about 5.7. In some embodiments, the pharmaceutical composition provided herein has a pH of about 5.8. In some embodiments, the pharmaceutical composition provided herein has a pH of about 5.9. In some embodiments, the pharmaceutical composition provided herein has a pH of about 6.0. In some embodiments, the pharmaceutical composition provided herein has a pH of about 6.1. In some embodiments, the pharmaceutical composition provided herein has a pH of about 6.2. In some embodiments, the pharmaceutical composition provided herein has a pH of about 6.3.

In some embodiments, the pH is taken at room temperature. In other embodiments, the pH is taken at 15° C. to 27° C. In yet other embodiments, the pH is taken at 4° C. In yet other embodiments, the pH is taken at 25° C.

In some embodiments, the pH is adjusted by HCl. In some embodiments, the pharmaceutical composition comprises HCl, and the pharmaceutical composition has a pH in a range of 5.5 to 6.5 at room temperature. In some embodiments, the pharmaceutical composition comprises HCl, and the pharmaceutical composition has a pH in a range of 5.7 to 6.3 at room temperature. In some more specific embodiments, the pharmaceutical composition comprises HCl, and the pharmaceutical composition has a pH of about of 5.7 at room temperature. In some more specific embodiments, the pharmaceutical composition comprises HCl, and the pharmaceutical composition has a pH of about of 5.8 at room temperature. In some more specific embodiments, the pharmaceutical composition comprises HCl, and the pharmaceutical composition has a pH of about of 5.9 at room temperature. In some more specific embodiments, the pharmaceutical composition comprises HCl, and the pharmaceutical composition has a pH of about of 6.0 at room temperature. In some more specific embodiments, the pharmaceutical composition comprises HCl, and the pharmaceutical composition has a pH of about of 6.1 at room temperature. In some more specific embodiments, the pharmaceutical composition comprises HCl, and the pharmaceutical composition has a pH of about of 6.2 at room temperature. In some more specific embodiments, the pharmaceutical composition comprises HCl, and the pharmaceutical composition has a pH of about of 6.3 at room temperature.

In some embodiments, the pharmaceutical composition comprises HCl, and the pharmaceutical composition has a pH in a range of 5.5 to 6.5 at 15° C. to 27° C. In some embodiments, the pharmaceutical composition comprises HCl, and the pharmaceutical composition has a pH in a range of 5.7 to 6.3 at 15° C. to 27° C. In some more specific embodiments, the pharmaceutical composition comprises HCl, and the pharmaceutical composition has a pH of about of 5.7 at 15° C. to 27° C. In some more specific embodiments, the pharmaceutical composition comprises HCl, and the pharmaceutical composition has a pH of about of 5.8 at 15° C. to 27° C. In some more specific embodiments, the pharmaceutical composition comprises HCl, and the pharmaceutical composition has a pH of about of 5.9 at 15° C. to 27° C. In some more specific embodiments, the pharmaceutical composition comprises HCl, and the pharmaceutical composition has a pH of about of 6.0 at 15° C. to 27° C. In some more specific embodiments, the pharmaceutical composition comprises HCl, and the pharmaceutical composition has a pH of about of 6.1 at 15° C. to 27° C. In some more specific embodiments, the pharmaceutical composition comprises HCl, and the pharmaceutical composition has a pH of about of 6.2 at 15° C. to 27° C. In some more specific embodiments, the pharmaceutical composition comprises HCl, and the pharmaceutical composition has a pH of about of 6.3 at 15° C. to 27° C.

In some embodiments, the pH is adjusted by succinic acid. In some embodiments, the pharmaceutical composition comprises succinic acid, and the pharmaceutical composition has a pH in a range of 5.5 to 6.5 at room temperature. In some embodiments, the pharmaceutical composition comprises succinic acid, and the pharmaceutical composition has a pH in a range of 5.7 to 6.3 at room temperature. In some more specific embodiments, the pharmaceutical composition comprises succinic acid, and the pharmaceutical composition has a pH of about of 5.7 at room temperature. In some more specific embodiments, the pharmaceutical composition comprises succinic acid, and the pharmaceutical composition has a pH of about of 5.8 at room temperature. In some more specific embodiments, the pharmaceutical composition comprises succinic acid, and the pharmaceutical composition has a pH of about of 5.9 at room temperature. In some more specific embodiments, the pharmaceutical composition comprises succinic acid, and the pharmaceutical composition has a pH of about of 6.0 at room temperature. In some more specific embodiments, the pharmaceutical composition comprises succinic acid, and the pharmaceutical composition has a pH of about of 6.1 at room temperature. In some more specific embodiments, the pharmaceutical composition comprises succinic acid, and the pharmaceutical composition has a pH of about of 6.2 at room temperature. In some more specific embodiments, the pharmaceutical composition comprises succinic acid, and the pharmaceutical composition has a pH of about of 6.3 at room temperature.

In some embodiments, the pharmaceutical composition comprises succinic acid, and the pharmaceutical composition has a pH in a range of 5.5 to 6.5 at 15° C. to 27° C. In some embodiments, the pharmaceutical composition comprises succinic acid, and the pharmaceutical composition has a pH in a range of 5.7 to 6.3 at 15° C. to 27° C. In some more specific embodiments, the pharmaceutical composition comprises succinic acid, and the pharmaceutical composition has a pH of about of 5.7 at 15° C. to 27° C. In some more specific embodiments, the pharmaceutical composition comprises succinic acid, and the pharmaceutical composition has a pH of about of 5.8 at 15° C. to 27° C. In some more specific embodiments, the pharmaceutical composition comprises succinic acid, and the pharmaceutical composition has a pH of about of 5.9 at 15° C. to 27° C. In some more specific embodiments, the pharmaceutical composition comprises succinic acid, and the pharmaceutical composition has a pH of about of 6.0 at 15° C. to 27° C. In some more specific embodiments, the pharmaceutical composition comprises succinic acid, and the pharmaceutical composition has a pH of about of 6.1 at 15° C. to 27° C. In some more specific embodiments, the pharmaceutical composition comprises succinic acid, and the pharmaceutical composition has a pH of about of 6.2 at 15° C. to 27° C. In some more specific embodiments, the pharmaceutical composition comprises succinic acid, and the pharmaceutical composition has a pH of about of 6.3 at 15° C. to 27° C.

In some specific embodiments, the pharmaceutical composition provided herein comprises about 20 mM L-histidine, about 0.02% (w/v) TWEEN-20, and at least one of about 5.5% (w/v) trehalose dihydrate or about 5% (w/v) sucrose. In some embodiments, the pharmaceutical composition provided herein further comprises HCl or succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5.5% (w/v) trehalose dihydrate and HCl. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and HCl. In some embodiments, the pH is about 6.0 at room temperature. In some embodiments, the pH is about 6.0 at 25° C.

In other specific embodiments, the pharmaceutical composition provided herein comprises about 20 mM L-histidine, about 0.02% (w/v) TWEEN-20, about 5.5% (w/v) trehalose dihydrate and succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and succinic acid. In some embodiments, the pH is about 6.0 at room temperature. In some embodiments, the pH is about 6.0 at 25° C.

In a specific embodiment, provided herein comprises

(a) an antibody drug conjugate comprising the following structure:

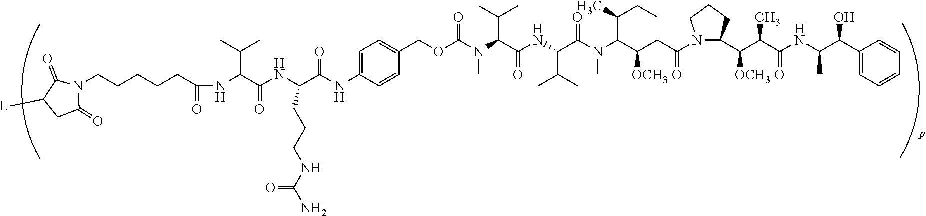

wherein L- represents the antibody or antigen binding fragment thereof and p is from 1 to 10; and
(b) a pharmaceutically acceptable excipient comprising about 20 mM L-histidine, about 0.02% (w/v) TWEEN-20, about 5.5% (w/v) trehalose dihydrate, and HCl, wherein the antibody drug conjugate is at the concentration of about 10 mg/mL, and wherein the pH is about 6.0 at 25° C.

In another specific embodiment, the pharmaceutical composition provided herein comprises:

(a) an antibody drug conjugate comprising the following structure:

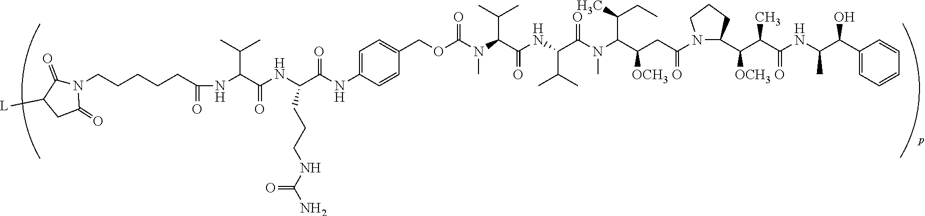

wherein L- represents the antibody or antigen binding fragment thereof and p is from 1 to 10; and
(b) a pharmaceutically acceptable excipient comprising about 20 mM L-histidine, about 0.02% (w/v) TWEEN-20, about 5.5% (w/v) trehalose dihydrate, and succinic acid, wherein the antibody drug conjugate is at the concentration of about 10 mg/mL, and wherein the pH is about 6.0 at 25° C.

In yet another specific embodiment, the pharmaceutical composition provided herein comprises:

(a) an antibody drug conjugate comprising the following structure:

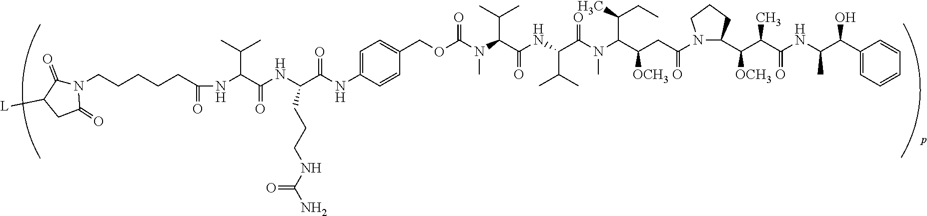

wherein L- represents the antibody or antigen binding fragment thereof and p is from 1 to 10; and
(b) a pharmaceutically acceptable excipient comprising about 20 mM L-histidine, about 0.02% (w/v) TWEEN-20, about 5.0% (w/v) sucrose, and HCl, wherein the antibody drug conjugate is at the concentration of about 10 mg/mL, and wherein the pH is about 6.0 at 25° C.

Although certain numbers (and numerical ranges thereof) are provided, it is understood that, in certain embodiments, numerical values within, e.g., 2%, 5%, 10%, 15% or 20% of said numbers (or numerical ranges) are also contemplated. Other exemplary pharmaceutical compositions are provided in the Experimental section below.

A primary solvent in a vehicle may be either aqueous or non-aqueous in nature. In addition, the vehicle may contain other pharmaceutically acceptable excipients for modifying or maintaining the pH, osmolarity, viscosity, sterility or stability of the pharmaceutical composition. In certain embodiments, the pharmaceutically acceptable vehicle is an aqueous buffer. In other embodiments, a vehicle comprises, for example, sodium chloride and/or sodium citrate.

Pharmaceutical compositions provided herein may contain still other pharmaceutically acceptable formulation agents for modifying or maintaining the rate of release of an antibody drug conjugate and/or an additional agent, as described herein. Such formulation agents include those substances known to artisans skilled in preparing sustained-release formulations. For further reference pertaining to pharmaceutically and physiologically acceptable formulation agents, see, for example, Remington's Pharmaceutical Sciences, 18th Ed. (1990, Mack Publishing Co., Easton, Pa. 18042) pages 1435-1712, The Merck Index, 12th Ed. (1996, Merck Publishing Group, Whitehouse, N.J.); and Pharmaceutical Principles of Solid Dosage Forms (1993, Technonic Publishing Co., Inc., Lancaster, Pa.). Additional pharmaceutical compositions appropriate for administration are known in the art and are applicable in the methods and compositions provided herein.

In some embodiments, the pharmaceutical composition provided herein is in a liquid form. In other embodiments, the pharmaceutical composition provided herein is lyophilized.

A pharmaceutical composition can be formulated to be compatible with its intended route of administration. Thus, pharmaceutical compositions include excipients suitable for administration by routes including parenteral (e.g., subcutaneous (s.c.), intravenous, intramuscular, or intraperitoneal), intradermal, oral (e.g., ingestion), inhalation, intracavity, intracranial, and transdermal (topical). Other exemplary routes of administration are set forth herein.

Pharmaceutical compositions may be in the form of a sterile injectable aqueous or oleagenous suspension. This suspension may be formulated using suitable dispersing or wetting agents and suspending agents disclosed herein or known to the skilled artisan. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed, including synthetic mono- or diglycerides. Moreover, fatty acids such as oleic acid find use in the preparation of injectables. Prolonged absorption of particular injectable formulations can be achieved by including an agent that delays absorption (e.g., aluminum monostearate or gelatin).

In one embodiment, the pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In one embodiment, the pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, e.g., Remington, The Science and Practice of Pharmacy, supra).

In one embodiment, the pharmaceutical compositions intended for parenteral administration may include one or more pharmaceutically acceptable excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

In one embodiment, suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

In one embodiment, suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

In one embodiment, the pharmaceutical compositions provided herein may be formulated for single or multiple dosage administration. The single dosage formulations are packaged in an ampoule, a vial, or a syringe. The multiple dosage parenteral formulations may contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

In one embodiment, the pharmaceutical compositions provided herein may be formulated as immediate or modified release dosage forms, including delayed-, sustained, pulsed-, controlled, targeted-, and programmed-release forms.

Dispersible powders and granules suitable for preparation of an aqueous suspension by addition of water provide the active ingredient in admixture with a dispersing or wetting agent, suspending agent and one or more preservatives. Suitable dispersing or wetting agents and suspending agents are exemplified herein.

Pharmaceutical compositions can also include excipients to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, may be employed. Prolonged absorption of injectable pharmaceutical compositions can be achieved by including an agent that delays absorption, for example, aluminum monostearate or gelatin. Prevention of the action of microorganisms can be achieved by various antibacterial and antifungal agents, for example, parabens, chlorobutanol, phenol, ascorbic acid, thimerosal, and the like.

The pharmaceutical composition provided herein may be stored at −80° C., 4° C., 25° C. or 37° C.

A lyophilized composition can be made by freeze-drying the liquid pharmaceutical composition provided herein. In a specific embodiment, the pharmaceutical composition provided here is a lyophilized pharmaceutical composition. In some embodiments, the pharmaceutical formulations are lyophilized powders, which can be reconstituted for administration as solutions, emulsions and other mixtures. They may also be reconstituted and formulated as solids or gels.

In some embodiments, preparation of the lyophilized formulation provided herein involves batching of the formulated bulk solution for lyophilization, aseptic filtration, filling in vials, freezing vials in a freeze-dryer chamber, followed by lyophilization, stoppering and capping.

A lyophilizer can be used in preparing the lyophilized formulation. For example, a VirTis Genesis Model EL pilot unit can be employed. The unit incorporates a chamber with three working shelves (to a total usable shelf area of ca 0.4 square meters), an external condenser, and a mechanical vacuum pumping system. Cascaded mechanical refrigeration allows the shelves to be cooled to −70° C. or lower, and the external condenser to −90° C. or lower. Shelf temperature and chamber pressure were controlled automatically to +/−0.5° C. and +/−2 microns (milliTorr), respectively. The unit was equipped with a capacitance manometer vacuum gauge, a Pirani vacuum gauge, a pressure transducer (to measure from 0 to 1 atmosphere), and a relative humidity sensor.

The lyophilized powder can be prepared by dissolving an antibody drug conjugate provided herein, or a pharmaceutically acceptable derivative thereof, in a suitable solvent. In some embodiments, the lyophilized powder is sterile. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the antibody drug conjugate. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable excipient. Such amount can be empirically determined and adjusted according to specific needs.

An exemplary reconstitution procedure is illustrated as follows: (1) fit the 5 mL or 3 mL syringe with a with a 18 or 20 Gauge needle and filled the syringe with water of the grade Water for Injection (WFI); (2) measure appropriate amount of WFI using the syringe graduations, ensuring that the syringe was free of air bubbles; (3) inserted the needle through the rubber stopper; (4) dispense the entire contents of the syringe into the container down the vial wall, removed the syringe and needle and put into the sharp container; (4) swirl the vial continuously to carefully solubilize the entire vial contents until fully reconstituted (e.g., about 20-40 seconds) and minimize excessive agitation of the protein solution that could result in foaming.

### 5.5 Methods of Using the Pharmaceutical Compositions in a Combination Therapy

The method for inhibiting growth of tumor cells using the pharmaceutical composition provided herein in combination with chemotherapy or radiation or both comprises administering the present pharmaceutical composition before, during, or after commencing chemotherapy or radiation therapy, as well as any combination thereof (i.e. before and during, before and after, during and after, or before, during, and after commencing the chemotherapy and/or radiation therapy). Depending on the treatment protocol and the specific patient needs, the method is performed in a manner that will provide the most efficacious treatment and ultimately prolong the life of the patient.

The administration of chemotherapeutic agents can be accomplished in a variety of ways including systemically by the parenteral and enteral routes. In one embodiment, the chemotherapeutic agent is administered separately. Particular examples of chemotherapeutic agents or chemotherapy include cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel (taxol), docetaxel (taxotere), aldesleukin, asparaginase, busulfan, carboplatin, cladribine, dacarbazine, floxuridine, fludarabine, hydroxyurea, ifosfamide, interferon alpha, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, gemcitabine, chlorambucil, taxol and combinations thereof.

The source of radiation, used in combination with the pharmaceutical composition provided herein, can be either external or internal to the patient being treated. When the source is external to the patient, the therapy is known as external beam radiation therapy (EBRT). When the source of radiation is internal to the patient, the treatment is called brachytherapy (BT).

The above described therapeutic regimens may be further combined with additional cancer treating agents and/or regimes, for example additional chemotherapy, cancer vaccines, signal transduction inhibitors, agents useful in treating abnormal cell growth or cancer, antibodies (e.g. Anti-CTLA-4 antibodies as described in WO/2005/092380 (Pfizer)) or other ligands that inhibit tumor growth by binding to IGF-1R, and cytokines.

When the mammal is subjected to additional chemotherapy, chemotherapeutic agents described above may be used. Additionally, growth factor inhibitors, biological response modifiers, anti-hormonal therapy, selective estrogen receptor modulators (SERMs), angiogenesis inhibitors, and anti-androgens may be used. For example, anti-hormones, for example anti-estrogens such as Nolvadex (tamoxifen) or, anti-androgens such as Casodex (4′-cyano-3-(4-fluorophenylsulphonyl)-2-hydroxy-2-methyl-3-′-(trifluoromethyl)propionanilide) may be used.

In some embodiments, the pharmaceutical provided herein in used in combination with a second therapeutic agent, e.g., for treating a cancer.

In some embodiments, the second therapeutic agent is an immune checkpoint inhibitor. As used herein, the term “immune checkpoint inhibitor” or “checkpoint inhibitor” refers to molecules that totally or partially reduce, inhibit, interfere with or modulate one or more checkpoint proteins. Without being limited by a particular theory, checkpoint proteins regulate T-cell activation or function. Numerous checkpoint proteins are known, such as CTLA-4 and its ligands CD80 and CD86; and PD-1 with its ligands PD-L1 and PD-L2 (Pardo11, Nature Reviews Cancer, 2012, 12, 252-264). These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In one embodiment, the checkpoint inhibitor is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In one embodiment, the checkpoint inhibitor is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In one embodiment, the checkpoint inhibitor is a PD-1 inhibitor. In one embodiment, the PD-1 inhibitor is an anti-PD-1 antibody. In one embodiment, the anti-PD-1 antibody is BGB-A317, nivolumab (also known as ONO-4538, BMS-936558, or MDX1106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity.

In one embodiment, the checkpoint inhibitor is a PD-L1 inhibitor. In one embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®).

In one embodiment, the checkpoint inhibitor is a PD-L2 inhibitor. In one embodiment, the PD-L2 inhibitor is an anti-PD-L2 antibody. In one embodiment, the anti-PD-L2 antibody is rHIgM12B7A.

In one embodiment, the checkpoint inhibitor is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211). In another embodiment, the LAG-3 inhibitor is BMS-986016.

In one embodiment, the checkpoint inhibitors is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint inhibitors is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In one embodiment, the checkpoint inhibitor is an OX40 (CD134) agonist. In one embodiment, the checkpoint inhibitor is an anti-OX40 antibody. In one embodiment, the anti-OX40 antibody is anti-OX-40. In another embodiment, the anti-OX40 antibody is MEDI6469.

In one embodiment, the checkpoint inhibitor is a GITR agonist. In one embodiment, the checkpoint inhibitor is an anti-GITR antibody. In one embodiment, the anti-GITR antibody is TRX518.

In one embodiment, the checkpoint inhibitor is a CD137 agonist. In one embodiment, the checkpoint inhibitor is an anti-CD137 antibody. In one embodiment, the anti-CD137 antibody is urelumab. In another embodiment, the anti-CD137 antibody is PF-05082566.

In one embodiment, the checkpoint inhibitor is a CD40 agonist. In one embodiment, the checkpoint inhibitor is an anti-CD40 antibody. In one embodiment, the anti-CD40 antibody is CF-870,893.

In one embodiment, the checkpoint inhibitor is recombinant human interleukin-15 (rhIL-15).

In one embodiment, the checkpoint inhibitor is an IDO inhibitor. In one embodiment, the IDO inhibitor is INCB024360. In another embodiment, the IDO inhibitor is indoximod.

In certain embodiments, the combination therapies provided herein include two or more of the checkpoint inhibitors described herein (including checkpoint inhibitors of the same or different class). Moreover, the combination therapies described herein can be used in combination with one or more second active agents as described herein where appropriate for treating diseases described herein and understood in the art.

In some embodiments, the checkpoint inhibitor is administered prior to the administration of the present pharmaceutical composition. In other embodiments, the checkpoint inhibitor is administered simultaneously (e.g., in the same dosing period) with the pharmaceutical composition provided herein. In yet other embodiments, the checkpoint inhibitor is administered after the administration of the pharmaceutical composition provided herein.

In some embodiments, the amount of the checkpoint inhibitor can be determined by standard clinical techniques.

A dosage of the checkpoint inhibitor results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, such as at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention and/or treatment of a cancer. It is to be understood that the precise dose of the checkpoint inhibitor to be employed will also depend on the route of administration, and the seriousness of a cancer in a subject, and should be decided according to the judgment of the practitioner and each patient's circumstances.

In some embodiments, the dosage of the checkpoint inhibitor (e.g., a PD-1 inhibitor or a PD-L1 inhibitor) administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 1 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight. In some embodiments, dosage administered to a patient is about 9.0 mg/kg of the subject's body weight. In some embodiments, dosage administered to a patient is about 10.0 mg/kg of the subject's body weight. In some embodiments, dosage administered to a patient is about 15.0 mg/kg of the subject's body weight. In some embodiments, dosage administered to a patient is about 20.0 mg/kg of the subject's body weight.

In some embodiments, the pharmaceutical composition provided herein is supplied as a dry sterilized lyophilized powder or water free concentrate in a hermetically sealed container and can be reconstituted, e.g., with water or saline to the appropriate concentration for administration to a subject. In certain embodiments, the antibody drug conjugate is supplied as a dry sterile lyophilized powder in a hermetically sealed container at a unit dosage of at least 0.1 mg, at least 0.5 mg, at least 1 mg, at least 2 mg, at least 3 mg, at least 5 mg, at least 10 mg, at least 15 mg, at least 25 mg, at least 30 mg, at least 35 mg, at least 45 mg, at least 50 mg, at least 60 mg, at least 75 mg, at least 80 mg, at least 85 mg, at least 90 mg, at least 95 mg, or at least 100 mg. The lyophilized antibody drug conjugate can be stored at between 2 and 8° C. in its original container and the antibody drug conjugate can be administered within 12 hours, such as within 6 hours, within 5 hours, within 3 hours, or within 1 hour after being reconstituted. In an alternative embodiment, the pharmaceutical composition comprising the antibody drug conjugate provided herein is supplied in liquid form in a hermetically sealed container indicating the quantity and concentration of the antibody drug conjugate. In certain embodiments, the liquid form of the antibody drug conjugate is supplied in a hermetically sealed container at least 0.1 mg/ml, at least 0.5 mg/ml, at least 1 mg/ml, at least 5 mg/ml, at least 10 mg/ml, at least 15 mg/ml, at least 25 mg/ml, at least 30 mg/ml, at least 40 mg/ml, at least 50 mg/ml, at least 60 mg/ml, at least 70 mg/ml, at least 80 mg/ml, at least 90 mg/ml, or at least 100 mg/ml.

### 5.6 Dosage of the ADCs for the Methods

In some embodiments, the amount of a prophylactic or therapeutic agent (e.g., an antibody drug conjugate provided herein), or a pharmaceutical composition provided herein that will be effective in the prevention and/or treatment of a cancer can be determined by standard clinical techniques.

Accordingly, a dosage of an antibody drug conjugate in the pharmaceutical composition that results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, such as at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention and/or treatment of a cancer. It is to be understood that the precise dose to be employed in the formulation will also depend on the route of administration, and the seriousness of a cancer in a subject, and should be decided according to the judgment of the practitioner and each patient's circumstances.

Effective doses may be extrapolated from dose-response curves derived from in vitro or animal model test systems.

For the pharmaceutical composition comprising the antibody drug conjugate provided herein, the dosage of the antibody drug conjugate administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 0.5 mg/kg of the subject's body weight. In some embodiments, dosage administered to a patient is about 0.75 mg/kg of the subject's body weight. In some embodiments, dosage administered to a patient is about 1 mg/kg of the subject's body weight. In some embodiments, dosage administered to a patient is about 1.25 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight.

In some embodiments, the antibody drug conjugate formulated in the pharmaceutical composition provided herein is administered based on the patient's actual body weight at baseline and doses will not change unless the patient's weight changes by ≥10% from baseline of the previous cycle, or the dose adjustment criteria is met. In some embodiments, actual weight will be used except for patients weighing greater than 100 kg, in such cases, the dose will be calculated based on a weight of 100 kg. In some embodiments, the maximum doses are 100 mg for patients receiving the 1.00 mg/kg dose level and 125 mg for patients receiving the 1.25 mg/kg dose level.

In one embodiment, approximately 100 mg/kg or less, approximately 75 mg/kg or less, approximately 50 mg/kg or less, approximately 25 mg/kg or less, approximately 10 mg/kg or less, approximately 5 mg/kg or less, approximately 1.5 mg/kg or less, approximately 1.25 mg/kg or less, approximately 1 mg/kg or less, approximately 0.75 mg/kg or less, approximately 0.5 mg/kg or less, or approximately 0.1 mg/kg or less of an antibody drug conjugate formulated in the present pharmaceutical composition is administered 5 times, 4 times, 3 times, 2 times or 1 time to treat a cancer. In some embodiments, the pharmaceutical composition comprising the antibody drug conjugate provided herein is administered about 1-12 times, wherein the doses may be administered as necessary, e.g., weekly, biweekly, monthly, bimonthly, trimonthly, etc., as determined by a physician. In some embodiments, a lower dose (e.g., 0.1-15 mg/kg) can be administered more frequently (e.g., 3-6 times). In other embodiments, a higher dose (e.g., 25-100 mg/kg) can be administered less frequently (e.g., 1-3 times).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every two-week cycle (e.g., about 14 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every three-week cycle (e.g., about 21 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every four-week cycle (e.g., about 28 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, 6, 7, 8, 9, 10, 11, or 12 times at about monthly (e.g., about 30 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, or 6 times at about bi-monthly (e.g., about 60 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In yet another embodiment, a single dose of an antibody drug conjugate formulated in the pharmaceutical composition provided herein is administered to patient to prevent and/or treat a cancer 1, 2, 3 or 4 times at about tri-monthly (e.g., about 120 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In certain embodiments, the route of administration for a dose of an antibody drug conjugate formulated in the pharmaceutical composition provided herein to a patient is intranasal, intramuscular, intravenous, or a combination thereof, but other routes described herein are also acceptable. Each dose may or may not be administered by an identical route of administration. In some embodiments, an antibody drug conjugate formulated in the pharmaceutical composition provided herein may be administered via multiple routes of administration simultaneously or subsequently to other doses of one or more additional therapeutic agents.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion over about 30 minutes twice every three-week cycle. In some embodiments, the antibody drug conjugate formulated in the pharmaceutical composition is administered by an intravenous (IV) injection or infusion over about 30 minutes on Days 1 and 8 of every three-week cycle. In some embodiments, the method further comprises administering an immune checkpoint inhibitor by an intravenous (IV) injection or infusion one or more times in each three-week cycle. In some embodiments, the method further comprises administering an immune checkpoint inhibitor by an intravenous (IV) injection or infusion on Day 1 of every three-week cycle. In some embodiments, the immune checkpoint inhibitor is pembrolizumab, and wherein pembrolizumab is administered at amount of about 200 mg over about 30 minutes. In other embodiments, the immune checkpoint inhibitor is atezolizumab, and wherein atezolizumab is administered at amount of about 1200 mg over about 60 minutes or 30 minutes. In some embodiments, the antibody drug conjugate is administered to patients with urothelial or bladder cancer who have shown disease progression or relapse during or after treatment with an immune checkpoint inhibitor. In some embodiments, the antibody drug conjugate is administered to patients with metastatic urothelial or bladder cancer who have shown disease progression or relapse during or after treatment with an immune checkpoint inhibitor. In some embodiments, the antibody drug conjugate is administered to patients with locally advanced urothelial or bladder cancer who have shown disease progression or relapse during or after treatment with an immune checkpoint inhibitor.

In other more specific embodiments, the antibody drug conjugate formulated in the pharmaceutical composition provided herein is administered at a dose of about about 0.5 mg/kg, about 0.75 mg/kg, 1 mg/kg, about 1.25 mg/kg, or about 1.5 mg/kg of the subject's body weight by an intravenous (IV) injection or infusion over about 30 minutes three times every four-week cycle. In some embodiments, the antibody drug conjugate formulated in the pharmaceutical composition is administered on Days 1, 8 and 15 of every 28-day (four-week) cycle. In some embodiments, the antibody drug conjugate formulated in the pharmaceutical composition is administered by an intravenous (IV) injection or infusion over about 30 minutes on Days 1, 8 and 15 of every 28-day (four-week) cycle. In some embodiments, the method further comprises administering an immune checkpoint inhibitor by an intravenous (IV) injection or infusion one or more times in each four-week cycle. In some embodiments, the immune checkpoint inhibitor is pembrolizumab. In other embodiments, the immune checkpoint inhibitor is atezolizumab. In some embodiments, the antibody drug conjugate is administered to patients with urothelial or bladder cancer who have shown disease progression or relapse during or after treatment with an immune checkpoint inhibitor. In some embodiments, the antibody drug conjugate is administered to patients with metastatic urothelial or bladder cancer who have shown disease progression or relapse during or after treatment with an immune checkpoint inhibitor. In some embodiments, the antibody drug conjugate is administered to patients with locally advanced urothelial or bladder cancer who have shown disease progression or relapse during or after treatment with an immune checkpoint inhibitor.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

Particular embodiments of this invention are described herein, including the best mode known to the inventors for carrying out the invention. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law.

Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference in its entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

A number of embodiments of the invention have been described. Nevertheless, it will be understood that various modifications may be made without departing from the spirit and scope of the invention. Accordingly, the descriptions in the Experimental section are intended to illustrate but not limit the scope of invention described in the claims.

### 6. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, temperature, etc.), but some experimental errors and deviations should be accounted for.

### 6.1 Example 1—Ha22-2(2,4)6.1vcMMAE Inhibit Growth of Tumors In Vivo

The significant expression of 191P4D12 on the cell surface of tumor tissues, together with its restrictive expression in normal tissues makes 191P4D12 a good target for antibody therapy and similarly, therapy via ADC. Thus, the therapeutic efficacy of Ha22-2(2,4)6.1vcMMAE in human bladder, lung, breast, and pancreatic cancer xenograft mouse models is evaluated.

Antibody drug conjugate efficacy on tumor growth and metastasis formation is studied in mouse cancer xenograft models (e.g. subcutaneous and orthotopically).

Subcutaneous (s.c.) tumors are generated by injection of 5×10⁴- 10⁶ cancer cells mixed at a 1:1 dilution with Matrigel (Collaborative Research) in the right flank of male SCID mice. To test ADC efficacy on tumor formation, ADC injections are started on the same day as tumor-cell injections. As a control, mice are injected with either purified human IgG or PBS; or a purified MAb that recognizes an irrelevant antigen not expressed in human cells. In preliminary studies, no difference is found between control IgG or PBS on tumor growth. Tumor sizes are determined by caliper measurements, and the tumor volume is calculated as width×Length/2, wherein width is the smallest dimension and length is the largest dimension. Mice with subcutaneous tumors greater than 1.5 cm in diameter are sacrificed.

An advantage of xenograft cancer models is the ability to study neovascularization and angiogenesis. Tumor growth is partly dependent on new blood vessel development. Although the capillary system and developing blood network is of host origin, the initiation and architecture of the neovasculature is regulated by the xenograft tumor (Davidoff et al., Clin Cancer Res. (2001) 7:2870; Solesvik et al., Eur J Cancer Clin Oncol. (1984) 20:1295). The effect of antibody and small molecule on neovascularization is studied in accordance with procedures known in the art, such as by IHC analysis of tumor tissues and their surrounding microenvironment.

191P4D12 ADCs:

Monoclonal antibodies against 191P4D12 and its conjugation to MMAE are described above. The Ha22-2(2,4)6.1vcMMAE is characterized by FACS, and other methods known in the art to determine its capacity to bind 191P4D12.

Cell Lines and Xenografts:

The BT-483 and HPAC cells are maintained in DMEM, supplemented with L-glutamine and 10% FBS, as known in the art. AG-L4 xenografts are maintained by serial propagation in SCID mice.

Efficacy of Ha22-2(2,4)6.1-vcMMAE in Subcutaneous Established Human Lung Cancer Xenograft AG-L4 in SCID Mice

In another experiment, patient-derived lung cancer xenograft AG-L4 was maintained by serial passages in SCID mice. Stock tumors were harvested sterilely and minced into 1 mm³ pieces. Six (6) pieces were implanted into the flank of individual SCID mice. Tumors were allowed to grow untreated until they reached an approximate volume of 200 mm³. The Ha22-2(2,4)6.1vcMMAE and the control ADC were dosed at 10 mg/kg every seven (7) days for two doses by intravenous bolus injection. The amount of ADC administered was based on the individual body weight of each animal obtained immediately prior to dosing. Tumor growth was monitored using caliper measurements every 3 to 4 days. Tumor volume was calculated as Width²×Length/2, where width is the smallest dimension and length is the largest dimension.

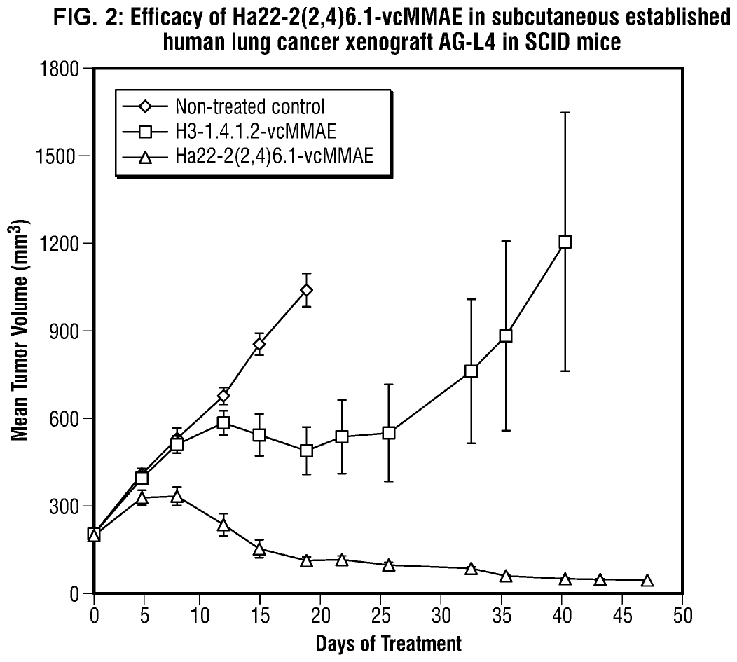

The results show that treatment with Ha22-2(2,4)6.1-vcMMAE significantly inhibited the growth of AG-L4 lung cancer xenografts implanted subcutaneously in nude mice compared to the control ADC. (FIG. 2). Additionally, other 191P4D12 MAbs were utilized in this study. The results are not shown.

Efficacy of Ha22-2(2,4)6.1-vcMMAE in Subcutaneous Established Human Breast Cancer Xenograft BT-483 in SCID Mice

In this experiment, human breast cancer BT-483 cells were used to generate stock xenografts, which were maintained by serial passages in SCID mice. Stock tumors were harvested sterilely and minced into 1 mm³ pieces. Six (6) pieces were implanted into the flank of individual SCID mice. Tumors were allowed to grow untreated until they reached an approximate volume of 100 mm³. The Ha22-2(2,4)6.1vcMMAE and the control ADC were dosed at 5 mg/kg every four (4) days for four (4) doses by intravenous bolus injection. The amount of ADC administered was based on the individual body weight of each animal obtained immediately prior to dosing. Tumor growth was monitored using caliper measurements every 3 to 4 days. Tumor volume was calculated as Width²×Length/2, where width is the smallest dimension and length is the largest dimension.

The results show that treatment with Ha22-2(2,4)6.1-vcMMAE significantly inhibited the growth of BT-483 breast tumor xenografts implanted subcutaneously in SCID mice compared to the control ADC. (FIG. 3). Additionally, other 191P4D12 MAbs were utilized in this study. The results are not shown.

Conclusion

In summary, FIGS. 2 and 3, show that the 191P4D12 ADC entitled Ha22-2(2,4)6.1vcMMAE significantly inhibited the growth of tumors cells that express 191P4D12 when compared to control ADCs. Thus, the Ha22-2(2,4)6.1vcMMAE can be used for therapeutic purposes to treat and manage various cancers.

### 6.2 Example 2—Detection of 191P4D12 Protein in Cancer Patient Specimens by IHC

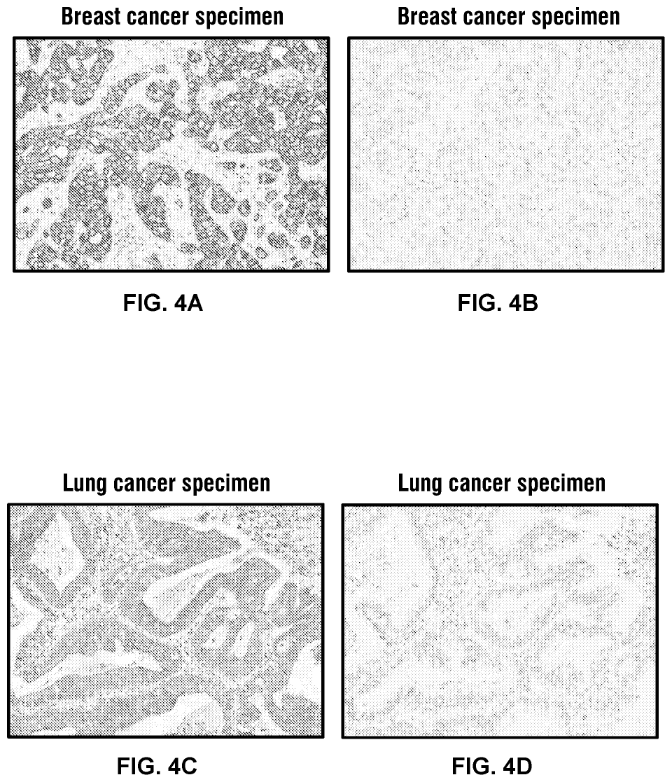

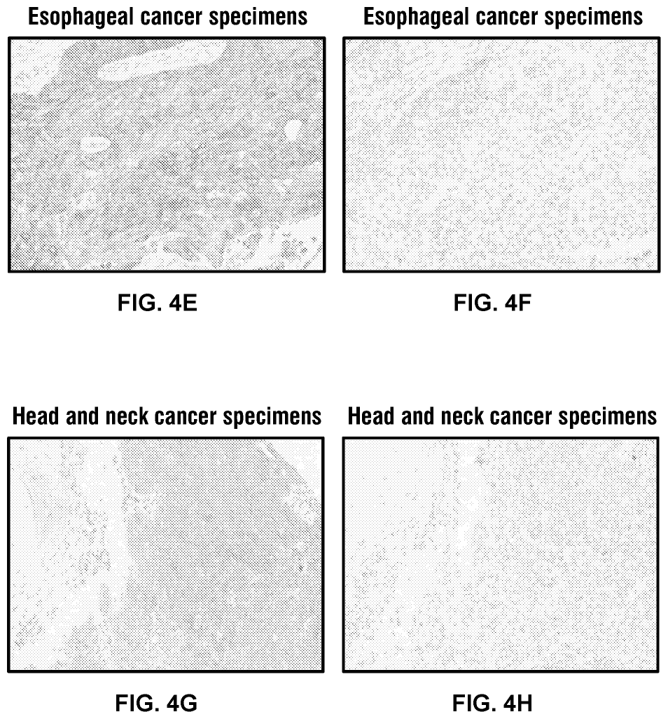

Expression of 191P4D12 protein by immunohistochemistry was tested in patient tumor specimens from (i) breast, (ii) lung, (iii) esophageal, and (iv) head and neck patients. Briefly, formalin fixed, paraffin wax-embedded tissues were cut into four (4) micron sections and mounted on glass slides. The sections were de-waxed, rehydrated and treated with EDTA antigen retrieval solution (Biogenex, San Ramon, Calif.) in the EZ-Retriever microwave (Biogenex, San Ramon, Calif.) for 30 minutes at 95° C. Sections were then treated with 3% hydrogen peroxide solution to inactivate endogenous peroxidase activity. Serum-free protein block (Dako, Carpenteria, Calif.) was used to inhibit non-specific binding prior to incubation with monoclonal mouse anti-191P4D12 antibody or an isotype control. Subsequently, the sections were treated with the Super Sensitive™ Polymer-horseradish peroxidase (HRP) Detection System which consists of an incubation in Super Enhancer™ reagent followed by an incubation with polymer-HRP secondary antibody conjugate (BioGenex, San Ramon, Calif.). The sections were then developed using the DAB kit (BioGenex, San Ramon, Calif.). Nuclei were stained using hematoxylin, and analyzed by bright field microscopy. Specific staining was detected in patient specimens using the 191P4D12 immunoreactive antibody, as indicated by the brown staining. (See, FIGS. 4A, 4C, 4E, and 4G). In contrast, the control antibody did not stain either patient specimen. (See, FIGS. 4B, 4D, 4F, and 4H).

The results show expression of 191P4D12 in the tumor cells of patient bladder, breast, pancreatic, lung, ovarian, esophageal, and head and neck cancer tissues. These results indicate that 191P4D12 is expressed in human cancers and that antibodies directed to this antigen and the antibody drug conjugate designated Ha22-2(2,4)6.1vcMMAE) are useful for diagnostic and therapeutic purposes. (FIGS. 4A-H).

### 6.3 Example 3—Treatment of Adult Patients with Locally Advanced or Metastatic Urothelial Cancer (mUC) Who have Received a PD-1 or PD-L1 Inhibitor and Who have Received a Platinum-Containing Chemotherapy in the Neoadjuvant/Adjuvant, Locally Advanced or Metastatic Setting

### 6.3.1 Description of the ADC Tested

In one embodiment, the ADC provided herein is enfortumab vedotin-ejfv, also known as PADCEV. In one specific embodiment tested in this example (6.3), the enfortumab vedotin-ejfv includes an anti-191P4D12 antibody, wherein the antibody or antigen binding fragment thereof comprises a heavy chain comprising amino acid residue 20 to amino acid residue 466 of SEQ ID NO: 7 and a light chain comprising amino acid residue 23 to amino acid residue 236 of SEQ ID NO:8.

Enfortumab vedotin-ejfv is a Nectin-4 directed antibody -drug conjugate (ADC) comprised of a fully human anti-nectin-4 IgG1 kappa monoclonal antibody (AGS-22C₃) conjugated to the small molecule microtubule disrupting agent, monomethyl auristatin E (MMAE) via a protease-cleavable maleimidocaproyl valine-citrulline (vc) linker (SGD-1006). Conjugation takes place on cysteine residues that comprise the interchain disulfide bonds of the antibody to yield a product with a drug-to-antibody ratio of approximately 3.8:1. The molecular weight is approximately 152 kDa.

Enfortumab vedotin-ejfv has the following structural formula:

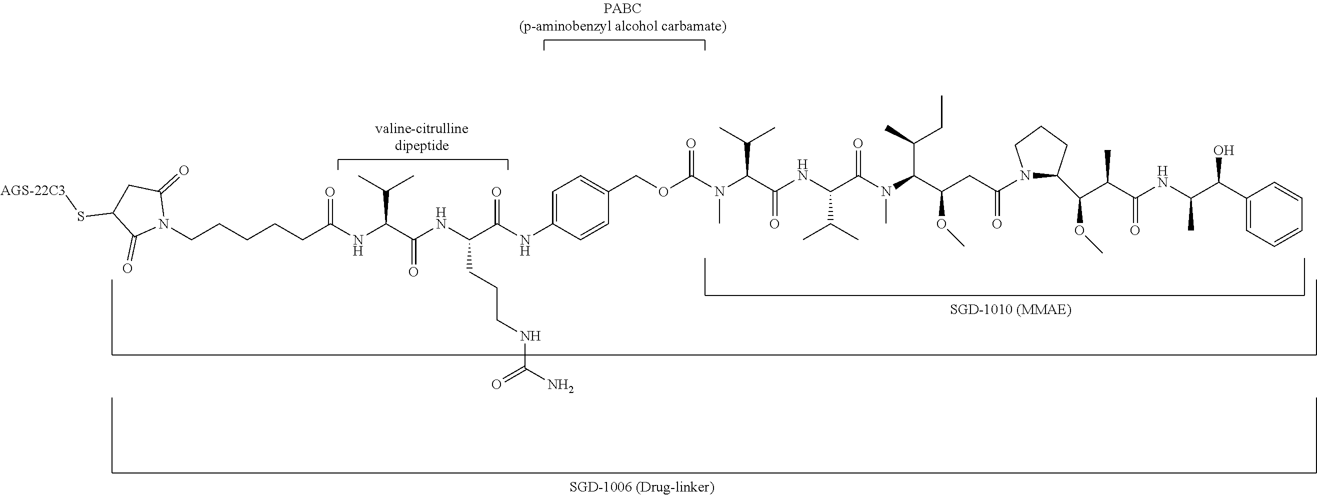

Approximately 4 molecules of MMAE are attached to each antibody molecule. Enfortumab vedotin-ejfv is produced by chemical conjugation of the antibody and small molecule components. The antibody is produced by mammalian (Chinese hamster ovary) cells and the small molecule components are produced by chemical synthesis.

PADCEV (enfortumab vedotin-ejfv) for injection is provided as a sterile, preservative-free, white to off-white lyophilized powder in single-dose vials for intravenous use. PADCEV is supplied as a 20 mg per vial and a 30 mg per vial and requires reconstitution with Sterile Water for Injection, USP, (2.3 mL and 3.3 mL, respectively) resulting in a clear to slightly opalescent, colorless to slightly yellow solution with a final concentration of 10 mg/mL [see Dosage and Administration (6.3.4.3)]. After reconstitution, each vial allows the withdrawal of 2 mL (20 mg) and 3 mL (30 mg). Each mL of reconstituted solution contains 10 mg of enfortumab vedotin-ejfv, histidine (1.4 mg), histidine hydrochloride monohydrate (2.31 mg), polysorbate 20 (0.2 mg) and trehalose dihydrate (55 mg) with a pH of 6.0.

### 6.3.2 Clinical Studies

6.3.2.1 Metastatic Urothelial Cancer

The efficacy of PADCEV was evaluated in EV-201 (NCT03219333), single-arm, multicenter trial that enrolled 125 patients with locally advanced or metastatic urothelial cancer who received prior treatment with a PD-1 or PD-L1 inhibitor and platinum-based chemotherapy. Patients were excluded if they had active CNS metastases, ongoing sensory or motor neuropathy >Grade 2, or uncontrolled diabetes defined as hemoglobin A1C (HbA1c)≥8% or HbA1c≥7% with associated diabetes symptoms.

The median age was 69 years (range: 40 to 84 years), 70% were male, and 85% were Caucasian. All patients had a baseline Eastern Cooperative Oncology Group (ECOG) performance status of 0 (32%) or 1 (68%). Ninety percent of patients had visceral metastases including 40% with liver metastases. Two-thirds of patients had pure transitional cell carcinoma (TCC) histology; 33% had TCC with other histologic variants. An immunohistochemistry clinical trial assay was used to assess patients with tumor tissue available, and detected Nectin-4 expression in all patients tested (n=120). The median number of prior systemic therapies was 3 (range: 1 to 6). Forty-six percent of patients received prior PD-1 inhibitor, 42% received prior PD-L1 inhibitor, and an additional 13% received both PD-1 and PD-L1 inhibitors. Sixty-six percent of patients received prior cisplatin-based regimens, 26% received prior carboplatin-based regimens, and an additional 8% received both cisplatin and carboplatin-based regimens.

The major efficacy outcome measures were confirmed objective response rate (ORR) and duration of response (DOR) assessed by blinded independent central review (BICR) using RECIST v1.1.

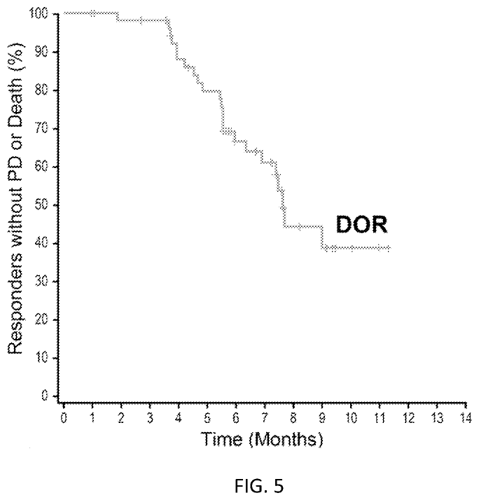

Efficacy results are presented in Table 21 and FIG. 5. According to FIG. 5, which is from an ongoing clinical trial, DOR in patients with complete responses ranged from 3.6+ to 11.6+ months. The median time to response was 1.84 months (range: 1.2 to 9.2). 44% of responders in FIG. 5 are still being followed.

TABLE 21
Efficacy Results in EV201 (BICR Assessment)

| | PADCEV |
|---|---|
| Endpoint | n = 125* |
| Confirmed ORR^{†} | 44% |
| (95% CI) | (35.1, 53.2) |
| Complete Response Rate (CR) | 12% |
| Partial Response Rate (PR) | 32% |
| Median Duration of Response, months | 7.6 |
| (95% CI) | (6.3, NE)^{‡} |

NE = not estimable
*Median follow-up duration of 10.2 months
^{†}Kaplan-Meier estimate.
^{‡}Based on patients (n = 55) with a response by BICR.

### 6.3.3 Indications and Usage

PADCEV is indicated for the treatment of adult patients with locally advanced or metastatic urothelial cancer (mUC) who have received a PD-1 or PD-L1 inhibitor and who have received a platinum-containing chemotherapy in the neoadjuvant/adjuvant, locally advanced or metastatic setting.

This indication is FDA approved under accelerated approval based on tumor response rate [see Clinical Studies (6.3.2.1)].

### 6.3.4 Dosage and Administration

6.3.4.1 Recommended Dosage

The recommended dose of PADCEV is 1.25 mg/kg (up to a maximum of 125 mg for patients ≥100 kg) administered as an intravenous infusion over 30 minutes on Days 1, 8 and 15 of a 28-day cycle until disease progression or unacceptable toxicity.

6.3.4.2 Dose Modifications (Table 22 and Table 23)

TABLE 22
Dose Modifications

| Adverse Reaction | Severity* | Dose Modification* |
|---|---|---|
| Hyperglycemia | Blood glucose | Withhold until elevated blood glucose |
| [see Warnings and | >250 mg/dL | has improved to ≤250 mg/dL, then |
| Precautions | | resume treatment at the same dose |
| (6.3.6.1)] | | level. |
| Peripheral | Grade 2 | Withhold until Grade ≤ 1, then |
| Neuropathy | | resume treatment at the same dose |
| [see Warnings | | level (if first occurrence). For a |
| and Precautions | | recurrence, withhold until Grade ≤ 1 |
| (6.3.6.2)] | | then, resume treatment |
| | | reduced by one dose level. |
| | Grade ≥ 3 | Permanently discontinue. |
| Skin Reactions | Grade 3 | Withhold until Grade ≤ 1, then resume |
| [see Warnings | (severe) | treatment at the same dose level or |
| and Precautions | | consider dose reduction by one dose |
| (6.3.6.4)] | | level. |
| | Grade 4 or | Permanently discontinue. |
| | recurrent | |
| | Grade 3 | |
| Other | Grade 3 | Withhold until Grade ≤ 1, then |
| nonhematologic | | resume treatment at the same dose |
| toxicity | | level or consider dose reduction by |
| | | one dose level |
| | Grade 4 | Permanently discontinue. |
| Hematologic | Grade 3, or | Withhold until Grade ≤ 1, then resume |
| toxicity | Grade 2 | treatment at the same dose level or |
| | thrombo- | consider dose reduction by one dose |
| | cytopenia | level. |
| | Grade 4 | Withhold until Grade ≤ 1, then |
| | | reduce dose by one dose level or |
| | | discontinue treatment. |

*Grade 1 is mild, Grade 2 is moderate, Grade 3 is severe, Grade 4 is life-threatening

TABLE 23
Recommended Dose Reduction Schedule

| | Dose Level |
|---|---|
| Starting dose | 1.25 mg/kg up to 125 mg |
| First dose reduction | 1.0 mg/kg up to 100 mg |
| Second dose reduction | 0.75 mg/kg up to 75 mg |
| Third dose reduction | 0.5 mg/kg up to 50 mg |

The results based on the dose modification schedule as provided are as follows.

Among patients who experienced, 93% of patients had resolution or improvement, 73% achieved complete resolution and 20% experienced some improvement. Of those with ongoing rash, most (75%) were Grade 1

Among patients who experienced peripheral neuropathy, most patients (76%) had resolution or Grade 1 peripheral neuropathy after the dose modification.

Among patients who experienced hyperglycemia, 57% achieved complete resolution and 14% experienced some improvement.

6.3.4.3 Instructions for Preparation and Administration

Administer PADCEV as an intravenous infusion only.

PADCEV is a cytotoxic drug. Follow applicable special handling and disposal procedures.

Prior to administration, the PADCEV vial is reconstituted with Sterile Water for Injection (SWFI). The reconstituted solution is subsequently diluted in an intravenous infusion bag containing either 5% Dextrose Injection, USP, 0.9% Sodium Chloride Injection, USP, or Lactated Ringer's Injection, USP.

Reconstitution in Single-Dose Vial

1. Follow procedures for proper handling and disposal of anticancer drugs.

2. Use appropriate aseptic technique for reconstitution and preparation of dosing solutions.

3. Calculate the recommended dose based on the patient's weight to determine the number and strength (20 mg or 30 mg) of vials needed.

4. Reconstitute each vial as follows and, if possible, direct the stream of SWFI along the walls of the vial and not directly onto the lyophilized powder:
a. 20 mg vial: Add 2.3 mL of SWFI, resulting in 10 mg/mL PADCEV.
b. 30 mg vial: Add 3.3 mL of SWFI, resulting in 10 mg/mL PADCEV.

5. Slowly swirl each vial until the contents are completely dissolved. Allow the reconstituted vial(s) to settle for at least 1 minute until the bubbles are gone. DO NOT SHAKE THE VIAL. Do not expose to direct sunlight.

6. Parenteral drug products should be inspected visually for particulate matter and discoloration prior to administration, whenever solution and container permit. The reconstituted solution should be clear to slightly opalescent, colorless to light yellow and free of visible particles. Discard any vial with visible particles or discoloration.

7. Based upon the calculated dose amount, the reconstituted solution from the vial(s) should be added to the infusion bag immediately. This product does not contain a preservative. If not used immediately, reconstituted vials may be stored for up to 4 hours in refrigeration at 2° C. to 8° C. (36° F. to 46° F.). DO NOT FREEZE. Discard unused vials with reconstituted solution beyond the recommended storage time.

Dilution in Infusion Bag

8. Withdraw the calculated dose amount of reconstituted solution from the vial(s) and transfer into an infusion bag.

9. Dilute PADCEV with either 5% Dextrose Injection, 0.9% Sodium Chloride Injection, or Lactated Ringer's Injection. The infusion bag size should allow enough diluent to achieve a final concentration of 0.3 mg/mL to 4 mg/mL PADCEV.

10. Mix diluted solution by gentle inversion. DO NOT SHAKE THE BAG. Do not expose to direct sunlight.

11. Visually inspect the infusion bag for any particulate matter or discoloration prior to use. The reconstituted solution should be clear to slightly opalescent, colorless to light yellow and free of visible particles. DO NOT USE the infusion bag if particulate matter or discoloration is observed.

12. Discard any unused portion left in the single-dose vials.

Administration

13. Immediately administer the infusion over 30 minutes through an intravenous line.

14. If the infusion is not administered immediately, the prepared infusion bag should not be stored longer than 8 hours at 2° C. to 8° C. (36° F. to 46° F.). DO NOT FREEZE.

DO NOT administer PADCEV as an intravenous push or bolus.

DO NOT mix PADCEV with, or administer as an infusion with, other medicinal products.

### 6.3.5 Dosage Forms and Strengths

For Injection: 20 mg and 30 mg of enfortumab vedotin-ejfv as a white to off-white lyophilized powder in a single-dose vial for reconstitution.

### 6.3.6 Warnings and Precautions

6.3.6.1 Hyperglycemia

Hyperglycemia occurred in patients treated with PADCEV, including death, and diabetic ketoacidosis (DKA) in those with and without pre-existing diabetes mellitus. The incidence of Grade 3-4 hyperglycemia increased consistently in patients with higher body mass index and in patients with higher baseline A1C. In EV-201, 8% of patients developed Grade 3-4 hyperglycemia. In this trial, patients with baseline hemoglobin A1C≥8% were excluded. Closely monitor blood glucose levels in patients with, or at risk for, diabetes mellitus or hyperglycemia. If blood glucose is elevated (>250 mg/dL), withhold PADCEV [see Dosage and Administration (6.3.4.2)].

6.3.6.2 Peripheral Neuropathy

Peripheral neuropathy, predominantly sensory, occurred in 49% of the 310 patients treated with PADCEV in clinical trials; 2% experienced Grade 3 reactions.

In study EV-201, peripheral neuropathy occurred in patients treated with PADCEV with or without preexisting peripheral neuropathy. The median time to onset of Grade ≥2 was 3.8 months (range: 0.6 to 9.2). Neuropathy led to treatment discontinuation in 6% of patients. At the time of their last evaluation, 19% had complete resolution, and 26% had partial improvement.

Monitor patients for symptoms of new or worsening peripheral neuropathy and consider dose interruption or dose reduction of PADCEV when peripheral neuropathy occurs. Permanently discontinue PADCEV in patients that develop Grade ≥3 peripheral neuropathy [see Dosage and Administration (6.3.4.2)].

6.3.6.3 Ocular Disorders

Ocular disorders occurred in 46% of the 310 patients treated with PADCEV. Majority of these events involved the cornea and included keratitis, blurred vision, limbal stem cell deficiency and other events associated with dry eyes. Dry eye symptoms occurred in 19% of patients, and blurred vision occurred in 14% of patients, during treatment with PADCEV. The median time to onset onset to symptomatic vision disorder was 3.1 months (range: 1 to 6).

Monitor patients for ocular disorders. Consider artificial tears for prophylaxis of dry eyes and ophthalmologic evaluation if ocular symptoms occur or do not resolve. Consider treatment with ophthalmic topical steroids, if indicated after an ophthalmic exam. Consider dose interruption or dose reduction of PADCEV for symptomatic ocular disorders.

6.3.6.4 Skin Reactions

Skin reactions occurred in 54% of the 310 patients treated with PADCEV in clinical trials. Twenty-six percent (26%) of patients had maculopapular rash and 30% had pruritus. Grade 3-4 skin reactions occurred in 10% of patients and included symmetrical drug-related intertriginous and flexural exanthema (SDRIFE), bullous dermatitis, exfoliative dermatitis, and palmar-plantar erythrodysesthesia.

In study EV-201, the median time to onset of severe skin reactions was 0.8 months (range: 0.2 to 5.3). Of the patients who experienced rash, 65% had complete resolution and 22% had partial improvement.

Monitor patients for skin reactions. Consider appropriate treatment, such as topical corticosteroids and antihistamines for skin reactions, as clinically indicated. For severe (Grade 3) skin reactions, withhold PADCEV until improvement or resolution and administer appropriate medical treatment. Permanently discontinue PADCEV in patients that develop Grade 4 or recurrent Grade 3 skin reactions [see Dosage and Administration (6.3.4.2)].

6.3.6.5 Infusion Site Extravasation

Skin and soft tissue reactions secondary to extravasation have been observed after administration of PADCEV. Of the 310 patients, 1.3% of patients experienced skin and soft tissue reactions. Reactions may be delayed.

Erythema, swelling, increased temperature, and pain worsened until 2-7 days after extravasation and resolved within 1-4 weeks of peak. One percent of patients developed extravasation reactions with secondary cellulitis, bullae, or exfoliation. Ensure adequate venous access prior to starting PADCEV and monitor for possible extravasation during administration. If extravasation occurs, stop the infusion and monitor for adverse reactions.

6.3.6.6 Embryo-Fetal Toxicity

Based on the mechanism of action and findings in animals, PADCEV can cause fetal harm when administered to a pregnant woman. In animal reproduction studies, administration of enfortumab vedotin to pregnant rats during the period of organogenesis caused maternal toxicity, embryo-fetal lethality, structural malformations and skeletal anomalies at maternal exposures approximately similar to the clinical exposures at the recommended human dose of 1.25 mg/kg.

Advise patients of the potential risk to the fetus. Advise female patients of reproductive potential to use effective contraception during PADCEV treatment and for 2 months after the last dose of PADCEV. Advise male patients with female partners of reproductive potential to use effective contraception during treatment with PADCEV and for 4 months after the last dose [see Use in Specific Populations (6.3.9.1, 6.3.9.3) and Clinical Pharmacology (6.3.10.1)].

### 6.3.7 Adverse Reactions

The following serious adverse reactions are described elsewhere in the specification:

Hyperglycemia [see Warnings and Precautions (6.3.6.1)]

Peripheral Neuropathy [see Warnings and Precautions (6.3.6.2)]

Ocular Disorders [see Warnings and Precautions (6.3.6.3)]

Skin Reactions [see Warnings and Precautions (6.3.6.4)]

Infusion Site Extravasation [see Warnings and Precautions (6.3.6.5)]

6.3.7.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice. The data in the WARNINGS AND PRECAUTIONS section reflect exposure to PADCEV as a single agent at 1.25 mg/kg in 310 patients in EV-201, EV-101 (NCT02091999), and EV-102 (NCT03219333). Among 310 patients receiving PADCEV, 30% were exposed for ≥6 months and 8% were exposed for ≥12 months.

The data described in this section reflect exposure to PADCEV from EV-201, a single arm study in patients (n=125) with locally advanced or metastatic urothelial cancer who had received prior treatment with a PD-1 or PD-L1 inhibitor and platinum-based chemotherapy. Patients received PADCEV 1.25 mg/kg on Days 1, 8 and 15 of a 28-day cycle until disease progression or unacceptable toxicity. The median duration of exposure to PADCEV was 4.6 months (range: 0.5-15.6).

Serious adverse reactions occurred in 46% of patients treated with PADCEV. The most common serious adverse reactions (≥2%) were diarrhea (4%), rash (3%), nausea (2%), vomiting (2%) and fatigue (2%). Fatal adverse reactions occurred in 3.2% of patients, including acute respiratory failure, aspiration pneumonia, cardiac disorder, and sepsis (each 0.8%).

Adverse reactions leading to discontinuation occurred in 16% of patients; the most common adverse reaction leading to discontinuation was peripheral neuropathy (6%). Adverse reactions leading to dose interruption occurred in 64% of patients; the most common adverse reactions leading to dose interruption were peripheral neuropathy (18%), rash (9%) and fatigue (6%). Adverse reactions leading to dose reduction occurred in 34% of patients; the most common adverse reactions leading to dose reduction were peripheral neuropathy (12%), rash (6%) and fatigue (4%).

The most common adverse reactions (≥20%) were fatigue, peripheral neuropathy, decreased appetite, rash, alopecia, nausea, dysgeusia, diarrhea and dry skin. The most common Grade ≥3 adverse reaction (≥5%) were rash, diarrhea, and fatigue.

Table 24 summarizes the all grade and Grade ≥3 adverse reactions reported in patients in EV-201.

TABLE 24
Adverse Reactions Reported in ≥15% (Any Grade) or ≥5% (Grade ≥ 3)
of Patients Treated with PADCEV in EV-201

| | PADCEV | |
|---|---|---|
| | n = 125 | |
| | All Grades | Grade ≥ 3 |
| Adverse Reaction | % | % |
| Any | 100 | 73 |

General disorders and administration site conditions

| | | |
|---|---|---|
| Fatigue* | 56 | 6 |

Nervous system disorders

| | | |
|---|---|---|
| Peripheral neuropathy^{†} | 56 | 4 |
| Dysgeusia | 42 | 0 |

Metabolism and nutrition disorders

| | | |
|---|---|---|
| Decreased appetite | 52 | 2 |

Skin and subcutaneous tissue disorders

| | | |
|---|---|---|
| Rash^{‡} | 52 | 13 |
| Alopecia | 50 | 0 |
| Dry skin | 26 | 0 |
| Pruritus | 26 | 2 |

Gastrointestinal disorders

| | | |
|---|---|---|
| Nausea | 45 | 3 |
| Diarrhea^{§} | 42 | 6 |
| Vomiting | 18 | 2 |

*Includes: asthenia and fatigue
^{†}Includes: hypoesthesia, gait disturbance, muscular weakness, neuralgia, paresthesia, peripheral motor neuropathy, peripheral sensory neuropathy and peripheral sensorimotor neuropathy.
^{‡}Includes: dermatitis acneiform, dermatitis bullous, dermatitis contact, dermatitis exfoliative, drug eruption, erythema, erythema multiforme, exfoliative rash, palmar-plantar erythrodysesthesia syndrome, photosensitivity reaction, rash, rash erythematous, rash generalized, rash macular, rash maculo-papular, rash papular, rash pustular, rash pruritic, rash vesicular, skin exfoliation, stasis dermatitis, and symmetrical drug-related intertriginous and flexural exanthema (SDRIFE) and urticaria.
^{§}Includes: pruritus and pruritus generalized
^{¶}Includes: colitis, diarrhea and enterocolitis

Other clinically significant adverse reactions (≤15%) include: herpes zoster (3%) and infusion site extravasation (2%). Table 25 shows a summary of selected laboratory abnormalities reported in ≥10% (Grades 2-4) or ≥5% (Grade 3-4) of patients treated with PADCEV in EV-201

TABLE 25
Selected Laboratory Abnormalities Reported in ≥10% (Grades 2-4) or ≥5%
(Grade 3-4) of Patients Treated with PADCEV in EV-201

| | PADCEV | |
|---|---|---|
| | Grades 2-4* | Grade 3-4* |
| Adverse Reaction | % | % |

Hematology

| | | |
|---|---|---|
| Hemoglobin decreased | 34 | 10 |
| Lymphocytes decreased | 32 | 10 |
| Neutrophils decreased | 14 | 5 |
| Leukocytes decreased | 14 | 4 |

Chemistry

| | | |
|---|---|---|
| Phosphate decreased | 34 | 10 |
| Creatinine increased | 20 | 2 |
| Lipase increased | 14 | 9 |
| Glucose increased | —^{†} | 8 |
| Sodium decreased | 8 | 8 |
| Urate increased | 7 | 7 |

*Denominator for each laboratory parameter is based on the number of patients with a baseline and post-treatment laboratory value available for 121 or 122 patients.
^{†}CTCAE Grade 2 is defined as fasting glucose >160-250 mg/dL. Fasting glucose levels were not measured in EV-201. However, 23 (19%) patients had non-fasting glucose >160-250 mg/dL.

6.3.7.2 Immunogenicity

As with all therapeutic proteins, there is a potential for immunogenicity. The detection of antibody formation is highly dependent on the sensitivity and specificity of the assay. Additionally, the observed incidence of antibody (including neutralizing antibody) positivity in an assay may be influenced by several factors including assay methodology, sample handling, timing of sample collection, concomitant medications, and underlying disease. For these reasons, comparison of the incidence of antibodies in the studies described below with the incidence of antibodies in other studies or other enfortumab vedotin products may be misleading.

A total of 365 patients were tested for immunogenicity to PADCEV; 4 patients (1%) were confirmed to be transiently positive for anti-therapeutic antibody (ATA), and 1 patient (0.3%) was confirmed to be persistently positive for ATA at any post-baseline time point. No impact of ATA on efficacy, safety and pharmacokinetics was observed.

### 6.3.8 Drug Interactions

6.3.8.1 Effects of Other Drugs on PADCEV

Strong CYP3A4 Inhibitors

Concomitant use with a strong CYP3A4 inhibitor may increase free MMAE exposure [see Clinical Pharmacology (6.3.10.3)], which may increase the incidence or severity of PADCEV toxicities. Closely monitor patients for signs of toxicity when PADCEV is given concomitantly with strong CYP3A4 inhibitors.

### 6.3.9 Use in Specific Populations

6.3.9.1 Pregnancy

Risk Summary

Based on the mechanism of action and findings in animals, PADCEV can cause fetal harm when administered to a pregnant woman [see Clinical Pharmacology (6.3.10.1)]. There are no available human data on PADCEV use in pregnant women to inform a drug-associated risk. In an animal reproduction study, administration of enfortumab vedotin-ejfv to pregnant rats during organogenesis caused maternal toxicity, embryo-fetal lethality, structural malformations and skeletal anomalies at maternal exposures approximately similar to the exposures at the recommended human dose of 1.25 mg/kg (see Animal Data). Advise patients of the potential risk to the fetus.

The background risk of major birth defects and miscarriage for the indicated population is unknown. In the U.S. general population, the estimated background risk of major birth defects and miscarriage in clinically recognized pregnancies is 2%-4% and 15%-20%, respectively.

Animal Data

In a rat pilot embryo-fetal development study, administration of enfortumab vedotin-ejfv on gestation day 6 and 13 during the period of organogenesis resulted in a complete litter loss in all pregnant rats at the maternally toxic dose of 5 mg/kg (approximately 3 times the exposure at the recommended human dose). A dose of 2 mg/kg (approximately similar to the exposure at the recommended human dose) resulted in maternal toxicity, embryo-fetal lethality and structural malformations that included gastroschisis, malrotated hindlimb, absent forepaw, malpositioned internal organs and fused cervical arch. Additionally, skeletal anomalies (asymmetric, fused, incompletely ossified, and misshapen sternebrae, misshapen cervical arch, and unilateral ossification of the thoracic centra) and decreased fetal weight were observed.

6.3.9.2 Lactation

Risk Summary

There are no data on the presence of enfortumab vedotin-ejfv in human milk, the effects on the breastfed child, or the effects on milk production. Because of the potential for serious adverse reactions in a breastfed child, advise lactating women not to breastfeed during treatment with PADCEV and for at least 3 weeks after the last dose.

6.3.9.3 Females and Males of Reproductive Potential

Pregnancy Testing

Verify pregnancy status in females of reproductive potential prior to initiating PADCEV treatment [see Use in Specific Populations (6.3.9.1)].

Contraception

Females

PADCEV can cause fetal harm when administered to a pregnant woman [see Use in Specific Populations (6.3.9.1)]. Advise females of reproductive potential to use effective contraception during PADCEV treatment and for 2 months after the last dose.

Males

Advise male patients with female partners of reproductive potential to use effective contraception during treatment with PADCEV and for 4 months after the last dose.

Infertility

Males

Based on findings from animal studies, PADCEV may impair male fertility. [see Nonclinical Toxicology (6.3.11.1)].

6.3.9.4 Pediatric Use

Safety and effectiveness of PADCEV in pediatric patients have not been established.

6.3.9.5 Geriatric Use

Of the 310 patients treated with PADCEV in clinical studies, 187 (60%) were 65 years or older and 80 (26%) were 75 years or older. No overall differences in safety or effectiveness were observed between these patients and younger patients [see Clinical Pharmacology (6.3.10.3)].

6.3.9.6 Hepatic Impairment

Avoid the use of PADCEV in patients with moderate or severe hepatic impairment. PADCEV has not been studied in patients with moderate or severe hepatic impairment [see Clinical Pharmacology (6.3.10.3)]. In another ADC that contains MMAE, the frequency of ≥Grade 3 adverse reactions and deaths was greater in patients with moderate (Child-Pugh B) or severe (Child-Pugh C) hepatic impairment compared to patients with normal hepatic function. No adjustment in the starting dose is required when administering PADCEV to patients with mild hepatic impairment.

6.3.9.7 Renal Impairment

No dose adjustment is required in patients with mild (CrCL>60-90 mL/min), moderate (CrCL 30-60 mL/min) or severe (CrCL<30 mL/min) renal impairment [see Clinical Pharmacology (6.3.10.3)].

### 6.3.10 Clinical Pharmacology

6.3.10.1 Mechanism of Action

Enfortumab vedotin-ejfv is an ADC. The antibody is a human IgG1 directed against Nectin-4, an adhesion protein located on the surface of cells. The small molecule, MMAE, is a microtubule-disrupting agent, attached to the antibody via a protease-cleavable linker. Nonclinical data suggest that the anticancer activity of enfortumab vedotin-ejfv is due to the binding of the ADC to Nectin-4-expressing cells, followed by internalization of the ADC-Nectin-4 complex, and the release of MMAE via proteolytic cleavage. Release of MMAE disrupts the microtubule network within the cell, subsequently inducing cell cycle arrest and apoptotic cell death.

6.3.10.2 Pharmacodynamics

In an exposure-response analysis, higher enfortumab vedotin exposure was associated with higher incidence of some adverse reactions (e.g., Grade ≥2 peripheral neuropathy, Grade ≥3 hyperglycemia) and a lower exposure was associated with lower efficacy.

Cardiac Electrophysiology

At the recommended dose, PADCEV had no large QTc prolongation (>20 msec).

6.3.10.3 Pharmacokinetics

Population pharmacokinetic analysis included data from 369 patients based on three Phase 1 studies and one Phase 2 study. Enfortumab vedotin-ejfv pharmacokinetics were characterized after single and multiple doses in patients with locally advanced or metastatic urothelial carcinoma and other solid tumors.

The exposure parameters of ADC and unconjugated MMAE (the cytotoxic component of enfortumab vedotin-ejfv) are summarized in Table 26 below. Peak ADC concentrations were observed near the end of intravenous infusion while peak MMAE concentrations were observed approximately 2 days after enfortumab vedotin-ejfv dosing. Minimal accumulation of the ADC and MMAE was observed following repeat administration of enfortumab vedotin-ejfv in patients. Steady-state concentrations of ADC and MMAE were reached after 1 treatment cycle.

TABLE 26
Exposure parameters of ADC and unconjugated MMAE
after first treatment cycle of 1.25 mg/kg of enfortumab
vedotin-ejfv dose of Days 1, 8 and 15

| | ADC | | Unconjugated | |
|---|---|---|---|---|
| | Mean (±SD) | | MMAE Mean (±SD) | |
| Cₘₐₓ | 28 (6.8) | μg/mL | 4.8 (2.7) | ng/mL |
| AUC_{0-28 d} | 111 (38) | μg · d/mL | 69 (42) | ng · d/mL |
| C_{trough, 0-28 d} | 0.27 (0.22) | μg/mL | 0.57 (0.58) | ng/mL |

Cₘₐₓ = maximum concentration,
AUC_{0-28 d} = area under the concentration-time curve from time zero to 28 days,
C_{trough, 0-28 d} = pre-dose concentration on day 28

Distribution

The estimated mean steady-state volume of distribution of ADC was 11 liters following administration of enfortumab vedotin-ejfv. Plasma protein binding of MMAE ranged from 68% to 82%, in vitro.

Elimination

ADC and MMAE exhibited multi-exponential declines with an elimination half-life of 3.4 days and 2.4 days, respectively. The mean clearance (CL) of enfortumab vedotin-ejfv and free MMAE in patients was 0.10 L/h and 2.7 L/h, respectively, in patients. Elimination of MMAE appeared to be limited by its rate of release from enfortumab vedotin-ejfv.

Metabolism

Enfortumab vedotin-ejfv catabolism has not been studied in humans; however, it is expected to undergo catabolism to small peptides, amino acids, unconjugated MMAE, and unconjugated MMAE-related catabolites. Enfortumab vedotin-ejfv releases MMAE via proteolytic cleavage, and MMAE is primarily metabolized by CYP3A4 in vitro.

Excretion

The excretion of enfortumab vedotin-ejfv is not fully characterized. Following a single-dose of another ADC that contains MMAE, 17% of the total MMAE administered was recovered in feces and 6% in urine over a 1- week period, primarily as unchanged drug. A similar excretion profile of MMAE is expected after enfortumab vedotin-ejfv administration.

Specific Populations

Based on population pharmacokinetic analysis, no clinically significant differences in the pharmacokinetics of enfortumab vedotin-ejfv were observed based on age (24 to 87 years), sex, or race/ethnicity (Caucasian, Asian, Black, or others).

Hepatic Impairment

Based on population pharmacokinetics analysis, there was a 48% AUC increase in unconjugated MMAE exposure observed in patients with mild hepatic impairment (bilirubin of 1 to 1.5×ULN and AST<ULN, or bilirubin ≤ULN and AST>ULN, n=31) compared to normal hepatic function. The effect of moderate or severe hepatic impairment (AST or ALT>2.5×ULN or total bilirubin >1.5×ULN) or liver transplantation on the pharmacokinetics of ADC or unconjugated MMAE is unknown.

Renal Impairment

The pharmacokinetics of enfortumab vedotin-ejfv and MMAE were evaluated after the administration of 1.25 mg/kg of enfortumab vedotin-ejfv to patients with mild (creatinine clearance; CrCL>60-90 mL/min; n=135), moderate (CrCL 30-60 mL/min; n=147) and severe (CrCL<30 mL/min; n=8) renal impairment. No significant differences in exposure (AUC) of ADC and MMAE were observed in patients with mild, moderate or severe renal impairment compared to patients with normal renal function. The effect of end stage renal disease with or without dialysis on the pharmacokinetics of ADC or unconjugated MMAE is unknown.

Drug Interaction Studies

Clinical Studies

No clinical studies evaluating the drug-drug interaction potential of enfortumab vedotin-ejfv have been conducted. To characterize the drug-drug interaction potential of free MMAE, clinical studies with another ADC that contains MMAE are described below.

Strong CYP3A4 Inhibitors: Another ADC that contains MMAE co-administered with ketoconazole (a strong CYP3A4 inhibitor) increased MMAE Cmax by 25% and AUC by 34%, with no change in ADC exposure. The concomitant use of strong inhibitors of CYP3A4 with PADCEV would likely result in similar effects on free MMAE and ADC.

Strong CYP3A4 Inducers: Another ADC that contains MMAE co-administered with rifampin (a strong CYP3A4 inducer) decreased MMAE Cmax by 44% and AUC by 46%, with no change in ADC exposure. The concomitant use of strong inducers of CYP3A4 with PADCEV would likely result in similar effects on free MMAE and ADC.

Sensitive CYP3A4 Substrates: Another ADC that contains MMAE co-administered with midazolam (a sensitive CYP3A4 substrate) did not affect the exposure of midazolam. Similarly, PADCEV is not expected to alter the exposure of drugs that are metabolized by CYP3A4 enzymes.

In Vitro Studies

Transporter Systems: MMAE is a substrate of P-glycoprotein (P-gp), but not an inhibitor of P-gp.

6.3.11 Nonclinical Toxicology

6.3.11.1 Carcinogenesis, Mutagenesis, Impairment of Fertility

Carcinogenicity studies with enfortumab vedotin-ejfv or the small molecule cytotoxic agent (MMAE) have not been conducted.

MMAE was genotoxic in the rat bone marrow micronucleus study through an aneugenic mechanism. This effect is consistent with the pharmacological effect of MMAE as a microtubule-disrupting agent. MMAE was not mutagenic in the bacterial reverse mutation assay (Ames test) or the L5178Y mouse lymphoma forward mutation assay.

Fertility studies with enfortumab vedotin-ejfv or MMAE have not been conducted. However, results of repeat-dose toxicity studies in rats indicate the potential for enfortumab vedotin-ejfv to impair male reproductive function and fertility.

In repeat-dose toxicology studies conducted in rats (for up to 13 weeks), doses ≥2 mg/kg enfortumab vedotin-ejfv (at exposures similar to the exposures at the recommended human dose) resulted in decreases in testes and epididymis weights, seminiferous tubule degeneration, spermatid/spermatocyte depletion in the testes and cell debris, sperm granuloma and hypospermia/abnormal spermatids in the epididymis. Findings in the testes and epididymis did not reverse by the end of the recovery period.

### 6.3.12 References

1. “OSHA Hazardous Drugs.” OSHA. http://www.osha.gov/SLTC/hazardousdrugs/index.html

### 6.3.13 how Supplied/Storage and Handling

6.3.13.1 How Supplied

PADCEV (enfortumab vedotin-ejfv) 20 mg and 30 mg are supplied as a sterile, preservative-free, white to off-white lyophilized powder in single-dose vials. PADCEV vials are available in the following packages:

Carton of one 20 mg single-dose vial (NDC 51144-020-01)

Carton of one 30 mg single-dose vial (NDC 51144-030-01)

6.3.13.2 Storage

Store PADCEV vials refrigerated at 2° C. to 8° C. (36° F. to 46° F.) in the original carton. Do not freeze. Do not shake.

6.3.13.3 Special Handling

PADCEV is a cytotoxic drug. Follow applicable special handling and disposal procedures.¹

6.3.14 Patient Counseling Information

Advise the patient to read the FDA-approved patient labeling (Patient Information).

Hyperglycemia

Inform patients about the risk of hyperglycemia and how to recognize associated symptoms [see Warnings and Precautions (6.3.6.1)].

Peripheral Neuropathy

Inform patients to report to their healthcare provider any numbness and tingling of the hands or feet or muscle weakness [see Warnings and Precautions (6.3.6.2)].

Ocular disorders:

Advise patients to contact their healthcare provider if they experience any visual changes [see Warnings and Precautions (6.3.6.3)]. In order to prevent or treat dry eyes, advise patients to use artificial tear substitutes.

Skin Reactions

Inform patients that rashes and severe skin reactions have occurred after administration of PADCEV. Advise patients to contact their healthcare provider for signs and symptoms of progressive or intolerable skin reactions [see Warnings and Precautions (6.3.6.4)].

Infusion Site Extravasation

Inform patients that infusion site reactions have occurred after administration of PADCEV. These reactions generally occurred immediately after administration but, in some instances, had a delayed onset (e.g., 24 hours). Instruct patients to contact their healthcare provider immediately if they experience an infusion site reaction [see Warnings and Precautions (6.3.6.5)].

Embryo-Fetal Toxicity

Advise pregnant women and females of reproductive potential of the potential risk to the fetus. Advise females to inform their healthcare providers of a known or suspected pregnancy [see Warning and Precautions (6.3.6.6) and Use in Specific Population (6.3.9.1)].

Females and Males of Reproductive Potential

Advise female patients of reproductive potential to use effective contraception during treatment with PADCEV and for 2 months after the last dose. Advise male patients with female partners of reproductive potential to use effective contraception during treatment with PADCEV and for 4 months after the last dose [see Use in Specific Populations (6.3.9.3)].

Lactation

Advise women not to breastfeed during treatment with PADCEV and for 3 weeks after the last dose. [see Use in Specific Populations (6.3.9.2)].

Infertility

Advise males of reproductive potential that PADCEV may impair fertility [see Use in Specific Populations (6.3.9.3)].

What is claimed is:

1. A method of preventing or treating cancer in a human subject, comprising (a) administering to the subject a first regimen comprising an effective amount of an antibody drug conjugate (ADC),
wherein the ADC comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23;
wherein the subject has urothelial cancer; and
wherein the subject has received an immune checkpoint inhibitor therapy and received a chemotherapy.

2. The method of claim 1, wherein the ADC is administered three times within a 28 day cycle.

3. The method of claim 1 or 2, wherein the ADC is administered on Days 1, 8 and 15 of a 28 day cycle.

4. The method of any one of claims 1 to 3, wherein the urothelial cancer is locally advanced urothelial cancer.

5. The method of any one of claims 1 to 3, wherein the urothelial cancer is metastatic urothelial cancer.

6. The method of any one of claims 1 to 5, wherein the immune checkpoint inhibitor therapy is a programmed death receptor-1 (PD-1) inhibitor.

7. The method of any one of claims 1 to 5, wherein the immune checkpoint inhibitor therapy is programmed death-ligand 1 (PD-L1) inhibitor.

8. The method of any one of claims 1 to 7, wherein the chemotherapy is platinum-containing chemotherapy.

9. The method of claim 8, wherein the platinum-containing chemotherapy is platinum-containing chemotherapy in a neoadjuvant setting.

10. The method of claim 8, wherein the platinum-containing chemotherapy is platinum-containing chemotherapy in an adjuvant setting.

11. The method of any one of claims 8 to 10, wherein the platinum-containing chemotherapy is platinum-containing chemotherapy in a locally advanced setting.

12. The method of any one of claims 8 to 10, wherein the platinum-containing chemotherapy is platinum-containing chemotherapy in a metastatic setting.

13. The method of any one of claims 1 to 12, wherein the first regimen comprises an ADC dose of about 1.25 milligram/kilogram (mg/kg) of the subject's body weight.

14. The method of claim 13, wherein the subject has a body weight of less than 100 kg.

15. The method of any one of claims 1 to 12, wherein the first regimen comprises an ADC dose of about 125 mg to the subject, wherein the subject has a body weight of no less than 100 kg.

16. The method of any one of claims 1 to 15, further comprising
(b) determining blood glucose level in the subject, and
(c) if the blood glucose level from (b) is higher than 250 mg/dL, withholding the administration of the antibody drug conjugate.

17. The method of claim 16, further comprising
(d) waiting for a period sufficient for the blood glucose level to reduce to no more than 250 mg/dL.

18. The method of claim 16 or 17, further comprising
(e) determining blood glucose level in the subject, and
(f) if the blood glucose level from (e) is no more than 250 mg/dL, administering to the subject a second regimen comprising an effective amount of the antibody drug conjugate.

19. The method of any one of claims 16 to 18, wherein if the blood glucose level from (b) or (e) is more than 500 mg/dL, discontinuing the administration of the ADC permanently.

20. The method of any one of claims 16 to 19, further comprising repeating from (a) to (f).

21. The method of any one of claims 16 to 20, wherein the subject has hyperglycemia.

22. The method of claim 21, wherein the subject has diabetic ketoacidosis (DKA).

23. The method of any one of claims 16 to 22, wherein the subject additionally has higher body mass index and/or higher baseline A1C.

24. The method of any one of claims 18 to 23, wherein second regimen is identical to the first regimen.

25. The method of any one of claims 16 to 24, wherein the blood glucose level is determined daily.

26. The method of any one of claims 16 to 24, wherein the blood glucose level is determined once every two days, once every three days, once every four days, or once every five days, once every six days.

27. The method of any one of claims 16 to 24, wherein the blood glucose level is determined weekly, bi-weekly, once every three weeks, or once every four weeks.

28. The method of any one of claims 16 to 24, wherein the blood glucose level is determined monthly, once every two months, or once every three months.

29. The method of any one of claims 1 to 28, further comprising
(g) determining peripheral neuropathy in the subject, and
(h) if the peripheral neuropathy from (g) is no less than Grade 2, withholding the administration of the antibody drug conjugate.

30. The method of claim 29, further comprising
(i) waiting for a period sufficient for the peripheral neuropathy to reduce to no more than Grade 1.

31. The method of claim 29 or 30, further comprising
(j) determining peripheral neuropathy in the subject, and
(k) if the peripheral neuropathy (j) is no more than Grade 1, administering to the subject a second regimen comprising an effective amount of the ADC, wherein the second regimen comprises an ADC dose equal to or lower than the first regimen.

32. The method of any one of claims 29 to 31, wherein if the peripheral neuropathy from (g) or (j) is no less than Grade 3, discontinuing the administration of the ADC permanently.

33. The method of any one of claims 29 to 32, wherein the peripheral neuropathy is predominantly sensory neuropathy.

34. The method of any one of claims 29 to 31, and 33, further comprising repeating from (g) to (k).

35. The method of any one of claims 31, and 33 to 34, further comprising determining the number of times the condition for the administration of the second regimen has been satisfied.

36. The method of any one of claims 31, and 33 to 35, wherein in (k) if the second regimen is administered for the first time, the second regimen is identical to the first regimen.

37. The method of any one of claims 31, and 33 to 36, wherein in (k) if the second regimen has been administered once and the subject has a body weight of less than 100 kg, the ADC dose in the second regimen is lowered to about 1.0 mg/kg of the subject's body weight.

38. The method of any one of claims 31, and 33 to 36, wherein in (k) if the second regimen has been administered once and the subject has a body weight of no less than 100 kg, the ADC dose in the second regimen is lowered to about 100 mg to the subject.

39. The method of any one of claims 31, and 33 to 38, wherein in (k) if the second regimen has been administered twice and the subject has a body weight of less than 100 kg, the ADC dose in the second regimen is lowered to about 0.75 mg/kg of the subject's body weight.

40. The method of any one of claims 31, and 33 to 38, wherein in (k) if the second regimen has been administered once and the subject has a body weight of no less than 100 kg, the ADC dose in the second regimen is lowered to about 75 mg to the subject.

41. The method of any one of claims 31, and 33 to 40, wherein in (k) if the second regimen has been administered three times and the subject has a body weight of less than 100 kg, the ADC dose in the second regimen is lowered to about 0.5 mg/kg of the subject's body weight.

42. The method of any one of claims 31, and 33 to 40, wherein in (k) if the second regimen has been administered three times and the subject has a body weight of no less than 100 kg, the ADC dose in the second regimen is lowered to about 50 mg to the subject.

43. The method of any one of claims 31 and 33 to 42, wherein the ADC dose in the second regimen is increased by an amount of about 0.25 mg/kg for the subject having a body weight of less than 100 kg or increased by an amount of about 25 mg for the subject having a body weight of no less than 100 kg, if
(1) the administration of the ADC has not been discontinued permanently,
(2) the ADC dose in the second regimen is lower than the ADC dose in the first regimen, and
(3) the peripheral neuropathy has returned to no more than Grade 1.

44. The method of any one of claims 29 to 43, wherein the peripheral neuropathy is determined daily.

45. The method of any one of claims 29 to 43, wherein the peripheral neuropathy is determined once every two days, once every three days, once every four days, or once every five days, once every six days.

46. The method of any one of claims 29 to 43, wherein the peripheral neuropathy is determined weekly, bi-weekly, once every three weeks, or once every four weeks.

47. The method of any one of claims 29 to 43, wherein the peripheral neuropathy is determined monthly, once every two months, or once every three months.

48. The method of any one of claims 1 to 47, further comprising
(l) determining a skin reaction in the subject, and
(m) if the skin reaction from (l) is no less than Grade 3, withholding the administration of the ADC.

49. The method of claim 48, further comprising
(n) waiting for a period sufficient for the skin reaction to reduce to no more than Grade 1.

50. The method of claim 48 or 49, further comprising
(o) determining the skin reaction in the subject, and
(p) if the skin reaction in (o) is no more than Grade 1, administering to the subject a second regimen comprising an effective amount of the ADC, wherein the second regimen comprises an ADC dose equal to or lower than the first regimen.

51. The method of any one of claims 48 to 50, wherein if the skin reaction from (l) or (o) is no less than Grade 4, discontinuing the administration of the ADC permanently.

52. The method of any one of claims 48 to 51, wherein the skin reaction is selected from the group consisting of maculopapular rash, pruritus, symmetrical drug-related intertriginous, flexural exanthema (SDRIFE), bullous dermatitis, exfoliative dermatitis, and palmar-plantar erythrodysesthesia.

53. The method of any one of claims 48 to 51, wherein the no less than Grade 3 skin reaction is selected from the group consisting of symmetrical drug-related intertriginous, flexural exanthema (SDRIFE), bullous dermatitis, exfoliative dermatitis, and palmar-plantar erythrodysesthesia.

54. The method of any one of claims 48 to 50, and 52 to 53, further comprising repeating from (l) to (p).

55. The method of 54, wherein if Grade 3 skin reaction reoccurs in (l) or (o), discontinuing the administration of the ADC permanently.

56. The method of any one of claims 48 to 50, and 52 to 55, further comprising determining the number of times the condition for the administration of the second regimen has been satisfied.

57. The method of any one of claims 48 to 50, and 52 to 56, wherein in (p) if the second regimen is administered for the first time, the second regimen is identical to the first regimen.

58. The method of any one of claims 48 to 50, and 52 to 57, wherein in (p) if the second regimen has been administered one or more times and the subject has a body weight of less than 100 kg, the ADC dose in the second regimen is lowered to about 1.0 mg/kg of the subject's body weight.

59. The method of any one of claims 48 to 50, and 52 to 57, wherein in (p) if the second regimen has been administered one or more times and the subject has a body weight of no less than 100 kg, the ADC dose in the second regimen is lowered to about 100 mg to the subject.

60. The method of any one of claims 48 to 50, and 52 to 59, wherein in (p) if the second regimen has been administered two or more times and the subject has a body weight of less than 100 kg, the ADC dose in the second regimen is lowered to about 0.75 mg/kg of the subject's body weight.

61. The method of any one of claims 48 to 50, and 52 to 59, wherein in (p) if the second regimen has been administered two or more times and the subject has a body weight of no less than 100 kg, the ADC dose in the second regimen is lowered to about 75 mg to the subject.

62. The method of any one of claims 48 to 50, and 52 to 61, wherein in (p) if the second regimen has been administered three or more times and the subject has a body weight of less than 100 kg, the ADC dose in the second regimen is lowered to about 0.5 mg/kg of the subject's body weight.

63. The method of any one of claims 48 to 50, and 52 to 61, wherein in (p) if the second regimen has been administered three or more times and the subject has a body weight of no less than 100 kg, the ADC dose in the second regimen is lowered to about 50 mg to the subject.

64. The method of any one of claims 48 to 50, and 52 to 56, wherein in (p) if the subject has a body weight of less than 100 kg, the second regimen comprises an ADC dose of about 1.0 mg/kg of the subject's body weight.

65. The method of any one of claims 48 to 50, and 52 to 56, wherein in (p) if the subject has a body weight of no less than 100 kg, the second regimen comprises an ADC dose of about 100 mg to the subject.

66. The method of any one of claims 48 to 50, 52 to 56, and 64 to 65, wherein in (p) if the second regimen has been administered one or more times and the subject has a body weight of less than 100 kg, the ADC dose in the second regimen is lowered to about 0.75 mg/kg of the subject's body weight.

67. The method of any one of claims 48 to 50, 52 to 56, and 64 to 65, wherein in (p) if the second regimen has been administered one or more times and the subject has a body weight of no less than 100 kg, the ADC dose in the second regimen is lowered to about 75 mg to the subject.

68. The method of any one of claims 48 to 50, 52 to 56, and 64 to 67, wherein in (p) if the second regimen has been administered two or more times and the subject has a body weight of less than 100 kg, the ADC dose in the second regimen is lowered to about 0.5 mg/kg of the subject's body weight.

69. The method of any one of claims 48 to 50, 52 to 56, and 64 to 67, wherein in (p) if the second regimen has been administered two or more times and the subject has a body weight of no less than 100 kg, the ADC dose in the second regimen is lowered to about 50 mg to the subject.

70. The method of any one of claims 50 and 52 to 69, wherein the ADC dose in the second regimen is increased by an amount of about 0.25 mg/kg for the subject having a body weight of less than 100 kg or increased by an amount of about 25 mg for the subject having a body weight of no less than 100 kg, if
(1) the administration of the ADC has not been discontinued permanently,
(2) the ADC dose in the second regimen is lower than the ADC dose in the first regimen, and
(3) the skin reaction has returned to no more than Grade 1.

71. The method of any one of claims 48 to 70, wherein the skin reaction is determined daily.

72. The method of any one of claims 48 to 70, wherein the skin reaction is determined once every two days, once every three days, once every four days, or once every five days, once every six days.

73. The method of any one of claims 48 to 70, wherein the skin reaction is determined weekly, bi-weekly, once every three weeks, or once every four weeks.

74. The method of any one of claims 48 to 70, wherein the skin reaction is determined monthly, once every two months, or once every three months.

75. The method of any one of claims 1 to 74, further comprising
(q) determining non-hematologic toxicity in the subject, and
(s) if the non-hematologic toxicity from (q) is no less than Grade 3, withholding the administration of the ADC.

76. The method of claim 75, further comprising
(t) waiting for a period sufficient for the non-hematologic toxicity to reduce to no more than Grade 1.

77. The method of claim 75 or 76, further comprising
(u) determining the non-hematologic toxicity in the subject, and
(v) if the non-hematologic toxicity in (u) is no more than Grade 1, administering to the subject a second regimen comprising an effective amount of the ADC, wherein the second regimen comprises an ADC dose equal to or lower than the first regimen.

78. The method of any one of claims 75 to 77, wherein if the non-hematologic toxicity in (q) or (u) is no less than Grade 4, discontinuing the administration of the ADC permanently.

79. The method of any one of claims 75 to 78, wherein the non-hematologic toxicity is dysgeusia.

80. The method of any one of claims 75 to 78, wherein the non-hematologic toxicity is anorexia.

81. The method of any one of claims 75 to 78, wherein the non-hematologic toxicity is loss of appetite.

82. The method of any one of claims 75 to 78, wherein the non-hematologic toxicity is an ocular disorder.

83. The method of claim 79, wherein the ocular disorder is one or more selected from the group consisting of punctate keratitis, keratitis, keratopathy, limbal stem cell deficiency, dry eye, and blurred vision.

84. The method of any one of claims 75 to 77, and 79 to 83, further comprising repeating from (q) to (v).

85. The method of any one of claims 75 to 77, and 79 to 84, further comprising determining the number of times the condition for the administration of the second regimen has been satisfied.

86. The method of any one of claims 75 to 77, and 79 to 85, wherein in (v) the second regimen is identical to the first regimen.

87. The method of any one of claims 75 to 77, and 79 to 86, wherein in (v) if the second regimen has been administered one or more times and the subject has a body weight of less than 100 kg, the ADC dose in the second regimen is lowered to about 1.0 mg/kg of the subject's body weight.

88. The method of any one of claims 75 to 77, and 79 to 86, wherein in (v) if the second regimen has been administered one or more times and the subject has a body weight of no less than 100 kg, the ADC dose in the second regimen is lowered to about 100 mg to the subject.

89. The method of any one of claims 75 to 77, and 79 to 88, wherein in (v) if the second regimen has been administered two or more times and the subject has a body weight of less than 100 kg, the ADC dose in the second regimen is lowered to about 0.75 mg/kg of the subject's body weight.

90. The method of any one of claims 75 to 77, and 79 to 88, wherein in (v) if the second regimen has been administered two or more times and the subject has a body weight of no less than 100 kg, the ADC dose in the second regimen is lowered to about 75 mg to the subject.

91. The method of any one of claims 75 to 77, and 79 to 90, wherein in (v) if the second regimen has been administered three or more times and the subject has a body weight of less than 100 kg, the ADC dose in the second regimen is lowered to about 0.5 mg/kg of the subject's body weight.

92. The method of any one of claims 75 to 77, and 79 to 90, wherein in (v) if the second regimen has been administered three or more times and the subject has a body weight of no less than 100 kg, the ADC dose in the second regimen is lowered to about 50 mg to the subject.

93. The method of any one of claims 75 to 77, and 79 to 85, wherein in (v) if the subject has a body weight of less than 100 kg, the second regimen comprises an ADC dose of about 1.0 mg/kg of the subject's body weight.

94. The method of any one of claims 75 to 77, and 79 to 85, wherein in (v) if the subject has a body weight of no less than 100 kg, the second regimen comprises an ADC dose of about 100 mg to the subject.

95. The method of any one of claims 75 to 77, and 79 to 85, 93 to 94, wherein in (v) if the second regimen has been administered one or more times and the subject has a body weight of less than 100 kg, the ADC dose in the second regimen is lowered to about 0.75 mg/kg of the subject's body weight.

96. The method of any one of claims 75 to 77, and 79 to 85, 93 to 94, wherein in (v) if the second regimen has been administered one or more times and the subject has a body weight of no less than 100 kg, the ADC dose in the second regimen is lowered to about 75 mg to the subject.

97. The method of any one of claims 75 to 77, and 79 to 85, 93 to 96, wherein in (v) if the second regimen has been administered two or more times and the subject has a body weight of less than 100 kg, the ADC dose in the second regimen is lowered to about 0.5 mg/kg of the subject's body weight.

98. The method of any one of claims 75 to 77, and 79 to 85, 93 to 96, wherein in (v) if the second regimen has been administered two or more times and the subject has a body weight of no less than 100 kg, the ADC dose in the second regimen is lowered to about 50 mg to the subject.

99. The method of any one of claims 77 and 79 to 98, wherein the ADC dose in the second regimen is increased by an amount of about 0.25 mg/kg for the subject having a body weight of less than 100 kg or increased by an amount of about 25 mg for the subject having a body weight of no less than 100 kg, if
(1) the administration of the ADC has not been discontinued permanently,
(2) the ADC dose in the second regimen is lower than the ADC dose in the first regimen, and
(3) the non-hematologic toxicity has returned to no more than Grade 1.

100. The method of any one of claims 75 to 99, wherein the non-hematologic toxicity is determined daily.

101. The method of any one of claims 75 to 99, wherein the non-hematologic toxicity is determined once every two days, once every three days, once every four days, or once every five days, once every six days.

102. The method of any one of claims 75 to 99, wherein the non-hematologic toxicity is determined weekly, bi-weekly, once every three weeks, or once every four weeks.

103. The method of any one of claims 75 to 99, wherein the non-hematologic toxicity is determined monthly, once every two months, or once every three months.

104. The method of any one of claims 1 to 103, further comprising
(w) determining hematologic toxicity in the subject, and
(x) if the hematologic toxicity from (w) is no less than Grade 2, withholding the administration of the ADC.

105. The method of claim 104, further comprising
(y) waiting for a period sufficient for the hematologic toxicity to reduce to no more than Grade 1.

106. The method of claim 104 or 105, further comprising
(z) determining the hematologic toxicity in the subject, and
(aa) if the hematologic toxicity in (z) is no more than Grade 1, administering to the subject a second regimen comprising an effective amount of the ADC, wherein the second regimen comprises an ADC dose equal to or lower than the first regimen.

107. The method of any one of claims 104 to 106, wherein if the hematologic toxicity in (w) or (z) is no less than Grade 4, discontinuing the administration of the ADC permanently.

108. The method of any one of claims 104 to 107, wherein the hematologic toxicity is thrombocytopenia.

109. The method of any one of claims 104 to 107, wherein the hematologic toxicity is selected from the group consisting of anemia, thrombocytopenia, neutropenia, and febrile neutropenia.

110. The method of any one of claims 104 to 106, and 108 to 109, further comprising repeating from (w) to (aa).

111. The method of any one of claims 106, and 108 to 110, wherein if the hematologic toxicity in (w) is no less than Grade 4 and the subject has a body weight of less than 100 kg, the ADC dose in the second regimen is lowered to about 1.0 mg/kg of the subject's body weight.

112. The method of any one of claims 106, and 108 to 110, wherein if the hematologic toxicity in (w) is no less than Grade 4 and the subject has a body weight of no less than 100 kg, the ADC dose in the second regimen is lowered to about 100 mg to the subject.

113. The method of any one of claims 106, and 108 to 110, wherein the hematologic toxicity in (w) is Grade 3 or Grade 2.

114. The method of any one of claims 106, and 108 to 110, wherein the hematologic toxicity in (w) is Grade 3 thrombocytopenia or Grade 2 thrombocytopenia.

115. The method of claim 113 or 114, further comprising determining the number of times the condition for the administration of the second regimen has been satisfied.

116. The method of any one of claims 113 to 115, wherein in (aa) the second regimen is identical to the first regimen.

117. The method of any one of claims 113 to 116, wherein in (aa) if the subject has a body weight of less than 100 kg, the ADC dose in the second regimen is lowered to about 1.0 mg/kg of the subject's body weight.

118. The method of any one of claims 113 to 116, wherein in (aa) if the subject has a body weight of no less than 100 kg, the ADC dose in the second regimen is lowered to about 100 mg to the subject.

119. The method of any one of claims 113 to 118, wherein in (aa) if the second regimen has been administered at the ADC dose of about 1.0 mg/kg or 100 mg and if the subject has a body weight of less than 100 kg, the ADC dose in the second regimen is lowered to about 0.75 mg/kg of the subject's body weight.

120. The method of any one of claims 113 to 118, wherein in (aa) if the second regimen has been administered at the ADC dose of about 1.0 mg/kg or 100 mg and if the subject has a body weight of no less than 100 kg, the ADC dose in the second regimen is lowered to about 75 mg to the subject.

121. The method of any one of claims 113 to 120, wherein in (aa) if the second regimen has been administered at the ADC dose of about 0.75 mg/kg or 75 mg and if the subject has a body weight of less than 100 kg, the ADC dose in the second regimen is lowered to about 0.5 mg/kg of the subject's body weight.

122. The method of any one of claims 113 to 120, wherein in (aa) if the second regimen has been administered at the ADC dose of about 0.75 mg/kg or 75 mg and if the subject has a body weight of no less than 100 kg, the ADC dose in the second regimen is lowered to about 50 mg to the subject.

123. The method of any one of claims 106 and 108 to 122, wherein the ADC dose in the second regimen is increased by an amount of about 0.25 mg/kg for the subject having a body weight of less than 100 kg or increased by an amount of about 25 mg for the subject having a body weight of no less than 100 kg, if
(1) the administration of the ADC has not been discontinued permanently,
(2) the ADC dose in the second regimen is lower than the ADC dose in the first regimen, and
(3) the hematologic toxicity has returned to no more than Grade 1.

124. The method of any one of claims 104 to 123, wherein the hematologic toxicity is determined daily.

125. The method of any one of claims 104 to 123, wherein the hematologic toxicity is determined once every two days, once every three days, once every four days, or once every five days, once every six days.

126. The method of any one of claims 104 to 123, wherein the hematologic toxicity is determined weekly, bi-weekly, once every three weeks, or once every four weeks.

127. The method of any one of claims 104 to 123, wherein the hematologic toxicity is determined monthly, once every two months, or once every three months.

128. The method of any one of claims 1 to 127, further comprising
(ab) determining fatigue in the subject, and
(ac) if the fatigue from (ab) is no less than Grade 3, withholding the administration of the ADC.

129. The method of claim 128, further comprising
(ad) waiting for a period sufficient for the fatigue to reduce to no more than Grade 1.

130. The method of claim 128 or 129, further comprising
(ae) determining the fatigue in the subject, and
(af) if the fatigue in (ae) is no more than Grade 1, administering to the subject a second regimen comprising an effective amount of the ADC, wherein the second regimen comprises an ADC dose equal to or lower than the first regimen.

131. The method of any one of claims 128 to 130, wherein if the fatigue in (ab) or (ae) is no less than Grade 4, discontinuing the administration of the ADC permanently.

132. The method of any one of claims 128 to 130, further comprising repeating from (ab) to (af).

133. The method of any one of claims 128 to 130 and 132, further comprising determining the number of times the condition for the administration of the second regimen has been satisfied.

134. The method of any one of claims 128 to 130 and 132 to 133, wherein in if the fatigue in (ab) is Grade 3, the second regimen is identical to the first regimen.

135. The method of any one of claims 128 to 130 and 132 to 134, wherein if the fatigue in (ab) is Grade 3 and the subject has a body weight of less than 100 kg, the ADC dose in the second regimen is lowered to about 1.0 mg/kg of the subject's body weight.

136. The method of any one of claims 128 to 130 and 132 to 134, wherein if the fatigue in (ab) is Grade 3 and the subject has a body weight of no less than 100 kg, the ADC dose in the second regimen is lowered to about 100 mg to the subject.

137. The method of any one of claims 128 to 130 and 132 to 136, wherein in (af) if the second regimen has been administered at the ADC dose of about 1.0 mg/kg or 100 mg and if the subject has a body weight of less than 100 kg, the ADC dose in the second regimen is lowered to about 0.75 mg/kg of the subject's body weight.

138. The method of any one of claims 128 to 130 and 132 to 136, wherein in (af) if the second regimen has been administered at the ADC dose of about 1.0 mg/kg or 100 mg and if the subject has a body weight of no less than 100 kg, the ADC dose in the second regimen is lowered to about 75 mg to the subject.

139. The method of any one of claims 128 to 130 and 132 to 138, wherein in (af) if the second regimen has been administered at the ADC dose of about 0.75 mg/kg or 75 mg and if the subject has a body weight of less than 100 kg, the ADC dose in the second regimen is lowered to about 0.5 mg/kg of the subject's body weight.

140. The method of any one of claims 128 to 130 and 132 to 138, wherein in (af) if the second regimen has been administered at the ADC dose of about 0.75 mg/kg or 75 mg and if the subject has a body weight of no less than 100 kg, the ADC dose in the second regimen is lowered to about 50 mg to the subject.

141. The method of any one of claims 130 and 132 to 140, wherein the ADC dose in the second regimen is increased by an amount of about 0.25 mg/kg for the subject having a body weight of less than 100 kg or increased by an amount of about 25 mg for the subject having a body weight of no less than 100 kg, if
(1) the administration of the ADC has not been discontinued permanently,
(2) the ADC dose in the second regimen is lower than the ADC dose in the first regimen, and
(3) the fatigue has returned to no more than Grade 1.

142. The method of any one of claims 128 to 141, wherein the fatigue is determined daily.

143. The method of any one of claims 128 to 141, wherein the fatigue is determined once every two days, once every three days, once every four days, or once every five days, once every six days.

144. The method of any one of claims 128 to 141, wherein the fatigue is determined weekly, bi-weekly, once every three weeks, or once every four weeks.

145. The method of any one of claims 128 to 141, wherein the fatigue is determined monthly, once every two months, or once every three months.

146. The method of any one of claims 1 to 145, further comprising
(ag) determining diarrhea in the subject, and
(ah) if the diarrhea from (ag) is no less than Grade 3, withholding the administration of the ADC.

147. The method of claim 146, further comprising
(ai) waiting for a period sufficient for the diarrhea to reduce to no more than Grade 1.

148. The method of claim 146 or 147, further comprising
(aj) determining the diarrhea in the subject, and
(ak) if the diarrhea in (aj) is no more than Grade 1, administering to the subject a second regimen comprising an effective amount of the ADC, wherein the second regimen comprises an ADC dose equal to or lower than the first regimen.

149. The method of any one of claims 146 to 148, wherein if the diarrhea in (ag) or (ai) is no less than Grade 4 and the diarrhea does not improve to no more than Grade 2 within 72 hours with supportive management, discontinuing the administration of the ADC permanently.

150. The method of any one of claims 146 to 148, further comprising repeating from (ag) to (ak).

151. The method of any one of claims 146 to 148 and 150, further comprising determining the number of times the condition for the administration of the second regimen has been satisfied.

152. The method of any one of claims 146 to 148 and 150 to 151, wherein in (ak) the second regimen is identical to the first regimen.

153. The method of any one of claims 146 to 148 and 150 to 152, wherein in (ak) if the second regimen has been administered one or more times and the subject has a body weight of less than 100 kg, the ADC dose in the second regimen is lowered to about 1.0 mg/kg of the subject's body weight.

154. The method of any one of claims 146 to 148 and 150 to 152, wherein in (ak) if the second regimen has been administered one or more times and the subject has a body weight of no less than 100 kg, the ADC dose in the second regimen is lowered to about 100 mg to the subject.

155. The method of any one of claims 146 to 148 and 150 to 154, wherein in (ak) if the second regimen has been administered two or more times and the subject has a body weight of less than 100 kg, the ADC dose in the second regimen is lowered to about 0.75 mg/kg of the subject's body weight.

156. The method of any one of claims 146 to 148 and 150 to 154, wherein in (ak) if the second regimen has been administered two or more times and the subject has a body weight of no less than 100 kg, the ADC dose in the second regimen is lowered to about 75 mg to the subject.

157. The method of any one of claims 146 to 148 and 150 to 156, wherein in (ak) if the second regimen has been administered three or more times and the subject has a body weight of less than 100 kg, the ADC dose in the second regimen is lowered to about 0.5 mg/kg of the subject's body weight.

158. The method of any one of claims 146 to 148 and 150 to 156, wherein in (ak) if the second regimen has been administered three or more times and the subject has a body weight of no less than 100 kg, the ADC dose in the second regimen is lowered to about 50 mg to the subject.

159. The method of any one of claims 146 to 148 and 150 to 151, wherein in (ak) if the subject has a body weight of less than 100 kg, the second regimen comprises an ADC dose of about 1.0 mg/kg of the subject's body weight.

160. The method of any one of claims 146 to 148 and 150 to 151, wherein in (ak) if the subject has a body weight of no less than 100 kg, the second regimen comprises an ADC dose of about 100 mg to the subject.

161. The method of any one of claims 146 to 148, 150 to 151, and 159 to 160, wherein in (ak) if the second regimen has been administered one or more times and the subject has a body weight of less than 100 kg, the ADC dose in the second regimen is lowered to about 0.75 mg/kg of the subject's body weight.

162. The method of any one of claims 146 to 148, 150 to 151, and 159 to 160, wherein in (ak) if the second regimen has been administered one or more times and the subject has a body weight of no less than 100 kg, the ADC dose in the second regimen is lowered to about 75 mg to the subject.

163. The method of any one of claims 146 to 148, 150 to 151, and 159 to 162, wherein in (ak) if the second regimen has been administered two or more times and the subject has a body weight of less than 100 kg, the ADC dose in the second regimen is lowered to about 0.5 mg/kg of the subject's body weight.

164. The method of any one of claims 146 to 148, 150 to 151, and 159 to 162, wherein in (ak) if the second regimen has been administered two or more times and the subject has a body weight of no less than 100 kg, the ADC dose in the second regimen is lowered to about 50 mg to the subject.

165. The method of any one of claims 148 and 150 to 164, wherein the ADC dose in the second regimen is increased by an amount of about 0.25 mg/kg for the subject having a body weight of less than 100 kg or increased by an amount of about 25 mg for the subject having a body weight of no less than 100 kg, if
(1) the administration of the ADC has not been discontinued permanently,
(2) the ADC dose in the second regimen is lower than the ADC dose in the first regimen, and
(3) the diarrhea has returned to no more than Grade 1.

166. The method of any one of claims 146 to 165, wherein the diarrhea is determined daily.

167. The method of any one of claims 146 to 165, wherein the diarrhea is determined once every two days, once every three days, once every four days, or once every five days, once every six days.

168. The method of any one of claims 146 to 165, wherein the diarrhea is determined weekly, bi-weekly, once every three weeks, or once every four weeks.

169. The method of any one of claims 146 to 165, wherein the diarrhea is determined monthly, once every two months, or once every three months.

170. The method of any one of claims 1 to 169, wherein the antibody or antigen binding fragment thereof comprises CDR H1 comprising the amino acid sequence of SEQ ID NO:9, CDR H2 comprising the amino acid sequence of SEQ ID NO:10, CDR H3 comprising the amino acid sequence of SEQ ID NO:11; CDR L1 comprising the amino acid sequence of SEQ ID NO:12, CDR L2 comprising the amino acid sequence of SEQ ID NO:13, and CDR L3 comprising the amino acid sequence of SEQ ID NO:14.

171. The method of any one of claims 1 to 169, wherein the antibody or antigen binding fragment thereof comprises CDR H1 comprising the amino acid sequence of SEQ ID NO:16, CDR H2 comprising the amino acid sequence of SEQ ID NO:17, CDR H3 comprising the amino acid sequence of SEQ ID NO:18; CDR L1 comprising the amino acid sequence of SEQ ID NO:19, CDR L2 comprising the amino acid sequence of SEQ ID NO:20, and CDR L3 comprising the amino acid sequence of SEQ ID NO:21.

172. The method of any one of claims 1 to 169, wherein the antibody or antigen binding fragment thereof comprises CDR H1 consisting of the amino acid sequence of SEQ ID NO:9, CDR H2 consisting of the amino acid sequence of SEQ ID NO:10, CDR H3 consisting of the amino acid sequence of SEQ ID NO:11; CDR L1 consisting of the amino acid sequence of SEQ ID NO:12, CDR L2 consisting of the amino acid sequence of SEQ ID NO:13, and CDR L3 consisting of the amino acid sequence of SEQ ID NO:14.

173. The method of any one of claims 1 to 169, wherein the antibody or antigen binding fragment thereof comprises CDR H1 consisting of the amino acid sequence of SEQ ID NO:16, CDR H2 consisting of the amino acid sequence of SEQ ID NO:17, CDR H3 consisting of the amino acid sequence of SEQ ID NO:18; CDR L1 consisting of the amino acid sequence of SEQ ID NO:19, CDR L2 consisting of the amino acid sequence of SEQ ID NO:20, and CDR L3 consisting of the amino acid sequence of SEQ ID NO:21.

174. The method of any one of claims 1 to 173, wherein the antibody or antigen binding fragment thereof comprises a heavy chain variable region comprising the amino acid sequence of SEQ ID NO:22 and a light chain variable region comprising the amino acid sequence of SEQ ID NO:23.

175. The method of any one of claims 1 to 174, wherein the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

176. The method of any one of claims 1 to 175, wherein the antigen binding fragment is an Fab, F(ab′)2, Fv or scFv fragment.

177. The method of any one of claims 1 to 176, wherein the antibody is a fully human antibody.

178. The method of any one of claims 1 to 177, wherein the antibody or antigen binding fragment thereof is recombinantly produced.

179. The method of any one of claims 1 to 178, wherein the antibody or antigen binding fragment is linked to each unit of monomethyl auristatin E (MMAE) via a linker.

180. The method of claim 179, wherein the linker is an enzyme-cleavable linker, and wherein the linker forms a bond with a sulfur atom of the antibody or antigen binding fragment thereof.

181. The method of claim 179 or 180, wherein the linker has a formula of: -Aa-Ww-Yy-; wherein -A- is a stretcher unit, a is 0 or 1; —W— is an amino acid unit, w is an integer ranging from 0 to 12; and —Y— is a spacer unit, y is 0, 1, or 2.

182. The method of claim 181, wherein the stretcher unit has the structure of Formula (1) below; the amino acid unit is valine citrulline; and the spacer unit is a PAB group comprising the structure of Formula (2) below:

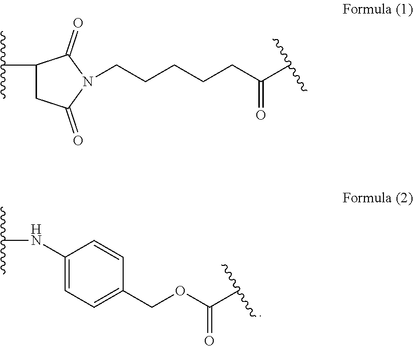 [SMILES: *C(=O)CCCCCN1C(=O)CC(*)C1=O ; *Nc1ccc(COC(*)=O)cc1]

183. The method of claim 181 or 182, wherein the stretcher unit forms a bond with a sulfur atom of the antibody or antigen binding fragment thereof; and wherein the spacer unit is linked to MMAE via a carbamate group.

184. The method of any one of claims 1 to 183, wherein the antibody is a fully human monoclonal antibody and wherein the antibody is an IgG1.

185. The method of any one of claims 1 to 184, wherein the ADC comprises from 1 to 10 units of MMAE per antibody or antigen binding fragment thereof.

186. The method of any one of claims 1 to 185, wherein the ADC comprises from 2 to 8 units of MMAE per antibody or antigen binding fragment thereof.

187. The method of any one of claims 1 to 186, wherein the ADC comprises from 3 to 5 units of MMAE per antibody or antigen binding fragment thereof.

188. The method of any one of claims 1 to 187, wherein the ADC comprises from 3 to 4 units of MMAE per antibody or antigen binding fragment thereof.

189. The method of any one of claims 1 to 188, wherein the ADC comprises about 4 units of MMAE per antibody or antigen binding fragment thereof.

190. The method of any one of claims 1 to 185, wherein the ADC has the following structure: 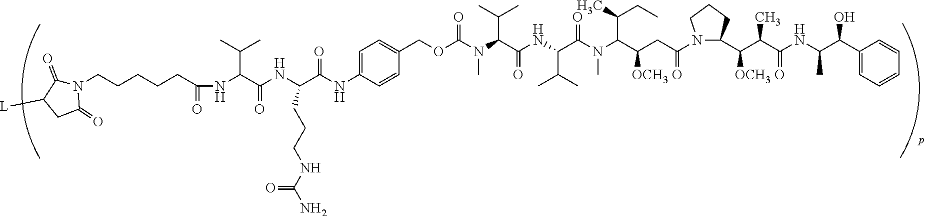
wherein L- represents the antibody or antigen binding fragment thereof and p is from 1 to 10.

191. The method of claim 190, wherein p is from 2 to 8.

192. The method of claim 190 or 191, wherein p is from 3 to 5.

193. The method of claims 190 to 192, wherein p is from 3 to 4.

194. The method of claims 190 to 193, wherein p is about 4.

195. The method of claims 190 to 193, wherein p is about 3.8.

196. The method of any one of claims 1 to 195, wherein the ADC is formulated in a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.

197. The method of any one of claims 1 to 195, wherein the ADC is formulated in a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.

198. The method of any one of claims 1 to 195, wherein the ADC is formulated at about 10 mg/ml in a pharmaceutical composition comprising about 1.4 mg/ml histidine, about 2.31 mg/ml histidine hydrochloride monohydrate, about 0.2 mg/ml polysorbate 20 (TWEEN-20), and about 55 mg/ml trehalose dihydrate, and wherein the pH of the pharmaceutical composition is about 6.0 at 25° C.

199. The method of any one of claims 1 to 195, wherein the ADC is formulated in a vial comprising a pharmaceutical composition comprising about 20 mg of the ADC, about 2.8 mg histidine, about 4.62 mg histidine hydrochloride monohydrate, about 0.4 mg polysorbate 20 (TWEEN-20), and about 110 mg trehalose dihydrate.

200. The method of any one of claims 1 to 195, wherein the ADC is formulated in a vial comprising a pharmaceutical composition comprising about 30 mg of the ADC, about 4.2 mg histidine, about 6.93 mg histidine hydrochloride monohydrate, about 0.6 mg polysorbate 20 (TWEEN-20), and about 165 mg trehalose dihydrate.

201. The method of any one of claims 1 to 200, wherein the ADC is administered by an intravenous (IV) injection or infusion.

202. The method of any one of claims 1 to 201, wherein the ADC or the ADC formulated in the pharmaceutical composition is administered by an intravenous (IV) injection or infusion over about 30 minutes.

203. A method for treating cancer in a subject, comprising administering a treatment regimen to the subject, wherein the treatment regimen comprises:
a. administering one or more doses of an antibody drug conjugate (ADC) to the subject, wherein the one or more doses are administered at a first dose level that contains an effective amount of the ADC;
b. determining whether the subject experiences an adverse reaction in response to administration of the ADC in (a), wherein the adverse reaction is selected from the group consisting of hyperglycemia, peripheral neuropathy, a skin reaction, a nonhematologic toxicity, and a hematologic toxicity;
c. administering one or more subsequent doses of the ADC, each containing an effective amount of the ADC, or discontinuing administration of the ADC based upon the determination in (b), wherein
i. if the subject is determined not to have experienced an adverse reaction to the ADC or the adverse reaction is determined to be below a defined level, then the one or more subsequent doses of the ADC are administered to the subject at the first dose level;
ii. if the subject is determined to have experienced an adverse reaction to the ADC at or above a defined level, the treatment regimen is permanently discontinued or administration of the one or more subsequent doses of the ADC are withheld for a period of time sufficient to reduce the adverse reaction to a desired level and then administration of the one or more subsequent doses of ADC are administered at the first dose level or a reduced dose level that is reduced relative to the first dose level; and
d. optionally repeating (a)-(c) one or more times, each repetition of (a)-(c) defining a treatment round, wherein the first dose level in (a) of each subsequent treatment round is either the first dose level from (a) from the immediately preceding round or the reduced dose level of c(ii) from the immediately preceding round, and wherein if the subject is found to have a recurrence of the adverse reaction in two successive treatment rounds, the one or more subsequent doses of the ADC administered in c(ii) is reduced relative to the dose administered in (a) during that treatment round, or administration of the ADC is permanently discontinued; and wherein:
i. the subject has urothelial cancer, optionally selected from the group of locally advanced or metastatic urothelial cancer, and has previously been treated with an immune checkpoint inhibitor and a chemotherapy agent, wherein the immune checkpoint inhibitor is optionally a programmed death receptor-1 (PD-1) inhibitor, or a programmed death-ligand 1 (PD-L1) inhibitor, and wherein the immune checkpoint inhibitor was optionally administered in a neoadjuvant or adjuvant setting; and
ii. the ADC comprises an antibody or antigen binding fragment thereof that binds to 191P4D12 and is conjugated to one or more units of monomethyl auristatin E (MMAE), wherein the antibody or antigen binding fragment thereof comprises a heavy chain variable region comprising complementarity determining regions (CDRs) comprising the CDRs of the heavy chain variable region set forth in SEQ ID NO:22 and a light chain variable region comprising the CDRs the light chain variable region set forth in SEQ ID NO:23.

204. The method of claim 203, wherein
A. the treatment regimen comprises (a)-(d);
B. the first dose level for the initial treatment round is the starting dose level as indicated in the dose reduction schedule below; and
C. the reduced dose level in c(ii) for each treatment round is reduced to the first dose reduction, the second dose reduction, or the third dose reduction level as set forth in the dose reduction schedule below depending upon whether the dose reduction in c(ii) is the first, second or third dose reduction in the collective treatment rounds, respectively.

| | Dose Level |
|---|---|
| Starting Dose | 1.25 mg/kg if the subject weighs less than 100 kg and |
| | up to 125 mg if the subject weighs 100 kg or more |
| First Dose | 1.0 mg/kg if the subject weighs less than 100 kg, and |
| Reduction | up to 100 mg if the subject weighs 100 kg or more |
| Second Dose | 0.75 mg/kg if the subject weighs less than 100 kg, and |
| Reduction | up to 75 mg if the subject weighs 100 kg or more |
| Third Dose | 0.5 mg/kg if the subject weighs less than 100 kg, and |
| Reduction | up to 50 mg if the subject weighs 100 kg or more |

205. The method of claim 203 or 204, wherein
I. the adverse reaction in (b) is hyperglycemia and determining comprises determining the blood glucose level of the subject;
II. the determination to continue or discontinue administration of the ADC in (c) is made as follows:
i. if the blood glucose level of the subject is equal to or below 250 mg/dL, then the one or more subsequent doses are administered at the first dose level;
ii. if the blood glucose level of the subject is greater than 250 mg/dL, then administration of the one or more subsequent doses of the ADC are withheld for a period of time sufficient to reduce the blood glucose level to less than or equal to 250 mg/dL, and then the one or more subsequent doses of the ADC are administered at the first dose level; and
iii. if the blood glucose level of the subject is greater than 500 mg/dL, then the treatment regimen is permanently discontinued.

206. The method of claim 204, wherein
I. the determination of an adverse reaction in (b) comprises determining if the subject experiences new or worsening symptoms of peripheral neuropathy; and
II. the determination to continue or discontinue administration of the ADC in (c) is made as follows:
i. if the subject experiences no symptoms of peripheral neuropathy or has symptoms of peripheral neuropathy below Grade 2, then the one or more subsequent doses of the ADC are administered at the first dose level;
ii. if the subject experiences a first occurrence of symptoms of Grade 2 peripheral neuropathy at the first dose level administered in (a), then administration of the one or more subsequent doses of the ADC are withheld for a period sufficient to reduce the symptoms of peripheral neuropathy to Grade 1 or lower, and then administration of the one or more subsequent doses of the ADC at the dose level administered in (a) is resumed;
iii. if the subject has recurrent symptoms of peripheral neuropathy after two successive treatment rounds at the same dose level in (a), then the dose is reduced by one dose level in accordance with the dose reduction schedule; and
iv. If the subject experiences symptoms of peripheral neuropathy at Grade 3 or higher, then the treatment regimen is permanently discontinued.

207. The method of claim 204, wherein
I. the determination of an adverse reaction in (b) comprises determining if the subject experiences a skin reaction; and
II. the decision to continue or discontinue administration of the ADC in (c) is made as follows:
i. if the subject experiences no skin reaction or has a skin reaction below Grade 3, then the one or more subsequent doses of the ADC are administered at the first dose level;
ii. if the subject experiences a Grade 3 skin reaction, then the one or more subsequent doses of the ADC are withheld for a period sufficient to reduce the skin reaction to Grade 1 or less and then administration of the one or more subsequent doses of the ADC at the dose level administered in (a) is resumed or reduced by one dose level in accordance with the dose reduction schedule;
iii. if the subject experiences a Grade 4 skin reaction or has recurrent Grade 3 skin reactions following multiple administrations of the ADC, then the treatment regimen is permanently discontinued.

208. The method of claim 204, wherein
I. the determination of an adverse reaction in (b) comprises determining if the subject has symptoms of a nonhematologic toxicity; and
II. the decision to continue or discontinue administration of the ADC in (c) is made as follows:
i. if the subject experiences a nonhematological toxicity that is below Grade 3, then the one or more subsequent doses of the ADC are administered at the first dose level;
ii. if the subject experiences a Grade 3 nonhematological toxicity, then the one or more subsequent doses of the ADC are withheld for a period sufficient to reduce the nonhematological to Grade 1 or less and then administration of the one or more subsequent doses of the ADC at the dose level administered in (a) is resumed or reduced by one dose level in accordance with the dose reduction schedule;
iii. if the subject experiences a Grade 4 nonhematological toxicity, then the treatment regimen is permanently discontinued.

209. The method of claim 204, wherein
I. the determination of an adverse reaction in (b) comprises determining if the subject has symptoms of a hematologic toxicity, wherein the hematological toxicity is optionally thrombocytopenia; and
II. the decision to continue or discontinue administration of the ADC in (c) is made as follows:
i. if the subject experiences a hematological toxicity that is below Grade 3 and the hematological toxicity is not thrombocytopenia, then the one or more subsequent doses of the ADC are administered at the first dose level;
ii. if the subject experiences a Grade 2 or Grade 3 hematological toxicity, wherein the hematological toxicity is thrombocytopenia, then the one or more subsequent doses of the ADC are withheld for a period sufficient to reduce the thrombocytopenia to Grade 1 or less and then administration of the one or more subsequent doses of the ADC at the dose level administered in (a) is resumed or reduced by one dose level in accordance with the dose reduction schedule;
iii. if the subject experiences a Grade 4 nonhematological toxicity that is not thrombocytopenia, then administration of the one or more subsequent doses of the ADC at the dose level administered in (a) is reduced by one dose level in accordance with the dose reduction schedule or the treatment regimen is permanently discontinued.

210. The method of any one of claims 203 to 209, wherein the antibody or antigen binding fragment thereof comprises a CDR H1 comprising the amino acid sequence of SEQ ID NO:9, a CDR H2 comprising the amino acid sequence of SEQ ID NO:10, a CDR H3 comprising the amino acid sequence of SEQ ID NO:11; a CDR L1 comprising the amino acid sequence of SEQ ID NO:12, a CDR L2 comprising the amino acid sequence of SEQ ID NO:13, and a CDR L3 comprising the amino acid sequence of SEQ ID NO:14.

211. The method of any one of claims 203 to 209, wherein the antibody or antigen binding fragment thereof comprises a CDR H1 comprising the amino acid sequence of SEQ ID NO:16, a CDR H2 comprising the amino acid sequence of SEQ ID NO:17, a CDR H3 comprising the amino acid sequence of SEQ ID NO:18; a CDR L1 comprising the amino acid sequence of SEQ ID NO:19, a CDR L2 comprising the amino acid sequence of SEQ ID NO:20, and a CDR L3 comprising the amino acid sequence of SEQ ID NO:21.

212. The method of any one of claims 203 to 209, wherein the antibody or antigen binding fragment thereof comprises a CDR H1 consisting of the amino acid sequence of SEQ ID NO:9, a CDR H2 consisting of the amino acid sequence of SEQ ID NO:10, a CDR H3 consisting of the amino acid sequence of SEQ ID NO:11; a CDR L1 consisting of the amino acid sequence of SEQ ID NO:12, a CDR L2 consisting of the amino acid sequence of SEQ ID NO:13, and a CDR L3 consisting of the amino acid sequence of SEQ ID NO:14.

213. The method of any one of claims 203 to 209, wherein the antibody or antigen binding fragment thereof comprises a CDR H1 consisting of the amino acid sequence of SEQ ID NO:16, a CDR H2 consisting of the amino acid sequence of SEQ ID NO:17, a CDR H3 consisting of the amino acid sequence of SEQ ID NO:18; a CDR L1 consisting of the amino acid sequence of SEQ ID NO:19, a CDR L2 consisting of the amino acid sequence of SEQ ID NO:20, and a CDR L3 consisting of the amino acid sequence of SEQ ID NO:21.

214. The method of any one of claims 203 to 213, wherein the antibody or antigen binding fragment thereof comprises a heavy chain variable region comprising the amino acid sequence of SEQ ID NO:22 and a light chain variable region comprising the amino acid sequence of SEQ ID NO:23.

215. The method of any one of claims 203 to 214, wherein the ADC has the following structure: 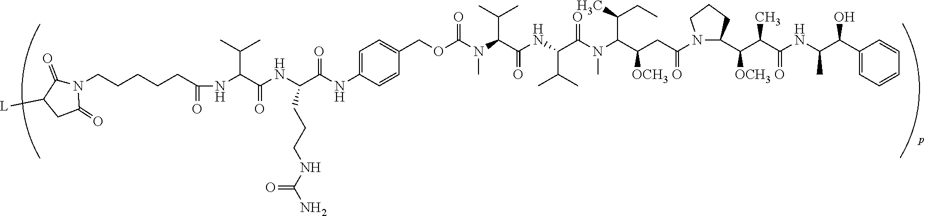
wherein L- represents the antibody or antigen binding fragment thereof and p is from 1 to 10.

216. The method of claim 215, wherein p is from 3 to 5.

217. The method of claim 215 or 216, wherein p is from 3 to 4.

218. The method of any one of claims 215 to 217, wherein p is about 4.

219. The method of any one of claims 215 to 217, wherein p is about 3.8.


SEQUENCE LISTING (23 sequences)

SEQ ID NO: 1 - DNA, 3464 bp, Homo sapiens
  CDS (264)...(1796)
  misc_feature (1)...(3464): 191P4D12
ggccgtcgttgttggccacagcgtgggaagcagctctgggggagctcggagctcccgatcacggcttcttgggggtagctacggctgggtgtgtagaacggggccggggctggggctgggtcccctagtggagacccaagtgcgagaggcaagaactctgcagcttcctgccttctgggtcagttccttattcaagtctgcagccggctcccagggagatctcggtggaacttcagaaacgctgggcagtctgcctttcaaccatgcccctgtccctgggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctgacccaggcctgcctcccttccctaggcctggctccttctgttgacatgggagattttagctcatcttgggggcctccttaaacacccccatttcttgcggaagatgctccccatcccactgactgcttgacctttacctccaacccttctgttcatcgggagggctccaccaattgagtctctcccaccatgcatgcaggtcactgtgtgtgtgcatgtgtgcctgtgtgagtgttgactgactgtgtgtgtgtggaggggtgactgtccgtggaggggtgactgtgtccgtggtgtgtattatgctgtcatatcagagtcaagtgaactgtggtgtatgtgccacgggatttgagtggttgcgtgggcaacactgtcagggtttggcgtgtgtgtcatgtggctgtgtgtgacctctgcctgaaaaagcaggtattttctcagaccccagagcagtattaatgatgcagaggttggaggagagaggtggagactgtggctcagacccaggtgtgcgggcatagctggagctggaatctgcctccggtgtgagggaacctgtctcctaccacttcggagccatgggggcaagtgtgaagcagccagtccctgggtcagccagaggcttgaactgttacagaagccctctgccctctggtggcctctgggcctgctgcatgtacatattttctgtaaatatacatgcgccgggagcttcttgcaggaatactgctccgaatcacttttaatttttttcttttttttttcttgccctttccattagttgtattttttatttatttttatttttatttttttttagagatggagtctcactatgttgctcaggctggccttgaactcctgggctcaagcaatcctcctgcctcagcctccctagtagctgggactttaagtgtacaccactgtgcctgctttgaatcctttacgaagagaaaaaaaaaattaaagaaagcctttagatttatccaatgtttactactgggattgcttaaagtgaggcccctccaacaccagggggttaattcctgtgattgtgaaaggggctacttccaaggcatcttcatgcaggcagccccttgggagggcacctgagagctggtagagtctgaaattagggatgtgagcctcgtggttactgagtaaggtaaaattgcatccaccattgtttgtgataccttagggaattgcttggacctggtgacaagggctcctgttcaatagtggtgttggggagagagagagcagtgattatagaccgagagagtaggagttgaggtgaggtgaaggaggtgctgggggtgagaatgtcgcctttccccctgggttttggatcactaattcaaggctcttctggatgtttctctgggttggggctggagttcaatgaggtttatttttagctggcccacccagatacactcagccagaatacctagatttagtacccaaactcttcttagtctgaaatctgctggatttctggcctaagggagaggctcccatccttcgttccccagccagcctaggacttcgaatgtggagcctgaagatctaagatcctaacatgtacattttatgtaaatatgtgcatatttgtacataaaatgatattctgtttttaaataaacagacaaaacttgaaaaa

SEQ ID NO: 2 - protein, 510 aa, Homo sapiens
  misc_feature (1)...(510): 191P4D12
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 3 - DNA, 1432 bp, Homo sapiens
  CDS (32)...(1432)
  misc_feature (1)...(1432): Ha22-2(2,4)6.1 heavy chain
ggtgatcagcactgaacacagaggactcaccatggagttggggctgtgctgggttttccttgttgctattttagaaggtgtccagtgtgaggtgcagctggtggagtctgggggaggcttggtacagcctggggggtccctgagactctcctgtgcagcctctggattcaccttcagtagctataacatgaactgggtccgccaggctccagggaaggggctggagtgggtttcatacattagtagtagtagtagtaccatatactacgcagactctgtgaagggccgattcaccatctccagagacaatgccaagaactcactgtctctgcaaatgaacagcctgagagacgaggacacggctgtgtattactgtgcgagagcatactactacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 4 - protein, 466 aa, Homo sapiens
  misc_feature (1)...(466): Ha22-2(2,4)6.1 heavy chain
MELGLCWVFLVAILEGVQCEVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 735 bp, Homo sapiens
  CDS (25)...(735)
  misc_feature (1)...(735): Ha22-2(2,4)6.1 light chain
agtcagacccagtcaggacacagcatggacatgagggtccccgctcagctcctggggctcctgctgctctggttcccaggttccagatgcgacatccagatgacccagtctccatcttccgtgtctgcatctgttggagacagagtcaccatcacttgtcgggcgagtcagggtattagcggctggttagcctggtatcagcagaaaccagggaaagcccctaagttcctgatctatgctgcatccactttgcaaagtggggtcccatcaaggttcagcggcagtggatctgggacagatttcactctcaccatcagcagcctgcagcctgaagattttgcaacttactattgtcaacaggctaacagtttccctcccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 6 - protein, 236 aa, Homo sapiens
  misc_feature (1)...(236): Ha22-2(2,4)6.1 light chain
MDMRVPAQLLGLLLLWFPGSRCDIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 466 aa, Homo sapiens; identical to SEQ ID NO: 4
  misc_feature (1)...(466): Ha22-2(2,4)6.1 heavy chain

SEQ ID NO: 8 - protein, 236 aa, Homo sapiens; identical to SEQ ID NO: 6
  misc_feature (1)...(236): Ha22-2(2,4)6.1 light chain

SEQ ID NO: 9 - protein, 5 aa, Homo sapiens
  CDR1 of heavy chain
SYNMN

SEQ ID NO: 10 - protein, 17 aa, Homo sapiens
  CDR2 of heavy chain
YISSSSSTIYYADSVKG

SEQ ID NO: 11 - protein, 8 aa, Homo sapiens
  CDR3 of heavy chain
AYYYGMDV

SEQ ID NO: 12 - protein, 11 aa, Homo sapiens
  CDR1 of light chain
RASQGISGWLA

SEQ ID NO: 13 - protein, 7 aa, Homo sapiens
  CDR2 of light chain
AASTLQS

SEQ ID NO: 14 - protein, 9 aa, Homo sapiens
  CDR3 of light chain
QQANSFPPT

SEQ ID NO: 15 - protein, 4 aa, Artificial Sequence
  linker
GFLG

SEQ ID NO: 16 - protein, 8 aa, Artificial Sequence
  VH CDR1 according to IMGT
GFTFSSYN

SEQ ID NO: 17 - protein, 8 aa, Artificial Sequence
  VH CDR2 according to IMGT
ISSSSSTI

SEQ ID NO: 18 - protein, 10 aa, Artificial Sequence
  VH CDR3 according to IMGT
ARAYYYGMDV

SEQ ID NO: 19 - protein, 6 aa, Artificial Sequence
  VL CDR1 according to IMGT
QGISGW

SEQ ID NO: 20 - protein, 3 aa, Artificial Sequence
  VL CDR2 according to IMGT
AAS

SEQ ID NO: 21 - protein, 9 aa, Artificial Sequence
  VL CDR3 according to IMGT
QQANSFPPT

SEQ ID NO: 22 - protein, 117 aa, Artificial Sequence
  heavy chain variable region (VH), the 20th amino acid (glutamic acid) to the 136th amino acid (serine) of SEQ ID NO7
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 23 - protein, 108 aa, Artificial Sequence
  light chain variable region (VL), the 23rd amino acid (aspartic acid) to the 130th amino acid (arginine) of SEQ ID NO8
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKR